SEQUENCE LISTING (83 sequences)

SEQ ID NO: 1 - DNA, 21290 bp, Severe acute respiratory syndrome coronavirus 2
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 2 - DNA, 50 bp, Artificial
  Synthetic nucleic acid sequence
agtaccggcagcacaagacatctgtcgtagtgcaacaggactaagctcat

SEQ ID NO: 3 - DNA, 50 bp, Artificial
  Synthetic nucleic acid sequence
atcgaagccaatccatgcacgtacatgtcttatagcttcttcgcgggtga

SEQ ID NO: 4 - DNA, 50 bp, Artificial
  Synthetic nucleic acid sequence
ctgatgaggttccacctggtttaacatatagtgaaccgccacacatgacc

SEQ ID NO: 5 - DNA, 50 bp, Artificial; identical to SEQ ID NO: 2
  Synthetic nucleic acid sequence

SEQ ID NO: 6 - DNA, 50 bp, Artificial
  Synthetic nucleic acid sequence
gactttagatcggcgccgtaactatggccaccagctcctttattaccgtt

SEQ ID NO: 7 - DNA, 50 bp, Artificial
  Synthetic nucleic acid sequence
ctgcagcaatcaatgggcaagctttgtcattagtataactaccaccacgc

SEQ ID NO: 8 - DNA, 50 bp, Artificial
  Synthetic nucleic acid sequence
aggatccacaagaacaacagcccttgagacaactacagcaactggtcata

SEQ ID NO: 9 - DNA, 50 bp, Artificial
  Synthetic nucleic acid sequence
agtactactactctgtatggttggtaaccaacaccattagtgggttggaa

SEQ ID NO: 10 - DNA, 50 bp, Artificial
  Synthetic nucleic acid sequence
cctagtgatgttaatacctattggcaaatctaccaatggttctaaagccg

SEQ ID NO: 11 - DNA, 50 bp, Artificial
  Synthetic nucleic acid sequence
ggattaacaactccggatgaaccgtcgattgtgtgaatttggacatgttc

SEQ ID NO: 12 - DNA, 50 bp, Artificial
  Synthetic nucleic acid sequence
cagtataaccaccaatctggtagtcatgttcagaaataggacttgttgtg

SEQ ID NO: 13 - DNA, 50 bp, Artificial
  Synthetic nucleic acid sequence
agtctgaagtgaagtaactgtgtaatacaacacagtcttttactccagat

SEQ ID NO: 14 - DNA, 50 bp, Artificial
  Synthetic nucleic acid sequence
caatattgcagcagtacgcacacaatcgaagcgcagtaaggatggctagt

SEQ ID NO: 15 - DNA, 50 bp, Artificial
  Synthetic nucleic acid sequence
agcgtcctagatggtgtccagcaatacgaagatgtccacgaaggatcaca

SEQ ID NO: 16 - DNA, 50 bp, Artificial
  Synthetic nucleic acid sequence
atttgtaataagaaagcgttcgtgatgtagcaacagtgatttctttaggc

SEQ ID NO: 17 - DNA, 50 bp, Artificial
  Synthetic nucleic acid sequence
atagcaattccaccggtgatccaatttattctgtaaacagcagcaagcac

SEQ ID NO: 18 - DNA, 50 bp, Artificial
  Synthetic nucleic acid sequence
atcagctagaggatgaaatggtgaattgccctcgtatgttccagaagagc

SEQ ID NO: 19 - DNA, 50 bp, Artificial
  Synthetic nucleic acid sequence
actgttgtacctctaacacactcttggtagtgataaagctcacaagtagc

SEQ ID NO: 20 - DNA, 50 bp, Artificial
  Synthetic nucleic acid sequence
cttttgagtgtgaagcaaagtgttataaacactattgccgcaacaataag

SEQ ID NO: 21 - DNA, 50 bp, Artificial; identical to SEQ ID NO: 20
  Synthetic nucleic acid sequence

SEQ ID NO: 22 - DNA, 50 bp, Artificial
  Synthetic nucleic acid sequence
cttcatagaacgaacaacgcactacaagactacccaatttaggttcctgg

SEQ ID NO: 23 - DNA, 50 bp, Artificial
  Synthetic nucleic acid sequence
accgatatcgatgtactgaatgggtgatttagaaccagcctcatccacgc

SEQ ID NO: 24 - DNA, 50 bp, Artificial
  Synthetic nucleic acid sequence
gatgttgagtacatgactgtaaactacattcttggtgaaatgcagctaca

SEQ ID NO: 25 - DNA, 50 bp, Artificial
  Synthetic nucleic acid sequence
gggaatttaaggtcttccttgccatgttgagtgagagcggtgaaccaaga

SEQ ID NO: 26 - DNA, 50 bp, Artificial
  Synthetic nucleic acid sequence
aatttccttgggtttgttctggaccacgtctgccgaaagcttgtgttaca

SEQ ID NO: 27 - DNA, 50 bp, Artificial
  Synthetic nucleic acid sequence
accaccacgaattcgtctggtagctcttcggtagtagccaatttggtcat

SEQ ID NO: 28 - DNA, 50 bp, Artificial
  Synthetic nucleic acid sequence
tgcacaagagtagactatatatcgtaaacggaaaagcgaaaacgtttata

SEQ ID NO: 29 - DNA, 46 bp, Artificial
  Synthetic nucleic acid sequence
ccttggcacccgagaattccaagtaccggcagcacaagacatctgt

SEQ ID NO: 30 - DNA, 55 bp, Artificial
  Synthetic nucleic acid sequence
cgtagtgcaacaggactaagctcataaaaaaaaaaaaaaaaaaaaaaaaaaaaaa

SEQ ID NO: 31 - DNA, 46 bp, Artificial
  Synthetic nucleic acid sequence
ccttggcacccgagaattccaatcgaagccaatccatgcacgtaca

SEQ ID NO: 32 - DNA, 55 bp, Artificial
  Synthetic nucleic acid sequence
tgtcttatagcttcttcgcgggtgaaaaaaaaaaaaaaaaaaaaaaaaaaaaaaa

SEQ ID NO: 33 - DNA, 46 bp, Artificial
  Synthetic nucleic acid sequence
ccttggcacccgagaattccactgatgaggttccacctggtttaac

SEQ ID NO: 34 - DNA, 55 bp, Artificial
  Synthetic nucleic acid sequence
atatagtgaaccgccacacatgaccaaaaaaaaaaaaaaaaaaaaaaaaaaaaaa

SEQ ID NO: 35 - DNA, 46 bp, Artificial; identical to SEQ ID NO: 29
  Synthetic nucleic acid sequence

SEQ ID NO: 36 - DNA, 55 bp, Artificial; identical to SEQ ID NO: 30
  Synthetic nucleic acid sequence

SEQ ID NO: 37 - DNA, 46 bp, Artificial
  Synthetic nucleic acid sequence
ccttggcacccgagaattccagactttagatcggcgccgtaactat

SEQ ID NO: 38 - DNA, 55 bp, Artificial
  Synthetic nucleic acid sequence
ggccaccagctcctttattaccgttaaaaaaaaaaaaaaaaaaaaaaaaaaaaaa

SEQ ID NO: 39 - DNA, 46 bp, Artificial
  Synthetic nucleic acid sequence
ccttggcacccgagaattccactgcagcaatcaatgggcaagcttt

SEQ ID NO: 40 - DNA, 55 bp, Artificial
  Synthetic nucleic acid sequence
gtcattagtataactaccaccacgcaaaaaaaaaaaaaaaaaaaaaaaaaaaaaa

SEQ ID NO: 41 - DNA, 46 bp, Artificial
  Synthetic nucleic acid sequence
ccttggcacccgagaattccaaggatccacaagaacaacagccctt

SEQ ID NO: 42 - DNA, 55 bp, Artificial
  Synthetic nucleic acid sequence
gagacaactacagcaactggtcataaaaaaaaaaaaaaaaaaaaaaaaaaaaaaa

SEQ ID NO: 43 - DNA, 46 bp, Artificial
  Synthetic nucleic acid sequence
ccttggcacccgagaattccaagtactactactctgtatggttggt

SEQ ID NO: 44 - DNA, 55 bp, Artificial
  Synthetic nucleic acid sequence
aaccaacaccattagtgggttggaaaaaaaaaaaaaaaaaaaaaaaaaaaaaaaa

SEQ ID NO: 45 - DNA, 46 bp, Artificial
  Synthetic nucleic acid sequence
ccttggcacccgagaattccacctagtgatgttaatacctattggc

SEQ ID NO: 46 - DNA, 55 bp, Artificial
  Synthetic nucleic acid sequence
aaatctaccaatggttctaaagccgaaaaaaaaaaaaaaaaaaaaaaaaaaaaaa

SEQ ID NO: 47 - DNA, 46 bp, Artificial
  Synthetic nucleic acid sequence
ccttggcacccgagaattccaggattaacaactccggatgaaccgt

SEQ ID NO: 48 - DNA, 55 bp, Artificial
  Synthetic nucleic acid sequence
cgattgtgtgaatttggacatgttcaaaaaaaaaaaaaaaaaaaaaaaaaaaaaa

SEQ ID NO: 49 - DNA, 46 bp, Artificial
  Synthetic nucleic acid sequence
ccttggcacccgagaattccacagtataaccaccaatctggtagtc

SEQ ID NO: 50 - DNA, 55 bp, Artificial
  Synthetic nucleic acid sequence
atgttcagaaataggacttgttgtgaaaaaaaaaaaaaaaaaaaaaaaaaaaaaa

SEQ ID NO: 51 - DNA, 46 bp, Artificial
  Synthetic nucleic acid sequence
ccttggcacccgagaattccaagtctgaagtgaagtaactgtgtaa

SEQ ID NO: 52 - DNA, 55 bp, Artificial
  Synthetic nucleic acid sequence
tacaacacagtcttttactccagataaaaaaaaaaaaaaaaaaaaaaaaaaaaaa

SEQ ID NO: 53 - DNA, 46 bp, Artificial
  Synthetic nucleic acid sequence
ccttggcacccgagaattccacaatattgcagcagtacgcacacaa

SEQ ID NO: 54 - DNA, 55 bp, Artificial
  Synthetic nucleic acid sequence
tcgaagcgcagtaaggatggctagtaaaaaaaaaaaaaaaaaaaaaaaaaaaaaa

SEQ ID NO: 55 - DNA, 46 bp, Artificial
  Synthetic nucleic acid sequence
ccttggcacccgagaattccaagcgtcctagatggtgtccagcaat

SEQ ID NO: 56 - DNA, 55 bp, Artificial
  Synthetic nucleic acid sequence
acgaagatgtccacgaaggatcacaaaaaaaaaaaaaaaaaaaaaaaaaaaaaaa

SEQ ID NO: 57 - DNA, 46 bp, Artificial
  Synthetic nucleic acid sequence
ccttggcacccgagaattccaatttgtaataagaaagcgttcgtga

SEQ ID NO: 58 - DNA, 55 bp, Artificial
  Synthetic nucleic acid sequence
tgtagcaacagtgatttctttaggcaaaaaaaaaaaaaaaaaaaaaaaaaaaaaa

SEQ ID NO: 59 - DNA, 46 bp, Artificial
  Synthetic nucleic acid sequence
ccttggcacccgagaattccaatagcaattccaccggtgatccaat

SEQ ID NO: 60 - DNA, 55 bp, Artificial
  Synthetic nucleic acid sequence
ttattctgtaaacagcagcaagcacaaaaaaaaaaaaaaaaaaaaaaaaaaaaaa

SEQ ID NO: 61 - DNA, 46 bp, Artificial
  Synthetic nucleic acid sequence
ccttggcacccgagaattccaatcagctagaggatgaaatggtgaa

SEQ ID NO: 62 - DNA, 55 bp, Artificial
  Synthetic nucleic acid sequence
ttgccctcgtatgttccagaagagcaaaaaaaaaaaaaaaaaaaaaaaaaaaaaa

SEQ ID NO: 63 - DNA, 46 bp, Artificial
  Synthetic nucleic acid sequence
ccttggcacccgagaattccaactgttgtacctctaacacactctt

SEQ ID NO: 64 - DNA, 55 bp, Artificial
  Synthetic nucleic acid sequence
ggtagtgataaagctcacaagtagcaaaaaaaaaaaaaaaaaaaaaaaaaaaaaa

SEQ ID NO: 65 - DNA, 46 bp, Artificial
  Synthetic nucleic acid sequence
ccttggcacccgagaattccacttttgagtgtgaagcaaagtgtta

SEQ ID NO: 66 - DNA, 55 bp, Artificial
  Synthetic nucleic acid sequence
taaacactattgccgcaacaataagaaaaaaaaaaaaaaaaaaaaaaaaaaaaaa

SEQ ID NO: 67 - DNA, 46 bp, Artificial
  Synthetic nucleic acid sequence
ccttggcacccgagaattccaaggcgtgacaagtttcattatgatc

SEQ ID NO: 68 - DNA, 55 bp, Artificial
  Synthetic nucleic acid sequence
ttgcagttcaagtgagaaccaaaagaaaaaaaaaaaaaaaaaaaaaaaaaaaaaa

SEQ ID NO: 69 - DNA, 46 bp, Artificial
  Synthetic nucleic acid sequence
ccttggcacccgagaattccacttcatagaacgaacaacgcactac

SEQ ID NO: 70 - DNA, 55 bp, Artificial
  Synthetic nucleic acid sequence
aagactacccaatttaggttcctggaaaaaaaaaaaaaaaaaaaaaaaaaaaaaa

SEQ ID NO: 71 - DNA, 46 bp, Artificial
  Synthetic nucleic acid sequence
ccttggcacccgagaattccaaccgatatcgatgtactgaatgggt

SEQ ID NO: 72 - DNA, 55 bp, Artificial
  Synthetic nucleic acid sequence
gatttagaaccagcctcatccacgcaaaaaaaaaaaaaaaaaaaaaaaaaaaaaa

SEQ ID NO: 73 - DNA, 46 bp, Artificial
  Synthetic nucleic acid sequence
ccttggcacccgagaattccagatgttgagtacatgactgtaaact

SEQ ID NO: 74 - DNA, 55 bp, Artificial
  Synthetic nucleic acid sequence
acattcttggtgaaatgcagctacaaaaaaaaaaaaaaaaaaaaaaaaaaaaaaa

SEQ ID NO: 75 - DNA, 46 bp, Artificial
  Synthetic nucleic acid sequence
ccttggcacccgagaattccagggaatttaaggtcttccttgccat

SEQ ID NO: 76 - DNA, 55 bp, Artificial
  Synthetic nucleic acid sequence
gttgagtgagagcggtgaaccaagaaaaaaaaaaaaaaaaaaaaaaaaaaaaaaa

SEQ ID NO: 77 - DNA, 46 bp, Artificial
  Synthetic nucleic acid sequence
ccttggcacccgagaattccaaatttccttgggtttgttctggacc

SEQ ID NO: 78 - DNA, 55 bp, Artificial
  Synthetic nucleic acid sequence
acgtctgccgaaagcttgtgttacaaaaaaaaaaaaaaaaaaaaaaaaaaaaaaa

SEQ ID NO: 79 - DNA, 46 bp, Artificial
  Synthetic nucleic acid sequence
ccttggcacccgagaattccaaccaccacgaattcgtctggtagct

SEQ ID NO: 80 - DNA, 55 bp, Artificial
  Synthetic nucleic acid sequence
cttcggtagtagccaatttggtcataaaaaaaaaaaaaaaaaaaaaaaaaaaaaa

SEQ ID NO: 81 - DNA, 46 bp, Artificial
  Synthetic nucleic acid sequence
ccttggcacccgagaattccatgcacaagagtagactatatatcgt

SEQ ID NO: 82 - DNA, 55 bp, Artificial
  Synthetic nucleic acid sequence
aaacggaaaagcgaaaacgtttataaaaaaaaaaaaaaaaaaaaaaaaaaaaaaa

SEQ ID NO: 83 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

(19) United States
(12) Patent Application Publication
(10) Pub. No.: US 2024/0368711 A1
(43) Pub. Date: Nov. 7, 2024

(54) SPATIAL DETECTION OF SARS-COV-2 USING TEMPLATED LIGATION

(71) Applicant: 10x Genomics, Inc., Pleasanton, CA (US)

(72) Inventors: Stefania Giacomello, Stockholm (SE); Hailey Elizabeth Sounart, Stockholm (SE); Andrew John Hill, Pleasanton, CA (US); Erik Leonard Henrik Borgstrom, Årsta (SE); Aleksandra Jurek, Stockholm (SE); James Michael Chell, Stockholm (SE); Yuvarani Masarapu, Uppsala (SE)

(21) Appl. No.: 18/573,162

(22) PCT Filed: Jun. 22, 2022
(86) PCT No.: PCT/US2022/034520

§ 371 Date: Dec. 21, 2023

Related U.S. Application Data
(60) Provisional application No. 63/291,040, filed on Dec. 17, 2021.
(60) Provisional application No. 63/276,202, filed on Nov. 5, 2021.
(60) Provisional application No. 63/246,581, filed on Sep. 21, 2021.
(60) Provisional application No. 63/213,582, filed on Jun. 22, 2021.

(51) Int. Cl. C12Q 1/70; C12Q 1/6818; C12Q 1/6841

(52) U.S. Cl. CPC C12Q 1/701; C12Q 1/6841; C12Q 1/6818

(57) ABSTRACT

Provided herein are methods, compositions and kits for detecting SARS-CoV-2 and host nucleic acids in a tissue sample.

### CROSS-REFERENCE TO RELATED APPLICATIONS

This application claims the benefit of U.S. Provisional Application Ser. No. 63/213,582, filed Jun. 22, 2021, 63/246,581 filed Sep. 21, 2021, 63/276,202, filed Nov. 5, 2021, and 63/291,040, filed Dec. 17, 2021, the entire contents of which are incorporated by reference herein.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Generally, targeting a particular analyte in a biological sample utilizes a capture probe that targets a common transcript sequence such as a poly(A) mRNA-like tail. However, this approach is capable of detecting a high number of off target analytes. Methods such as templated ligation offer an alternative to indiscriminate capture of a common transcript sequence. See, e.g., Yeakley, PLoS One, 25; 12(5):e0178302 (2017), which is incorporated by reference in its entirety. However, there remains a need to develop an alternative to common transcript sequence (e.g., poly(A) mRNA-like tail) capture of target analytes that is capable of detecting an analyte(s) in an entire transcriptome while providing information regarding the spatial location and abundance of a target analyte.

Severe acute respiratory syndrome coronavirus 2 (SARS-CoV-2) has been declared a high-risk global health emergency by the World Health Organization (WHO) and has, as of Jun. 10, 2022, caused 531,550,610 cases of respiratory disease and 6,302,982 deaths worldwide. New strategies for the detection and spatial analysis of expression of viral threats such as SARS-CoV-2, and other potential life threatening emerging viral threats, are necessary in order to develop prophylaxis and/or therapeutic treatment regimens to effectively mitigate current and future viral outbreaks.

Genetic material, and related gene and protein expression, influences cellular fate and behavior. The spatial heterogeneity in developing systems has typically been studied via RNA hybridization, immunohistochemistry, fluorescent reporters, or purification or induction of pre-defined subpopulations and subsequent genomic profiling (e.g., RNA-seq). Such approaches, however, rely on a small set of pre-defined markers, therefore introducing selection bias that limits discovery and making it costly and laborious to localize RNA transcriptome-wide. There is a need to determine non-biased and non-limited spatial expression of biomarkers associated with a pathology (e.g., SARS-CoV-2 infection).

### SUMMARY

Targeted RNA capture is an attractive alternative to poly(A) mRNA capture in order to interrogate spatial gene expression in a sample (e.g., an FFPE tissue). Compared to poly(A) mRNA capture, targeted RNA capture as described herein is less affected by RNA degradation associated with FFPE fixation compared to methods dependent on oligo-dT capture and reverse transcription of mRNA. Further targeted RNA capture as described herein allows for sensitive measurement of specific genes of interest that otherwise might be missed with a whole transcriptomic approach. Targeted RNA capture can be used to capture a defined set of RNA molecules of interest, or it can be used at a whole transcriptome level, or anything in between. When combined with the spatial methods disclosed herein, the location and abundance of the RNA targets can be determined.

In one aspect of the disclosure, provided herein are methods for determining the abundance and/or location of a coronavirus nucleic acid in a biological sample. In some instances, the methods comprise (a) contacting the biological sample with a substrate comprising a plurality of capture probes, wherein a capture probe of the plurality of capture probes comprises a spatial barcode and a capture domain; (b) contacting the biological sample with a plurality of first coronavirus probes and second coronavirus probes, wherein a first coronavirus probe and a second coronavirus probe comprise a sequence that is substantially complementary to sequences of the coronavirus nucleic acid, and wherein the second probe comprises a capture probe capture domain that is complementary to all or a portion of the capture domain of a capture probe; (c) hybridizing the first coronavirus probe and the second coronavirus probe to the coronavirus nucleic acid; (d) ligating the first coronavirus probe and the second coronavirus probe, thereby generating a coronavirus ligation product; (e) releasing the ligation product from the coronavirus nucleic acid; and (f) determining (i) all or part of the sequence of the ligation product, or a complement thereof, and (ii) the spatial barcode, or a complement thereof, and using the determined sequence of (i) and (ii) to determine the abundance and location of the coronavirus nucleic acid in the biological sample.

In some instances, the plurality of first coronavirus probes and second coronavirus probes comprises two or more coronavirus probe pairs. In some instances, the plurality of first coronavirus probes and second coronavirus probes comprises two or more of SEQ ID NOs: 29-82. In some instances, the first coronavirus probe and the second coronavirus probe are substantially complementary to adjacent sequences of the coronavirus nucleic acid.

In some instances, the first coronavirus probe and the second coronavirus probe hybridize to sequences that are not adjacent to each other on the coronavirus nucleic acid. In some instances, the first coronavirus probe is extended with a DNA polymerase, thereby (i) filling in a gap between the first coronavirus probe and the second coronavirus probe.

In some instances, the ligating the first coronavirus probe and the second coronavirus probe utilizes enzymatic ligation or chemical ligation, wherein the enzymatic ligation utilizes a ligase, wherein the ligase is one or more of a T4 RNA ligase (Rnl2), a SplintR® ligase, a PBCV-1 DNA ligase, a Chlorella virus DNA ligase, a single stranded DNA ligase, or a T4 DNA ligase. In some instances, the first coronavirus probe comprises a primer sequence. In some instances, the first coronavirus probe and/or the second coronavirus probe is a DNA probe. In some instances, the releasing the ligation product from the coronavirus nucleic acid comprises contacting the biological sample with an RNase H enzyme.

In some instances, the determining step (f) comprises amplifying all or part of the ligation product. In some instances, the amplified ligation product comprises (i) all or part of the sequence of the ligation product, or a complement thereof, and (ii) the spatial barcode, or a complement thereof. In some instances, the determining step (f) comprises sequencing (i) all or a part of the sequence of the ligation product, or a complement thereof, and (ii) the spatial barcode, or a complement thereof.

In some instances, the coronavirus nucleic acid is an RNA molecule. In some instances, the coronavirus nucleic acid is from SARS-CoV-2.

In some instances, also disclosed herein are methods to determine the abundance and/or location of a host nucleic acid in the biological sample, the method comprising: (a) contacting a first host probe and a second host probe with the biological sample, wherein the first host probe and the second host probe each comprise one or more sequences that are substantially complementary to sequences of the host nucleic acid, and wherein the second host probe comprises a capture domain that is complementary to all or a portion of the capture domain of a capture probe; (b) hybridizing the first host probe and the second host probe to the host nucleic acid; (c) ligating the first host probe and the second host probe, thereby generating a host ligation product; (d) releasing the host ligation product from the host nucleic acid; (e) hybridizing the host ligation product to the capture domain; and (f) determining (i) all or part of the sequence of the host ligation product, or a complement thereof, and (ii) the spatial barcode, or a complement thereof, and using the determined sequences of (i) and (ii) to identify determine the location of the host nucleic acid in the biological sample.

In some instances, the steps of contacting the biological sample with the plurality of first coronavirus probes and second coronavirus probes and contacting the first host probe and the second host probe with the biological sample are performed substantially concurrently. In some instances, the steps of hybridizing the first coronavirus probe and the second coronavirus probe to the coronavirus nucleic acid and hybridizing the first host probe and the second host probe to the host nucleic acid are performed substantially concurrently. In some instances, the steps of ligating the first coronavirus probe and the second coronavirus probe and ligating the first host probe and the second host probe are performed substantially concurrently. In some instances, the steps of releasing the ligation product from the coronavirus nucleic acid and releasing the host ligation product from the host nucleic acid are performed substantially concurrently. In some instances, the steps of hybridizing the ligation product to the capture domain and hybridizing the host ligation product to the capture domain are performed substantially concurrently. In some instances, the biological sample is contacted with 5000 or more probe pairs.

In some instances, the first host probe and the second host probe are substantially complementary to adjacent sequences of the host nucleic acid. In some instances, the first host probe and the second host probe hybridize to sequences that are not adjacent to each other on the host nucleic acid. In some instances, the first probe is extended with a DNA polymerase, thereby (i) filling a gap between the first host probe and the second host probe. In some instances, the ligating the first host probe and the second host probe utilizes enzymatic ligation or chemical ligation, wherein the enzymatic ligation utilizes a ligase, wherein the ligase is one or more of a T4 RNA ligase (Rnl2), a SplintR® ligase, a PBCV-1 DNA ligase, a Chlorella virus DNA ligase, a single stranded DNA ligase, or a T4 DNA ligase. In some instances, the first host probe comprises a primer sequence. In some instances, the first host probe and/or the second host probe is a DNA probe.

In some instances, the releasing the host ligation product from the host nucleic acid comprises contacting the biological sample with an RNase H enzyme.

In some instances, the determining step (f) comprises amplifying all or part of the host ligation product.

In some instances, the amplified host ligation product comprises (i) all or part of sequence of the host ligation product, or a complement thereof, and (ii) the spatial barcode, or a complement thereof.

In some instances, the determining step (f) comprises sequencing (i) all or a part of the sequence of the host ligation product, or a complement thereof, and (ii) the spatial barcode, or a complement thereof.

In some instance, the host nucleic acid is one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LASIL, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof.

In some instances, also provided herein are methods of assessing expression levels of one or more nucleic acids in a subject suspected of having a coronavirus infection, comprising: (a) obtaining a biological sample from the subject; and (b) determining an expression level of a host nucleic acid from the one or more host nucleic acids, wherein the host nucleic acid is selected from the group consisting of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LASIL, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof.

In some instances, the obtaining step (a) comprises serially obtaining a biological sample from the subject at a plurality of time points and the determining step (b) comprises determining the expression levels in the serially obtained biological samples from the subject.

In some instance, also provided here in are methods of diagnosing a subject as having a heart disease or disorder or having an increased likelihood of developing a viral infection, wherein the method comprises: (a) determining an expression level of one or more host analytes selected from the group consisting of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, or a byproduct, a degradation product, or a precursor thereof, and any combination thereof, in a biological sample from the subject; and (b) identifying the subject having an elevated expression level of the one or more host analyte, and byproducts, precursors, and degradation products thereof of step (a), as compared to the reference expression level(s) of the one or more host analyte and byproducts, precursors, and degradation products thereof as having the viral infection or having an increased likelihood of developing the viral infection.

In some instances, also provided herein are methods of diagnosing a subject as having a heart disease or disorder or having an increased likelihood of developing a viral infection, wherein the method comprises: (a) determining an expression level of one or more host analytes selected from the group consisting of NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, in a biological sample from the subject; and (b) identifying the subject having a decreased expression level of the one or more host analyte, and byproducts, precursors, and degradation products thereof of step (a), as compared to the reference expression level(s) of the one or more host analyte and byproducts, precursors, and degradation products thereof as having the viral infection or having an increased likelihood of developing the viral infection.

In some instances, also provided herein are methods of monitoring risk of having a viral infection (e.g. a coronavirus infection) in a subject over time, wherein the method comprises: (a) determining a first expression level of one or more host analytes selected from the group consisting of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, or a byproduct, a degradation product, or a precursor thereof, and any combination thereof in a first biological sample obtained from a subject at a first time point; (b) determining a second expression level of the one or more host analytes and byproducts, precursors, and degradation products thereof of step (a), in a second biological sample obtained from the subject at a second time point; (c) identifying the subject as having an increased second expression level of the one or more host analytes, byproducts, precursors, and degradation products thereof of step (a), or as having an increasing risk of having the viral infection, or (d) identifying the subject as having about the same or a decreased second expression level of the one or more host analytes and byproducts, precursors, and degradation products thereof of step (a), as compared to the first expression level of the one or more host analytes, and byproducts, precursors, and degradation products thereof as having about the same or a decreasing risk of having the viral infection.

In some instances, also provided herein are methods of monitoring risk of having a viral infection (e.g. a coronavirus infection) in a subject over time, wherein the method comprises: (a) determining a first expression level of one or more host analytes selected from the group consisting of NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LASIL, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof in a first biological sample obtained from a subject at a first time point; (b) determining a second expression level of the one or more host analytes and byproducts, precursors, and degradation products thereof of step (a), in a second biological sample obtained from the subject at a second time point; (c) identifying the subject as having a decreased second expression level of the one or more host analytes, byproducts, precursors, and degradation products thereof of step (a), or as having an increasing risk of having the viral infection, or (d) identifying the subject as having increased second expression level of the one or more host analytes and byproducts, precursors, and degradation products thereof of step (a), as compared to the first expression level of the one or more host analytes, and byproducts, precursors, and degradation products thereof as having about the same or a decreasing risk of having the viral infection.

In some instances, also provided herein are methods of determining efficacy of a treatment for reducing the risk of having a viral infection (e.g. a coronavirus infection) in a subject, wherein the method comprises: (a) determining a first expression level of one or more host analytes selected from the group consisting of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, or a byproduct, a degradation product, or a precursor thereof, and any combination thereof, in a first biological sample obtained from a subject at a first time point; (b) determining a second expression level of the one or more of the host analytes of step (a) in a second biological sample obtained from the subject at a second time point, wherein the subject is administered one or more doses of a treatment for reducing the risk of having the viral infection between the first and second time points; and (c) identifying: (1) the treatment as being effective in the subject having about the same or a decreased second expression level of one or more host analytes, and byproducts, precursors, and degradation products thereof of step (a), or (2) the treatment as not being effective in the subject having increased second expression level of the one or more host analytes, and byproducts, precursors, and degradation products thereof of step (a).

In some instances, also provided herein are methods of determining efficacy of a treatment for reducing the risk of having a viral infection (e.g. a coronavirus infection) in a subject, wherein the method comprises: (a) determining a first expression level of one or more host analytes selected from the group consisting of NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, in a first biological sample obtained from a subject at a first time point; (b) determining a second expression level of the one or more of the host analytes of step (a) in a second biological sample obtained from the subject at a second time point, wherein the subject is administered one or more doses of a treatment for reducing the risk of having the viral infection between the first and second time points; and (c) identifying: (1) the treatment as being effective in the subject having increased second expression level of one or more host analytes, and byproducts, precursors, and degradation products thereof of step (a), or (2) the treatment as not being effective in the subject having the same or decreased second expression level of the one or more host analytes, and byproducts, precursors, and degradation products thereof of step (a).

In some instances, the viral infection is a SARS-CoV-2 viral infection, or a variant thereof.

In some instances, any of the methods described herein are further comprising administering a treatment for reducing the risk of having the viral infection to the subject, adjusting a dosage of a treatment for reducing the risk of having the viral infection for the subject, or adjusting a treatment for reducing the risk of having the viral infection for the subject.

In some instance, the levels of at least two, at least three, or at least four of the host analytes are determined.

In some instance, in any of the methods described herein, the determining steps comprise contacting the biological sample with a substrate comprising a plurality of capture probes and hybridizing the ligation product to the capture domain, wherein a capture probe of the plurality comprises a spatial barcode and a capture domain; contacting the biological sample with a plurality of first coronavirus probes and second coronavirus probes, wherein a first coronavirus probe and a second coronavirus probe of the plurality of first coronavirus probes and second coronavirus probes each comprise a sequence that is substantially complementary to sequences of the coronavirus nucleic acid, and wherein the second coronavirus probe comprises a capture probe capture domain that is complementary to all or a portion of the capture domain; hybridizing the first coronavirus probe and the second coronavirus probe to the coronavirus nucleic acid; ligating the first coronavirus probe and the second coronavirus probe, thereby generating a coronavirus ligation product; releasing the coronavirus ligation product from the coronavirus nucleic acid; and determining (i) all or part of the sequence of the coronavirus ligation product specifically bound to the capture domain, or a complement thereof, and (ii) the spatial barcode, or a complement thereof, and using the determined sequence of (i) and (ii) to determine the abundance and location of the coronavirus nucleic acid in the biological sample.

In some instances, the biological sample is a tissue sample. In some instances, the biological sample is a lung tissue sample. In some instances, the biological sample is a mammalian tissue sample. In some instances, the biological sample is a human tissue sample. In some instances, the tissue sample is a formalin-fixed, paraffin-embedded (FFPE) tissue sample, a fresh tissue sample, or a frozen tissue sample. In some instances, the tissue sample is the FFPE tissue sample. In some instances, the biological sample was previously stained using immunofluorescence, immunohistochemistry, or hematoxylin and eosin. In some instances, the biological sample was previously stained for a coronavirus protein. In some instances, the biological sample was previously stained for a coronavirus nucleic acid.

In some instances, the methods further include contacting the biological sample with a permeabilization agent, wherein the permeabilization agent is selected from an organic solvent, a detergent, an enzyme, or a combination thereof. In some instances, the permeabilization agent comprises proteinase K or pepsin.

In some instances, the host nucleic acid is RNA. In some instances, the RNA is an mRNA.

Also provided herein are kits. In some instances, the kits include (a) a substrate comprising a plurality of capture probes, wherein a capture probe of the plurality of capture probes comprises a spatial barcode and a capture domain; (b) a plurality of first RNA template ligation (RTL) probes and second RTL probes, wherein a first RTL probe and a second RTL probe of the plurality of first RTL probes and second RTL probes each comprise a sequence that is substantially complementary to sequences of a host analyte; and wherein one of the first RTL probe or the second RTL probe comprises a capture probe capture domain that is complementary to the capture domain; (c) a plurality of first coronavirus RTL probes and second coronavirus RTL probes, wherein a first coronavirus RTL probe and a second coronavirus RTL probe of the plurality of first coronavirus RTL probes and second coronavirus RTL probes each comprise a sequence that is substantially complementary to sequences of a coronavirus nucleic acid; and wherein one of the first coronavirus RTL probe or the second coronavirus RTL probe comprises a capture coronavirus RTL probe capture domain that is complementary to the capture domain; and (d) instructions for performing any of the methods provided herein. Throughout this document, RTL probe can also refer to viral probes (e.g. coronavirus probes) or host probes. Furthermore, phrases referring to probes or RTL probes are interchangeable if referring to viral probes, such as a coronavirus probe, or host probes.

In some instances the kits include (a) an antibody that binds specifically to one or more analytes selected from the group consisting of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LASIL, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof; and (b) instructions for performing any of the methods described herein.

Also provided herein are compositions. In some instances, the compositions comprise (a) a spatial array comprising a plurality of capture probes, wherein a capture probe of the plurality of capture probes comprises a spatial barcode and a capture domain; (b) a biological sample on the spatial array wherein the biological sample comprises a plurality of nucleic acids; (c) a plurality of first probes and second probes, wherein a first probe and a second probe of the plurality of first probes and second probes each comprise a sequence that is substantially complementary to sequences of a host nucleic acid; and wherein one of the first probe or the second probe comprises a capture probe capture domain that is complementary to the capture domain; and (d) a plurality of first coronavirus probes and second coronavirus probes, wherein a first coronavirus probe and a second coronavirus probe of the plurality of first coronavirus probes and second coronavirus probes each comprise a sequence that is substantially complementary to sequences of a coronavirus nucleic acid; and wherein one of the first coronavirus probe or the second coronavirus probe comprises a capture coronavirus probe capture domain that is complementary to the capture domain.

Also provided herein are methods of identifying the presence or absence of a coronavirus infection in a subject. In some instances, the methods comprise (a) isolating a biological sample from the subject; (b) contacting the biological sample with a substrate comprising a plurality of capture probes, wherein a capture probe comprises a spatial barcode and a capture domain; (c) contacting the biological sample with a plurality of first coronavirus probes and second coronavirus probes, wherein a first coronavirus probe and a second coronavirus probe comprise a sequence that is substantially complementary to sequences of the coronavirus nucleic acid, and wherein the second coronavirus probe comprises a capture domain that is complementary to all or a portion of the capture domain of a capture probe; (d) hybridizing the first coronavirus probe and the second coronavirus probe to the coronavirus nucleic acid; (e) ligating the first coronavirus probe to the second coronavirus probe, thereby generating a coronavirus ligation product; (f) releasing the coronavirus ligation product from the coronavirus nucleic acid; and (g) determining (i) all or part of the sequence of the coronavirus ligation product, or a complement thereof, and (ii) the spatial barcode, or a complement thereof, and using the determined sequence of (i) and (ii) to identify the presence or absence of a coronavirus infection in a subject.

In some instances, the coronavirus nucleic acid is one or more of ORF1ab, S, N, ORF8, E, ORF3a, ORF7a, ORF6, M, ORF10, ORF7b.

Also provided herein are methods of treating a coronavirus infection in a subject. In some instances, the methods include (a) isolating a biological sample from the subject; (b) contacting the biological sample with a substrate comprising a plurality of capture probes and hybridizing the coronavirus ligation product to the capture domain, wherein a capture probe of the plurality comprises a spatial barcode and a capture domain; (c) contacting the biological sample with a plurality of first coronavirus RTL probes and second coronavirus RTL probes, wherein a first coronavirus RTL probe and a second coronavirus RTL probe of the plurality of first coronavirus RTL probes and second coronavirus RTL probes each comprise a sequence that is substantially complementary to sequences of the coronavirus nucleic acid, and wherein the second RTL probe comprises a capture probe capture domain that is complementary to all or a portion of the capture domain; (d) hybridizing the first coronavirus RTL probe and the second coronavirus RTL probe to the coronavirus nucleic acid; (e) ligating the first coronavirus RTL probe and the second coronavirus RTL probe, thereby generating a coronavirus ligation product; (f) releasing the coronavirus ligation product from the coronavirus nucleic acid; (g) determining (i) all or part of the sequence of the coronavirus ligation product specifically bound to the capture domain, or a complement thereof, and (ii) the spatial barcode, or a complement thereof, and using the determined sequence of (i) and (ii) to identify the coronavirus infection in a subject; and (h) administering to the subject one or more of: dexamethasone, remdesivir, baricitinib in combination with remdesivir, favipiravir, merimepodib, an anticoagulation drug selected from low-dose heparin or enoxaparin, bamlanivimab, a combination of bamlanivimab and etesevimab, a combination of casirivimab and imdevimab, convalescent plasma, a furin inhibitor, an mRNA SARS-CoV-2 vaccine, an attenuated SARS-CoV-2 virus vaccine, a dead SARS-CoV-2 virus vaccine, a viral vaccine against SARS-CoV-2 such as an adenoviral vaccine, an antibody or fragment thereof or a small molecule that blocks interaction between human ACE2 and the spike protein of SARS-CoV-2, a protease inhibitor targeting SARS-CoV-2 S cleavage sites, EK1C4, nelfinavir mesylate, or unmethylated CpG dinucleotides in combination with any of these agents.

Also provided herein are methods of monitoring efficacy of treatment of a coronavirus infection in a subject over time. In some instances, the methods comprise (a) determining a first level of one or more of ORF1a polyprotein (ORF1ab), surface glycoprotein (S), nucleocapsid phosphoprotein (N), ORF8 protein (ORF8), envelope protein (E), ORF3a protein (ORF3a), ORF7a protein (ORF7a), ORF6 protein (ORF6), membrane glycoprotein (M), ORF10 protein (ORF10), and ORF7b in a first biological sample obtained from a subject at a first time point; (b) determining a second level of one or more of ORF1ab, S, N, ORF8, E, ORF3a, ORF7a, ORF6, M, ORF10, or ORF7b in a second biological sample obtained from the subject at a second time point; (c) identifying: (i) a subject having an increased second level, as compared to the first level, as not having efficacy of treatment of the coronavirus infection, or (ii) a subject having about the same or a decreased second level as compared to the first level as having efficacy of treatment of the coronavirus infection.

In some instances, the determining the first level comprises (a) isolating the first biological sample from the subject; (b) contacting the first biological sample with a substrate comprising a plurality of capture probes and hybridizing the coronavirus ligation product to the capture domain, wherein a capture probe of the plurality comprises a spatial barcode and a capture domain; (c) contacting the first biological sample with a plurality of first coronavirus RTL probes and second coronavirus RTL probes, wherein a first coronavirus RTL probe and a second coronavirus RTL probe of the plurality of first coronavirus RTL probes and second coronavirus RTL probes each comprise a sequence that is substantially complementary to sequences of the coronavirus nucleic acid, and wherein the second RTL probe comprises a capture probe capture domain that is complementary to all or a portion of the capture domain; (d) hybridizing the first coronavirus RTL probe and the second coronavirus RTL probe to the coronavirus nucleic acid; (e) ligating the first coronavirus RTL probe and the second coronavirus RTL probe, thereby generating a coronavirus ligation product; (f) releasing the coronavirus ligation product from the coronavirus nucleic acid; and (g) determining (i) all or part of the sequence of the coronavirus ligation product specifically bound to the capture domain, or a complement thereof, and (ii) the spatial barcode, or a complement thereof, and using the determined sequence of (i) and (ii) to determine the first level in the subject.

In some instances, the determining the second level comprises (a) isolating the second biological sample from the subject; (b) contacting the second biological sample with a substrate comprising a plurality of capture probes and hybridizing the coronavirus ligation product to the capture domain, wherein a capture probe of the plurality comprises a spatial barcode and a capture domain; (c) contacting the second biological sample with the plurality of first coronavirus RTL probes and second coronavirus RTL probes; (d) hybridizing the first coronavirus RTL probe and the second coronavirus RTL probe to the coronavirus nucleic acid; (e) ligating the first coronavirus RTL probe and the second coronavirus RTL probe, thereby generating the coronavirus ligation product; (f) releasing the coronavirus ligation product from the coronavirus nucleic acid; and (g) determining (i) all or part of the sequence of the coronavirus ligation product specifically bound to the capture domain, or a complement thereof, and (ii) the spatial barcode, or a complement thereof, and using the determined sequence of (i) and (ii) to determine the second level in the subject.

In some instances, the plurality of first coronavirus RTL probes and second coronavirus RTL probes comprises two or more coronavirus RTL probe pairs. In some instances, the plurality of first coronavirus RTL probes and second coronavirus RTL probes comprises two or more of SEQ ID NOs: 29-82.

In some instances, the subject is human. In some instances, the biological sample, the first biological sample, and/or the second biological sample is a nasal sample, a nasopharyngeal sample, an oral sample, or a lung tissue sample. In some instances, the biological sample is a lung tissue sample. In some instances, the coronavirus nucleic acid is from SARS-CoV-2.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof “A and/or B” is used herein to include all of the following alternatives: “A”, “B”, “A or B”, and “A and B”.

As used herein, the term “about” can mean up to +10% of a measurement or value. For example, about 25° C. mean between, and including, 22.5° C.-27.5° C.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary spatial analysis workflow.

FIG. 2 is a schematic diagram showing an example of a barcoded capture probe, as described herein.

FIG. 3 is a schematic illustrating a cleavable capture probe, wherein the cleaved capture probe can enter into a non-permeabilized cell and bind to target analytes within the sample.

FIG. 4 is a schematic diagram of an exemplary multiplexed spatially-barcoded feature.

FIG. 5 is a schematic showing the exemplary arrangement of barcoded features within an array.

FIG. 6 is a schematic diagram showing an exemplary workflow for templated ligation.

FIG. 7 is a schematic diagram showing an exemplary workflow for capturing a ligation product on a substrate that includes capture probes.

FIG. 8 is a schematic diagram showing an example of a first RTL probe that includes a linker sequence and a second RTL probe.

FIG. 9 is a schematic diagram showing an example of a second RTL probe that includes a linker sequence and a first RTL probe.

FIG. 10 is a schematic diagram showing an example of a first RTL probe, a second RTL probe, and a spanning probe.

FIG. 11 is a schematic diagram showing an example of a templated ligation using a first RTL probe, a second RTL probe, and a third probe.

FIGS. 12A-12E show various approaches for chemically-mediated nucleic acid ligation. FIG. 12A illustrates formation of a triazole bond. FIG. 12B illustrates formation of a phosphorothioate bond. FIG. 12C illustrates formation of an amide bond. FIG. 12D illustrates a formation of a phosphoramidite molecule. FIG. 12E illustrates a conjugation reaction.

FIG. 13 shows an exemplary templated ligation workflow.

FIG. 14 shows representative images of UMI counts using only host (i.e., human-specific) probes (top image) and both host and viral (i.e., SARS-CoV-2-specific) probes (bottom image). White arrows in the bottom image show representative dark spots indicating changes in UMI counts.

FIG. 15 shows representative images of gene clustering using only host (i.e., human-specific) probes (top image) and both host and viral (i.e., SARS-CoV-2-specific) probes (bottom image).

FIG. 16 shows t-SNE projection of spots by clustering from FIG. 15 when samples were treated using only host (i.e., human-specific) probes (top image) and both host and viral (i.e., SARS-CoV-2-specific) probes (bottom image). Clusters indicated with numbered circles.

FIG. 17 shows specific ligation reads (left images) and specific ligation UMIs (right image) in two SARS-CoV-2-positive biological samples in which both host and viral (i.e., SARS-CoV-2-specific) probes were added to each sample.

FIG. 18 shows specific ligation reads (left images) and specific ligation UMIs (right image) in two SARS-CoV-2-negative biological samples in which both host and viral (i.e., SARS-CoV-2-specific) probes were added to each sample.

FIG. 19 shows B2M (left panel), SARS-CoV-2 (center panel), and B2M/SARS-CoV-2 combined (right panel) expression and co-localization in a lung sample.

FIG. 20 shows CTSB (left panel), SARS-CoV-2 (center panel), and CTSB/SARS-CoV-2 combined (right panel) expression and co-localization in a lung sample.

FIG. 21 shows IGHG3 (left panel), SARS-CoV-2 (center panel), and IGHG3/SARS-CoV-2 combined (right panel) expression and co-localization in a lung sample.

FIG. 22 shows JCHAIN (left panel), SARS-CoV-2 (center panel), and JCHAIN/SARS-CoV-2 combined (right panel) expression and co-localization in a lung sample.

FIG. 23 shows IGHM (left panel), SARS-CoV-2 (center panel), and IGHM/SARS-CoV-2 combined (right panel) expression and co-localization in a lung sample.

FIG. 24 shows IGKC (left panel), SARS-CoV-2 (center panel), and IGKC/SARS-CoV-2 combined (right panel) expression and co-localization in a lung sample.

FIG. 25 shows AGER (left panel), SARS-CoV-2 (center panel), and AGER/SARS-CoV-2 combined (right panel) expression and co-localization in a lung sample.

FIG. 26 shows BTNL9 (left panel), SARS-CoV-2 (center panel), and BTNL9/SARS-CoV-2 combined (right panel) expression and co-localization in a lung sample.

FIG. 27 shows DUSP1 (left panel), SARS-CoV-2 (center panel), and DUSP1/SARS-CoV-2 combined (right panel) expression and co-localization in a lung sample.

FIG. 28 shows NR4A1 (left panel), SARS-CoV-2 (center panel), and NR4A1/SARS-CoV-2 combined (right panel) expression and co-localization in a lung sample.

FIG. 29 shows RHOB (left panel), SARS-CoV-2 (center panel), and RHOB/SARS-CoV-2 combined (right panel) expression and co-localization in a lung sample.

FIG. 30 shows ZFP36 (left panel), SARS-CoV-2 (center panel), and ZFP36/SARS-CoV-2 combined (right panel) expression and co-localization in a lung sample.

FIG. 31 shows ANKRD1 expression (left panel), SARS-CoV-2 expression (center panel—white arrows indicate SARS-CoV-2 positive spots), and ANKRD1/SARS-CoV-2 combined expression (right panel) in a heart sample of a human subject positive for a SARS-CoV-2 infection.

FIG. 32 shows CKM expression (left panel), SARS-CoV-2 expression (center panel—white arrows indicate SARS-CoV-2 positive spots), and CKM/SARS-CoV-2 combined expression (right panel) in a heart sample of a human subject positive for a SARS-CoV-2 infection.

FIG. 33 shows TTN expression (left panel), SARS-CoV-2 expression (center panel—white arrows indicate SARS-CoV-2 positive spots), and TTN/SARS-CoV-2 combined expression (right panel) in a heart sample of a human subject positive for a SARS-CoV-2 infection.

FIG. 34 shows CCDC69 expression (left panel), SARS-CoV-2 expression (center panel—white arrows indicate SARS-CoV-2 positive spots), and CCDC69/SARS-CoV-2 combined expression (right panel) in a heart sample of a human subject positive for a SARS-CoV-2 infection.

FIG. 35 shows CSDE1 expression (left panel), SARS-CoV-2 expression (center panel—white arrows indicate SARS-CoV-2 positive spots), and CSDE1/SARS-CoV-2 combined expression (right panel) in a heart sample of a human subject positive for a SARS-CoV-2 infection.

FIG. 36 shows NDUFS1 expression (left panel), SARS-CoV-2 expression (center panel—white arrows indicate SARS-CoV-2 positive spots), and NDUFS1/SARS-CoV-2 combined expression (right panel) in a heart sample of a human subject positive for a SARS-CoV-2 infection.

FIG. 37 shows HIPK1 expression (left panel), SARS-CoV-2 expression (center panel—white arrows indicate SARS-CoV-2 positive spots), and HIPK1/SARS-CoV-2 combined expression (right panel) in a heart sample of a human subject positive for a SARS-CoV-2 infection.

FIG. 38 shows MLXIP expression (left panel), SARS-CoV-2 expression (center panel—white arrows indicate SARS-CoV-2 positive spots), and MLXIP/SARS-CoV-2 combined expression (right panel) in a heart sample of a human subject positive for a SARS-CoV-2 infection.

FIG. 39 shows SLC11A2 expression (left panel), SARS-CoV-2 expression (center panel—white arrows indicate SARS-CoV-2 positive spots), and SLC11A2/SARS-CoV-2 combined expression (right panel) in a heart sample of a human subject positive for a SARS-CoV-2 infection.

FIGS. 40A-40B show detection of SARS-CoV-2 genes by UMI counts (unique molecular identifiers) per spot (FIG. 40A) and in genes per spot (FIG. 40B)) in control and COVID-positive lung tissue samples. CON: control sample followed by sample number; COVID: SARS-CoV-2 positive sample followed by sample number; Sec: tissue section followed by serial section number.

FIGS. 41A-41B show detection of human transcriptome genes by UMIs counts per spot (FIG. 41A) and in genes per spot (FIG. 41B)) in control and COVID-positive lung tissue samples.

FIG. 42 shows representative detection of SARS-CoV-2 genes in human lung sample positive for SARS-CoV-2.

FIGS. 43A-43C show correlation of detection of human transcriptome gene expression between SARS-CoV-2-positive serial sections.

FIG. 43D shows correlation of detection of SARS-CoV-2 gene expression between SARS-CoV-2-positive serial sections.

FIG. 44 shows abundance of different SARS-CoV-2 genes by total UMI count in all tested samples.

FIG. 45 shows comparison of detection of SARS-CoV-2 genes using RNAScope, the spatial transcriptomics methods described herein, or an overlap of both methods.

FIG. 46 shows detection of E gene using in situ sequencing (left images) and spatial transcriptomics methods disclosed herein (right images).

FIGS. 47A and B shows t-SNE plots of clustering of human transcriptome data across SARS-CoV-2 positive and SARS-CoV-2 negative sections revealing A) six distinct clusters with B) SARS-CoV-2 expression distribution throughout the clusters.

FIG. 48 shows a heatmap of gene expression in each of the six clusters shown in FIG. 47.

FIG. 49 shows representative spatial transcriptomics images for each of the six clusters shown in FIG. 47A.

FIG. 50 shows differentially expressed genes in areas of biological samples where SARS-CoV-2 was detected.

FIG. 51 shows representatively H&E images of SARS-CoV-2 positive and SARS-CoV-2 negative lung tissues.

FIGS. 52A-52C show correlation of detection of SARS-CoV-2 (FIG. 52A) and human transcriptome (FIGS. 52B-52C) gene expression between SARS-CoV-2-positive serial sections.

FIGS. 53A-53B show RNA quality compared to human genes captured (FIG. 53A) and survival of patients compared to viral load (FIG. 53B).

FIG. 54 shows detection of SARS-CoV-2 transcript detection in H&E sections. Dots in each section represent detection of SARS-CoV-2.

FIG. 55A-55F shows detection of SARS-CoV-2 transcript detection in various SARS-CoV-2 genes, including ORFab, S, E, M, N, ORF3a, ORF7a, ORF7b, ORF8, and ORF10.

FIG. 56A-56C shows human transcriptome cluster expression in SARS-CoV-2-positive and SARS-CoV-2-negative samples.

### DETAILED DESCRIPTION

Disclosed herein are methods of spatial analysis to detect viral (e.g., SARS-CoV-2) and host analytes using templated ligation (e.g., targeted RNA capture). Targeted RNA capture is an attractive alternative to poly(A) mRNA capture for interrogating spatial gene expression in FFPE tissue. Compared to poly(A) mRNA capture, targeted RNA capture is less affected by RNA degradation associated with FFPE fixation compared to methods dependent on oligo-dT capture and reverse transcription of mRNA; targeted RNA capture allows for sensitive measurement of specific genes of interest that otherwise might be missed with a whole transcriptomic approach; targeted RNA capture is scalable, with demonstrated probes targeting a large and/or targeted fraction of a transcriptome.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., WO 2020/176788 and/or U.S. Patent Application Publication No. 2020/0277663. Generation of capture probes can be achieved by any appropriate method, including those described in WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in WO 2020/176788 and/or U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). In some instances, the spatially-barcoded array populated with capture probes (as described further herein) is contacted with a biological sample, and the biological sample is permeabilized, allowing the analyte to migrate away from the sample and toward the array. The analyte interacts with a capture probe on the spatially-barcoded array. Another method is to cleave spatially-barcoded capture probes from an array and promote the spatially-barcoded capture probes towards and/or into or onto the biological sample. In some instances, the spatially-barcoded array populated with capture probes (as described further herein) can be contacted with a sample. The spatially-barcoded capture probes are cleaved and then interact with cells within the provided biological sample. The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed to obtain spatially-resolved information about the tagged cell.

In some instances, sample preparation may include placing the sample on a slide, fixing the sample, and/or staining the biological sample for imaging. The stained sample can be then imaged on the array using both brightfield (to image the sample hematoxylin and eosin stain) and/or fluorescence (to image features) modalities. Optionally, the sample can be destained prior to permeabilization. In some embodiments, analytes are then released from the sample and capture probes forming the spatially-barcoded array hybridize or bind the released analytes. The sample is then removed from the array and the capture probes cleaved from the array. The biological sample and array are then optionally imaged a second time in one or both modalities while the analytes are reverse transcribed into cDNA, and an amplicon library is prepared and sequenced. Images are then spatially-overlaid in order to correlate spatially-identified biological sample information. When the sample and array are not imaged a second time, a spot coordinate file is supplied instead. The spot coordinate file replaces the second imaging step. Further, amplicon library preparation can be performed with a unique PCR adapter and sequenced.

In some instances, disclosed is another exemplary workflow that utilizes a spatially-barcoded array on a substrate, where spatially-barcoded capture probes are clustered at areas called features. The spatially-barcoded capture probes can include a cleavage domain, one or more functional domains, a spatial barcode, a unique molecular identifier, and a capture domain. The spatially-barcoded capture probes can also include a 5′ end modification for reversible attachment to the substrate. The spatially-barcoded array is contacted with a biological sample, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, wherein the sample is sandwiched between the diffusion-resistant medium and/or barrier and the array-containing substrate. The analytes are migrated toward the spatially-barcoded capture array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatially-barcoded capture probes, the capture probes can hybridize or otherwise bind a target analyte. The biological sample can be optionally removed from the array.

The capture probes can be optionally cleaved from the array, and the captured analytes can be spatially-barcoded by performing a reverse transcriptase first strand cDNA reaction. A first strand cDNA reaction can be optionally performed using template switching oligonucleotides. For example, a template switching oligonucleotide can hybridize to a poly(C) tail added to a 3′end of the cDNA by a reverse transcriptase enzyme in a template independent manner. The original mRNA template and template switching oligonucleotide can then be denatured from the cDNA and the spatially-barcoded capture probe can then hybridize with the cDNA and a complement of the cDNA can be generated. The first strand cDNA can then be purified and collected for downstream amplification steps. The first strand cDNA can be amplified using PCR, where the forward and reverse primers flank the spatial barcode and analyte regions of interest, generating a library associated with a particular spatial barcode. In some embodiments, the library preparation can be quantitated and/or quality controlled to verify the success of the library preparation steps. In some embodiments, the cDNA comprises a sequencing by synthesis (SBS) primer sequence. The library amplicons are sequenced and analyzed to decode spatial information.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain. In some instances, the capture probe can include functional sequences that are useful for subsequent processing. In some instances, a capture probe can be reversibly attached to a substrate via a linker. The capture probe can include one or more functional sequences, which can include a sequencer specific flow cell attachment sequence, e.g., a P5 or P7 sequence, as well as functional sequence, which can include sequencing primer sequences, e.g., a R1 primer binding site, a R2 primer binding site. In some embodiments, sequence is a P7 sequence and sequence is a R2 primer binding site. A capture probe can additionally include a spatial barcode and/or unique molecular identifier and a capture domain. The different sequences of the capture probe need not be in the sequential manner as depicted in this example; however, the capture domain should be placed in a location on the barcode wherein analyte capture and extension of the capture domain to create a copy of the analyte can occur.

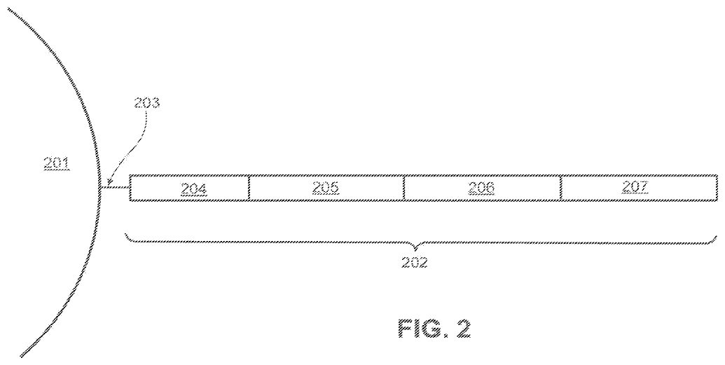

FIG. 2 is a schematic diagram showing an example of a capture probe, as described herein. As shown, the capture probe 202 is optionally coupled to a feature 201 by a cleavage domain 203, such as a disulfide linker. The capture probe can include functional sequences that are useful for subsequent processing, such as functional sequence 204, which can include a sequencer specific flow cell attachment sequence, e.g., a P5 or P7 sequence, as well as functional sequence 205, which can include sequencing primer sequences, e.g., a R1 primer binding site. In some embodiments, sequence 204 is a P7 sequence and sequence 205 is a R1 primer binding site. A spatial barcode 206 can be included within the capture probe for use in barcoding the target analyte. The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems.

In some embodiments, the spatial barcode 206, functional sequences 204 (e.g., flow cell attachment sequence) and 205 (e.g., sequencing primer sequences) can be common to all of the probes attached to a given feature. The spatial barcode can also include a capture domain 207 to facilitate capture of a target analyte.

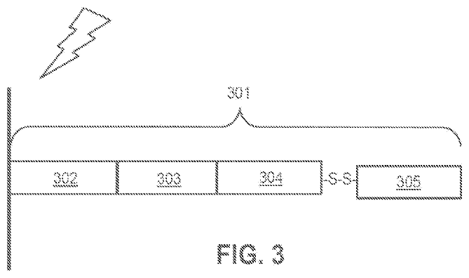

In some cases, capture probes are introduced into the cell using a cell-penetrating peptide. FIG. 3 is a schematic illustrating a cleavable capture probe that includes a cell-penetrating peptide, wherein the cleaved capture probe can enter into a non-permeabilized cell and bind to analytes within the sample. The capture probe 301 contains a cleavage domain 302, a cell penetrating peptide 303, a reporter molecule 304, and a disulfide bond (—S—S—). 305 represents all other parts of a capture probe, for example a spatial barcode and a capture domain.

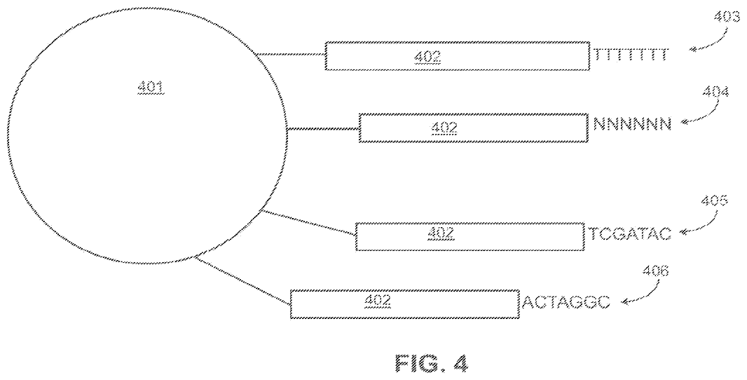

In some instances, the disclosure provides multiplexed spatially-barcoded features. FIG. 4 is a schematic diagram of an exemplary multiplexed spatially-barcoded feature. In FIG. 4, the feature 401 (e.g., a bead, a location on a slide or other substrate, a well on a slide or other substrate, a partition on a slide or other substrate, etc.) can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 402. One type of capture probe associated with the feature includes the spatial barcode 402 in combination with a poly(T) capture domain 403, designed to capture mRNA target analytes. A second type of capture probe associated with the feature includes the spatial barcode 402 in combination with a random N-mer capture domain 404 for gDNA analysis. A third type of capture probe associated with the feature includes the spatial barcode 402 in combination with a capture domain complementary to the analyte capture agent of interest 405. A fourth type of capture probe associated with the feature includes the spatial barcode 402 in combination with a capture probe that can specifically bind a nucleic acid molecule 406 that can function in a CRISPR assay (e.g., CRISPR/Cas9). While only four different capture probe-barcoded constructs are shown in FIG. 4, capture-probe barcoded constructs can be tailored for analyses of any given analyte associated with a nucleic acid and capable of binding with such a construct. For example, the schemes shown in FIG. 4 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

Additional features of capture probes are described in WO 2020/176788 and/or U.S. Patent Application Publication No. 2020/0277663, each of which is incorporated by reference in its entirety. Generation of capture probes can be achieved by any appropriate method, including those described in WO 2020/176788 and/or U.S. Patent Application Publication No. 2020/0277663, each of which is incorporated by reference in its entirety.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in WO 2020/176788 and/or U.S. Patent Application Publication No. 2020/0277663. Some quality control measures are described in WO 2020/176788 and/or U.S. Patent Application Publication No. 2020/0277663.

Spatial information can provide information of biological and/or medical importance. For example, the methods and compositions described herein can allow for: identification of one or more nucleic acids (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. A disease or disorder can include SARS-CoV-2 infection or symptoms.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder nucleic acids).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in WO 2020/176788 and/or U.S. Patent Application Publication No. 2020/0277663. Exemplary features and geometric attributes of an array can be found in WO 2020/176788 and/or U.S. Patent Application Publication No. 2020/0277663.

FIG. 5 depicts an exemplary arrangement of barcoded features within an array. From left to right, FIG. 5 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (labelled as ID578, ID579, ID560, etc.).

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells, areas on a substrate) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Contacting the biological sample with probes (e.g. a plurality of coronavirus probes, or the host probes) can be performed substantially concurrently (e.g. simultaneously or quickly in succession without additional steps in between). Analyte capture is further described in WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using templated ligation or RNA-templated ligation (RTL). Methods of templated ligation have been described previously. See, e.g., Credle et al., Nucleic Acids Res. 2017 Aug. 21; 45(14):e128. Typically, templated ligation includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR® ligase, a PBCV-1 DNA ligase, or a Chlorella virus DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in WO 2020/176788 and/or U.S. Patent Application Publication No. 2020/0277663, or any of one or more of the devices or methods described in WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### I. SARS-CoV-2 and Host Analyte Capture Using Templated Ligation

### (a) Introduction

Provided herein are methods, compositions, systems, and kits for detection of one or more viral analytes (e.g., one or more coronavirus analytes; e.g., one or more SARS-CoV-2 analytes (also known as COVID or COVID-19 or C19); e.g., an analyte that is a variant of SARS-CoV-2) and one or more host analytes (e.g., one or more analytes from a human sample infected with SARS-CoV-2). The methods provided herein are useful to specifically detect a virus analyte and/or a host analyte (e.g. viral or host nucleic acid) by detecting particular sequences of one or more analytes using templated ligation—or RNA templated ligation (RTL)—using RTL probes, where multiple probes or RTL probes hybridize to adjacent sequences of the analyte and are ligated, wherein if only one probe or RTL probe hybridizes, but the other does not, no ligation occurs. Because of this additional step, specificity of analyte detection is enhanced compared to methods that use one probe for the hybridization step. Throughout this document, RTL probe can also refer to viral probes (e.g. coronavirus probes) or host probes. Furthermore, phrases referring to probes or RTL probes are interchangeable if referring to viral probes, such as a coronavirus probe, or host probes.

Although techniques such as whole genome sequencing and whole exome sequencing are available, these techniques have drawbacks in that they provide an abundance of information and increase costs for an experiment. In situations where querying a more limited number of analytes is desired, the methods herein are useful as they provide more discrete targeted analyte capture. In particular, capturing a derivative or proxy of an analyte (e.g., a ligation product) provides enhanced specificity with respect to detection of an analyte. This is because at least two probes specific for a target are required to hybridize to the target in order to create a ligation product that is specific for that targeted analyte followed by ultimate capture of the ligation product for spatial detection and determination.

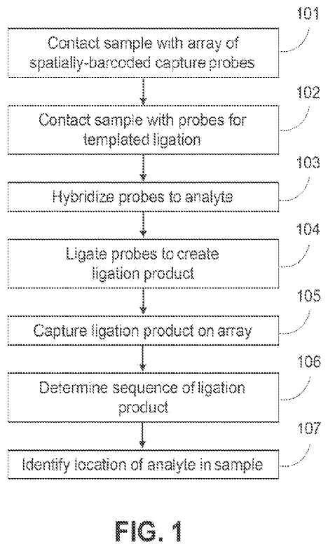

Referring to FIG. 1, in an exemplary embodiment of the disclosure, provided are methods for identifying a location of an analyte (e.g., a SARS-CoV-2 analyte) in a biological sample (e.g., a host sample infected with SARS-CoV-2). In some instances, the methods include 101 contacting a biological sample with an array of spatially-barcoded capture probes. In some instances, the array is on a substrate and the array includes a plurality of capture probes, wherein a capture probe of the plurality includes: (i) a spatial barcode and (ii) a capture domain. After placing the biological sample on the array, the biological sample 102 is contacted with a first RTL probe (e.g., a first SARS-CoV-2 RTL probe) and a second RTL probe (e.g., a second SARS-CoV-2 RTL probe), wherein the first RTL probe (e.g., a first SARS-CoV-2 RTL probe) and the second RTL probe (e.g., a second SARS-CoV-2 RTL probe) each include one or more sequences that are substantially complementary to target sequences of the analyte (e.g., a SARS-CoV-2 nucleic acid sequence(s)), and wherein the second RTL probe (e.g., a second SARS-CoV-2 RTL probe) includes a capture probe capture domain; the first RTL probe (e.g., a first SARS-CoV-2 RTL probe) and the second RTL probe (e.g., a second SARS-CoV-2 RTL probe) 103 hybridize to complementary sequences in the analyte. After hybridization, a ligation product (e.g., a SARS-CoV-2 ligation product) comprising the first RTL probe (e.g., a first SARS-CoV-2 RTL probe) and the second RTL probe (e.g., a second SARS-CoV-2 RTL probe) 104 is generated, and the ligation product (e.g., a SARS-CoV-2 ligation product) is released from the analyte. The liberated ligation product (e.g., a liberated SARS-CoV-2 ligation product) 105 is hybridized to the capture domain of a capture probe on the array. After capture, (i) all or a part of the sequence of the ligation product (e.g., a SARS-CoV-2 ligation product) specifically bound to the capture domain, or a complement thereof, and (ii) the spatial barcode, or a complement thereof 106 can be determined, and the determined sequence of (i) and (ii) 107 can be used to identify the location of the analyte in the biological sample.

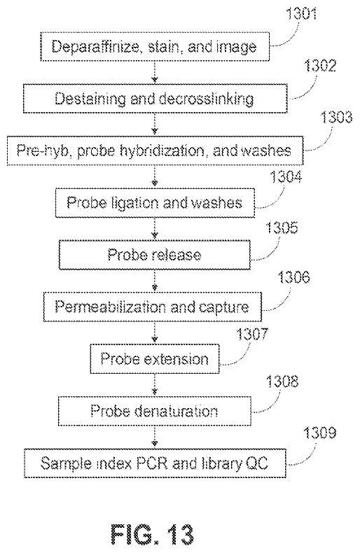

Referring to FIG. 13, in another non-limiting example, a FFPE biological sample is deparaffinized, stained, and imaged 1301. After destaining and decrosslinking 1302, RTL probes (e.g., SARS CoV-2 RTL probes and/or host RTL probes) are added to the sample and are hybridized to an analyte 1303. In some instances, the RTL probes are DNA probes. In some instances, the RTL probes are diribo-containing probes. Probes (e.g., SARS CoV-2 RTL probes and/or host RTL probes) are ligated 1304 and released using an endonuclease such as RNAse H 1305. Ligated RTL probes (e.g., ligated SARS CoV-2 RTL probes and/or ligated RTL host RTL probes) are captured on an array by a capture probe capture domain 1306, extended using a polymerase 1307 and denatured 1308. After quality control cleanup 1309, the abundance and/or location of an analyte in the sample is determined.

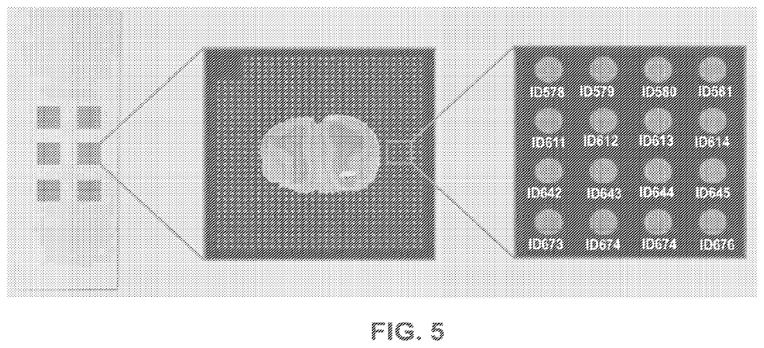

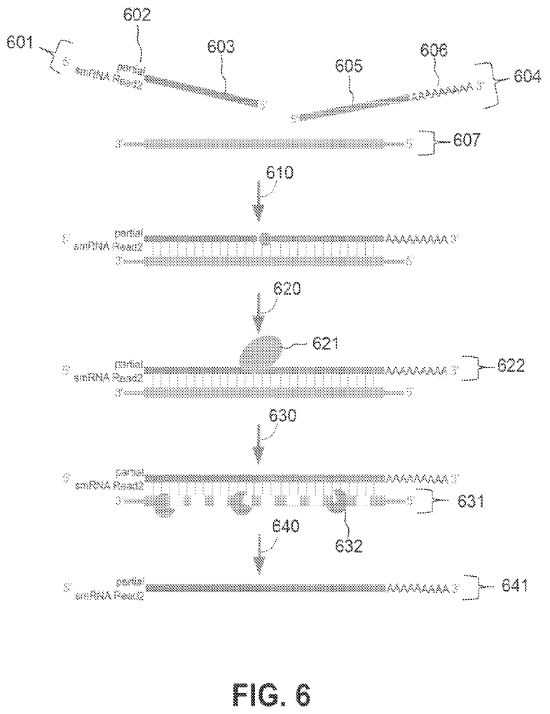

Another non-limiting example of the methods disclosed herein is depicted in FIG. 6. After a biological sample is contacted with a substrate including a plurality of capture probes and contacted with (a) a first RTL probe (e.g., a first SARS-CoV-2 RTL probe) 601 having a target-hybridization sequence 603 and a primer sequence 602 and (b) a second RTL probe (e.g., a second SARS-CoV-2 RTL probe) 604 having a target-hybridization sequence 605 and a capture domain (e.g., a poly-A sequence) 606, the first RTL probe (e.g., a first SARS-CoV-2 RTL probe) 601 and the second RTL probe (e.g., a second SARS-CoV-2 RTL probe) 604 hybridize 610 to an analyte (e.g., a SARS-CoV-2 nucleic acid sequence) 607. The analyte 607 can be a viral analyte (e.g., from SARS-CoV-2) or a host analyte (e.g., from a human subject). A ligase 621 ligates 620 the first RTL probe (e.g., a first SARS-CoV-2 RTL probe) to the second RTL probe (e.g., a second SARS-CoV-2 RTL probe) thereby generating a ligation product (e.g., a SARS-CoV-2 ligation product) 622 that is a proxy for the target analyte sequence. The ligation product is released 630 from the analyte 631 by digesting the analyte using an endoribonuclease 632. The sample is permeabilized 640 and the ligation product 641 is able to hybridize to a capture domain on a capture probe on the substrate.

Also provided herein are methods for identifying a location of an analyte in a biological sample that includes a second RTL probe including a pre-adenylated phosphate group at its 5′ end, which enables the ligating to use a ligase that does not require adenosine triphosphate for ligase activity.

Further provided herein are methods for identifying a location of an analyte in a biological sample that includes one or more spanning probes, in addition to the first and second RTL probes. Using a spanning probe enables greater flexibility in designing templated ligation probes, primarily by increasing the sequences within the analyte that can be used as optional target sequences.

Also provided herein are methods for identifying a location of an analyte in a biological sample that includes optimized hybridizing, washing, and releasing steps.

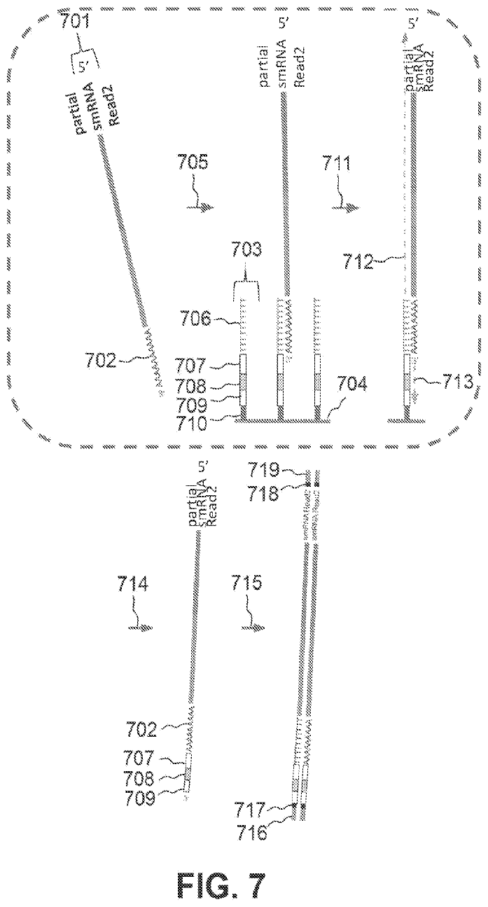

In some embodiments, as shown in FIG. 7, the ligation product 701 (e.g., a proxy of a viral analyte or from a host analyte) includes a capture probe capture domain 702, which can bind to a capture probe 703 (e.g., a capture probe immobilized, directly or indirectly, on a substrate 704). In some embodiments, methods provided herein include contacting 705 a biological sample with a substrate 704, wherein the capture probe 703 is affixed to the substrate (e.g., immobilized to the substrate, directly or indirectly). In some embodiments, the capture probe capture domain 702 of the ligated product specifically binds to the capture domain 706 of the capture probe. The capture probe can also include a unique molecular identifier (UMI) 707, a spatial barcode 708, a functional sequence 709, and a cleavage domain 710.

In some embodiments, methods provided herein include permeabilization of the biological sample such that the capture probe can more easily hybridize to the ligation product (i.e., compared to no permeabilization). In some embodiments, polymerase or reverse transcription (RT) reagents can be added to permeabilized biological samples. Incubation with the polymerase or RT reagents can extend the capture probes 711 to produce spatially-barcoded extended capture probe 712 and 713 from the viral or host ligation product.

In some embodiments, the extended ligation product can be denatured 714 from the capture probe template and transferred (e.g., to a clean tube) for amplification, and/or library construction. Sample indexing (i.e., sample index PCR) can be performed to add P5, i5, P7, and i7 sequences, and then the resulting product can be sequenced.

### (b) SARS-CoV-2 Detection Probes for Templated Ligation

The disclosure provided SARS-CoV-2 detection RTL probes for templated ligation. As used herein, SARS-CoV-2 detection RTL probes (also interchangeably called “SARS-CoV-2 RTL probes,” “SARS-CoV-2 probes,” SARS-CoV-2 probe oligonucleotides,” and the like) detect one or more SARS-CoV-2 nucleic acid sequences. In some embodiments, a SARS-CoV-2 sequence can be a SARS-CoV-2 viral RNA sequence. In some embodiments, a SARS-CoV-2 sequence can include an alteration in a nucleic acid (e.g., an insertion, a deletion, and/or a point mutation (collectively also referred to as a “variant”)). In some instances, SARS-CoV-2 nucleic acid sequences comprise the Genome Reference Sequence (NC_045512), the sequence of which is incorporated by reference in its entirety. In some instances, SARS-CoV-2 nucleic acid sequences comprise the Ensembl Reference Accession Sequence (GCA_009858895; GCA_009858895.3), the sequence of which is incorporated by reference in its entirety.

Non-limiting target sequences (i.e., genes and derivatives thereof) of SARS-CoV-2 are provided in Table 1 and include but are not limited to ORF1a polyprotein (also called ORF1ab polyprotein, ORF1a, or ORF1ab), surface glycoprotein (S), nucleocapsid phosphoprotein (N), ORF8 protein (ORF8), envelope protein (E), ORF3a protein (ORF3a), ORF7a protein (ORF7a), ORF6 protein (ORF6), membrane glycoprotein (M), ORF10 protein (ORF10), and ORF7b.

TABLE 1
SARS-CoV-2 Target Sequences

| | | NCBI | |
|---|---|---|---|
| | | Reference | |
| Name/ | | Sequence and | |
| Gene ID | Description | Location | Aliases |
| ORF1ab; | ORF1a | NC_045512.2 | GU280_gp01 |
| ID: 43740578 | polyprotein; | (266 . . . 21555) | |
| | ORF1ab | | |
| | polyprotein | | |
| S | surface | NC_045512.2 | GU280_gp02, |
| ID: 43740568 | glycoprotein | (21563 . . . 25384) | spike |
| | | | glycoprotein |
| N | nucleocapsid | NC_045512.2 | GU280_gp10 |
| ID: 43740575 | phosphoprotein | (28274 . . . 29533) | |
| ORF8 | ORF8 protein | NC_045512.2 | GU280_gp09 |
| ID: 43740577 | | (27894 . . . 28259) | |
| E | envelope | NC_045512.2 | GU280_gp04 |
| ID: 43740570 | protein | (26245 . . . 26472) | |
| ORF3a | ORF3a | NC_045512.2 | GU280_gp03 |
| ID: 43740569 | protein | (25393 . . . 26220) | |
| ORF7a | ORF7a | NC_045512.2 | GU280_gp07 |
| ID: 43740573 | protein | (27394 . . . 27759) | |
| ORF6 | ORF6 | NC_045512.2 | GU280_gp06 |
| ID: 43740572 | protein | (27202 . . . 27387) | |
| M | membrane | NC_045512.2 | GU280_gp05 |
| ID: 43740571 | glycoprotein | (26523 . . . 27191) | |
| ORF10 | ORF10 protein | NC_045512.2 | GU280_gp11 |
| ID: 43740576 | | (29558 . . . 29674) | |
| ORF7b | ORF7b | NC_045512.2 | GU280_gp08 |
| ID: 43740574 | | (27756 . . . 27887) | |

An exemplary complete genome of SARS-CoV-2 is provided as SEQ ID NO:1 and SEQ ID NO:83. Variants of the SARS-CoV-2 genome or probes directed toward the variants are appropriate to use in any of the compositions, methods, or kits described herein. In some instances, SARS-CoV-2 probes are designed to hybridize to adjacent sequences of one of the target sequences (i.e., genes) in Table 1. In some instances, probes are designed to target one or more of ORF1ab, S, N, ORF8, E, ORF3a, ORF7a, ORF6, M, ORF10, ORF7b, and any combination thereof. In some instances, the probes comprise SEQ ID NOs: 29-82 (and any combination thereof), as shown in Table 2. Any of methods, compositions, kits, and systems relating to templated ligation described herein can utilize SEQ ID NOs: 29-82 (and any combination thereof), as shown in Table 2.

TABLE 2
Probes Targeting SARS-CoV-2 Nucleic Acids

| | | | | | Second RTL probe |
|---|---|---|---|---|---|
| | | | | | Sequence |
| | | SARS- | | | (Sequence |
| | SARS- | CoV-2 | | | identifier |
| | CoV-2 | Gene | Complement of | First RTL | refers to |
| Probe | Gene | Name | SARS-CoV-2 | probe | sequence after |
| Set ID | ID | (Target) | Target Sequence | Sequence | “/5Phos/”) |
| ENSSASG00 | ENSS | ORF1ab | AGTACCGGCAGC | CCTTGGCACCCGA | /5Phos/CGTAGTGCA |
| 005000002\|O | ASG00 | | ACAAGACATCTG | GAATTCCAAGTAC | ACAGGACTAAGCTC |
| RF1ab\|4a8f9d | 005000 | | TCGTAGTGCAAC | CGGCAGCACAAGA | ATAAAAAAAAAAA |
| b | 002 | | AGGACTAAGCTC | CATCTGT (SEQ ID | AAAAAAAAAAAAA |
| | | | AT (SEQ ID NO: 2) | NO: 29) | AAAAAA (SEQ ID |
| | | | | | NO: 30) |
| ENSSASG00 | ENSS | ORF1ab | ATCGAAGCCAAT | CCTTGGCACCCGA | /5Phos/TGTCTTATAG |
| 005000002\|O | ASG00 | | CCATGCACGTAC | GAATTCCAATCGA | CTTCTTCGCGGGTG |
| RF1ab\|f0ec24 | 005000 | | ATGTCTTATAGC | AGCCAATCCATGC | AAAAAAAAAAAAA |
| 4 | 002 | | TTCTTCGCGGGT | ACGTACA (SEQ ID | AAAAAAAAAAAAA |
| | | | GA (SEQ ID NO: 3) | NO: 31) | AAAAA (SEQ ID |
| | | | | | NO: 32) |
| ENSSASG00 | ENSS | ORF1ab | CTGATGAGGTTC | CCTTGGCACCCGA | /5Phos/ATATAGTGA |
| 005000002\|O | ASG00 | | CACCTGGTTTAA | GAATTCCACTGAT | ACCGCCACACATGA |
| RF1ab\|0b67d | 005000 | | CATATAGTGAAC | GAGGTTCCACCTG | CCAAAAAAAAAAA |
| 29 | 002 | | CGCCACACATGA | GTTTAAC (SEQ ID | AAAAAAAAAAAAA |
| | | | CC (SEQ ID NO: 4) | NO: 33) | AAAAAA (SEQ ID |
| | | | | | NO: 34) |
| ENSSASG00 | ENSS | ORF1ab | AGTACCGGCAGC | CCTTGGCACCCGA | /5Phos/CGTAGTGCA |
| 005000003\|O | ASG00 | | ACAAGACATCTG | GAATTCCAAGTAC | ACAGGACTAAGCTC |
| RF1ab\| | 005000 | | TCGTAGTGCAAC | CGGCAGCACAAGA | ATAAAAAAAAAAA |
| 4a8f9db | 003 | | AGGACTAAGCTC | CATCTGT (SEQ ID | AAAAAAAAAAAAA |
| | | | AT (SEQ ID NO: 5) | NO: 35) | AAAAAA (SEQ ID |
| | | | | | NO: 36) |
| ENSSASG00 | ENSS | ORF1ab | GACTTTAGATCG | CCTTGGCACCCGA | /5Phos/GGCCACCAG |
| 005000003\|O | ASG00 | | GCGCCGTAACTA | GAATTCCAGACTT | CTCCTTTATTACCG |
| RF1ab\|38b88 | 005000 | | TGGCCACCAGCT | TAGATCGGCGCCG | TTAAAAAAAAAAA |
| 47 | 003 | | CCTTTATTACCG | TAACTAT (SEQ ID | AAAAAAAAAAAAA |
| | | | TT (SEQ ID NO: 6) | NO: 37) | AAAAAA (SEQ ID |
| | | | | | NO: 38) |
| ENSSASG00 | ENSS | ORF1ab | CTGCAGCAATCA | CCTTGGCACCCGA | /5Phos/GTCATTAGT |
| 005000003\|O | ASG00 | | ATGGGCAAGCTT | GAATTCCACTGCA | ATAACTACCACCAC |
| RF1ab\|6770b | 005000 | | TGTCATTAGTAT | GCAATCAATGGGC | GCAAAAAAAAAAA |
| d7 | 003 | | AACTACCACCAC | AAGCTTT (SEQ ID | AAAAAAAAAAAAA |
| | | | GC (SEQ ID NO: 7) | NO: 39) | AAAAAA (SEQ ID |
| | | | | | NO: 40) |
| ENSSASG00 | ENSS | S | AGGATCCACAA | CCTTGGCACCCGA | /5Phos/GAGACAACT |
| 005000004\|S\| | ASG00 | | GAACAACAGCC | GAATTCCAAGGAT | ACAGCAACTGGTCA |
| 946aa68 | 005000 | | CTTGAGACAACT | CCACAAGAACAAC | TAAAAAAAAAAAA |
| | 004 | | ACAGCAACTGGT | AGCCCTT (SEQ ID | AAAAAAAAAAAAA |
| | | | CATA (SEQ ID | NO: 41) | AAAAAA (SEQ ID |
| | | | NO: 8) | | NO: 42) |
| ENSSASG00 | ENSS | S | AGTACTACTACT | CCTTGGCACCCGA | /5Phos/AACCAACAC |
| 005000004\|S\| | ASG00 | | CTGTATGGTTGG | GAATTCCAAGTAC | CATTAGTGGGTTGG |
| 88d7b4c | 005000 | | TAACCAACACCA | TACTACTCTGTAT | AAAAAAAAAAAAA |
| | 004 | | TTAGTGGGTTGG | GGTTGGT (SEQ ID | AAAAAAAAAAAAA |
| | | | AA (SEQ ID NO: 9) | NO: 43) | AAAAAA (SEQ ID |
| | | | | | NO: 44) |
| ENSSASG00 | ENSS | S | CCTAGTGATGTT | CCTTGGCACCCGA | /5Phos/AAATCTACC |
| 005000004\|S\| | ASG00 | | AATACCTATTGG | GAATTCCACCTAG | AATGGTTCTAAAGC |
| a28c1fl | 005000 | | CAAATCTACCAA | TGATGTTAATACC | CGAAAAAAAAAAA |
| | 004 | | TGGTTCTAAAGC | TATTGGC (SEQ ID | AAAAAAAAAAAAA |
| | | | CG (SEQ ID | NO: 45) | AAAAAA (SEQ ID |
| | | | NO: 10) | | NO: 46) |
| ENSSASG00 | ENSS | ORF3a | GGATTAACAACT | CCTTGGCACCCGA | /5Phos/CGATTGTGT |
| 005000006\|O | ASG00 | | CCGGATGAACCG | GAATTCCAGGATT | GAATTTGGACATGT |
| RF3a\|c1172e2 | 005000 | | TCGATTGTGTGA | AACAACTCCGGAT | TCAAAAAAAAAAA |
| | 006 | | ATTTGGACATGT | GAACCGT (SEQ ID | AAAAAAAAAAAAA |
| | | | TC (SEQ ID NO: 11) | NO: 47) | AAAAAA (SEQ ID |
| | | | | | NO: 48) |
| ENSSASG00 | ENSS | ORF3a | CAGTATAACCAC | CCTTGGCACCCGA | /5Phos/ATGTTCAGA |
| 005000006\|O | ASG00 | | CAATCTGGTAGT | GAATTCCACAGTA | AATAGGACTTGTTG |
| RF3a\|6b88af0 | 005000 | | CATGTTCAGAAA | TAACCACCAATCT | TGAAAAAAAAAAA |
| | 006 | | TAGGACTTGTTG | GGTAGTC (SEQ ID | AAAAAAAAAAAAA |
| | | | TG (SEQ ID | NO: 49) | AAAAAA (SEQ ID |
| | | | NO: 12) | | NO: 50) |
| ENSSASG00 | ENSS | ORF3a | AGTCTGAAGTGA | CCTTGGCACCCGA | /5Phos/TACAACACA |
| 005000006\|O | ASG00 | | AGTAACTGTGTA | GAATTCCAAGTCT | GTCTTTTACTCCAG |
| RF3a\|bca673c | 005000 | | ATACAACACAGT | GAAGTGAAGTAAC | ATAAAAAAAAAAA |
| | 006 | | CTTTTACTCCAG | TGTGTAA (SEQ ID | AAAAAAAAAAAAA |
| | | | AT (SEQ ID | NO: 51) | AAAAAA (SEQ ID |
| | | | NO: 13) | | NO: 52) |
| ENSSASG00 | ENSS | E | CAATATTGCAGC | CCTTGGCACCCGA | /5Phos/TCGAAGCGC |
| 005000010\|E\| | ASG00 | | AGTACGCACACA | GAATTCCACAATA | AGTAAGGATGGCT |
| 3d7b3b9 | 005000 | | ATCGAAGCGCA | TTGCAGCAGTACG | AGTAAAAAAAAAA |
| | 010 | | GTAAGGATGGCT | CACACAA (SEQ ID | AAAAAAAAAAAAA |
| | | | AGT (SEQ ID | NO: 53) | AAAAAAA (SEQ ID |
| | | | NO: 14) | | NO: 54) |
| ENSSASG00 | ENSS | M | AGCGTCCTAGAT | CCTTGGCACCCGA | /5Phos/ACGAAGATG |
| 005000007\|M | ASG00 | | GGTGTCCAGCAA | GAATTCCAAGCGT | TCCACGAAGGATCA |
| \|3b7d5a5 | 005000 | | TACGAAGATGTC | CCTAGATGGTGTC | CAAAAAAAAAAAA |
| | 007 | | CACGAAGGATC | CAGCAAT (SEQ ID | AAAAAAAAAAAAA |
| | | | ACA (SEQ ID | NO: 55) | AAAAAA (SEQ ID |
| | | | NO: 15) | | NO: 56) |
| ENSSASG00 | ENSS | M | ATTTGTAATAAG | CCTTGGCACCCGA | /5Phos/TGTAGCAAC |
| 005000007\|M | ASG00 | | AAAGCGTTCGTG | GAATTCCAATTTG | AGTGATTTCTTTAG |
| \|ad93723 | 005000 | | ATGTAGCAACAG | TAATAAGAAAGCG | GCAAAAAAAAAAA |
| | 007 | | TGATTTCTTTAG | TTCGTGA (SEQ ID | AAAAAAAAAAAAA |
| | | | GC (SEQ ID | NO: 57) | AAAAAA (SEQ ID |
| | | | NO: 16) | | NO: 58) |
| ENSSASG00 | ENSS | M | ATAGCAATTCCA | CCTTGGCACCCGA | /5Phos/TTATTCTGTA |
| 005000007\|M | ASG00 | | CCGGTGATCCAA | GAATTCCAATAGC | AACAGCAGCAAGC |
| \|23567ff | 005000 | | TTTATTCTGTAA | AATTCCACCGGTG | ACAAAAAAAAAAA |
| | 007 | | ACAGCAGCAAG | ATCCAAT (SEQ ID | AAAAAAAAAAAAA |
| | | | CAC (SEQ ID | NO: 59) | AAAAAA (SEQ ID |
| | | | NO: 17) | | NO: 60) |
| ENSSASG00 | ENSS | ORF7a | ATCAGCTAGAGG | CCTTGGCACCCGA | /5Phos/TTGCCCTCGT |
| 005000009\|O | ASG00 | | ATGAAATGGTGA | GAATTCCAATCAG | ATGTTCCAGAAGAG |
| RF7a\|e9eca5f | 005000 | | ATTGCCCTCGTA | CTAGAGGATGAAA | CAAAAAAAAAAAA |
| | 009 | | TGTTCCAGAAGA | TGGTGAA (SEQ ID | AAAAAAAAAAAAA |
| | | | GC (SEQ ID | NO: 61) | AAAAA (SEQ ID |
| | | | NO: 18) | | NO: 62) |
| ENSSASG00 | ENSS | ORF7a | ACTGTTGTACCT | CCTTGGCACCCGA | /5Phos/GGTAGTGAT |
| 005000009\|O | ASG00 | | CTAACACACTCT | GAATTCCAACTGT | AAAGCTCACAAGT |
| RF7a\|2a60f86 | 005000 | | TGGTAGTGATAA | TGTACCTCTAACA | AGCAAAAAAAAAA |
| | 009 | | AGCTCACAAGTA | CACTCTT (SEQ ID | AAAAAAAAAAAAA |
| | | | GC (SEQ ID | NO: 63) | AAAAAAA (SEQ ID |
| | | | NO: 19) | | NO: 64) |
| ENSSASG00 | ENSS | ORF7a | CTTTTGAGTGTG | CCTTGGCACCCGA | /5Phos/TAAACACTA |
| 005000009\|O | ASG00 | | AAGCAAAGTGTT | GAATTCCACTTTT | TTGCCGCAACAATA |
| RF7a\|a45aa6b | 005000 | | ATAAACACTATT | GAGTGTGAAGCAA | AGAAAAAAAAAAA |
| | 009 | | GCCGCAACAATA | AGTGTTA (SEQ ID | AAAAAAAAAAAAA |
| | | | AG (SEQ ID | NO: 65) | AAAAAA (SEQ ID |
| | | | NO: 20) | | NO: 66) |
| ENSSASG00 | ENSS | ORF7b | AGGCGTGACAA | CCTTGGCACCCGA | /5Phos/TTGCAGTTCA |
| 005000012\|O | ASG00 | | GTTTCATTATGA | GAATTCCAAGGCG | AGTGAGAACCAAA |
| RF7b\|7b9495 | 005000 | | TCTTGCAGTTCA | TGACAAGTTTCAT | AGAAAAAAAAAAA |
| 9 | 012 | | AGTGAGAACCA | TATGATC (SEQ ID | AAAAAAAAAAAAA |
| | | | AAAG (SEQ ID | NO: 67) | AAAAAA (SEQ ID |
| | | | NO: 21) | | NO: 68) |
| ENSSASG00 | ENSS | ORF8 | CTTCATAGAACG | CCTTGGCACCCGA | /5Phos/AAGACTACC |
| 005000008\|O | ASG00 | | AACAACGCACTA | GAATTCCACTTCA | CAATTTAGGTTCCT |
| RF8\|e86bd4e | 005000 | | CAAGACTACCCA | TAGAACGAACAAC | GGAAAAAAAAAAA |
| | 008 | | ATTTAGGTTCCT | GCACTAC (SEQ ID | AAAAAAAAAAAAA |
| | | | GG (SEQ ID | NO: 69) | AAAAAA (SEQ ID |
| | | | NO: 22) | | NO: 70) |
| ENSSASG00 | ENSS | ORF8 | ACCGATATCGAT | CCTTGGCACCCGA | /5Phos/GATTTAGAA |
| 005000008\|O | ASG00 | | GTACTGAATGGG | GAATTCCAACCGA | CCAGCCTCATCCAC |
| RF8\|f6d38b5 | 005000 | | TGATTTAGAACC | TATCGATGTACTG | GCAAAAAAAAAAA |
| | 008 | | AGCCTCATCCAC | AATGGGT (SEQ ID | AAAAAAAAAAAAA |
| | | | GC (SEQ ID | NO: 71) | AAAAAA (SEQ ID |
| | | | NO: 23) | | NO: 72) |
| ENSSASG00 | ENSS | ORF8 | GATGTTGAGTAC | CCTTGGCACCCGA | /5Phos/ACATTCTTGG |
| 005000008\|O | ASG00 | | ATGACTGTAAAC | GAATTCCAGATGT | TGAAATGCAGCTAC |
| RF8\|386b3cf | 005000 | | TACATTCTTGGT | TGAGTACATGACT | AAAAAAAAAAAAA |
| | 008 | | GAAATGCAGCTA | GTAAACT (SEQ ID | AAAAAAAAAAAAA |
| | | | CA (SEQ ID | NO: 73) | AAAAA(SEQ ID |
| | | | NO: 24) | | NO: 74) |
| ENSSASG00 | ENSS | N | GGGAATTTAAGG | CCTTGGCACCCGA | /5Phos/GTTGAGTGA |
| 005000005\|N\| | ASG00 | | TCTTCCTTGCCA | GAATTCCAGGGAA | GAGCGGTGAACCA |
| 5fd066e | 005000 | | TGTTGAGTGAGA | TTTAAGGTCTTCCT | AGAAAAAAAAAAA |
| | 005 | | GCGGTGAACCA | TGCCAT (SEQ ID | AAAAAAAAAAAAA |
| | | | AGA (SEQ ID | NO: 75) | AAAAAAA (SEQ ID |
| | | | NO: 25 | | NO: 76) |
| ENSSASG00 | ENSS | N | AATTTCCTTGGG | CCTTGGCACCCGA | /5Phos/ACGTCTGCC |
| 005000005\|N\| | ASG00 | | TTTGTTCTGGAC | GAATTCCAAATTT | GAAAGCTTGTGTTA |
| 824a9ef | 005000 | | CACGTCTGCCGA | CCTTGGGTTTGTTC | CAAAAAAAAAAAA |
| | 005 | | AAGCTTGTGTTA | TGGACC (SEQ ID | AAAAAAAAAAAAA |
| | | | CA (SEQ ID | NO: 77) | AAAAAA (SEQ ID |
| | | | NO: 26) | | NO: 78) |
| ENSSASG00 | ENSS | N | ACCACCACGAAT | CCTTGGCACCCGA | /5Phos/CTTCGGTAGT |
| 005000005\|N\| | ASG00 | | TCGTCTGGTAGC | GAATTCCAACCAC | AGCCAATTTGGTCA |
| aa4877b | 005000 | | TCTTCGGTAGTA | CACGAATTCGTCT | TAAAAAAAAAAAA |
| | 005 | | GCCAATTTGGTC | GGTAGCT (SEQ ID | AAAAAAAAAAAAA |
| | | | AT (SEQ ID | NO: 79) | AAAAA (SEQ ID |
| | | | NO: 27) | | NO: 80) |
| ENSSASG00 | ENSS | ORF10 | TGCACAAGAGTA | CCTTGGCACCCGA | /5Phos/AAACGGAAA |
| 005000013\|O | ASG00 | | GACTATATATCG | GAATTCCATGCAC | AGCGAAAACGTTTA |
| RF10\|446785 | 005000 | | TAAACGGAAAA | AAGAGTAGACTAT | TAAAAAAAAAAAA |
| 2 | 013 | | GCGAAAACGTTT | ATATCGT (SEQ ID | AAAAAAAAAAAAA |
| | | | ATA (SEQ ID | NO: 81) | AAAAAA (SEQ ID |
| | | | NO: 28) | | NO: 82) |

In some instances, the SARS-CoV-2 RTL probes can be used to detect additional viral sequences. For example, the SARS-CoV-2 RTL probes in Table 2 can be used to detect variants of SARS-CoV-2 such as alpha variants (e.g., B.1.1.7 and Q lineages); beta variants (e.g., B.1.351, B.1.351.2, or B.1.351.3 and other descendent lineages); gamma variants (e.g., P.1, P.1.1, or P.1.2 and other descendent lineages); delta variants (e.g., B.1.617.2, AY.1, AY.2, or AY.3 and other descendent lineages), epsilon variants (e.g., B.1.427 and B.1.429 or other descendent lineages), eta variants (e.g., B.1.525 or descendent lineages), iota variants, (e.g., B.1.526 or descendent lineages), kappa variants (e.g., B.1.617.1 or descendent lineages), mu variants (e.g., B.1.621, B.1.621.1 or descendent variants), zeta variants (e.g., P.2 or descendent lineages) or omicron variants (e.g., B.1.1.529, BA.1, BA.1.1, BA. 2, BA.3, BA.4, BA.5 or descendent lineages), or combinations thereof. In some instances, the probes listed in Table 2 can be used to detect infection of SARS-CoV-1. In some instances, the probes listed in Table 2 can be used to detect infection of MERS.

(c) Methods of Diagnosis of SARS-CoV-2 using Detection RTL Probes for Templated Ligation

Any of the exemplary methods described herein can be used to determine an abundance and/or location of one or more SARS-CoV-2 sequences (e.g., using one or more of the RTL probe pairs listed in Table 2). In some embodiments, the methods can include delivering a plurality of RTL probes from Table 2 to a sample (e.g., a tissue sample, for instance, affixed to a support), wherein an RTL probe of the plurality of RTL probes specifically binds to at least one SARS-CoV-2 sequence listed in Table 1.

In some instances, provided herein are methods of diagnosing a subject as having a SARS-CoV-2 infection. In some embodiments, the methods can include determining abundance and/or location of one or more of the SARS-CoV-2 sequences from Table 1 detected using one or more of the RTL probes in Table 2. In some instances, the target sequence is a SARS-CoV-2 sequence selected from ORF1ab, S, ORF3a, E, M, ORF7a, ORF7b, ORF8, N, ORF10, or any combination thereof. In some instances, the methods include detecting an elevated abundance of one or more of the SARS-CoV-2 target sequences compared to a reference level (e.g., a sample known to be negative for SARS-CoV-2). In instances where the abundance of one or more SARS-CoV-2 target sequences is elevated, the subject can be confirmed to be diagnosed with a SARS-CoV-2 infection. It is appreciated that the templated ligation methods and spatial analysis described herein can be used to diagnose (e.g., detect and confirm diagnosis) a subject with SARS-CoV-2.

Disclosed herein are methods of identifying co-localized sequences associated with SARS-CoV-2. In some instances, the methods include diagnosing a subject as having a SARS-CoV-2 infection by detection of one or more ligated probes in the biological sample and co-localizing the expression with a sequence associated with SARS-CoV-2 (e.g., a protein specific to the virus or a protein expressed in a host sample). In some instances, these methods include one or more steps of determining the abundance of a sequence associated with SARS-CoV-2. In some instances, the methods include determining abundance and location of one or more of the SARS-CoV-2 target sequences selected from ORF1ab, S, ORF3a, E, M, ORF7a, ORF7b, ORF8, N, ORF10, or any combination thereof. In some instances, the methods further include detecting an elevated abundance of one or more of the SARS-CoV-2 sequences compared to a reference level (e.g., a sample known to be negative for SARS-CoV-2).

As used in this section, the methods of determining abundance and/or location of one or more of the SARS-CoV-2 sequences can be performed using templated ligation methods described in the disclosure.

In some embodiments, the methods can further include confirming a diagnosis of SARS-CoV-2 infection in the subject. Non-limiting examples of ways to confirm a diagnosis of SARS-CoV-2 infection include obtaining an image of the subject's lungs (e.g., a CT, MRI, or PET scan), determining the levels of other target sequences of SARS-CoV-2 infection, or performing any test to detect a SARS-CoV-2 protein or a SARS-CoV-2 nucleic acid (e.g., rapid antigen test; PCR test). Other methods of confirming a diagnosis of SARS-CoV-2 infection will be apparent to one skilled in the field.

In some embodiments, provided herein are methods of monitoring progression of SARS-CoV-2 infection in a subject over time. In some embodiments, the methods can include (a) determining a first level of one or more of the SARS-CoV-2 target sequences in Table 1 in a first biological sample obtained from a subject at a first time point; (b) determining a second level of one or more of the SARS-CoV-2 target sequences in Table 1 in a second biological sample obtained from the subject at a second time point; (c) identifying: (i) a subject having an increased second level as compared to the first level, as having progressing SARS-CoV-2 infection, or (ii) a subject having about the same or a decreased second level as compared to the first level, as having static or regressing SARS-CoV-2 infection.

Any one of the detection methods can utilize a reference sample having a reference amount of detected SARS-CoV-2 analytes. A reference amount of a SARS-CoV-2 or host analyte (e.g. a nucleic acid or protein) can be any appropriate reference amount. In some embodiments, a reference amount of a SARS-CoV-2 or host nucleic acid can be determined based on an amount of the SARS-CoV-2 or host nucleic acid in a corresponding sample (e.g., a reference sample such as a control subject not diagnosed with a SARS-CoV-2 infection, not presenting with any of the symptoms of a SARS-CoV-2 infection, and not having any known risk factors of a SARS-CoV-2 infection) at a corresponding position. In some embodiments, a reference amount of a SARS-CoV-2 or host nucleic acid can be determined based on an amount of the SARS-CoV-2 or host nucleic acid in one or more other locations in a sample. In some embodiments, a reference amount of a SARS-CoV-2 or host nucleic acid can be a composite or averaged amount (e.g., the averaged amount of a population of persons having or not having a particular disorder).

In some embodiments, a reference amount can be based on a reference amount as published by an appropriate body (e.g., a government agency (e.g., the United States Food and Drug Administration) or a professional organization (e.g., the American Medical Association or American Psychiatric Association)), for example, a reference amount that is a threshold amount for a SARS-CoV-2 or host nucleic acid at the location in the tissue of a subject.

In some embodiments, a reference amount of a SARS-CoV-2 or host nucleic acid can be determined based on any appropriate criteria. For example, in some embodiments, a reference amount of a SARS-CoV-2 or host nucleic acid can come from an age-matched healthy subject. In some embodiments, a reference amount of a SARS-CoV-2 or host nucleic acid can come from a sex-matched healthy subject or a sex-matched healthy subject population. In some embodiments, a reference amount of a SARS-CoV-2 or host nucleic acid can come from an age-matched, sex-matched healthy subject or an age-matched, sex-matched healthy subject population. In some embodiments, a reference amount of a SARS-CoV-2 or host nucleic acid can come from an aggregate sample (e.g., an average of 2 or more individual) of healthy subjects (e.g., that are age-matched and/or sex-matched).

A healthy subject can be any appropriate healthy subject. In some embodiments, a healthy subject does not have a SARS-CoV-2 infection, does not have symptoms of a SARS-CoV-2 infection, does not have any behavior risk factors of a SARS-CoV-2 infection, or combinations thereof.

In some cases, an amount of a SARS-CoV-2 or host nucleic acid can be elevated relative to a reference amount (such as a reference expression level or threshold level of any of the host analytes or viral analytes or any byproducts, precursors, or degradation products thereof). For example, an amount of a SARS-CoV-2 or host nucleic acid can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) greater than a reference amount (e.g., any of the exemplary reference amounts described herein or known in the art).

In some cases, an amount of a SARS-CoV-2 or host nucleic acid/protein can be decreased relative to a reference amount. For example, an amount of a SARS-CoV-2 or host nucleic acid/protein can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) less than a reference amount (e.g., any of the exemplary reference amounts described herein or known in the art).

In some cases, an amount of a SARS-CoV-2 or host nucleic acid/protein can be elevated relative to a reference amount. For example, an amount of a SARS-CoV-2 or host nucleic acid can be at least 5% more, at least 10% more, at least 15% more, at least 20% more, at least 25% more, at least 30% more, at least 35% more, at least 40% more, at least 45% more, at least 50% more, at least 55%, at least 60% more, at least 65% more, at least 70% more, at least 75% more, at least 80% more, at least 85% more, at least 90% more, at least 95% elevated (e.g., about a 5% to about a 99% increase, about a 5% increase to about a 80% increase, about a 5% increase to about a 60% increase, about a 5% increase to about a 40% increase, about a 5% increase to about a 20% increase, about a 20% increase to about a 95% increase, about a 20% increase to about a 80% increase, about a 20% increase to about a 60% increase, about a 20% increase to about a 40% increase, about a 40% increase to about a 99% increase, about a 40% increase to about a 80% increase, about a 40% increase to about a 60% increase, about a 60% increase to about a 99% increase, about a 60% increase to about a 80% increase, about a 80% increase to about a 99% increase) as compared to a reference amount (e.g., any of the exemplary reference amounts described herein).

In some cases, an amount of a SARS-CoV-2 or host nucleic acid can be decreased relative to a reference amount. For example, an amount of a SARS-CoV-2 or host nucleic acid/protein can be at least 5% less, at least 10% less, at least 15% less, at least 20% less, at least 25% less, at least 30% less, at least 35% less, at least 40% less, at least 45% less, at least 50% less, at least 55%, at least 60% less, at least 65% less, at least 70% less, at least 75% less, at least 80% less, at least 85% less, at least 90% less, at least 95% decreased (e.g., about a 5% to about a 99% decrease, about a 5% decrease to about a 80% decrease, about a 5% decrease to about a 60% decrease, about a 5% decrease to about a 40% decrease, about a 5% decrease to about a 20% decrease, about a 20% decrease to about a 95% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease) as compared to a reference amount (e.g., any of the exemplary reference amounts described herein). Other suitable reference amounts and methods of determining the same will be apparent to those skilled in the field.

The predetermined level, such as a reference amount or a reference level, can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or the presence of disease in one defined group is a fold higher, or lower, (e.g., approximately 2-fold, 4-fold, 8-fold, 16-fold or more) than the risk of developing disease or the presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level (e.g. a reference amount or reference level) is a level or occurrence in the same subject, e.g., at a different time point, e.g., an earlier time point. In some embodiments, the predetermined level (e.g. a reference amount) is a level or occurrence in a different subject(s). A predetermined level in a different subject(s) can be from a different time point (e.g. an earlier time point, or a later time point).

### (d) Clusters

Many methods can be used to help identify a cluster of viral or host analytes that includes dysregulated (increased and/or decreased expression of) analytes. Non-limiting examples of such methods include nonlinear dimensionality reduction methods such as t-distributed stochastic neighbor embedding (t-SNE), global t-distributed stochastic neighbor embedding (g-SNE), and uniform manifold approximation and projection (UMAP).

Any number of clusters can be identified. In some embodiments, 2 to 500 clusters can be identified using the methods as described herein. For example, 2 to 10, 2 to 20, 2 to 50, 2 to 75, 2 to 100, 2 to 150, 2 to 200, 2 to 300, 2 to 400, 400 to 500, 300 to 500, 200 to 500, 100 to 500, 75 to 500, 50 to 500, or 25 to 200 clusters can be identified. In some embodiments, 25 to 75, 50 to 100, 50 to 150, 75 to 150, or 100 to 200 clusters can be identified.

Any number of nucleic acids can be sorted into a cluster. For example, a cluster can include about 1 to about 200,000 nucleic acids. In some embodiments, a cluster can include about 1 to about 150,000, about 1 to about 100,000, about 1 to about 75,000, about 1 to about 50,000, about 100,000 to about 200,000, or about 50,000 to about 200,000 nucleic acids. In some embodiments, a cluster includes about 2 to about 25,000 nucleic acids. For example, about 2 to about 50, about 2 to about 100, about 2 to about 500, about 2 to about 1,000, about 2 to about 5,000, about 2 to about 10,000, about 2 to about 15,000, about 2 to about 20,000, about 20,000 to about 25,000, about 15,000 to about 25,000, about 10,000 to about 25,000, about 5,000 to about 25,000, about 1,000 to about 25,000, about 500 to about 25,000, or about 100 to about 25,000 nucleic acids.

In some embodiments, a nucleic acid included in a cluster is different than each of the other nucleic acids in the cluster. For example, the nucleic acid has a sequence that is not identical to any of the other nucleic acids in the cluster. In some embodiments, a nucleic acid corresponds to a gene.

### (e) Biomarkers of SARS-CoV-2

In some instances of the present disclosure, the term “biomarkers of SARS-CoV-2” includes biomarkers associated with detection of SARS-CoV-2. Non-limiting biomarkers of the presence of a coronavirus (e.g., SARS-CoV-2) include one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof.

Some embodiments of any of the methods described herein can include the detection of a level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof.

Also provided herein are reference biomarkers that are not dysregulated in coronavirus detection. In some instances, the references include ZBTB16, SCARB1, AHNAK, ADAMTS1, AFDN, LRRC32 or a byproduct, a degradation product, or a precursor thereof, and any combination thereof. It is appreciated that any of these biomarkers can be used for any of the methods provided herein as a control for quality of a sample and quality control and/or detection of nucleic acids in a sample.

### (f) Methods of Detecting Biomarker(s) in a Location in a Sample

Any of the exemplary methods described herein can be used to determine an expression or activity level of one or more biomarkers (e.g., one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof or at a location in a sample (e.g., a lung tissue sample). In some embodiments, determining an expression or activity level of one or more biomarkers (e.g., one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LASIL, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof) can include any of the workflows described herein.

In some embodiments, the methods can include delivering a plurality of probes to a sample (e.g., a tissue sample, for instance, affixed to a support), wherein a probe of the plurality of probes includes a protein that specifically binds to a biomarker (e.g., one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof).

In some instances, the methods can include detecting expression of a first biomarker in a biological sample and then detecting colocalized expression of various second biomarkers with the first biomarker. For example in some instances, a first biomarker can be a protein or nucleic acid (i.e., mRNA) biomarker that is specific to a cell of interest. The methods include detecting dysregulated nucleic acid biomarker expression in the cell of interest.

In some instances, co-localized second biomarkers can be identified as expressed in the same spot on an array at a first biomarker, when the first biomarker is expressed at low abundances (i.e., less than 5%, less than 10%, less than 15%, less than 20%, less than 25%, or less than 30% expression) compared to the average expression of the average spot on a sample.

In some instances, co-localized second biomarkers can be identified as expressed in the same spot on an array at a first biomarker, when the first biomarker is expressed at high abundances (i.e., greater than 50%, 55%, 60%, 65%, 70%, 75%, 80%, 85%, 90%, 95%, 100% or more) compared to the average expression of the average spot on a sample.

### (g) Methods of Diagnosing SARS-CoV-2

Provided herein are methods of diagnosing a subject as having a viral infection (e.g., a SARS-CoV-2 infection). Also provided herein are methods of identifying a subject as having an increased likelihood of having a viral infection (e.g., a SARS-CoV-2 infection). Further provided herein are methods of monitoring the progression of a viral infection (e.g., a SARS-CoV-2 infection) in a subject. Also provided herein are methods for determining the efficacy of a treatment for a viral infection (e.g., a SARS-CoV-2 infection) in a subject. Further provided herein are methods for treating a viral infection (e.g., a SARS-CoV-2 infection) in a subject.

In any of these methods, a biological sample can be any appropriate biological sample. In some embodiments, a biological sample can be a sample comprising lung tissue. In some embodiments, the biological sample comprises tissue or cell culture samples from other than a lung derived sample. In some embodiments, the method can further include obtaining the sample from the subject. In some embodiments, the method can further include obtaining first and second biological samples from the subject.

In some embodiments, the methods of diagnosing a subject as having a viral infection (e.g., a SARS-CoV-2 infection), or an increased likelihood of having a viral infection (e.g., a SARS-CoV-2 infection) can include (a) determining a level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, in a biological sample from a subject; and (b) identifying a subject having an elevated level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, or a byproduct, a degradation product, or a precursor thereof, and any combination thereof, as compared to a reference level, as having the viral infection (e.g., SARS-CoV-2 infection), or a byproduct, a degradation product, or a precursor thereof, and any combination thereof, as compared to a reference level, as having the viral infection (e.g., SARS-CoV-2 infection), or having an increased likelihood of having the viral infection (e.g., SARS-CoV-2 infection).

In some embodiments, the methods of diagnosing a subject as having a viral infection (e.g., a SARS-CoV-2 infection), or an increased likelihood of having a viral infection (e.g., a SARS-CoV-2 infection) can include (a) determining a level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, in a biological sample from a subject; and (b) identifying a subject having a decreased level of one or more of NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, as compared to a reference level, as having the viral infection (e.g., SARS-CoV-2 infection), or a byproduct, a degradation product, or a precursor thereof, and any combination thereof, as compared to a reference level, as having the viral infection (e.g., SARS-CoV-2 infection), or having an increased likelihood of having the viral infection (e.g., SARS-CoV-2 infection).

It is appreciated that the methods of diagnosing a subject as having a viral infection (e.g., a SARS-CoV-2 infection), or an increased likelihood of having a viral infection can include detection of analytes that are dysregulated (e.g., both upregulated or downregulated (and any combination thereof)).

In some embodiments, the methods can further include confirming a diagnosis of a viral infection (e.g., SARS-CoV-2 infection) in the subject. Non-limiting examples of ways to confirm a diagnosis of a viral infection (e.g., SARS-CoV-2 infection) include obtaining an image of the subject's lung (e.g., a CT, MRI, or PET scan), detecting viral nucleic acid or protein, and/or determining the levels of other biomarkers of a viral infection (e.g., SARS-CoV-2 infection). In some embodiments, the methods can further comprise monitoring the identified subject for the development of symptoms of a viral infection (e.g., SARS-CoV-2 infection).

In some embodiments, the methods can further include performing one or more tests to further determine the subject's risk of developing a viral infection (e.g., SARS-CoV-2 infection). Non-limiting examples of more tests to further determine the subject's risk of developing a viral infection (e.g., SARS-CoV-2 infection) include, detecting a genetic mutation associated with a viral infection (e.g., SARS-CoV-2 infection).

(h) Prophylactic and Therapeutic Uses after Detection of SARS-CoV-2 Using Probes for Templated Ligation

In some embodiments, the methods can further include selecting a treatment for the subject. In some embodiments, the methods can further include administering a treatment of SARS-CoV-2 infection to the subject. In some embodiments, a treatment of SARS-CoV-2 infection can be a treatment that reduces the rate of progression of SARS-CoV-2 infection.

In some instances, the methods disclosed herein include treating a subject having a SARS-CoV-2 infection with one or more therapeutic agents. In some instances, after detecting one or more SARS-CoV-2 analytes, the methods of treatment or prevention include providing treatment to the subject. In some instances, the therapy includes treatment with one or more of: dexamethasone, remdesivir, baricitinib in combination with remdesivir, favipiravir, merimepodib, an anticoagulation drug selected from low-dose heparin or enoxaparin, bamlanivimab, a combination of bamlanivimab and etesevimab, a combination of casirivimab and imdevimab, convalescent plasma, an mRNA SARS-CoV-2 vaccine (such as those produced by Moderna or Pfizer), an attenuated SARS-CoV-2 virus vaccine, or a dead SARS-CoV-2 virus vaccine. In some instances, the therapy comprises a viral vaccine against SARS-CoV-2 (e.g., an adenovirus vaccine such as those produced by Astra Zeneca and Johnson & Johnson. In some instances, the therapy comprises a monoclonal antibody that binds the coronavirus (e.g., SARS-CoV-2) and inhibits infection of a human subject. In some instances, the therapy comprises orthogonal entry inhibitors, such as antibodies, peptides, and small molecules; and furin inhibitors such as decanoyl-RVKR-chloromethylketone (CMK) and naphthofluorescein. See e.g., the agents described in Huang et al., Acta Pharmacologica Sinica (2020) 41:1141-1149; https://doi.org/10.1038/s41401-020-0485-4, which is incorporated by reference herein. In some instances, the therapy includes remdesivir, ritonavir-boosted nirmatrelvir (Paxlovid), molnupiravir, chloroquine or hydroxychloroquine and/or azithromycin, an interferon, ivermectin, lopinavir/ritonavir and other HIV protease inhibitors, or nitazoxanide. In some instances, the therapy includes colchicine, corticosteroids (systemic or inhaled), fluvoxamine, granulocyte-macrophage colony-stimulating factor inhibitors, non-SARS-CoV-2 Specific immunoglobulins, interleukin-1 Inhibitors, interleukin-6 Inhibitors, or kinase Inhibitors (e.g., Janus Kinase Inhibitors and Bruton's Tyrosine Kinase Inhibitors). In some instances, the therapy includes remdesivir. In some instances, the therapy includes ritonavir-boosted nirmatrelvir (Paxlovid). In some instances, the therapy includes molnupiravir. In some instances, the therapy includes chloroquine. In some instances, the therapy includes hydroxychloroquine. In some instances, the therapy includes azithromycin. In some instances, the therapy includes an interferon. In some instances, the therapy includes ivermectin. In some instances, the therapy includes lopinavir. In some instances, the therapy includes ritonavir. In some instances, the therapy includes HIV protease inhibitors. In some instances, the therapy includes nitazoxanide. In some instances, the therapy includes colchicine. In some instances, the therapy includes a corticosteroid. In some instances, the therapy includes a systemic corticosteroid. In some instances, the therapy includes an inhaled corticosteroid. In some instances, the therapy includes fluvoxamine. In some instances, the therapy includes a granulocyte-macrophage colony-stimulating factor inhibitor. In some instances, the therapy includes non-SARS-CoV-2 specific immunoglobulins. In some instances, the therapy includes interleukin-1 inhibitors. In some instances, the therapy includes interleukin-6 inhibitors. In some instances, the therapy includes kinase inhibitors. In some instances, the therapy includes Janus kinase inhibitors. In some instances, the therapy includes Bruton's tyrosine kinase inhibitors.

In some instances, any of the above treatment agents are provided before, substantially contemporaneous with, or after other modes of treatment, for example, surgery or the administration of a biologic, such as a therapeutic antibody. In some embodiments, the SARS-Co-2 infection has recurred or progressed following a previously used therapy.

In some embodiments, also provided herein are methods of determining efficacy of a treatment for a SARS-CoV-2 infection in a subject. In some embodiments, the method can include (a) determining a first abundance and/or location of one or more of the SARS-CoV-2 target sequences from Table 1 detected using one or more of the probes in Table 2 in a first biological sample obtained from a subject at a first time point; determining a second abundance and/or location of one or more of the SARS-CoV-2 target sequences from Table 1 detected using one or more of the probes in Table 2 in a second biological sample obtained from a subject at a second time point; and (c) identifying: (i) the therapeutic treatment as being effective in a subject having about the same or a decreased second abundance as compared to the first abundance, or (ii) the therapeutic treatment as not being effective in a subject having an increased second abundance as compared to the first abundance.

In some embodiments, the methods include identifying one or more therapeutic treatments as being effective in the subject. In some embodiments, the methods can further include selecting additional doses of the one or more therapeutic treatments for the subject. In some embodiments, the methods can further include administering additional doses of the one or more therapeutic treatments to the subject. In some embodiments, the methods can further include recording in the subject's clinical record that the one or more therapeutic treatments is effective in the subject.

In some embodiments, the methods include identifying the therapeutic treatment as not being effective in the subject. In some embodiments, the methods can further include selecting a different therapeutic treatment for the subject. In some embodiments, the methods can further include administering a different therapeutic treatment to the subject. In some embodiments, the methods can further include increasing the dose of the therapeutic treatment to be administered to the subject. In some embodiments, the methods can include administering one or more additional doses of the therapeutic treatment to the subject in combination with an additional therapeutic treatment. In some embodiments, the methods can further include ceasing administration of the therapeutic treatment to the subject.

In some embodiments, the methods can further include additional assessments of the efficacy of the therapeutic treatment. Non-limiting examples of ways to assess efficacy of the therapeutic treatment include obtaining an image of the subject's lungs (e.g., an x-ray, CT, MRI, or PET scan), testing of other nucleic acids, and performing respiratory testing on the subject.

In general, methods include selecting a subject and administering to the subject an effective amount of a therapy, and optionally repeating administration as required for the prevention or treatment of a coronavirus infection or a coronavirus disease and can be administered intranasally (e.g. nose spray), as an inhalant (e.g. nebulization to access the respiratory system), orally, intravenously or topically. A subject can be selected for treatment based on, e.g., determining that the subject has a SARS-CoV-2 infection.

In some embodiments, the methods can further comprise monitoring the identified subject for the development of symptoms of a SARS-CoV-2 infection.

### (i) Methods of Monitoring the Progression of a SARS-CoV-2 Infection in a Subject

In some embodiments, provided herein are methods of monitoring progression of a viral infection (e.g., a SARS-CoV-2 infection) in a subject over time. In some embodiments, the methods can include (a) determining a first level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, in a first biological sample obtained from a subject at a first time point; (b) determining a second level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, in a second biological sample obtained from the subject at a second time point; (c) identifying: (i) a subject having an increased second level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, or a byproduct, a degradation product, or a precursor thereof, and any combination thereof, as compared to the first level, as having progressing a viral infection (e.g., a SARS-CoV-2 infection), or (ii) a subject having about the same or a decreased second level ofNR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LASIL, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, as compared to the first level, as having static or regressing a viral infection (e.g., a SARS-CoV-2 infection).

In some embodiments, when the methods include identifying a subject as having progressing viral infection (e.g., a SARS-CoV-2 infection), the methods can further include administering a treatment for a viral infection (e.g., a SARS-CoV-2 infection) to the subject or increasing the dose of a previously administered treatment for a viral infection (e.g., a SARS-CoV-2 infection) to the subject. In some embodiments, the methods can further include selecting a treatment for a viral infection (e.g., a SARS-CoV-2 infection) for the subject. In some embodiments, the methods can further include administering a treatment of a viral infection (e.g., a SARS-CoV-2 infection) to the subject. In some embodiments, a treatment for a viral infection (e.g., a SARS-CoV-2 infection) can be a treatment that reduces the rate of progression of a viral infection (e.g., a SARS-CoV-2 infection).

### (j) Methods of Determining the Efficacy of a Treatment for a SARS-CoV-2 Infection

In some embodiments, provided herein are methods of determining efficacy of treatment of a treatment for a viral infection (e.g., a SARS-CoV-2 infection) in a subject. In some embodiments, the method can include (a) determining a first level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, or a byproduct or precursor or degradation product or fragment thereof, in a first biological sample obtained from a subject at a first time point; (b) determining a second level of one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof, or a byproduct or precursor or degradation product or fragment thereof, in a second biological sample obtained from the subject at a second time point, wherein the subject is administered one or more doses of a therapeutic treatment between the first and second time points; (c) identifying: (i) the therapeutic treatment as being effective in a subject having about the same or a decreased second level as compared to the first level, or (ii) the therapeutic treatment as not being effective in a subject having an increased second level as compared to the first level for one or more of the following biomarkers: IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, or a byproduct, a degradation product, or a precursor thereof, and any combination thereof; or (c2) identifying: (i) the therapeutic treatment as being effective in a subject having increased second level as compared to the first level, or (ii) the therapeutic treatment as not being effective in a subject having an decreased second level as compared to the first level for one or more of the following biomarkers: NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LASIL, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof.

In some embodiments, the methods include identifying the therapeutic treatment as not being effective in the subject. In some embodiments, the methods can further include selecting a different therapeutic treatment for the subject. In some embodiments, the methods can further include administering a different therapeutic treatment to the subject. In some embodiments, the methods can further include increasing the dose of the therapeutic treatment to be administered to the subject. In some embodiments, the methods can include administering one or more additional doses of the therapeutic treatment to the subject in combination with an additional therapeutic treatment. In some embodiments, the methods can further include ceasing administration of the therapeutic treatment to the subject. In some embodiments, the methods can further include recording in the subject's clinical record that the therapeutic treatment is not effective in the subject. In some embodiments, the methods can further include referring the patient for enrollment in a clinical trial of a different therapeutic agent.

### (k) Viral and Host RTL Probes for Templated Ligation

The methods provided herein utilize viral and host RTL probe pairs (or sets; the terms are interchangeable) to determine abundance and location of one or more viral or host analytes in a biological sample of a subject infected with SARS-CoV-2. It is appreciated that the viral RTL probe pairs target viral analytes (e.g., a SARS-CoV-2 analyte; e.g., any of the analytes in Table 1); and the host RTL probe pairs target host analytes. When reference is made herein to a “probe,” “RTL probe,” a “probe oligonucleotide,” a “probe set,” a “first RTL probe,” a “second RTL probe,” and the like, it is appreciated that the probe (and like terms) refers to elements or embodiments for a viral (e.g., SARS-CoV-2) RTL probe and a host (e.g., a human-specific) RTL probe unless the disclosure specifically discloses that a particular element or embodiment of a probe is specific to either a viral RTL probe or a host RTL probe. Examples of viral RTL probe pairs are provided above in Table 2.

In some instances, the RTL probe pairs are designed so that each probe hybridizes to a sequence in an analyte that is specific to the analyte (e.g., compared to the entire genome of the host). That is, in some instances, a single RTL probe pair can be specific to a single analyte.

In other embodiments, RTL probes can be designed so that one of the RTL probes of a pair is a probe that hybridizes to a specific sequence. Then, the other RTL probe can be designed to detect a mutation of interest. Accordingly, in some instances, multiple second RTL probes can be designed and can vary so that each binds to a specific sequence. For example, one second RTL probe can be designed to hybridize to a wild-type sequence, and another second RTL probe can be designed to detect a mutated sequence. Thus, in some instances, a probe set can include one first RTL probe and two second RTL probes (or vice versa).

On the other hand, in some instances, probes can be designed so that they cover conserved regions of an analyte. Thus, in some instances, a probe (or probe pair) can hybridize to similar analytes in a biological sample (e.g., to detect conserved or similar analytes) or in different biological samples (e.g., across different species).

In some embodiments, RTL probe sets cover all or nearly all of a genome (e.g., human genome). In instances where RTL probe sets are designed to cover an entire genome (e.g., the human genome), the methods disclosed herein can detect analytes in an unbiased manner. In some instances, one RTL probe pair is designed to cover one analyte (e.g., transcript). In some instances, more than one RTL probe pair (e.g., a probe pair comprising a first RTL probe and a second RTL probe) is designed to cover one analyte (e.g., transcript). For example, at least two, three, four, five, six, seven, eight, nine, ten, or more probe sets can be used to hybridize to a single analyte. Factors to consider when designing RTL probes is presence of variants (e.g., SNPs, mutations) or multiple isoforms expressed by a single gene. In some instances, the RTL probe pair does not hybridize to the entire analyte (e.g., a transcript), but instead the RTL probe pair hybridizes to a portion of the entire analyte (e.g., transcript).

In some instances, about 5000, 10,000, 15,000, 20,000, or more RTL probe pair (e.g., a probe pair comprising a first RTL probe and a second RTL probe) are used in the methods described herein. In some instances, about 20,000 RTL probe pairs are used in the methods described herein.

In some instances, RNA capture is targeted RNA capture. Targeted RNA capture using the methods disclosed herein allows for examination of a subset of RNA analytes from the entire transcriptome. In some embodiments, the subset of analytes includes an individual target RNA. In some embodiments, the subset of analytes includes two or more targeted RNAs. In some embodiments, the subset of analytes includes one or more mRNAs transcribed by one or more targeted genes. In some embodiments, the subset of analytes includes one or more mRNA splice variants of one or more targeted genes. In some embodiments, the subset of analytes includes non-polyadenylated RNAs in a biological sample. In some embodiments, the subset of analytes includes detection of mRNAs having one or more single nucleotide polymorphisms (SNPs) in a biological sample.

In some embodiments, the subset of analytes includes mRNAs that mediate expression of a set of genes of interest. In some embodiments, the subset of analytes includes mRNAs that share identical or substantially similar sequences, which mRNAs are translated into polypeptides having similar functional groups or protein domains. In some embodiments, the subset of analytes includes mRNAs that do not share identical or substantially similar sequences, which mRNAs are translated into proteins that do not share similar functional groups or protein domains. In some embodiments, the subset of analytes includes mRNAs that are translated into proteins that function in the same or similar biological pathways. In some embodiments, the biological pathways are associated with a pathologic disease. For example, targeted RNA capture can detect genes that are overexpressed or underexpressed in cancer.

In some embodiments, the subset of analytes includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, or about 1000 analytes.

In some instances, the methods disclosed herein can detect the abundance and location of at least 5,000, 10,000, 15,000, 20,000, or more different analytes.

In some embodiments, the subset of analytes detected by targeted RNA capture methods provided herein includes a large proportion of the transcriptome of one or more cells. For example, the subset of analytes detected by targeted RNA capture methods provided herein can include at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of the mRNAs present in the transcriptome of one or more cells.

In some instances, the probes are DNA probes. In some instances, the probes are diribo-containing probes.

Additional embodiments of probe(s) and probe set(s) are described herein.

### (i) First Host or Viral RTL Probe

In some embodiments, the methods described herein include a first RTL probe (e.g., a first viral RTL probe (e.g., a first SARS-CoV-2 RTL probe) or a first host RTL probe). As used herein, a “first RTL probe” can refer to a probe that hybridizes to all or a portion of an analyte and can be ligated to one or more additional probes (e.g., a second RTL probe or a spanning probe). In some embodiments, “first RTL probe” can be used interchangeably with “first RTL probe oligonucleotide.”

In some embodiments, the first RTL probe includes ribonucleotides, deoxyribonucleotides, and/or synthetic nucleotides that are capable of participating in Watson-Crick type or analogous base pair interactions. In some embodiments, the first RTL probe includes deoxyribonucleotides. In some embodiments, the first RTL probe includes deoxyribonucleotides and ribonucleotides. In some embodiments, the first RTL probe includes a deoxyribonucleic acid that hybridizes to an analyte, and includes a portion of the oligonucleotide that is not a deoxyribonucleic acid. For example, in some embodiments, the portion of the first oligonucleotide that is not a deoxyribonucleic acid is a ribonucleic acid or any other non-deoxyribonucleic acid nucleic acid as described herein. In some embodiments where the first RTL probe includes deoxyribonucleotides, hybridization of the first RTL probe to the mRNA molecule results in a DNA:RNA hybrid. In some embodiments, the first RTL probe includes only deoxyribonucleotides and upon hybridization of the first RTL probe to the mRNA molecule results in a DNA:RNA hybrid.

In some embodiments, the method includes a first RTL probe that includes one or more sequences that are substantially complementary to one or more sequences of an analyte. In some embodiments, a first RTL probe includes a sequence that is substantially complementary to a first target sequence in the analyte. In some embodiments, the sequence of the first RTL probe that is substantially complementary to the first target sequence in the analyte is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence in the analyte.

In some embodiments, a first RTL probe includes a sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

In some embodiments, a sequence of the first RTL probe that is substantially complementary to a sequence in the analyte includes a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some embodiments, a first RTL probe includes a functional sequence. In some embodiments, a functional sequence includes a primer sequence.

As shown in FIG. 6, a non-limiting example of a first RTL probe 601, includes a functional sequence 602, a sequence 603 that is substantially complementary to a first target sequence in the analyte 607. In some embodiments, a first RTL probe 601 can include one or more RNA bases at the 3′ end, while in other embodiments the 3′ end includes DNA.

In some embodiments, a first RTL probe includes an auxiliary sequence that does not hybridize to an analyte. In some embodiments, the auxiliary sequence can be used to hybridize to additional probes.

### (ii) Second Host or Viral RTL Probe

In some embodiments, the methods described herein include a second RTL probe (e.g., a first viral RTL probe (e.g., a first SARS-CoV-2 RTL probe) or a first host RTL probe). As used herein, a “second RTL probe” can refer to a probe that hybridizes to all or a portion of an analyte and can be ligated to one or more additional probes (e.g., a first RTL probe or a spanning probe). In some embodiments, “second RTL probe” can be used interchangeably with “second RTL probe oligonucleotide.” One of skill in the art will appreciate that the order of the probes is arbitrary, and thus the contents of the first RTL probe and/or second RTL probe as disclosed herein are interchangeable.

In some embodiments, the second RTL probe includes ribonucleotides, deoxyribonucleotides, and/or synthetic nucleotides that are capable of participating in Watson-Crick type or analogous base pair interactions. In some embodiments, the second RTL probe includes deoxyribonucleotides. In some embodiments, the second RTL probe includes deoxyribonucleotides and ribonucleotides. In some embodiments, the second RTL probe includes a DNA that hybridizes to an analyte and includes a portion of the oligonucleotide that is not DNA. For example, in some embodiments, the portion of the second RTL probe that is not a DNA is RNA or any other non-DNA nucleic acid as described herein. In some embodiments where the second RTL probe includes deoxyribonucleotides, hybridization of the second RTL probe to the mRNA molecule results in a DNA:RNA hybrid. In some embodiments, the second RTL probe includes only deoxyribonucleotides and upon hybridization of the first RTL probe to the mRNA molecule results in a DNA:RNA hybrid.

In some embodiments, the method includes a second RTL probe that includes one or more sequences that are substantially complementary to one or more sequences of an analyte. In some embodiments, a second RTL probe includes a sequence that is substantially complementary to a second target sequence in the analyte. In some embodiments, the sequence of the second RTL probe that is substantially complementary to the second target sequence in the analyte is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the second target sequence in the analyte.

In some embodiments, a second RTL probe includes a sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

In some embodiments, a sequence of the second RTL probe that is substantially complementary to a sequence in the analyte includes a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some embodiments, a second RTL probe includes a capture probe capture domain sequence. As used herein, a “capture probe capture domain” is a sequence, domain, or moiety that can bind specifically to a capture domain of a capture probe. In some embodiments, “capture domain capture domain” can be used interchangeably with “capture probe binding domain.” In some embodiments, a second RTL probe includes a sequence from 5′ to 3′: a sequence that is substantially complementary to a sequence in the analyte and a capture probe capture domain.

In some embodiments, a capture probe capture domain includes a poly(A) sequence. In some embodiments, the capture probe capture domain includes a poly-uridine sequence, a poly-thymidine sequence, or both. In some embodiments, the capture probe capture domain includes a random sequence (e.g., a random hexamer or octamer). In some embodiments, the capture probe capture domain is complementary to a capture domain in a capture probe that detects a particular target(s) of interest. In some embodiments, a capture probe capture domain blocking moiety that interacts with the capture probe capture domain is provided. In some embodiments, a capture probe capture domain blocking moiety includes a sequence that is complementary or substantially complementary to a capture probe capture domain. In some embodiments, a capture probe capture domain blocking moiety prevents the capture probe capture domain from binding the capture probe when present. In some embodiments, a capture probe capture domain blocking moiety is removed prior to binding the capture probe capture domain (e.g., present in a ligated probe) to a capture probe. In some embodiments, a capture probe capture domain blocking moiety includes a poly-uridine sequence, a poly-thymidine sequence, or both. In some embodiments, the capture probe capture domain sequence includes ribonucleotides, deoxyribonucleotides, and/or synthetic nucleotides that are capable of participating in Watson-Crick type or analogous base pair interactions. In some embodiments, the capture probe binding domain sequence includes at least 10, 11, 12, 13, 14, 15, 16, 17, 18, 19, or 20 nucleotides. In some embodiments, the capture probe binding domain sequence includes at least 25, 30, or 35 nucleotides.

In some embodiments, a second RTL probe includes a phosphorylated nucleotide at the 5′ end. The phosphorylated nucleotide at the 5′ end can be used in a ligation reaction to ligate the second RTL probe to the first RTL probe.

As shown in FIG. 6, a non-limiting example of a second RTL probe 604, includes a sequence 605 that is substantially complementary to a second target sequence on the analyte 607 and a capture probe capture domain 606.

In some embodiments, a second RTL probe includes an auxiliary sequence that does not hybridize to an analyte. In some embodiments, the auxiliary sequence can be used to hybridize to additional probes.

(iii) Multiple Host or Viral RTL Probes

In some embodiments, the methods of target RNA capture as disclosed herein include multiple RTL probes. In some embodiments, the methods include 2, 3, 4, or more RTL probe. In some embodiments, each of the RTL probes includes ribonucleotides, deoxyribonucleotides, and/or synthetic nucleotides that are capable of participating in Watson-Crick type or analogous base pair interactions. In some embodiments, each of the RTL probes includes DNA. In some embodiments, each of the RTL probe includes DNA and RNA.

In some instances, the multiple probes span different target sequences, and multiple, serial ligation steps are carried out to determine the location and abundance of an analyte.

In some instances, the methods including a first RTL probe and multiple second RTL probes (or vice versa) are used, with the multiple second RTL probes hybridizing to different sequences (e.g., wild-type versus mutant sequence, different isoforms, splice variants) in order to identify the sequence of an analyte. It is appreciated that this method can be utilized to detect single mutations (e.g., point mutations, SNPs, splice variants, etc.) or multi-nucleotide mutations (e.g., insertions, deletions, etc.).

Methods provided herein may be applied to a single nucleic acid molecule or a plurality of nucleic acid molecules. A method of analyzing a sample comprising a nucleic acid molecule may comprise providing a plurality of nucleic acid molecules (e.g., RNA molecules), where each nucleic acid molecule comprises a first target region (e.g., a first target sequence) and a second target region (e.g., a second target sequence), a plurality of first RTL probes, and a plurality of second RTL probes. In some cases, one or more target regions of nucleic acid molecules of the plurality of nucleic acid molecules may comprise the same sequence. The first and second target regions (e.g., the first and second target sequences) of a nucleic acid molecule of the plurality of nucleic acid molecules may be adjacent to one another.

(iv) First Host or Viral RTL Probe having a Linker Sequence

Also provided herein are methods for identifying a location of an analyte in a biological sample where the method includes a first RTL probe (e.g., a first viral RTL probe (e.g., a first SARS-CoV-2 RTL probe) or a first host RTL probe) that includes a linker and a second RTL probe. Using a pair of probes where the first RTL probe includes a linker sequence enables greater flexibility in designing templated ligation probes, primarily by increasing the sequences within the analyte that can be used as optional target sequences.

As used herein, a “linker sequence” can refer to one or more nucleic acids sequences on a probe (e.g., a first RTL probe, a second RTL probe, or a spanning probe) that are disposed between sequences that hybridize to an analyte, sequences that link together the analyte specific sequences of a probe. In some embodiments, a linker includes a sequence that is not substantially complementary either to the sequence of the target analyte or to the analyte specific sequences of a first RTL probe, a second RTL probe, or a spanning probe. In some embodiments, the linker sequence includes ribonucleotides, deoxyribonucleotides, and/or synthetic nucleotides, where the sequence within the linker is not substantially complementary to the target analyte or the analyte specific sequences of a first RTL probe, a second RTL probe, or a spanning probe.

In some embodiments where a first and/or a second RTL probe include a linker sequence, the linker sequence can include about 10 nucleotides to about 100 nucleotides, or any of the subranges described herein.

In some embodiments, a linker sequence includes a barcode sequence that serves as a proxy for identifying the analyte. In some embodiments, the barcode sequence is a sequence that is at least 70% identical (e.g., at least 75% identical, at least 80% identical, at least 85% identical, at least 90% identical, at least 95% identical, or at least 99% identical) to a sequence in the analyte. In some embodiments where a linker sequence includes a barcode sequence, the barcode sequence is located 5′ to the linker sequence. In some embodiments where a linker sequence includes a barcode sequence, the barcode sequence is located 3′ to the linker sequence. In some embodiments, the barcode sequence is disposed between two linker sequences. In such cases, the two linker sequences flanking the barcode sequence can be considered a part of the same linker sequence.

In some embodiments where a first and/or a second RTL probe include a linker sequence, the linker sequence can include ribonucleotides, deoxyribonucleotides, and/or synthetic nucleotides.

A non-limiting example of a method for identifying a location of an analyte in a biological sample includes a first RTL probe that includes a linker sequence and a second RTL probe comprising: (a) contacting the biological sample with a substrate including a plurality of capture probes, wherein a capture probe of the plurality of capture probes includes a capture domain and a spatial barcode; (b) contacting the biological sample with a first RTL probe and a second RTL probe, wherein a portion of the first RTL probe and a portion of the second RTL probe are substantially complementary to adjacent sequences of the analyte, wherein the first RTL probe includes: (i) a first sequence that is substantially complementary to a first target sequence of the analyte; (ii) a linker sequence; (iii) a second sequence that is substantially complementary to a second target sequence of the analyte; and wherein the second RTL probe includes a sequence that is substantially complementary to a third target sequence of the analyte and a capture probe capture domain that is capable of binding to a capture domain of a capture probe; (c) hybridizing the first RTL probe and the second RTL probe to the analyte; (d) ligating the first RTL probe and the second RTL probe, thereby creating a ligation product; (e) releasing the ligation product from the analyte; (f) hybridizing the capture probe binding domain to a capture domain; and (g) determining (i) all or a part of the sequence of the ligation product specifically bound to the capture domain, or a complement thereof, and (ii) the spatial barcode, or a complement thereof, and using the determined sequence of (i) and (ii) to identify the location of the analyte in the biological sample.

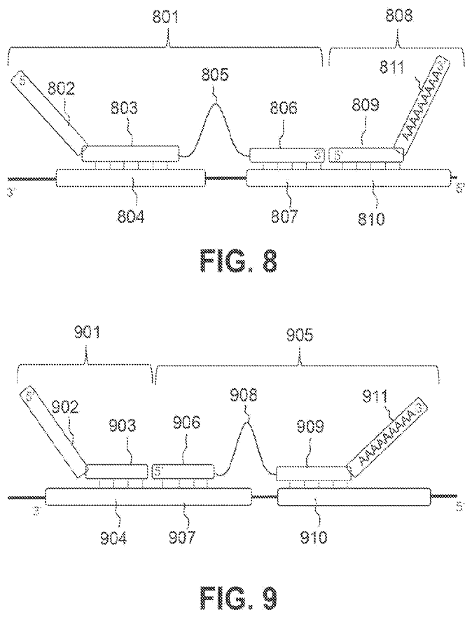

A non-limiting example of a method for identifying a location of an analyte in a biological sample where the method includes a first RTL probe that includes a linker and a second RTL probe can include the components as shown in FIG. 8. A first RTL probe 801 includes a functional sequence 802, a first sequence 803 that is substantially complementary to a first target sequence 804 of the analyte, a linker sequence 805; and a second sequence 806 that is substantially complementary to a second target sequence 807 of the analyte. A second RTL probe 808 includes a sequence 809 that is substantially complementary to a third target sequence 810 of the analyte and a capture probe capture domain 811 that is capable of binding to a capture domain of a capture probe.

### 1) First Host or Viral RTL Probe

In some embodiments where a first RTL probe includes a linker sequence, the first RTL probe (e.g., a first viral RTL probe (e.g., a first SARS-CoV-2 RTL probe) or a first host RTL probe) includes a first sequence that is substantially complementary to a first target sequence of the analyte, a linker sequence, and a second sequence that is substantially complementary to second target sequence of the analyte.

In some embodiments where a first RTL probe includes a linker sequence, the first RTL probe includes a functional sequence. In some embodiments, a first RTL probe includes a functional sequence, a first sequence that is substantially complementary to a first target sequence of the analyte, a linker sequence, and a second sequence that is substantially complementary to second target sequence of the analyte. In some embodiments, the functional sequence includes a primer sequence.

In some embodiments where a first RTL probe includes a linker sequence, a first RTL probe includes at least two ribonucleic acid bases at the 3′ end. In some embodiments, a first RTL probe includes at least three, at least four, at least five, at least six, at least seven, at least eight, at least nine, or at least ten ribonucleic acid bases at the 3′ end. In some embodiments, the 3′ ends of the first RTL probe includes DNA bases.

In some embodiments where a first RTL probe includes a linker, the first RTL probe includes a sequence that is about 10 nucleotides to about 300 nucleotides (e.g., a sequence of about 10 nucleotides to about 300 nucleotides, about 10 nucleotides to about 250 nucleotides, about 10 nucleotides to about 200 nucleotides, about 10 nucleotides to about 150 nucleotides, about 10 nucleotides to about 100 nucleotides, about 10 nucleotides to about 50 nucleotides, about 50 nucleotides to about 300 nucleotides, about 50 nucleotides to about 250 nucleotides, about 50 nucleotides to about 200 nucleotides, about 50 nucleotides to about 150 nucleotides, about 50 nucleotides to about 100 nucleotides, about 100 nucleotides to about 300 nucleotides, about 100 nucleotides to about 250 nucleotides, about 100 nucleotides to about 200 nucleotides, about 100 nucleotides to about 150 nucleotides, about 150 nucleotides to about 300 nucleotides, about 150 nucleotides to about 250 nucleotides, about 150 nucleotides to about 200 nucleotides, about 200 nucleotides to about 300 nucleotides, about 200 nucleotides to about 250 nucleotides, or about 250 nucleotides to about 300 nucleotides).

In some embodiments where a first RTL probe includes a linker sequence, the first RTL probe includes a first sequence that is substantially complementary to a first target sequence of the analyte. In some embodiments, the first sequence of the first RTL probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence in the analyte. In some embodiments, the first sequence of the first RTL probe that is substantially complementary to a first target sequence can include a sequence that is about 5 nucleotides to about 50 nucleotides, or any of the subranges described herein.

In some embodiments where a first RTL probe includes a linker, the first RTL probe includes a second sequence that is substantially complementary to a second target sequence of the analyte. In some embodiments, the second sequence of the first RTL probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the second target sequence in the analyte. In some embodiments, the second sequence of the first RTL probe that is substantially complementary to a second target sequence can include a sequence that is about 5 nucleotides to about 50 nucleotides, or any of the subranges described herein.

In some embodiments, a first RTL probe that includes a linker sequence includes ribonucleotides, deoxyribonucleotides, and/or synthetic nucleotides that are capable of participating in Watson-Crick type or analogous base pair interactions. In some embodiments, the first RTL probe that includes a linker sequence includes deoxyribonucleotides. In some embodiments, the first RTL probe that includes a linker sequence includes deoxyribonucleotides and ribonucleotides. In some embodiments where the first RTL probe that includes a linker sequence includes deoxyribonucleotides, hybridization of the first RTL probe to the mRNA molecule results in a DNA:RNA hybrid. In some embodiments, the first RTL probe that includes a linker sequence includes only deoxyribonucleotides and upon hybridization of the first RTL probe to the mRNA molecule results in a DNA:RNA hybrid.

### 2) Second Host or Viral RTL Probe

In some embodiments where a first RTL probe (e.g., a first viral RTL probe (e.g., a first SARS-CoV-2 RTL probe) or a first host RTL probe) includes a linker sequence, a second RTL probe (e.g., a second viral RTL probe (e.g., a second SARS-CoV-2 RTL probe) or a second host RTL probe) includes a sequence that is substantially complementary to a third target sequence of the analyte and a capture probe capture domain that is capable of binding to a capture domain of a capture probe. In some embodiments, a second RTL probe includes a sequence that is about 10 nucleotides to about 100 nucleotides, or any of the subranges described herein.

In some embodiments, the sequence of the second RTL probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the third target sequence in the analyte. In some embodiments, the sequence of the second RTL probe that is substantially complementary to a third target sequence can include a sequence that is about 5 nucleotides to about 50 nucleotides, or any of the subranges described herein.

In some embodiments where a first RTL probe includes a linker sequence, a first target sequence is not adjacent to a second target sequence. For example, the first target sequence and second target sequences are located on different exons of the same mRNA molecule. In another example, the first target sequence and the second target sequence are located on the same exon of the same mRNA molecule but are not adjacent. In some instances, the first RTL probe and the second RTL probe hybridize to sequences that at least about 1, 2, 3, 4, 5, 6, 7, 8, 9, 10, 11, 12, 13, 14, 15, 16, 17, 18, 19, 20, 21, 22, 23, 24, 25, or more nucleotides apart.

In some embodiments where a first RTL probe includes a linker sequence that is substantially complementary to a second target sequence, a second target sequence is directly adjacent to a third target sequence.

In some embodiments where a first RTL probe includes a linker sequence, the second RTL probe includes ribonucleotides, deoxyribonucleotides, and/or synthetic nucleotides that are capable of participating in Watson-Crick type or analogous base pair interactions. In some embodiments, the second RTL probe includes deoxyribonucleotides. In some embodiments, the second RTL probe includes deoxyribonucleotides and ribonucleotides. In some embodiments where the second RTL probe includes deoxyribonucleotides, hybridization of the second RTL probe to the mRNA molecule results in a DNA:RNA hybrid. In some embodiments, the second RTL probe includes only deoxyribonucleotides and upon hybridization of the second RTL probe to the mRNA molecule results in a DNA:RNA hybrid.

### (v) Second Host or Viral RTL Probe Having a Linker

Also provided herein are methods for identifying a location of an analyte in a biological sample where the method includes a first RTL probe and a second RTL probe that includes a linker sequence. Using a pair of probes where the second RTL probe includes a linker sequence enables greater flexibility in designing templated ligation probes, primarily by increasing the sequences within the analyte that can be used as optional target sequences.

A non-limiting example of a method for identifying a location of an analyte in a biological sample where the method includes a first RTL probe and a second RTL probe that includes a linker includes: (a) contacting the biological sample with a substrate including a plurality of capture probes, wherein a capture probe of the plurality of capture probes includes a capture domain and a spatial barcode; (b) contacting the biological sample with a first RTL probe and a second RTL probe, wherein a portion of the first RTL probe and a portion of the second RTL probe are substantially complementary to adjacent sequences of the analyte, wherein the first RTL probe includes a sequence that is substantially complementary to a first target sequence of the analyte, wherein the second RTL probe includes: (i) a first sequence that is substantially complementary to a second target sequence of the analyte; (ii) a linker sequence; (iii) a second sequence that is substantially complementary to a third target sequence of the analyte; and (iv) a capture probe binding domain that is capable of binding to a capture domain of a capture probe; (c) hybridizing the first RTL probe and the second RTL probe to the analyte; (d) ligating the first RTL probe and the second RTL probe, thereby creating a ligation product; (e) releasing the ligation product from the analyte; (f) hybridizing the capture probe binding domain to a capture domain; and (g) determining (i) all or a part of the sequence of the ligation product specifically bound to the capture domain, or a complement thereof, and (ii) the spatial barcode, or a complement thereof, and using the determined sequence of (i) and (ii) to identify the location of the analyte in the biological sample.

A non-limiting example of a method for identifying a location of an analyte in a biological sample where the method includes a first RTL probe and a second RTL probe that includes a linker can include the components as shown FIG. 9. A first RTL probe 901 includes a functional sequence 902 and a sequence 903 that is substantially complementary to a first target sequence 904 of the analyte. A second RTL probe 905 includes a first sequence 906 that is substantially complementary to a second target sequence 907 of the analyte, a linker sequence 908; and a second sequence 909 that is substantially complementary to a second target sequence 910 of the analyte, and a capture probe capture domain 911 that is capable of binding to a capture domain of a capture probe.

### 1) First Host or Viral RTL Probe

In some embodiments where a second RTL probe (e.g., a second viral RTL probe (e.g., a second SARS-CoV-2 RTL probe) or a second host RTL probe) includes a linker sequence, a first RTL probe (e.g., a first viral RTL probe (e.g., a first SARS-CoV-2 RTL probe) or a first host RTL probe) includes a sequence that is substantially complementary to a first target sequence of the analyte.

In some embodiments where a second RTL probe includes a linker sequence, a first RTL probe includes a functional sequence. In some embodiments, a first RTL probe includes a functional sequence and a sequence that is substantially complementary to a first target sequence of the analyte. In some embodiments, the functional sequence includes a primer sequence. In some embodiments, the first RTL probes includes from 5′ to 3′: a functional sequence, and a sequence that is substantially complementary to a first target sequence.

In some embodiments where a linker is on a second RTL probe, a first RTL probe includes a sequence that is about 10 nucleotides to about 100 nucleotides, or any of the subranges described herein.

In some embodiments, a sequence of a first RTL probe that is substantially complementary to a first target sequence of the analyte is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence in the analyte. In some embodiments, the sequence that is substantially complementary to a first target sequence can include a sequence that is about 5 nucleotides to about 50 nucleotides, or any of the subranges described herein.

In some embodiments, a first RTL probe includes at least two ribonucleic acid bases at the 3′ end. In some embodiments, a first RTL probe includes at least three, at least four, at least five, at least six, at least seven, at least eight, at least nine, or at least ten ribonucleic acid bases at the 3′ end.

In some embodiments where a second RTL probe includes a linker sequence, the first RTL probe includes ribonucleotides, deoxyribonucleotides, and/or synthetic nucleotides that are capable of participating in Watson-Crick type or analogous base pair interactions. In some embodiments, the first RTL probe includes deoxyribonucleotides. In some embodiments, the first RTL probe includes deoxyribonucleotides and ribonucleotides. In some embodiments where the first RTL probe includes deoxyribonucleotides, hybridization of the first RTL probe to the mRNA molecule results in a DNA:RNA hybrid. In some embodiments, the first RTL probe includes only deoxyribonucleotides and upon hybridization of the first RTL probe to the mRNA molecule results in a DNA:RNA hybrid.

### 2) Second Host or Viral RTL Probe

In some embodiments where a linker is on a second RTL probe (e.g., a first viral RTL probe (e.g., a first SARS-CoV-2 RTL probe) or a first host RTL probe), the second RTL probe includes (i) a first sequence that is substantially complementary to a second target sequence of the analyte; (ii) a linker sequence (e.g., any of the exemplary linker sequences described herein); (iii) a second sequence that is substantially complementary to third target sequence of the analyte; and (iv) a capture probe capture domain (e.g., any of the exemplary capture probe capture domains described herein) that is capable of binding to a capture domain of a capture probe.

In some embodiments where a second RTL probe includes a linker sequence, the second RTL probe includes a sequence that is about 10 nucleotides to about 300 nucleotides, or any of the subranges described herein.

In some embodiments where a second RTL probe includes a linker sequence, the second RTL probe includes a first sequence that is substantially complementary to a second target sequence of the analyte. In some embodiments, the first sequence of the second RTL probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the second target sequence in the analyte. In some embodiments, the first sequence of the second RTL probe that is substantially complementary to a second target sequence can include a sequence that is about 5 nucleotides to about 50 nucleotides, or any of the subranges described herein.

In some embodiments where a second RTL probe includes a linker sequence, the second RTL probe includes a second sequence that is substantially complementary to a third target sequence of the analyte. In some embodiments, the second sequence of the second RTL probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the third target sequence in the analyte. In some embodiments, the second sequence of the second RTL probe that is substantially complementary to a third target sequence can include a sequence that is about 5 nucleotides to about 50 nucleotides, or any of the subranges described herein.

In some embodiments where a second RTL probe includes a linker sequence, a second target sequence is not adjacent to a third target sequence in the mRNA molecule. For example, the second target sequence and third target sequence are located on different exons of the same mRNA molecule. In another example, the second target sequence and the third target sequence are located on the same exon of the same mRNA molecule but are not adjacent.

In some embodiments where a second RTL probe includes a linker sequence, a first target sequence is directly adjacent to a second target sequence.

In some embodiments, a second RTL probe that includes a linker sequence includes ribonucleotides, deoxyribonucleotides, and/or synthetic nucleotides that are capable of participating in Watson-Crick type or analogous base pair interactions. In some embodiments, the second RTL probe that includes a linker sequence includes deoxyribonucleotides. In some embodiments, the second RTL probe that includes a linker sequence includes deoxyribonucleotides and ribonucleotides. In some embodiments where the second RTL probe that includes a linker sequence includes deoxyribonucleotides, hybridization of the second RTL probe to the mRNA molecule results in a DNA:RNA hybrid. In some embodiments, the second RTL probe that includes a linker sequence includes only deoxyribonucleotides and upon hybridization of the second RTL probe to the mRNA molecule results in a DNA:RNA hybrid.

(vii) Probe Combination with Linkers on the First RTL Probe and the Second RTL Probe

Also provided herein are methods for identifying a location of an analyte in a biological sample where the method includes a first RTL probe that includes a linker sequence and a second RTL probe that includes a linker sequence. Using a pair of probes where the first RTL probe and second RTL probe each include a linker sequence enables greater flexibility in designing templated ligation probes, primarily by increasing the sequences within the analyte that can be used as optional target sequences.

(l) Probe Combinations including a First RTL probe, a Second RTL probe and a Spanning Probe

Also provided herein are methods for identifying a location of an analyte in a biological sample where the method includes a first RTL probe, a spanning probe, and a second RTL probe. Using a spanning probe enables greater flexibility in designing templated ligation probes, primarily by increasing the sequences within the analyte that can be used as optional target sequences. In some cases, using a spanning probe can also be used to interrogate the variants (e.g., splice variants) that span greater distances that can be interrogated using a first or second RTL probe with a linker sequence.

A non-limiting example of a method for identifying a location of an analyte in a biological sample where the method includes a first RTL probe, a spanning probe, and a second RTL probe, includes: (a) contacting the biological sample with a substrate including a plurality of capture probes, wherein a capture probe of the plurality of capture probes includes a capture domain and a spatial barcode; (b) contacting the biological sample with a first RTL probe, a second RTL probe, and one or more spanning probes, wherein the first RTL probe is substantially complementary to a first portion of the analyte, wherein the second RTL probe is substantially complementary to a second portion of the analyte and further includes a capture probe binding domain, and wherein the spanning probe includes: (i) a first sequence that is substantially complementary to a first target sequence of the analyte, and (ii) a second sequence that is substantially complementary to a second target sequence of the analyte; (c) hybridizing the first RTL probe, the second RTL probe, and the spanning probe to the analyte; (d) ligating the first RTL probe, the one or more spanning probes, and the second RTL probe, thereby creating a ligation product that is substantially complementary to the analyte; (e) releasing the ligation product from the analyte; (f) hybridizing the capture probe binding domain to a capture domain; and (g) determining (i) all or a part of the sequence of the ligation product specifically bound to the capture domain, or a complement thereof, and (ii) the spatial barcode, or a complement thereof, and using the determined sequence of (i) and (ii) to identify the location of the analyte in the biological sample.

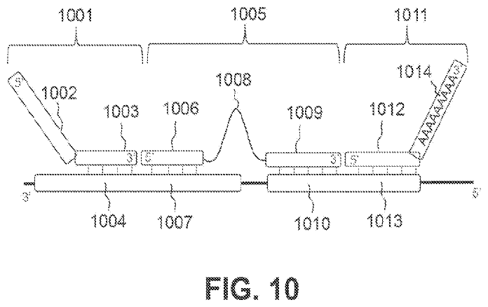

A non-limiting example of a method for identifying a location of an analyte in a biological sample where the method includes a first RTL probe, a second RTL probe, and a spanning probe can include the components as shown FIG. 10. A first RTL probe 1001 includes a functional sequence 1002, a sequence 1003 that is substantially complementary to a first portion 1004 of the analyte. A spanning probe 1005 includes a first sequence 1006 that is substantially complementary to a first target sequence 1007 of the analyte, a linker sequence 1008, and a second sequence 1009 that is substantially complementary to a second target sequence 1010 of the analyte. A second RTL probe 1011 includes a sequence 1012 that is substantially complementary to a second portion 1013 of the analyte and a capture probe capture domain 1014.

### (i) First Host or Viral RTL Probe

In some embodiments where the method includes a spanning probe, a first RTL probe includes a sequence that is substantially complementary to a first portion of the analyte. In some embodiments, a sequence of the first RTL probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a first portion of the analyte. In some embodiments, the sequence of the first RTL probe that is substantially complementary to a first portion of the analyte can include a sequence that is about 5 nucleotides to about 50 nucleotides, or any of the subranges described herein. In some embodiments, the first RTL probe includes a functional sequence. In some embodiments, the functional sequence is a primer sequence.

In some embodiments where the method includes a spanning probe, a first RTL probe includes at least two ribonucleic acid bases at the 3′ end. In some embodiments, a first RTL probe includes at least three, at least four, at least five, at least six, at least seven, at least eight, at least nine, or at least ten ribonucleic acid bases at the 3′ end.

In some embodiments where the method includes a spanning probe, a first RTL probe includes from 5′ to 3′: a functional sequence, a sequence that is substantially complementary to a first portion of the analyte, and two or more ribonucleic acid bases.

In some embodiments where the method includes a spanning probe, a first RTL probe includes ribonucleotides, deoxyribonucleotides, and/or synthetic nucleotides that are capable of participating in Watson-Crick type or analogous base pair interactions. In some embodiments, the first RTL probe includes deoxyribonucleotides. In some embodiments, the first RTL probe includes deoxyribonucleotides and ribonucleotides. In some embodiments where the first RTL probe includes deoxyribonucleotides, hybridization of the first RTL probe to the mRNA molecule results in a DNA:RNA hybrid. In some embodiments, the first RTL probe includes only deoxyribonucleotides and upon hybridization of the first RTL probe to the mRNA molecule results in a DNA:RNA hybrid.

### (ii) Spanning Probe

In some embodiments where the method includes a spanning probe, the spanning probe includes a first sequence that is substantially complementary to a first target sequence of the analyte, and a second sequence that is substantially complementary to a second target sequence of the analyte. In some embodiments, the spanning probe includes a first sequence that is substantially complementary to a first target sequence of the analyte, a functional sequence, and a second sequence that is substantially complementary to a second target sequence of the analyte. In some embodiments, the functional sequence is a linker sequence. The linker sequence can include a total of about 10 nucleotides to about 100 nucleotides, or any of the subranges described herein.

In some embodiments, the functional sequence includes a barcode sequence. In some embodiments, the spanning probe can include a linker and a barcode sequence. In such cases, linker sequences can flank the barcode, the barcode can be 5′ to a linker sequence, or the barcode can be 3′ to a linker sequence. In some embodiments, a barcode sequence is flanked by a 5′ linker sequence (e.g., any of the exemplary linker sequences described herein) and a 3′ linker sequence (e.g., any of the exemplary linker sequences described herein).

In some embodiments, the spanning probe includes from 5′ to 3′: a first sequence, a 5′ linker sequence, a barcode, a 3′ linker sequence, and a second sequence.

In some embodiments, the spanning probes includes a sequence that is about 10 nucleotides to about 300 nucleotides or any of the subranges described herein.

In some embodiments, a first sequence of the spanning probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence of the analyte. In some embodiments, a second sequence of the spanning probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the second target sequence of the analyte.

In some embodiments, the first sequence of the spanning probe and the second sequence of the spanning probe are substantially complementary to sequences within the same exon.

In some embodiments, the first target sequence of the analyte and the second target of the analyte are located within the same exon. In such cases, the first target sequence and the second target sequence are not directly adjacent.

In some embodiments, the first sequence of the spanning probe and the second sequence of the spanning probe are substantially complementary to sequences within the different exons of the same gene. In some embodiments, the first target sequence of the analyte and the second target sequence of the analyte are located on different exons of the same gene.

In some embodiments, the spanning probe includes ribonucleotides, deoxyribonucleotides, and/or synthetic nucleotides that are capable of participating in Watson-Crick type or analogous base pair interactions. In some embodiments, the spanning probe includes deoxyribonucleotides. In some embodiments, the spanning probe includes deoxyribonucleotides and ribonucleotides. In some embodiments where the spanning probe includes deoxyribonucleotides, hybridization of the spanning probe to the mRNA molecule results in a DNA:RNA hybrid. In some embodiments, the spanning probe includes only deoxyribonucleotides and upon hybridization of the spanning probe to the mRNA molecule results in a DNA:RNA hybrid.

(iii) Second Host or Viral RTL Probe

In some embodiments where the method includes a spanning probe, a second RTL probe includes a sequence that is substantially complementary to a second portion of the analyte and a capture probe capture domain (e.g., any of the exemplary capture probe capture domains described herein).

In some embodiments where the method includes a spanning probe, a sequence of the second RTL probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a second portion of the analyte. In some embodiments, the sequence of the second RTL probe that is substantially complementary to a second portion of the analyte can include a sequence that is about 5 nucleotides to about 50 nucleotides, or any of the subranges described herein.

In some embodiments where the method includes a spanning probe, the first portion of the analyte is directly adjacent to the first target sequence, and/or the second portion of the analyte is directly adjacent to the second target sequence. In such cases, the sequence of the first RTL probe is ligated to the first sequence of the spanning probe, and the sequence of the second RTL probe is ligated to the second sequence of the spanning probe. In some embodiments, the spanning probe includes at least two ribonucleic acid based at the 3′ end, the first RTL probe includes at least two ribonucleic acids at the 3′ end, or both. In some embodiments, the spanning probe includes a phosphorylated nucleotide at the 5′ end, the second RTL probe includes a phosphorylated nucleotide at the 5′ end, or both.

In some embodiments where the method includes a spanning probe, a second RTL probe includes ribonucleotides, deoxyribonucleotides, and/or synthetic nucleotides that are capable of participating in Watson-Crick type or analogous base pair interactions. In some embodiments, the second RTL probe includes deoxyribonucleotides. In some embodiments, the second RTL probe includes deoxyribonucleotides and ribonucleotides. In some embodiments where the second RTL probe includes deoxyribonucleotides, hybridization of the second RTL probe to the mRNA molecule results in a DNA:RNA hybrid. In some embodiments, the second RTL probe includes only deoxyribonucleotides and upon hybridization of the second RTL probe to the mRNA molecule results in a DNA:RNA hybrid.

### (iv) Probe Combinations Including a First Host or Viral RTL Probe, a Second Host or Viral RTL Probe, and Multiple Spanning Probes

Also provided herein are methods for identifying a location of an analyte in a biological sample where the method includes a first RTL probe, at least two spanning probes, and a second RTL probe. Using two or more spanning probes enables greater flexibility in designing templated ligation probes, primarily by increasing the sequences within the analyte that can be used as optional target sequences. In some cases, using two or more spanning probe can also be used to interrogate the variants (e.g., splice variants) that span greater distances that can be interrogated using one spanning probe.

A non-limiting example of a method for identifying a location of an analyte in a biological sample where the method includes a first RTL probe, two or more spanning probes, and a second RTL probe, includes: (a) contacting the biological sample with a substrate including a plurality of capture probes, wherein a capture probe of the plurality of capture probes includes a capture domain and a spatial barcode; (b) contacting the biological sample with a first RTL probe, a second RTL probe, and two spanning probes, wherein the first RTL probe is substantially complementary to a first portion of the analyte, wherein the second RTL probe is substantially complementary to a second portion of the analyte and further includes a capture probe binding domain, and wherein the first spanning probe includes: (i) a first sequence that is substantially complementary to a first target sequence of the analyte, and (ii) a second sequence that is substantially complementary to a second target sequence of the analyte; and the second spanning probe includes (i) a third sequence that is substantially complementary to a third target sequence of the analyte, and (ii) a fourth sequence that is substantially complementary to a fourth target sequence of the analyte; (c) hybridizing the first RTL probe, the second RTL probe, and the spanning probe to the analyte; (d) ligating the first RTL probe, the one or more spanning probes, and the second RTL probe, thereby creating a ligation product that is substantially complementary to the analyte; (e) releasing the ligation product from the analyte; (f) hybridizing the capture probe binding domain to a capture domain; and (g) determining (i) all or a part of the sequence of the ligation product specifically bound to the capture domain, or a complement thereof, and (ii) the spatial barcode, or a complement thereof, and using the determined sequence of (i) and (ii) to identify the location of the analyte in the biological sample.

In some embodiments, the methods that include one or more spanning probes include at least two, at least three, at least four, at least five, or more spanning probes. In such cases, the one or more spanning probes includes (i) a third sequence that is substantially complementary to a third target sequence of the analyte, and (ii) a fourth sequence that is substantially complementary to a fourth target sequence of the analyte.

In some embodiments where the method includes two (or more) spanning probes, the first target sequence is located in a first exon, the second target sequence is located in a second exon, and the third target sequence and the fourth target sequence are located in a third exon. In some embodiments where the method includes two (or more) spanning probes, the first target sequence is located in a first exon, the second target sequence is located in a second exon, and the third target sequence is located in a third exon, and the fourth target sequence is located in a fourth exon. In some embodiments where the method includes two (or more) spanning probes, the first target sequence and the second target sequences are located in a first exon, and the third target sequence and the fourth target sequence are located in a second exon. In some embodiments where the method includes two (or more) spanning probes, the first target sequence and the second target sequences are located in a first exon, and the third target sequence is located in a second exon, and the fourth target sequence is located in a third exon.

In some embodiments, where the methods include two (or more) spanning probes, the method includes ligating: the first RTL probe to the spanning probe, the spanning probe to the one or more additional spanning probes, and the one or more additional spanning probes spanning oligonucleotide to the second RTL probe, thereby creating a ligation product that includes one or more sequences that are substantially complementary to the analyte. In some embodiments, where the methods include two (or more) spanning probes, the method includes ligating: the first RTL probe to the one or more additional spanning probes, the one or more additional spanning probes to the spanning probe, and the spanning probe to the second RTL probe, thereby creating a ligation product that includes one or more sequences that are substantially complementary to the analyte.

In some embodiments, each additional spanning probe can include a functional sequence (e.g., any of the functional sequence described herein). For example, each additional spanning probe can include a linker sequence (e.g., any of the exemplary linker sequences described herein). In another example, each additional spanning probe can include a barcode sequence (e.g., any of the exemplary barcode sequences described herein) and a linker sequence (e.g., any of the linker sequences described herein). In some embodiments where an additional spanning probe includes a barcode and a linker, a linker sequences can flank the barcode, the barcode can be 5′ to a linker sequence, or the barcode can be 3′ to a linker sequence. In some embodiments, a barcode sequence is flanked by a 5′ linker sequence (e.g., any of the exemplary linker sequences described herein) and a 3′ linker sequence (e.g., any of the exemplary linker sequences described herein). In some embodiments, an additional spanning probe can include from 5′ to 3′: a first sequence, a 5′ linker sequence, a barcode, a 3′ linker sequence, and a second sequence.

### (m) Pre-Hybridization Methods

### (i) Imaging and Staining

Prior to addition of the host and/or viral RTL probes, in some instances, biological samples can be stained using a wide variety of stains and staining techniques. In some instances, the biological sample is a section on a slide (e.g., a 10 μm section). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3×SSC, 2% BSA, 0.1% Triton X, 1 U/μl RNAse inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

(ii) Preparation of Sample for Application of Host and/or Viral RTL Probes

In some instances, the biological sample is a FFPE sample and is deparaffinized prior to application of the probes. Deparaffinization can be achieved using any method known in the art. For example, in some instances, the biological samples is treated with a series of washes that include xylene and various concentrations of ethanol. In some instances, methods of deparaffinization include treatment of xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1M HCl for about 1 minute). After the decrosslinking step, the biological sample can be washed (e.g., with 1×PBST).

In some instances, the methods of preparing a biological sample for host and/or viral RTL probe application include permeabilizing the sample. In some instances, the biological sample is permeabilized using a phosphate buffer. In some instances, the phosphate buffer is PBS (e.g., 1×PBS). In some instances, the phosphate buffer is PBST (e.g., 1×PBST). In some instances, the permeabilization step is performed multiple times (e.g., 3 times at 5 minutes each).

In some instances, the methods of preparing a biological sample for host and viral RTL probe application include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

### (n) Hybridizing the Host and Viral RTL Probes

In some embodiments, the methods of targeted RNA capture provided herein include hybridizing a first host or viral RTL probe and a second host or viral RTL probes (e.g., a host or viral RTL probe pair). In some instances, the first and second host or viral RTL probes each include sequences that are substantially complementary to one or more sequences (e.g., one or more target sequences) of an analyte of interest. In some embodiments, the first host or viral RTL probe and the second host or viral RTL probe bind to complementary sequences that are completely adjacent (i.e., no gap of nucleotides) to one another or are on the same transcript.

In some instances, the methods include hybridization of host and/or viral RTL probe sets, wherein the host and/or viral RTL probe pairs are in a medium at a concentration of about 1 to about 100 nM. In some instances, the concentration of the RTL probe pairs is about 1, 2, 3, 4, 5, 6, 7, 8, 9, 10, 15, 20, 25, 30, 35, 40, 45, 50, 60, 70, 80, 90, 100, 200, 300, 400, or 500 nM. In some instances, the concentration of the RTL probe pairs is 5 nM. In some instances, the host or viral RTL probe sets are diluted in a hybridization (Hyb) buffer. In some instances, the host or viral RTL probe sets are at a concentration of 5 nM in Hyb buffer.

In some instances, host or viral RTL probe hybridization occurs at about 50° C. In some instances, the temperature of host or viral RTL probe hybridization ranges from about 30° C. to about 75° C., from about 35° C. to about 70° C., or from about 40° C. to about 65° C. In some embodiments, the temperature is about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C. In some instances, host or viral RTL probe hybridization occurs for about 30 minutes, about 1 hour, about 2 hours, about 2.5 hours, about 3 hours, or more. In some instances, host or viral RTL probe hybridization occurs for about 2.5 hours at 50° C.

In some instances, the hybridization buffer includes SSC (e.g., 1×SSC) or SSPE. In some instances, the hybridization buffer includes formamide. In some instances, the hybridization buffer includes one or more salts, like Mg salt for example MgCl₂, Na salt for example NaCl, Mn salt for example MnCl₂. In some instances, the hybridization buffer includes Denhardt's solution, dextran sulfate, ficoll, PEG or other hybridization rate accelerators. In some instances, the hybridization buffer includes a carrier such as yeast tRNA, salmon sperm DNA, and/or lambda phage DNA. In some instances, the hybridization buffer includes one or more blockers. In some instances, the hybridization buffer includes RNase inhibitor(s). In some instances, the hybridization buffer can include BSA, sequence specific blockers, non-specific blockers, EDTA, RNase inhibitor(s), betaine, TMAC, or DMSO. In some instances, a hybridization buffer can further include detergents such as Tween, Triton-X 100, sarkosyl, and SDS. In some instances, the hybridization buffer includes nuclease-free water, DEPC water.

In some embodiments, the complementary sequences to which the first host or viral RTL probe and the second host or viral RTL probe bind are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other. Gaps between the host or viral RTL probes may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, when the first and second host or viral RTL probes are separated from each other by one or more nucleotides, nucleotides are ligated between the first and second host or viral RTL probes. In some embodiments, when the first and second host or viral RTL probes are separated from each other by one or more nucleotides, deoxyribonucleotides are ligated between the first and second host or viral RTL probes.

In some instances, after hybridization, the biological sample is washed with a post-hybridization wash buffer. In some instances, the post-hybridization wash buffer includes one or more of SSC, yeast tRNA, formamide, and nuclease-free water.

Additional embodiments regarding probe hybridization are further provided.

### (i) Hybridizing Temperatures

In some embodiments, the method described utilizes host or viral RTL probes that include deoxyribonucleic acids (instead of strictly utilizing ribonucleotides) at the site of ligation. Utilizing deoxyribonucleic acids in the methods described herein create more uniform efficiency that can be readily-controlled and flexible for various applications.

In a non-limiting example, the methods disclosed herein include contacting a biological sample with a plurality of host and/or viral oligonucleotides (e.g., host or viral RTL probes) including a first host or viral oligonucleotide (e.g., a first host or viral RTL probe) and a second host or viral oligonucleotide (e.g., a second host or viral RTL probe), wherein the first host or viral oligonucleotide (e.g., the first host or viral RTL probe) and the second host or viral oligonucleotide (e.g., the second host or viral RTL probe) are complementary to a first host or viral sequence present in a host or viral analyte and a second host or viral sequence present in the host or viral analyte, respectively; hybridizing the first host or viral oligonucleotide (e.g., the first host or viral RTL probe) and the second host or viral oligonucleotide (e.g., the second host or viral RTL probe) to the host or viral analyte at a first temperature; hybridizing the first host or viral oligonucleotide (e.g., the first host or viral RTL probe) and the second host or viral oligonucleotide (e.g., the second host or viral RTL probe) to a third oligonucleotide (e.g., a splint oligonucleotide) at a second temperature such that the first host or viral oligonucleotide (e.g., the first host or viral RTL probe) and the second host or viral oligonucleotide (e.g., the second host or viral RTL probe) abut each other; ligating the first host or viral oligonucleotide (e.g., the first host or viral RTL probe) to the second host or viral oligonucleotide (e.g., the second host or viral RTL probe) to create a ligation product; contacting the biological sample with a substrate, wherein a capture probe is immobilized on the substrate, wherein the capture probe includes a spatial barcode and a capture domain; allowing the host or viral ligation product to specifically bind to the capture domain; and determining (i) all or a part of the sequence of the host or viral ligation product specifically bound to the capture domain, or a complement thereof, and (ii) the spatial barcode, or a complement thereof, and using the determined sequence of (i) and (ii) to identify the location and abundance of the host or viral analyte in the biological sample; wherein the first oligonucleotide (e.g., the first RTL probe), the second oligonucleotide (e.g., the second RTL probe), and the third oligonucleotide are DNA oligonucleotides, and wherein the first temperature is a higher temperature than the second temperature.

A non-limiting example of this method is shown in FIG. 11. A biological sample including a host and/or viral analyte 1101 is contacted with a host or viral first RTL probe 1102 and a second host or viral RTL probe 1103. The first host or viral RTL probe 1102 and the second host or viral RTL probe 1103 hybridize to the host or viral analyte at a first target sequence 1104 and a second target sequence 1105, respectively. The first host or viral RTL probe and the second host or viral RTL probe include free ends 1107-1110. As shown in FIG. 11, in this example the first and second target sequences are not directly adjacent in the analyte. After hybridization, unbound first and second host or viral RTL probes are washed away. A third oligonucleotide 1106 is added to the washed biological sample and the third oligonucleotide 1106 hybridizes to the first and the second host or viral RTL probes at 1108 and 1109, respectively. After hybridization, the first host or viral RTL probe is extended (arrow) and a host or viral ligation product is created that includes the first host or viral RTL probe sequence and the second host or viral RTL probe sequence. Alternatively, instead of extending the first host or viral RTL probe, the third oligonucleotide is used to “bind” the first host or viral RTL probe and the second host or viral RTL probe together. In such cases, the first host or viral RTL probe and the second host or viral RTL probe bound together by the third oligonucleotide can be referred to as a host or viral bound product. The host or viral ligation product or bound product is contacted with a substrate 1111, and the host or viral ligation product or bound product is hybridized to a capture probe of the substrate 1111 on the array at distinct spatial positions. In some embodiments, the biological sample is contacted with the substrate 1111 prior to being contacted with the first host or viral RTL probe and the second host or viral RTL probe.

In some embodiments, the first host or viral oligonucleotide (e.g., the first host or viral RTL probe) and the second host or viral oligonucleotide (e.g., the second host or viral RTL probe) hybridize to a host or viral analyte at a first temperature. In some embodiments, the first temperature ranges from about 50° C. to about 75° C., from about 55° C. to about 70° C., or from about 60° C. to about 65° C. In some embodiments, the first temperature is about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C.

In some embodiments, after the step of hybridizing the first host or viral oligonucleotide (e.g., the first host or viral RTL probe) and the second host or viral oligonucleotide (e.g., the second host or viral RTL probe) to the host or viral analyte, a wash step is performed to remove unbound oligonucleotides (e.g., probes). The wash step can be performed using any of the wash methods and solutions described herein.

In some embodiments, after the step of hybridizing the first host or viral oligonucleotide (e.g., the first host or viral RTL probe) and the second host or viral oligonucleotide (e.g., the second host or viral RTL probe) to the host or viral analyte, a third oligonucleotide (e.g., a splint oligonucleotide) is added to the analyte. In some embodiments, the third oligonucleotide is a nucleic acid sequence. In some embodiments, the third oligonucleotide is a DNA oligonucleotide.

In some embodiments, the third oligonucleotide includes a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a portion of the first host or viral RTL probe (e.g., a portion of the first host or viral RTL probe that is not hybridized to the host or viral analyte (e.g., an auxiliary sequence)). In some embodiments, the third oligonucleotide includes a sequence that is 100% complementary to a portion of the first host or viral oligonucleotide (e.g., the first RTL probe). In some embodiments, the third oligonucleotide includes a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a portion of the second host or viral RTL probe (e.g., a portion of the second host or viral RTL probe that is not hybridized to the host or viral analyte (e.g., an auxiliary sequence)). In some embodiments, the third oligonucleotide includes a sequence that is 100% complementary to a portion of the second host or viral oligonucleotide (e.g., the second host or viral RTL probe). In some embodiments, the third oligonucleotide hybridizes to the first host or viral oligonucleotide (e.g., the first host or viral RTL probe) at the complementary portion. In some embodiments, the third oligonucleotide hybridizes to the second host or viral oligonucleotide (e.g., the second host or viral RTL probe) at the complementary portion.

In some embodiments, the third oligonucleotide hybridizes to the first host or viral oligonucleotide (e.g., the first host or viral RTL probe) and to the second host or viral oligonucleotide (e.g., the second host or viral RTL probe) at a second temperature. In some embodiments, the second temperature is lower than the first temperature at which the first and second oligonucleotides (e.g., the first and second host or viral RTL probes) bind the host or viral analyte. In some embodiments, the second temperature ranges from about 15° C. to about 35° C., from about 20° C. to about 30° C., or from about 25° C. to about 30° C. In some embodiments, the first temperature is about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., or about 35° C. Methods including a third, or splint, oligonucleotide have been described in U.S. Patent Pub. No. 2019/0055594A1, which is herein incorporated by reference in its entirety.

In some embodiments, after the step of hybridizing the third oligonucleotide to the analyte, a wash step is performed to remove unbound third oligonucleotides. The wash step can be performed using any of the wash methods and solutions described herein. In some embodiments, after the washing step, the first and second host or viral oligonucleotides (e.g., the first and second host or viral RTL probes) are bound to (e.g., hybridized to) the analyte, and the third oligonucleotide is bound to (e.g., hybridized to) the first and second host or viral oligonucleotides (e.g., at portions of the first and second host or viral RTL probes that are not bound to the analyte).

In some embodiments, the first host or viral oligonucleotide (e.g., the first host or viral RTL probe), the second host or viral oligonucleotide (e.g., the second host or viral RTL probe), and the third oligonucleotide are added to the biological sample at the same time. Then, in some embodiments, the temperature is adjusted to the first temperature to allow the first host or viral oligonucleotide (e.g., the first host or viral RTL probe) and the second host or viral oligonucleotide (e.g., the second host or viral RTL probe) to hybridize to the host or viral analyte in the biological sample. Next, the temperature is adjusted to the second temperature to allow the third oligonucleotide to hybridize to the first host or viral oligonucleotide and the second host or viral oligonucleotide.

In some embodiments where a third oligonucleotide hybridizes to a first host or viral RTL probe and a second host or viral RTL probe that are hybridized to targets sequences that are not directly adjacent in the analyte, the third oligonucleotide is extended to fill the gap between the first host or viral RTL probe and the second host or viral RTL probe. In some instances, a polymerase (e.g., a DNA polymerase) can extend one of the host or viral RTL probes (e.g., the first host or viral RTL probe) prior to ligation. For example, as shown in FIG. 11, the first RTL probe 1102 is extended to fill the gap between the first RTL probe 1102 and the second RTL probe 1103.

In some embodiments, a ligation step is performed. Ligation can be performed using any of the methods described herein. In some embodiments, the step includes ligation of the first host or viral oligonucleotide (e.g., the first RTL probe) and the second host or viral oligonucleotide (e.g., the second RTL probe), forming a host or viral ligation product. In some embodiments, the third oligonucleotide serves as an oligonucleotide splint to facilitate ligation of the first host or viral oligonucleotide (e.g., the first host or viral RTL probe) and the second host or viral oligonucleotide (e.g., the second host or viral RTL probe). In some embodiments, ligation is chemical ligation. In some embodiments, ligation is enzymatic ligation. In some embodiments, the ligase is a T4 RNA ligase (Rnl2), a SplintR® ligase, a PBCV-1 DNA ligase, a Chlorella virus DNA ligase, a single stranded DNA ligase, or a T4 DNA ligase.

### (ii) Hybridization Buffer

In some embodiments, a first host or viral RTL probe and a second host or viral RTL probe are hybridized to the analyte in a hybridization buffer. In some instances, the hybridization buffer contains formamide. In other instances, the hybridization buffer is formamide free. Chemically, formamide can oxidize over time, thereby impacting reagent shelf life and, most importantly, reagent efficacy. As such, the methods described herein can include formamide-free buffers, including formamide-free hybridization buffer.

In some embodiments, the formamide-free hybridization buffer is a saline-sodium citrate (SSC) hybridization buffer. In some embodiment, the SSC is present in the SSC hybridization buffer from about 1×SSC to about 6×SSC (e.g., about 1×SSC to about 5×SSC, about 1×SSC to about 4×SSC, about 1×SSC to about 3×SSC, about 1×SSC to about 2×SSC, about 2×SSC to about 6×SSC, about 2×SSC to about 5×SSC, about 2×SSC to about 4×SSC, about 2×SSC to about 3×SSC, about 3×SSC to about 5×SSC, about 3×SSC to about 4×SSC, about 4×SSC to about 6×SSC, about 4×SSC to about 6×SSC, about 4×SSC to about 5×SSC, or about 5×SSC to about 6×SSC). In some embodiments, the SSC is present in the SSC hybridization buffer from about 2×SSC to about 4×SSC. In some embodiments, SSPE hybridization buffer can be used.

In some embodiments, the SSC hybridization buffer is at a temperature from about 40° C. to about 60° C. (e.g., about 40° C. to about 55° C., about 40° C. to about 50° C., about 40° C. to about 45° C., about 45° C. to about 60° C., about 45° C. to about 55° C., about 45° C. to about 50° C., about 50° C. to about 60° C., about 50° C. to about 55° C., or about 55° C. to about 60° C.). In some embodiments, the SSC hybridization buffer is at temperature from about 45° C. to about 55° C., or any of the subranges described herein. In some embodiments, the SSC hybridization buffer is at a temperature of about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In some embodiments, the SSC hybridization buffer is at a temperature of about 50° C.

In some embodiments, the SSC hybridization buffer further comprises one or more of a carrier, a crowder, or an additive. Non-limiting examples of a carrier that can be included in the hybridization buffer include: yeast tRNA, salmon sperm DNA, lambda phage DNA, glycogen, and cholesterol. Non-limiting examples of a molecular crowder that can be included in the hybridization buffer include: Ficoll, dextran, Denhardt's solution, and PEG. Non-limiting examples of additives that can be included in the hybridization buffer include: binding blockers, RNase inhibitors, Tm adjustors and adjuvants for relaxing secondary nucleic acid structures (e.g., betaine, TMAC, and DMSO). Further, a hybridization buffer can include detergents such as SDS, Tween, Triton-X 100, and sarkosyl (e.g., N-Lauroylsarcosine sodium salt). A skilled artisan would understand that a buffer for hybridization of nucleic acids could include many different compounds that could enhance the hybridization reaction.

### (o) Washing

In some embodiments, the methods disclosed herein also include a wash step. The wash step removes any unbound host or viral RTL probes. Wash steps could be performed between any of the steps in the methods disclosed herein. For example, a wash step can be performed after adding probes to the biological sample. As such, free/unbound probes are washed away, leaving only probes that have hybridized to an analyte. In some instances, multiple (i.e., at least 2, 3, 4, 5, or more) wash steps occur between the methods disclosed herein. Wash steps can be performed at times (e.g., 1, 2, 3, 4, or 5 minutes) and temperatures (e.g., room temperature; 4° C. known in the art and determined by a person of skill in the art.

In some instances, wash steps are performed using a wash buffer. In some instances, the wash buffer includes SSC (e.g., 1×SSC). In some instances, the wash buffer includes PBS (e.g., 1×PBS). In some instances, the wash buffer includes PBST (e.g., 1×PBST). In some instances, the wash buffer can also include formamide or be formamide free.

In some embodiments, the SSC wash buffer comprises a solvent. In some embodiments, the solvent comprises formamide. In some embodiments, formamide is present in the SSC wash buffer from about 10% (w/v) to about 25% (w/v), or any of the subranges described herein. In some embodiments, formamide is present in the SSC wash buffer from about 15% (w/v) to about 20% (w/v). In some embodiments, formamide is present in the SSC wash buffer at about 16% (w/v).

Additional embodiments regarding wash steps are provided herein.

### (i) Formamide Free Wash Buffer

In some embodiments, after ligating a first host or viral RTL probe and a host or viral second RTL probe, the one or more unhybridized first host or viral RTL probes, one or more unhybridized second host or viral RTL probes, or both, are removed from the array. In some embodiments, after ligating a first host or viral RTL probe, one or more spanning probes, and a second host or viral RTL probe, the one or more unhybridized first, second, and/or spanning probes, are removed from the array. In some embodiments, after ligating a first host or viral RTL probe, a host or viral second RTL probe, and a third oligonucleotide, the one or more unhybridized first RTL probes, one or more unhybridized second RTL probes, or one or more third oligonucleotides, or all the above, are removed from the array.

In some embodiments, a pre-hybridization buffer is used to wash the sample. In some embodiments, a phosphate buffer is used. In some embodiments, multiple wash steps are performed to remove unbound oligonucleotides.

In some embodiments, a first probe and a second probe are hybridized to the analyte in a hybridization buffer. In some instances, the hybridization buffer contains formamide. In other instances the hybridization buffer is formamide free. Formamide is not human friendly and it is a known health hazard. Chemically, it can oxidize over time, thereby impacting reagent shelf life and, most importantly, reagent efficacy. As such, the methods described herein can include formamide-free buffers, including formamide-free hybridization buffer.

In some embodiments, removing includes washing the one or more unhybridized probes (e.g., a first probe, a second probe, a first RTL probe, a second RTL probe, a spanning probe, additional spanning probes, and a third oligonucleotide) from the array in a formamide-free wash buffer.

In some embodiments, the formamide-free wash buffer is an SSC wash buffer. In some embodiments, SSC is present in the SSC wash buffer from about 0.01×SSC to about 1×SSC (e.g., about 0.01×SSC to about 0.5×SSC, 0.01×SSC to about 0.1×SSC, about 0.01×SSC to about 0.05×SSC, about 0.05×SSC to about 1×SSC, about 0.05×SSC to about 0.5×SSC, about 0.05×SSC to about 0.1×SSC, about 0.1×SSC to about 1×SSC, about 0.1×SSC to about 0.5×SSC, or about 0.5×SSC to about 1×SSC). In some embodiments, SSC is present in the SSC wash buffer at about 0.01×SSC, about 0.02×SSC, about 0.03×SSC, about 0.04×SSC, about 0.05×SSC, about 0.06×SSC, about 0.07×SSC, about 0.08×SSC, about 0.09×SSC, about 0.1×SSC, about 0.2×SSC, about 0.3×SSC, about 0.4×SSC, about 0.5×SSC, about 0.6×SSC, about 0.7×SSC, about 0.8×SSC, about 0.9×SSC, or about 0.1×SSC. In some embodiments, SSC is present in the SSC wash buffer at about 0.1×SSC.

In some embodiments, the SSC wash buffer comprises a detergent. In some embodiments, the detergent comprises sodium dodecyl sulfate (SDS). In some embodiments, SDS is present in the SSC wash buffer from about 0.01% (v/v) to about 0.5% (v/v) (e.g., about 0.01% (v/v) to about 0.4% (v/v), about 0.01% (v/v) to about 0.3% (v/v), about 0.01% (v/v) to about 0.2% (v/v), about 0.01% (v/v) to about 0.1% (v/v), about 0.05% (v/v) to about 0.5% (v/v), about 0.05% (v/v) to about 0.4% (v/v), about 0.05% (v/v) to about 0.3% (v/v), about 0.05% (v/v) to about 0.2% (v/v), about 0.05% (v/v) to about 0.1% (v/v), about 0.1% (v/v) to about 0.5% (v/v), about 0.1% (v/v) to about 0.4% (v/v), about 0.1% (v/v) to about 0.3% (v/v), about 0.1% (v/v) to about 0.2% (v/v), about 0.2% (v/v) to about 0.5% (v/v), about 0.2% (v/v) to about 0.4% (v/v), about 0.2% (v/v) to about 0.3% (v/v), about 0.3% (v/v) to about 0.5% (v/v), about 0.3% (v/v) to about 0.4% (v/v), or about 0.4% (v/v) to about 0.5% (v/v)). In some embodiments, the SDS is present the SSC wash buffer at about 0.01% (v/v), about 0.02% (v/v), about 0.03% (v/v), about 0.04% (v/v), about 0.05% (v/v), about 0.06% (v/v), about 0.07% (v/v), about 0.08% (v/v), about 0.09% (v/v), about 0.10% (v/v), about 0.2% (v/v), about 0.3% (v/v), about 0.4% (v/v), or about 0.5% (v/v), In some embodiments, the SDS is present in the SSC wash buffer at about 0.1% (v/v). In some embodiments, sarkosyl may be present in the SSC wash buffer.

In some embodiments, the SSC wash buffer is at a temperature from about 50° C. to about 70° C. (e.g., about 50° C. to about 65° C., about 50° C. to about 60° C., about 50° C. to about 55° C., about 55° C. to about 70° C., about 55° C. to about 65° C., about 55° C. to about 60° C., about 60° C. to about 70° C., about 60° C. to about 65° C., or about 65° C. to about 70° C.). In some embodiments, the SSC wash buffer is at a temperature from about 55° C. to about 65° C. In some embodiments, the SSC wash buffer is at a temperature about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C. In some embodiments, the SSC wash buffer is at a temperature of about 60° C.

In some embodiments, the method includes releasing the ligation product, where releasing is performed after the array is washed to remove the one or more unhybridized first and second RTL probes.

### (p) Ligation

In some embodiments, after hybridization of the host or viral RTL probes (e.g., a first host or viral RTL probe, a second host or viral RTL probe, a spanning probe, additional spanning probes, and/or a third oligonucleotide) to the host or viral analyte, the host or viral RTL probe (e.g., a first host or viral RTL probe, a second host or viral RTL probe, a spanning probe, additional spanning probes, and/or a third oligonucleotide) can be ligated together, creating a single ligation product that includes one or more sequences that are complementary to the analyte. Ligation can be performed enzymatically or chemically, as described herein.

In some instances, the ligation is an enzymatic ligation reaction, using a ligase (e.g., T4 RNA ligase (Rnl2), a SplintR® ligase, a PBCV-1 DNA ligase, a Chlorella virus DNA ligase, a single stranded DNA ligase, or a T4 DNA ligase). See, e.g., Zhang et al.; RNA Biol. 2017; 14(1): 36-44, which is incorporated by reference in its entirety, for a description of KOD ligase. Following the enzymatic ligation reaction, the probes (e.g., a first RTL probe, a second RTL probe, a spanning probe, additional spanning probes, and/or a third oligonucleotide) may be considered ligated.

In some embodiments, a polymerase catalyzes synthesis of a complementary strand of the ligation product, creating a double-stranded ligation product. In some instances, the polymerase is DNA polymerase. In some embodiments, the polymerase has 5′ to 3′ polymerase activity. In some embodiments, the polymerase has 3′ to 5′ exonuclease activity for proofreading. In some embodiments, the polymerase has 5′ to 3′ polymerase activity and 3′ to 5′ exonuclease activity for proofreading.

In some embodiments, the host or viral RTL probe (e.g., a first host or viral RTL probe, a second host or viral RTL probe, a spanning probe, additional spanning probes, and/or a third oligonucleotide) may each comprise a reactive moiety such that, upon hybridization to the target and exposure to appropriate ligation conditions, the host or viral RTL probes may ligate to one another. In some embodiments, probes that include a reactive moiety are ligated chemically. For example, a first host or viral RTL probe capable of hybridizing to a first target region (e.g., a first target sequence or a first portion) of a nucleic acid molecule may comprise a first reactive moiety, and a second host or viral RTL probes capable of hybridizing to a second target region (e.g., a second target sequence or a second portion) of the nucleic acid molecule may comprise a second reactive moiety. When the first and second host or viral RTL probes are hybridized to the first and second target regions (e.g., first and second target sequences) of the nucleic acid molecule, the first and second reactive moieties may be adjacent to one another. A reactive moiety of a probe may be selected from the non-limiting group consisting of azides, alkynes, nitrones (e.g., 1,3-nitrones), strained alkenes (e.g., trans-cycloalkenes such as cyclooctenes or oxanorbornadiene), tetrazines, tetrazoles, iodides, thioates (e.g., phosphorothioate), acids, amines, and phosphates. For example, the first reactive moiety of a first RTL probe may comprise an azide moiety, and a second reactive moiety of a second RTL probe may comprise an alkyne moiety. The first and second reactive moieties may react to form a linking moiety. A reaction between the first and second reactive moieties may be, for example, a cycloaddition reaction such as a strain-promoted azide-alkyne cycloaddition, a copper-catalyzed azide-alkyne cycloaddition, a strain-promoted alkyne-nitrone cycloaddition, a Diels-Alder reaction, a [3+2] cycloaddition, a [4+2] cycloaddition, or a [4+1] cycloaddition; a thiol-ene reaction; a nucleophilic substation reaction; or another reaction. In some cases, reaction between the first and second reactive moieties may yield a triazole moiety or an isoxazoline moiety. A reaction between the first and second reactive moieties may involve subjecting the reactive moieties to suitable conditions such as a suitable temperature, pH, or pressure and providing one or more reagents or catalysts for the reaction. For example, a reaction between the first and second reactive moieties may be catalyzed by a copper catalyst, a ruthenium catalyst, or a strained species such as a difluorooctyne, dibenzylcyclooctyne, or biarylazacyclooctynone. Reaction between a first reactive moiety of a first RTL probe hybridized to a first target region (e.g., a first target sequence or first portion) of the nucleic acid molecule and a second reactive moiety of a third probe hybridized to a second target region (e.g., a first target sequence or a first portion) of the nucleic acid molecule may link the first RTL probe and the second RTL probe to provide a ligated probe. Upon linking, the first and second RTL probe may be considered ligated. Accordingly, reaction of the first and second reactive moieties may comprise a chemical ligation reaction such as a copper-catalyzed 5′ azide to 3′ alkyne “click” chemistry reaction to form a triazole linkage between two probes. In other non-limiting examples, an iodide moiety may be chemically ligated to a phosphorothioate moiety to form a phosphorothioate bond, an acid may be ligated to an amine to form an amide bond, and/or a phosphate and amine may be ligated to form a phosphoramidate bond.

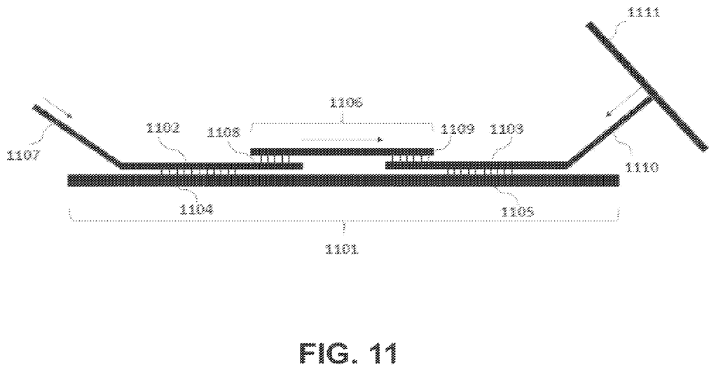

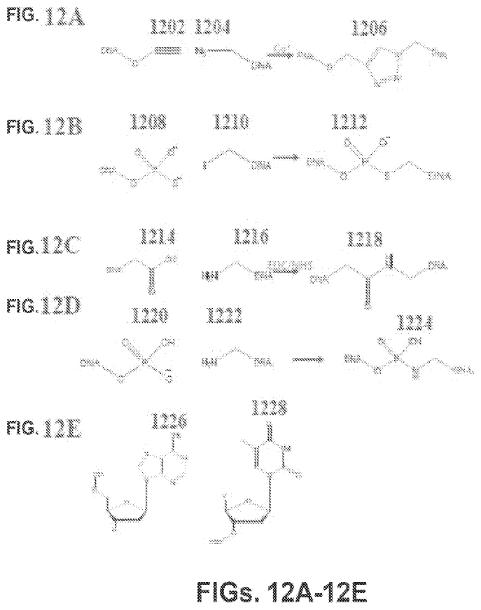

FIGS. 12A-12E illustrates examples of representative reactions. FIG. 12A shows a chemical ligation reaction of an alkyne moiety 1202 and an azide moiety 1204 reacting under copper-mediated cycloaddition to form a triazole linkage 1206. FIG. 12B shows a chemical ligation reaction of a phosphorothioate group 1208 with an iodide group 1210 to form a phosphorothioate linkage 1212. FIG. 12C shows a chemical ligation reaction of an acid 1214 and amine 1216 to form an amide linkage 1218. FIG. 12D shows a chemical ligation reaction of a phosphate moiety 1220 and an amine moiety 1222 to form a phosphoramidate linkage 1224. FIG. 12E shows a conjugation reaction of two species 1226 and 1228.

In some instances, ligation is performed in a ligation buffer. In instances where host or viral RTL probe ligation is performed on diribo-containing probes, the ligation buffer can include T4 RNA Ligase Buffer 2, enzyme (e.g., RNL2 ligase), and nuclease free water. In instances where probe ligation is performed on DNA probes, the ligation buffer can include Tris-HCl pH7.5, MnCl2, ATP, DTT, surrogate fluid (e.g., glycerol), enzyme (e.g., SplintR® ligase, a PBCV-1 DNA ligase, or a Chlorella virus DNA ligase), and nuclease-free water.

In some embodiments, the ligation buffer includes additional reagents. In some instances, the ligation buffer includes adenosine triphosphate (ATP) that is added during the ligation reaction. DNA ligase-catalyzed sealing of nicked DNA substrates is first activated through ATP hydrolysis, resulting in covalent addition of an AMP group to the enzyme. After binding to a nicked site in a DNA duplex, the ligase transfers the AMP to the phosphorylated 5′-end at the nick, forming a 5′-5′ pyrophosphate bond. Finally, the ligase catalyzes an attack on this pyrophosphate bond by the OH group at the 3′-end of the nick, thereby sealing it, whereafter ligase and AMP are released. If the ligase detaches from the substrate before the 3′ attack, e.g. because of premature AMP reloading of the enzyme, then the 5′ AMP is left at the 5′-end, blocking further ligation attempts. In some instances, ATP is added at a concentration of about 1 μM, about 10 μM, about 100 μM, about 1000 μM, or about 10000 μM during the ligation reaction.

In some embodiments, cofactors that aid in joining of the probes are added during the ligation process. In some instances, the cofactors include magnesium ions (Mg²⁺). In some instances, the cofactors include manganese ions (Mn²⁺). In some instances, Mg²⁺ is added in the form of MgCl₂. In some instances, Mn²⁺ is added in the form of MnCl₂. In some instances, the concentration of MgCl₂ is at about 1 mM, at about 10 mM, at about 100 mM, or at about 1000 mM. In some instances, the concentration of MnCl₂ is at about 1 mM, at about 10 mM, at about 100 mM, or at about 1000 mM.

In some embodiments, the ligation product includes a capture probe capture domain, which can bind to a capture probe (e.g., a capture probe immobilized, directly or indirectly, on a substrate). In some embodiments, methods provided herein include contacting a biological sample with a substrate, wherein the capture probe is affixed to the substrate (e.g., immobilized to the substrate, directly or indirectly). In some embodiments, the capture probe capture domain of the ligated probe specifically binds to the capture domain.

After ligation, in some instances, the biological sample is washed with a post-ligation wash buffer. In some instances, the post-ligation wash buffer includes one or more of SSC (e.g., 1×SSC), formamide, and nuclease free water. In some instances, the biological sample is washed at this stage at about 50° C. to about 70° C. In some instances, the biological sample is washed at about 60° C.

### (i) Ligation Including Pre-Adenylated 5′ Phosphate on Second RTL Probe

Provided herein are methods for determining a location of a host or viral nucleic acid in a biological sample that include: (a) contacting the biological sample with a substrate comprising a plurality of host or viral capture probes, where a capture probe of the plurality of capture probes comprises a capture domain and a spatial barcode; (b) hybridizing a target nucleic acid in the biological sample with a first host or viral RTL probe and a host or viral second RTL probe, where the first RTL probe comprises, from 3′ to 5′, a sequence substantially complementary to the capture domain and a sequence that is substantially complementary to a first sequence in the target nucleic acid and has a pre-adenylated phosphate group at its 5′ end; the second RTL probe comprises a sequence substantially complementary to a second sequence in the target nucleic acid; (c) generating a host or viral ligation product by ligating a 3′ end of the second RTL probe to the 5′ end of the first RTL probe using a ligase that does not require adenosine triphosphate for ligase activity; (d) releasing the host or viral ligation product from the target nucleic acid and binding the capture domain of the ligation product specifically to the capture domain of capture probe; and (e) determining (i) all or a part of a sequence corresponding to the host or viral ligation product, or a complement thereof, and (ii) the spatial barcode, or a complement thereof, and using the determined sequences of (i) and (ii) to identify the location of the host or viral nucleic acid in the biological sample

In some instances, the ligase that does not require adenosine triphosphate for ligase activity (e.g., thermostable 5′ AppDNA/RNA Ligase, truncated T4 RNA Ligase 2 (trRnl2), truncated T4 RNA Ligase 2 K227Q, truncated T4 RNA Ligase 2 KQ, Chlorella Virus PBCV-1 DNA Ligase, and combinations thereof). See, e.g., Nichols et al., “RNA Ligases,” Curr. Protocol. Molec. Biol. 84(1):3.15.1-.4 (2008); Viollet et al., “T4 RNA Ligase 2 Truncated Active Site Mutants: Improved Tools for RNA Analysis,” BMC Biotechnol. 11: 72 (2011); and Ho et al., “Bacteriophage T4 RNA Ligase 2 (gp24.1) Exemplifies a Family of RNA Ligases Found in All Phylogenetic Domains,” PNAS 99(20):12709-14 (2002), which are hereby incorporated by reference in their entirety for a description of T4 RNA Ligases and truncated T4 RNA Ligases. Thermostable 5′ AppDNA/RNA Ligase is an enzyme belonging to the Ligase family that catalyzes the ligation of the 3′ end of ssRNA or ssDNA to a 5′-adenylated ssDNA or 5′-adenylated ssRNA. Truncated T4 RNA Ligase 2 is an enzyme belonging to the Ligase family that catalyzes the ligation of dsRNA nicks and ssRNA to ssRNA. It can also ligate the 3′ end of RNA or DNA to a 5′-pDNA when annealed to an RNA complement, and the 3′ end of RNA to a 5′-pRNA when annealed to a DNA complement, with reduced efficiency. Truncated T4 RNA Ligase 2 K227Q is an enzyme belonging to the Ligase family that catalyzes the ligation of the 3′ end of ssRNA to 5′ adenylated ssDNA and 5′ adenylated ssRNA. It has a reduction of side products as compared to truncated T4 RNA Ligase 2. Truncated T4 RNA Ligase 2 KQ is an enzyme belonging to the Ligase family that catalyzes the ligation of the 3′ end of ssRNA to 5′ adenylated ssDNA and 5′ adenylated ssRNA. It is a preferred choice for ligation of ssRNA to preadenylated adapters and has a reduction of side products as compared to truncated T4 RNA Ligase 2.

In some embodiments, the T4 RNA Ligase comprises a K227Q mutation. See Viollet et al., “T4 RNA Ligase 2 Truncated Active Site Mutants: Improved Tools for RNA Analysis,” BMC Biotechnol. 11, which is hereby incorporated by reference in its entirety.

In some instances, cofactors that aid in ligation of the first and second host or viral RTL probe are added during ligation. In some instances, the cofactors include magnesium ions (Mg²⁺). In some instances, the cofactors include manganese ions (Mn²⁺). In some instances, Mg²⁺ is added in the form of MgCl₂. In some instances, Mn²⁺ is added in the form of MnCl₂. In some instances, the concentration of MgCl₂ is at about 1 mM to about 10 mM. In some instances, the concentration of MnCl₂ is at about 1 mM to about 10 mM.

In some instances, the ligation occurs at a pH in the range of about 6.5 to about 9.0, about 6.5 to about 8.0, or about 7.5 to about 8.0.

In some embodiments, the ligation buffer includes an enzyme storage buffer. In some embodiments, the enzymes storage buffer includes glycerol. In some embodiments, the ligation buffer is supplemented with glycerol. In some embodiments, the glycerol is present in the ligation buffer at a total volume of 15% v/v.

(q) Permeabilization of the Sample and Releasing the Host and/or Viral Ligation Products

In some embodiments, the methods provided herein include a permeabilizing step in order to allow the ligated host and/or viral RTL probe (e.g., comprising a sequence that includes the complement of a host or viral analyte) to migrate through the biological sample to the capture probe on the substrate. In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, after creating a host and/or viral ligation product (e.g., by ligating a first host or viral RTL probe and a second host or viral RTL probe that are hybridized to adjacent sequences in the host or viral analyte), the biological sample is permeabilized. In some embodiments, the biological sample is permeabilized contemporaneously with or prior to contacting the biological sample with a first host and/or viral RTL probe and a host and/or viral second RTL probe.

In some embodiments, methods provided herein include permeabilization of the biological sample such that the ligated probe can migrate more freely through a biological sample and hybridize to the capture domain of the capture probe (i.e., compared to no permeabilization).

In some instances, the permeabilization step includes application of a permeabilization buffer to the biological sample. In some instances, the permeabilization buffer includes a buffer (e.g., Tris pH 7.5), MgCl2, sarkosyl detergent (e.g., sodium lauroyl sarcosinate), enzyme (e.g., proteinase K), and nuclease free water. In some instances, the permeabilization step is performed at 37° C. In some instances, the permeabilization step is performed for about 20 minutes to 2 hours (e.g., about 20 minutes, about 30 minutes, about 40 minutes, about 50 minutes, about 1 hour, about 1.5 hours, or about 2 hours). In some instances, permeabilization of a biological sample is performed for about 40 minutes.

In some embodiments, after generating a host and/or viral ligation product, the ligation product is released from the host and/or viral analyte. In some embodiments, a host and/or viral analyte is digested using an analyte, releasing a host and/or viral ligation product from the host and/or viral analyte. In some embodiments, the endoribonuclease is RNase H, RNase A, RNase C, or RNase I. In some embodiments, the endoribonuclease is RNase H. RNase H is an endoribonuclease that specifically hydrolyzes the phosphodiester bonds of RNA, when hybridized to DNA thereby freeing the DNA from the hybrid RNA:DNA construct and allowing the freed DNA strand (i.e., ligation product) to migrate to the substrate for hybridization to the capture domain of the capture probe. RNase H is part of a conserved family of ribonucleases which are present in many different organisms. There are two primary classes of RNase H: RNase H1 and RNase H2. Retroviral RNase H enzymes are similar to the prokaryotic RNase H1. All of these enzymes share the characteristic that they are able to cleave the RNA component of an RNA:DNA heteroduplex. In some embodiments, the RNase H is RNase H1, RNase H2, or RNase H1, or RNase H2. In some embodiments, the RNase H includes but is not limited to RNase HII from Pyrococcus furiosus, RNase HII from Pyrococcus horikoshi, RNase HI from Thermococcus litoralis, RNase HI from Thermus thermophilus, RNAse HI from E. coli, or RNase HII from E. coli.

In some instances, digestion of the analyte to release the ligation product is performed using an endonuclease digestion buffer. In some instances, the endonuclease digestion buffer includes one or more of a buffer (e.g., Tris pH 7.5), enzyme (e.g., RNAse H) and nuclease-free water. In some instances, the digestion step is performed at 37° C. In some instances, the analyte digestion step is performed for about 20 minutes to 2 hours (e.g., about 20 minutes, about 30 minutes, about 40 minutes, about 50 minutes, about 1 hour, about 1.5 hours, or about 2 hours). In some instances, the analyte digestion step is performed for about 30 minutes.

In some instances, the analyte digestion step occurs before the permeabilization step. In some instances, the analyte digestion step occurs after the permeabilization step. In some instances, the analyte digestion step occurs at the same time as the permeabilization step (e.g., in the same buffer).

### (r) Biological Samples

Methods disclosed herein can be performed on any type of sample (also called a “biological sample” and is used interchangeably herein). In some embodiments, the sample is a fresh tissue or cell sample. In some embodiments, the sample is a frozen tissue or cell sample. In some embodiments, the tissue or cell sample was previously frozen. In some embodiments, the tissue sample is a formalin-fixed, paraffin embedded (FFPE) tissue or cell sample.

Subjects from which biological samples can be obtained can be healthy or asymptomatic individuals, individuals that have or are suspected of having a disease (e.g., a SARS-CoV-2 infection) or a pre-disposition to a disease (e.g., a SARS-CoV-2 infection), and/or individuals that are in need of therapy or suspected of needing therapy. In some instances, the biological sample is from a subject suspected of having a SARS-CoV-2 infection. In some instances, the biological sample is from a subject confirmed with having a SARS-CoV-2 infection. In some instances, the SARS-CoV-2 infection is from an infection caused by a SARS-CoV-2 variant. In some instances, the biological sample is from a subject confirmed with having a SARS-CoV-1, SARS-CoV-2, or Middle East Respiratory Syndrome (MERS) infection.

In some instances, the biological sample is a nasal sample, a nasopharyngeal sample, an oral sample, or a lung tissue sample. In some instances, the biological sample is a lung tissue sample.

In some instances, the biological sample is a tissue sample other than lung. For instance, in some embodiments, the biological sample is a post-mortem heart sample. In some instances, the biological samples are post-mortem samples are assayed to determine the presence of SARS-CoV-2, or to support the diagnosis of a subject having SARS-CoV-2, in a subject who is deceased.

Subjects from which biological samples can be obtained can be healthy or asymptomatic individuals, individuals that have or are suspected of having a disease (e.g., a SARS-CoV-2 infection) or a pre-disposition to a disease (e.g., a SARS-CoV-2 infection), and/or individuals that are in need of therapy or suspected of needing therapy. In some instances, the biological sample can include one or more diseased cells (e.g., a SARS-CoV-2 infected cell). A diseased cell can have altered metabolic properties, gene expression, protein expression, and/or morphologic features. In some instances, the biological sample is a heterogenous sample. In some instances, the biological sample is a heterogenous sample one or more sites of SARS-CoV-2 infection. In some instances, the biological sample comprises nucleic acids with one or more genetic variants. In some instances, the one or more genetic variants is associated with a disease or disease state.

In some instances, the subject can be an animal. In some embodiments, the subject is a mammal such as a non-primate (e.g., cow, pig, horse, cat, dog, rat, etc.) or a primate (e.g., monkey or human). In some instances, the subject is a domesticated animal (e.g., a dog or cat). In some instances, the subject is a bat. In some instances, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

(s) Determining the Sequence of the Host and/or Viral Ligation Product

After a target analyte proxy such as a host and/or viral ligation product has hybridized or otherwise been associated with a capture probe according to any of the methods described herein in connection with the general spatial cell-based analytical methodology, the spatially barcoded constructs that result from hybridization/association are analyzed.

In some embodiments, after contacting a biological sample with a substrate that includes capture probes, a removal step can optionally be performed to remove all or a portion of the biological sample from the substrate after the ligation products are hybridized or otherwise associated with the capture probes on the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein) in order to create a copy of the host or viral ligated probe. This process involves synthesis of a complementary strand of the host or viral ligated probe, e.g., generating a full length complement of the host or viral ligation product.

In some embodiments, a capture domain of a capture probe includes a primer for producing the complementary strand of a host or viral ligated probe hybridized to the capture probe, e.g., a primer for DNA polymerase. The nucleic acid, e.g., DNA and/or host or viral ligated probes or complement thereof, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA molecule is generated. In some embodiments, a “full-length” DNA molecule refers to the whole of the captured host or viral ligated product. For example, the full length DNA molecule refers to the whole of the host or viral ligation product released following RNase H digestion of the RNA from the RNA:DNA duplex (e.g., releasing the ligation probe from the RNA:DNA duplex).

In some embodiments, double-stranded extended capture probes (now containing the ligated probe) are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes containing the complement of the host or viral ligated probe are amplified to yield quantities that are sufficient for analysis, e.g., via DNA sequencing. In some embodiments, the host or viral extended ligation products (e.g., the capture probe plus the host or viral ligated probe) act as a template for the amplification reaction (e.g., a polymerase chain reaction).

In some embodiments, the amplification reaction incorporates an affinity group onto the host or viral extended ligation product using a primer including the affinity group. In some embodiments, the primer includes an affinity group and the host or viral extended ligation product includes the affinity group. The affinity group can correspond to any of the affinity groups described previously.

In some embodiments, the host or viral extended ligation products include the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended ligation products can function to release the products from the substrate, insofar as copies of the host or viral ligation extended products are not immobilized on the substrate.

In some embodiments, the host or viral extended ligation product or complement or amplicon thereof is released. The step of releasing from the surface of the substrate can be achieved in a number of ways. In some embodiments, an extended ligation product or a complement thereof is released from the array by nucleic acid cleavage and/or by denaturation (e.g., by heating to denature a double-stranded molecule).

In some embodiments, the host or viral extended ligation product or complement or amplicon thereof is released from the surface of the substrate (e.g., array) by physical means. For example, where the host or viral extended ligation product is indirectly immobilized on the array substrate, e.g., via hybridization to a surface probe, it can be sufficient to disrupt the interaction between the host or viral extended ligation product and the surface probe. Methods for disrupting the interaction between nucleic acid molecules include denaturing double stranded nucleic acid molecules are known in the art. A straightforward method for releasing the DNA molecules (i.e., of stripping the array of extended products) is to use a solution that interferes with the hydrogen bonds of the double stranded molecules. In some embodiments, the extended capture product is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended ligation product from the substrate.

In some embodiments, probes complementary to the host or viral extended ligation product can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to a host or viral extended ligation product can be washed away. In some embodiments, probes complementary to the host or viral extended ligation product can be detected on the substrate (e.g., imaging, any of the detection methods described herein).

In some embodiments, probes complementary to a host or viral extended ligation products can be about 4 nucleotides to about 100 nucleotides long. In some embodiments, probes (e.g., detectable probes) can be about 10 nucleotides to about 90 nucleotides long. In some embodiments, probes (e.g., detectable probes) can be about 20 nucleotides to about 80 nucleotides long. In some embodiments, probes (e.g., detectable probes) can be about 30 nucleotides to about 60 nucleotides long. In some embodiments, probes (e.g., detectable probes) can be about 40 nucleotides to about 50 nucleotides long. In some embodiments, probes (e.g., detectable probes) can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to a host or viral extended ligation product. In some embodiments, about 1 to about 10 probes can be contacted to the substrate and specifically bind (e.g., hybridize) to a host or viral extended ligation product. In some embodiments, about 10 to about 100 probes can be contacted to the substrate and specifically bind (e.g., hybridize) to an extended ligation product. In some embodiments, about 20 to about 90 probes can be contacted to the substrate. In some embodiments, about 30 to about 80 probes (e.g., detectable probes) can be contacted to the substrate. In some embodiments, about 40 to about 70 probes can be contacted to the substrate. In some embodiments, about 50 to about 60 probes can be contacted to the substrate. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to a host or viral extended ligation product.

In some embodiments, the probes can be complementary to a host or viral ligation product of a single analyte (e.g., a single gene). In some embodiments, the probes can be complementary to one or more host or viral ligation products (e.g., derived from analytes in a family of genes). In some embodiments, the probes (e.g., detectable probes) can be directed to ligation products for a panel of genes associated with a disease (e.g., SARS-CoV-2 infection).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

Methods disclosed herein can be used to help identify a cluster of expressed host and/or viral analytes. In some instances, the cluster includes both host analytes and viral analytes. Non-limiting examples of such methods include nonlinear dimensionality reduction methods such as t-distributed stochastic neighbor embedding (t-SNE), global t-distributed stochastic neighbor embedding (g-SNE), and uniform manifold approximation and projection (UMAP).

Any number of clusters can be identified. In some embodiments, 2 to 500 clusters can be identified using the methods as described herein. For example, 2 to 10, 2 to 20, 2 to 50, 2 to 75, 2 to 100, 2 to 150, 2 to 200, 2 to 300, 2 to 400, 400 to 500, 300 to 500, 200 to 500, 100 to 500, 75 to 500, 50 to 500, or 25 to 200 clusters can be identified. In some embodiments, 25 to 75, 50 to 100, 50 to 150, 75 to 150, or 100 to 200 clusters can be identified.

Any number of nucleic acids can be sorted into a cluster. For example, a cluster can include about 1 to about 200,000 nucleic acids. In some embodiments, a cluster can include about 1 to about 150,000, about 1 to about 100,000, about 1 to about 75,000, about 1 to about 50,000, about 100,000 to about 200,000, or about 50,000 to about 200,000 nucleic acids. In some embodiments, a cluster includes about 2 to about 25,000 nucleic acids. For example, about 2 to about 50, about 2 to about 100, about 2 to about 500, about 2 to about 1,000, about 2 to about 5,000, about 2 to about 10,000, about 2 to about 15,000, about 2 to about 20,000, about 20,000 to about 25,000, about 15,000 to about 25,000, about 10,000 to about 25,000, about 5,000 to about 25,000, about 1,000 to about 25,000, about 500 to about 25,000, or about 100 to about 25,000 nucleic acids.

In some embodiments, a nucleic acid included in a cluster is different than each of the other nucleic acids in the cluster. For example, the nucleic acid has a sequence that is not identical to any of the other nucleic acids in the cluster. In some embodiments, a nucleic acid corresponds to a gene.

### (t) Kits

In some embodiments, also provided herein are kits that include one or more reagents to detect one or more host or viral (e.g., SARS-CoV-2) analytes described herein. In some instances, the kit includes a substrate comprising a plurality of capture probes comprising a spatial barcode and the capture domain. In some instances, the kit includes a plurality of probes (e.g., a first RTL probe, a second RTL probe, one or more spanning probes, and/or a third oligonucleotide).

A non-limiting example of a kit used to perform any of the methods described herein includes: (a) a substrate comprising a plurality of capture probes comprising a spatial barcode and a capture domain; (b) a system comprising: a plurality of first viral (e.g., SARS-CoV-2) probes and second viral (e.g., SARS-CoV-2) probes, wherein a first viral (e.g., SARS-CoV-2) probe and a second viral (e.g., SARS-CoV-2) probe each comprises sequences that are substantially complementary to a viral analyte (e.g., a SARS-CoV-2 analyte or nucleic acid), and wherein the second RTL probe comprises a capture binding domain; (c) a system comprising: a plurality of first host (e.g., human-specific) probes and second host (e.g., human-specific) probes, wherein a first host (e.g., human-specific) probe and a second host (e.g., human-specific) probe each comprises sequences that are substantially complementary to a host analyte (e.g., a human analyte or nucleic acid), and wherein the second RTL probe comprises a capture binding domain; and (d) instructions for performing any of the methods disclosed herein.

In some instances, the kit further includes one or more of enzymes (e.g., ligase, polymerase, reverse transcriptase and reagents necessary to perform any of the methods disclosed herein.

In some embodiments of any of the kits described herein, the kit includes a second RTL probe that includes a preadenylated phosphate group at its 5′ end and a first RTL probe comprising at least two ribonucleic acid bases at the 3′ end.

In some embodiments, also provided herein are kits that include one or more reagents to detect a level of one or more of any of the biomarkers and/or candidate biomarkers described herein (e.g., IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LAS1L, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof).

In some embodiments, reagents can include one or more antibodies (and/or antigen-binding antibody fragments), labeled hybridization probes, and primers. For example, in some embodiments, an antibody (and/or antigen-binding antibody fragment) can be used for visualizing one or more features of a tissue sample (e.g., by using immunofluorescence or immunohistochemistry). In some embodiments, an antibody (and/or antigen-binding antibody fragment) can be an analyte binding moiety, for example, as part of an analyte capture agent. Useful commercially available antibodies will be apparent to one skilled in the art.

In some embodiments, labeled hybridization probes can be used for in situ sequencing of one or more biomarkers and/or candidate biomarkers. In some embodiments, primers can be used for amplification (e.g., clonal amplification) of a captured oligonucleotide analyte.

In some embodiments, a kit can further include instructions for performing any of the methods or steps provided herein.

### Additional Sequence(s)

Severe acute respiratory syndrome coronavirus 2 isolate Wuhan-Hu-1, complete genome is provided herein as an exemplary SARS-CoV-2 genomic sequence. Any variants of the SARS-CoV-2 genomic sequence are also disclosed herein. The NCBI Reference Sequence, NC_045512.2, is set forth in SEQ ID NO:1. Another NCBI Sequence is GCA_009858895.3, and is set forth in SEQ ID NO: 83.

### EXAMPLES

### Example 1. SARS-CoV-2 Detection and Spatial Gene Expression Analysis of FFPE-Fixed Samples Using Templated Ligation

As an overview, templated ligation on an FFPE-fixed sample was performed as described in FIG. 13. FFPE-fixed samples were deparaffinized, stained (e.g., H&E stain), and imaged 1301. Samples were destained (e.g., using HCl) and decrosslinked 1302. Following decrosslinking, samples were treated with a pre-hybridization buffer (e.g., a buffer comprising phosphate-buffered saline (PBS), polysorbate 20 (e.g., Tween®-20), nuclease free water, and proteinase K), probes were added to the sample, probes were allowed to hybridize to their targets, and samples were washed 1303. Probes added at 1303 included both SARS-CoV-2 probes as listed in Table 2, and human host RTL probes that targeted a subset of the human transcriptome. Ligase was added to the samples to ligate hybridized probes to generate a ligation product and samples were washed 1304. Probes were released from the analyte by contacting the biological sample with RNase H 1305. Samples were permeabilized to facilitate capture of the ligation product by the capture probes on the substrate 1306. Ligation products that hybridized to the capture probes were extended 1307. The extended capture probes were denatured 1308. Denatured, extended capture probes were amplified and indexed thereby creating a sequencing library and the sequencing library was subjected to quality control 1309 before being sequenced.

The biological samples used in this experiment included human lung tissue from a subject diagnosed with SARS-CoV-2 infection before death (n=1) and on a subject with no detected SARS-CoV-2 infection before death (n=1).

FFPE sectioned human lung tissue slides were deparaffinized, stained (e.g., H&E stain), imaged, and then slides were placed into a cassette and subjected to the methods described in Table 4. Decrosslinking the sample was performed using a TE decrosslinking reagent. For TE decrosslinking, 100 μl of TE buffer (pH 9.0) (Genemed 10-0046) was added per sample. Samples treated with TE were subjected to a thermal cycler protocol according to Table 3 for Step 6 of the method in Table 4.

TABLE 3
Decrosslinking Thermal Cycler Protocol
Decrosslinking: Lid Temp: 70 C., Volume: 100 uL

| Step | Temp | Time |
|---|---|---|
| Pre-heat | 70° C. | ∞ |
| Decrosslinking | 70° C. | 60 minutes |
| Hold | 22° C. | ∞ |

Following the thermal cycler protocol, the TE buffer (pH 9.0) was removed and 100 μl of pre-Hyb buffer was added per sample. Samples were incubated with a pre-Hyb buffer for 15 minutes at room temperature (step 8 of Table 4).

TABLE 4
Decrosslinking with TE*

| Step | Timing | |
|---|---|---|
| 1. Wash 1: Added 100 μl 0.1N HCl | 1 | minute |
| 2. Wash 2: Removed the HCl and add 100 μl HCl | 1 | minute |
| 3. Wash 3: Removed the HCl and add 100 μl HCl | 1 | minute |
| 4. Buffer exchange: Removed HCl and add 100 μl TE | | |
| buffer (pH 9.0) | | |
| 5. Removed TE and add 100 μl TE (pH 9.0). | 1 | hour |
| 6. Incubated at 70° C. | | |
| 7. Removed TE | 15 | minutes |
| 8. Incubated with pre-Hyb at room temperature | | |

*Note:
All liquid was removed at each step.

Templated ligation probes were designed to hybridize to adjacent sequences of viral (e.g., SARS-CoV-2) and hosts analytes (e.g., mRNA sequence), including estrogen receptor, progesterone receptor, and ERBB2, also known as HER2. Either (A) 20,056 host RTL probe pairs or (B) SARS-CoV-2 RTL probe pairs (as shown in Table 2; e.g., Surface glycoprotein (S), Envelope protein (E), Nucleocapsid phosphoprotein (N), Membrane glycoprotein (M), ORF1ab, ORF3a, ORF7a, ORF7b, ORF8, and ORF10) in conjunction with the 20,056 host RTL probe pairs were added to each tissue sample. For quality control, certain probe pairs directed to specific targets such as hemoglobin genes (e.g., HBQ1, HBA1, and HBA2) were omitted from analysis. The 20,056 host RTL probe pairs hybridize to 19,490 different genes. Two templated ligation probes (a left-hand side (LHS) probe and a right-hand side (RHS) probe (see e.g., FIGS. 6, 601 and 604) for each analyte were added simultaneously and hybridized to adjacent sequences of the target mRNA, forming RNA:DNA duplex structures. Following decrosslinking and pre-Hyb, the pre-Hyb buffer was removed and the DNA probes (1.2 nM of each probe) were added to the tissue samples in a hybridization buffer for hybridizing the DNA probes to their respective mRNA targets. The hybridization buffer included SSC, formamide or an equivalent, yeast tRNA as carrier, Denhardt's solution, nuclease free water, and the DNA RTL probe pairs.

One probe of the probe pair comprises a non-target functional sequence at its 5′ end while the other probe of the probe pair comprises a non-target polyA sequence at its 3′ end (see FIGS. 6, 602 and 606). The DNA probes were added to the tissue samples and incubated at 50° C. overnight (16-24 hours) according to thermal cycler protocol described in Table 5.

TABLE 5
Hybridization Protocol
Hybridization Thermal Cycler Protocol
Hyb: Lid Temp: 50 C., Volume: 100 uL

| Step | Temp | Time |
|---|---|---|
| Pre-heat | 50° C. | ∞ |
| Hybridize | 50° C. | overnight |
| | | (16-24 hours) |
| Hold for post hybridization washes | 50° C. | ∞ |

Following incubation at 50° C. overnight, the hybridization buffer was removed and the tissue was washed three times for 5 minutes at 50° C. with post-hybridization wash buffer that was pre-heated to 50° C. Post-hybridization wash buffer (Post-hyb buffer) included: SSC, yeast tRNA, and nuclease free water. Post-hyb buffer was removed, 2×SSC added, and the slide cooled to room temperature before proceeding to probe ligation.

To ligate two DNA host and/or viral RTL probes that adjacently hybridized on the target analytes as described above, SplintR® (NEB) in a ligation buffer was added to each sample. The ligase reaction mixture included: Tris-HCl (pH 7.5), MnCl₂, ATP, DTT, surrogate fluid, SplintR® ligase (NEB), and nuclease free water. 60 μl ligase reaction mixture was added per sample. The ligation and wash steps were performed as described in Table 6.

Following host and/or viral RTL probe ligation the tissue samples were washed twice for 5 minutes at 57° C. in a SSC/formamide post ligation wash buffer.

TABLE 6
Ligation Protocol
Probe Ligation

| Step | Time | |
|---|---|---|
| Removed all 2X SSC before adding Ligase Mix | | |
| Added Ligase Mix and Incubated at 37° C. | 1 | h |
| Wash 1: Removed Ligase Mix, added post-ligation | 5 | min |
| wash buffer*, and incubated at 57° C. for 5 minutes | | |
| Remaining wash buffer at heated to 57° C. | | |
| Wash 2: Removed post-ligation wash buffer, added | 5 | min |
| pre-heated post-ligation buffer and incubated | | |
| at 57° C. for 5 minutes. | | |
| Removed post-ligation wash buffer and added | | |
| 2X SSC. Repeated 2X SSC wash. | | |

*Post ligation wash buffer included (per sample): SSC, formamide or an equivalent, and nuclease free water.

Once the cassette had cooled to room temperature, RNase H was added to digest the RNA strand of the hybridized RNA:DNA duplex. Briefly, the RNA of the DNA:RNA hybrids was digested by incubating the samples with RNase H mix for 30 minutes at 37° C. Following the incubation and while the same remained at 37° C., the biological sample was permeabilized to release the ligated probes using 1.25 mg/mL Proteinase K. In particular, the Proteinase K solution included (per sample): Tris (pH 7.5), NaCl, Sarkosyl, or SDS, Proteinase K (Enzyme), and nuclease free water. The sample was incubated at 37° C. for 40 minutes in the Proteinase K solution. The samples were then washed three times with 2×SSC.

The released, ligated DNA probes that served as a proxy for the target mRNA were allowed to hybridize to the capture domain on the capture probe immobilized on the spatial array via the polyA tail on the 3′ end of one probe in the probes of the probe pair. The captured ligated probes were copied, using the capture probe as a template and the extension product was released from the spatial array. Briefly, the tissues were incubated with an extension mix comprising Kapa HiFi DNA polymerase for 15 minutes at 45° C. Following incubation, the extension mix was removed from the tissues and the tissues were washed with 2×SSC. A solution of KOH was added to the tissues, the tissues were incubated at room temperature for 10 minutes to release the extension product from the spatial array and the supernatant from each tissue sample was transferred off the array for quantification and library preparation. Sample quantification was performed using qPCR and KAPA SYBR FAST qPCR master mix according to the manufacturer's instructions. Briefly, KAPA SYBR master mix was prepared by adding qPCR primer cDNA F and qPCR primer sRNA_R2. The thermal cycler protocol included: 3 minutes at 98° C.; 30 cycles with 5 seconds at 98° C., and 30 seconds at 63° C. For library preparation, samples were indexed using an Amp Mix that included dual indexing primers and an Amp Mix.

Nucleic acids were then sequenced and analyzed. As an exemplary method, sequencing libraries were pooled and diluted with Elution Buffer (EB) to a final concentration of 10 nM, using a target sequencing depth of 50,000 mean read pairs/spot to determine the dilution for each sample. After sample pooling, pooled library concentrations were checked with qPCR (Bio-Rad) before loading into the NovaSeq (Illumina) sequencer for paired-end, dual indexed sequencing (run type and parameters [R1: 28 cycles, R2S: 50-52 cycles], with a spike-in of PhiX at 1% concentration, except one sample, P3CL, was run with R2S: 75 cycles).

TABLE 7
Results of Detection of Human and Viral RTL probes

| | | | Total |
|---|---|---|---|
| | Median | Median | Targeted |
| | Targeted | Targeted | Human |
| Sample and Probes | Genes | UMI Counts | Genes |
| Added to Sample | per Spot | per Spot | Detected* |
| Replicate 1 (Human only) | 3,286 | 7,812 | 18,419 |
| Replicate 2 (Human only) | 2,958 | 6,465 | 18,295 |
| Replicate 1 (H + V)⁺ | 2,453 | 5,027 | 17,848 |
| Replicate 2 (H + V)⁺ | 3,146 | 7,005 | 18,277 |

*Transcriptome-wide
⁺Human and Viral RTL probes Added to Sample

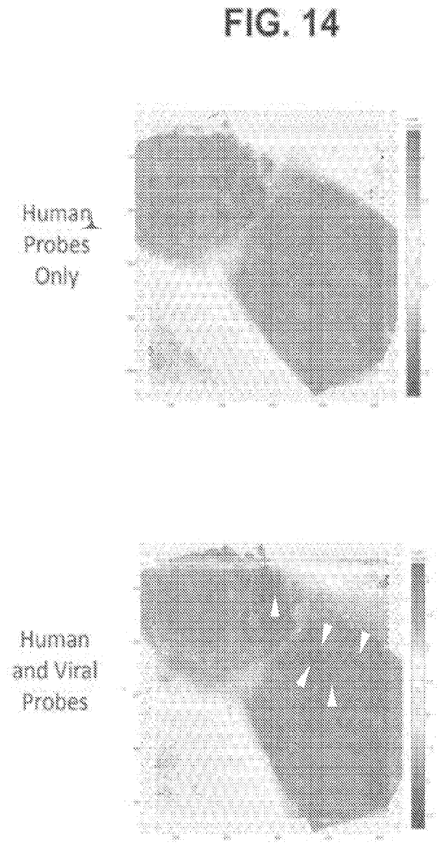

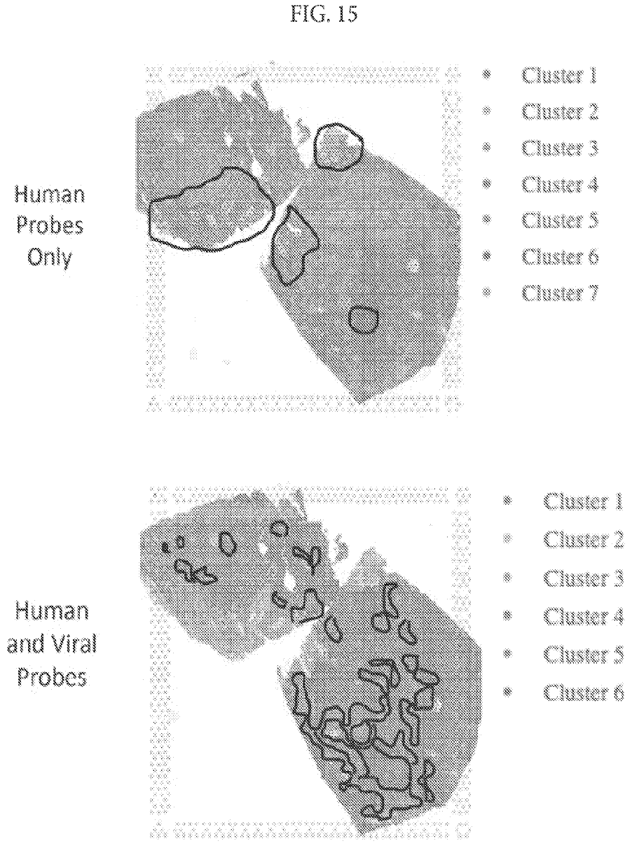

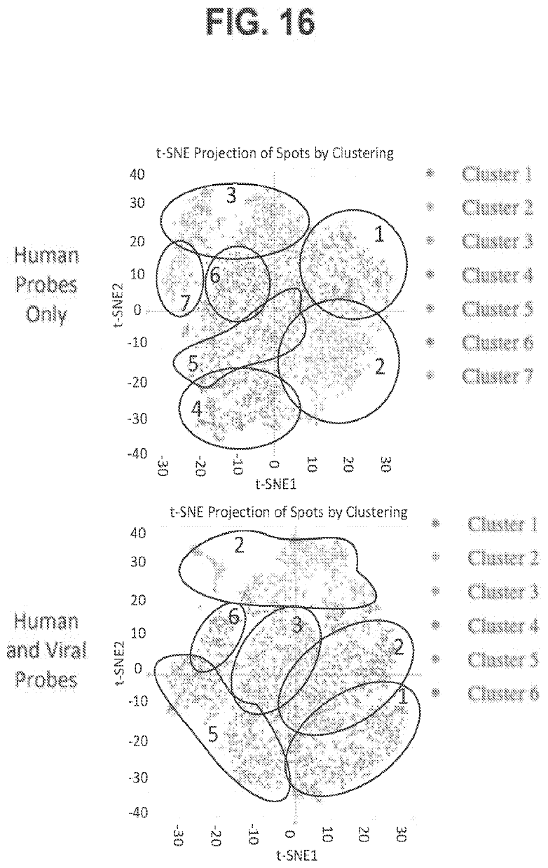

It was first determined whether both viral RTL probes and host RTL probes can be detected from the same experiment on the same sample. Two replicates from human lung samples from the subject diagnosed with SARS-CoV-2 infection before death were examined for quality of detected ligated probes. As shown in Table 7, the median targeted genes per spot, median targeted UMI counts per spot, and the total targeted genes detected in the human transcriptome showed no significant difference between the human only cohort of probes or the combined viral and human probes. Representative tissue images showing UMI counts were generated for each cohort to assess relative detection of the human ligated probes at each spot on the spatial array. FIG. 14, top picture, shows lung tissue gene expression UMI counts when only human transcriptome probes were used for identifying gene expression patterns. FIG. 14, bottom picture, shows lung tissue gene expression UMI counts when SAR-CoV-2 targeted probes were added to the human transcriptome probe set. The white arrows point to locations of increased gene expression via UMI counts where both human and SARS-CoV-2 gene expression was seen in this tissue sample. Further analysis of spatial location and abundance of each templated ligation probe that hybridized to the array revealed six or seven different clusters of host (e.g., human) gene expression, demonstrating that differential gene expression and location of the expressed genes can be determined using templated ligation probes, even in the presence of viral RTL probes. Some clusters were identified as physically clustered in the tissue image (e.g. cluster 4 of the human probes only image), others were more identified throughout the tissue (e.g. cluster 3 of the human and viral probes image). See FIGS. 15-16.

A collective total of five patient lung tissue samples, three from COVID-positive patients and two from COVID-negative patients, (including the samples shown in FIGS. 14-16) were examined for abundance of gene expression and detection of viral analytes using the methods described in this example. RNA quality from each sample was examined and confirmed to be consistent among samples with respect to both RNA integrity number and DV₂₀₀ (i.e., the percentage of RNA fragments>200 nucleotides) (data not shown). Human samples from autopsy cases were selected from patients who were hospitalized because of COVID-19 infection and subsequently died. Criteria for selection included: premortem positive (COVID positive cases) or negative (COVID negative cases) as found by SARS-CoV-2 PCR test, lack of malignancy of the lung, and less than 24 hours postmortem interval (PMI). All human samples were obtained, tested and de-identified as required per IRB protocols.

Relevant clinical parameters of the patients included in this study are summarized in Table 8.

TABLE 8
COVID-positive and COVID-negative patients.

| | | Survival after | | |
|---|---|---|---|---|
| | | COVID | Postmortem | Fixation |
| Patient | COVID/ | diagnosis | interval | time |
| ID | Control | (days) | (hours) | (hours) |
| P1 | COVID | 15 | 17 | 24 |
| P2 | COVID | 13 | 21 | 24 |
| P3 | COVID | 17 | 13 | 6 |
| P4 | Control | — | 21 | overnight |
| P5 | Control | — | 15 | overnight |

To understand if the addition of the SARS-CoV-2 probes impacted the quality of the human gene expression information captured, serial COVID-positive sections with human probes and spike-in SARS-CoV-2 probes versus only human probes were examined.

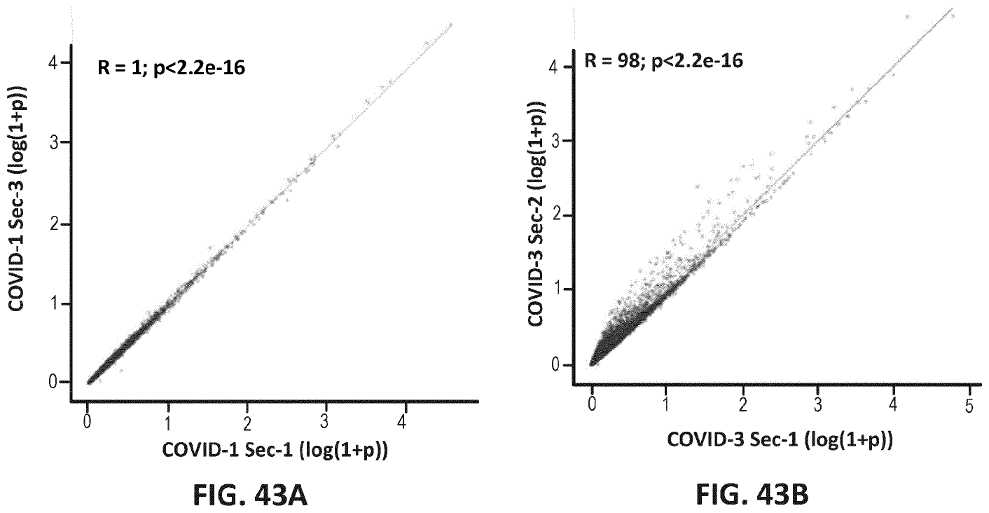

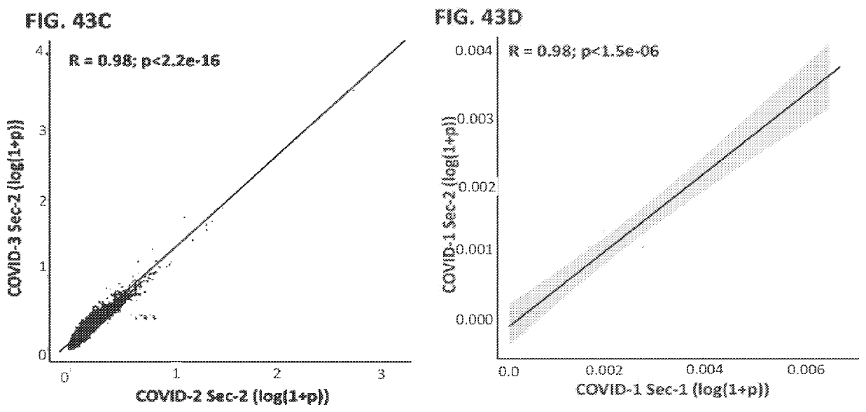

As shown in FIGS. 40A-40B, the SARS-CoV-2 probes were specific for capturing SARS-CoV-2 transcripts, even in the presence of human probes. Further, SARS-CoV-2 transcripts above background levels (1 unique molecule/section) were not detected in COVID-negative tissue sections, demonstrating the SARS-CoV-2 probes were specific for SARS-CoV-2. In serial sections, human gene expression profiles were consistent between COVID-positive sections with and without SARS-CoV-2 probes (r=0.98 to 1) (See FIGS. 43A-43D) and showed a similar distribution across the tissue sections, with an average of ˜4512 unique molecules per spot and ˜2228 unique genes per spot with SARS-CoV-2 probes and an average of ˜3739 unique molecules per spot and ˜1981 unique genes per spot without SARS-CoV-2 probes (FIGS. 41A, 41B, 42, and 52A).

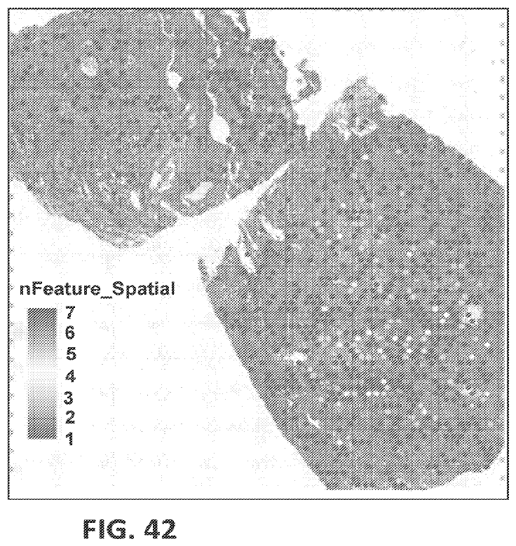

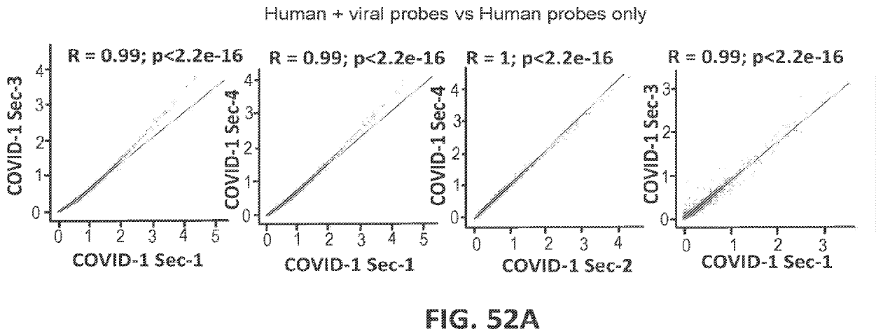

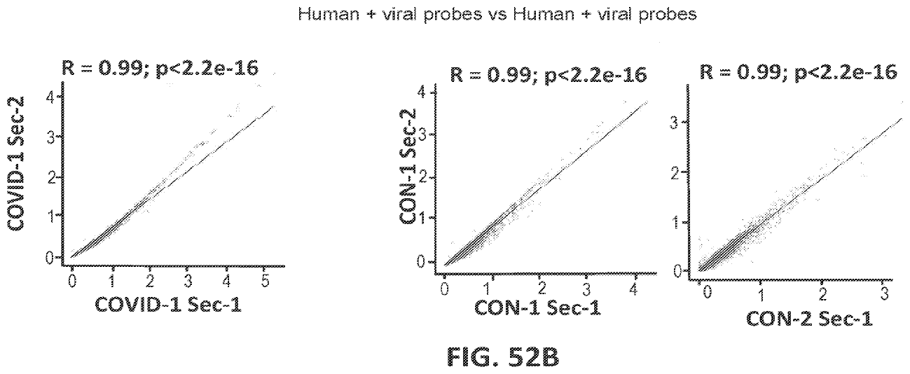

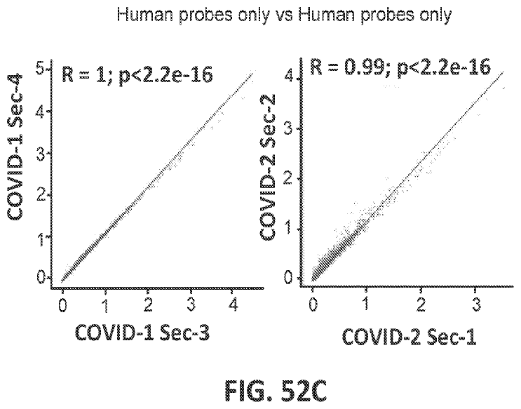

The consistency of the human gene expression information captured with and without the presence of the SAR-CoV-2 probes demonstrated the SARS-CoV-2 probes do not interfere with the capture efficiency of the human transcripts. Consistency among samples was observed, for instance, in similar number of reads, mean reads per spot, median genes per spot, and median UMI counts per spot (data not shown). Furthermore, the human gene expression profiles were consistent between replicates (either viral+human probes or only human probes) (r=0.9) with comparable distributions of unique genes and unique molecules per spot between most samples and across most sample replicates, demonstrating the high reproducibility of the method (FIG. 42, FIGS. 52B-52C). Across COVID-positive and COVID-negative tissue sections, a dataset consisting of a total of 37,754 spots, with an average of ˜2,013 unique human genes and ˜3,809 unique human molecules per spot, respectively, was generated. These data collectively demonstrate that both viral RTL probes and human probes can be used on the same sample without affecting detection of host (e.g., human) analytes.

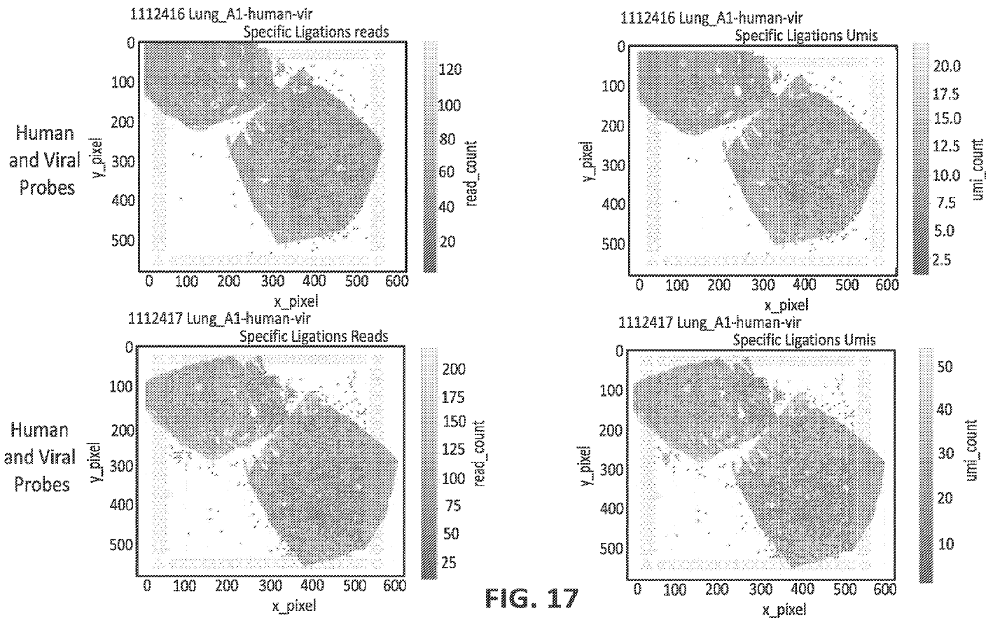

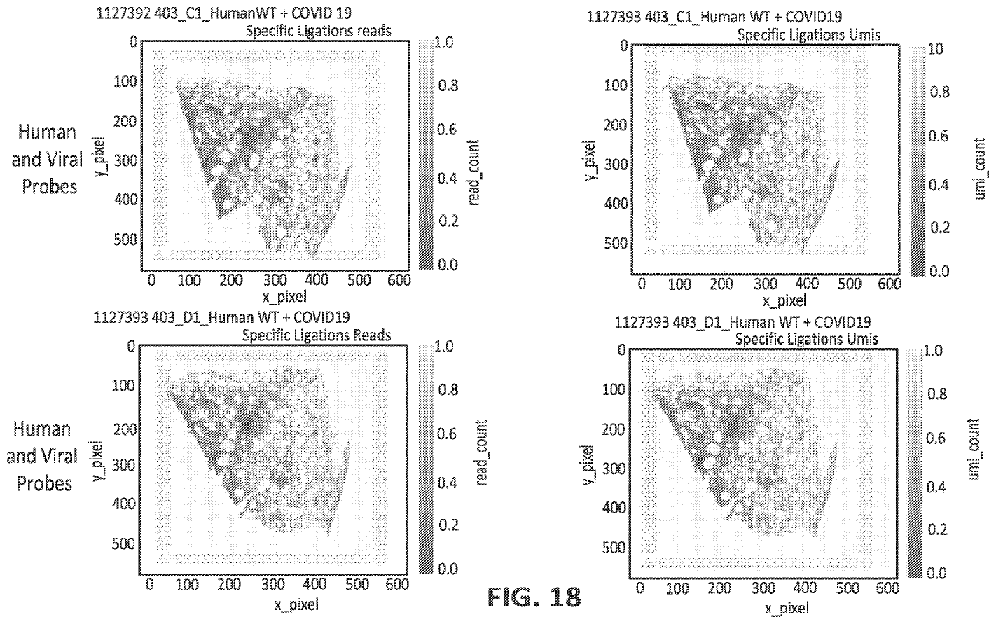

Next, it was examined whether the viral RTL probes could spatially detect SARS-CoV-2 in biological samples from the subject diagnosed with SARS-CoV-2 infection. Two replicates were performed in the sample from the subject diagnosed with SARS-CoV-2 infection and the same from the subject with no detected SARS-CoV-2 infection. As shown in FIG. 17, both reads (left images) and UMIs (right images) from ligated probes from SARS-CoV-2 were readily detected in both replicates from the subject diagnosed with SARS-CoV-2 infection, even in the presence of host (i.e., human) probes, and this detection was reproducible. See FIG. 43D. On the other hand, as shown in FIG. 18, neither reads (left images) nor UMIs (right images) from the SARS-CoV-2 ligated probes group were detected in two replicates from the subject without SARS-CoV-2 infection, even in the presence of host (i.e., human) probes.

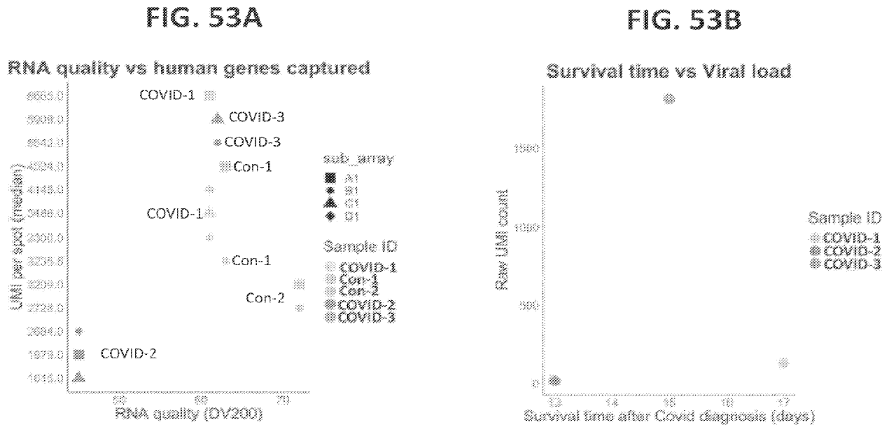

Additional samples were examined for detection of SARS-CoV-2. From COVID-positive tissue sections (n=3, described above), at least 1 SARS-CoV-2 gene was detected in 1,132 spots (3% of all spots) and captured up to 9 different SARS-CoV-2 genes (FIGS. 40A, 40B, and 42). From the SARS-CoV-2-positive spots across COVID-positive sections, an average of ˜1.7 unique molecules per spot and an average ˜1.5 unique genes per spot was obtained with reproducible capture of SARS-CoV-2 genes between consecutive sections (r=0.98, FIGS. 41A-41B, FIG. 43D). The low number of SARS-CoV-2-positive spots and low SARS-CoV-2 unique genes and molecules per spot averages indicate these patient samples show low levels of viral load, likely due to the longer disease duration (13-17 days) of these patients as several studies observed lower, or even undetectable, viral load in COVID-19 patients with longer survival times (FIG. 53B).

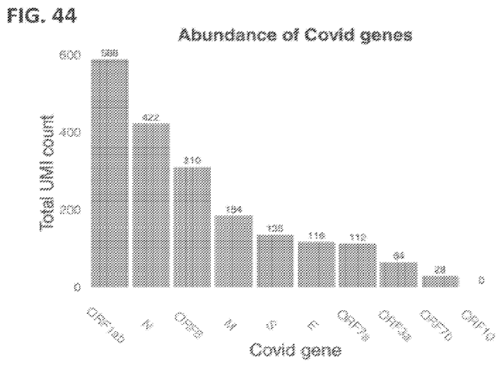

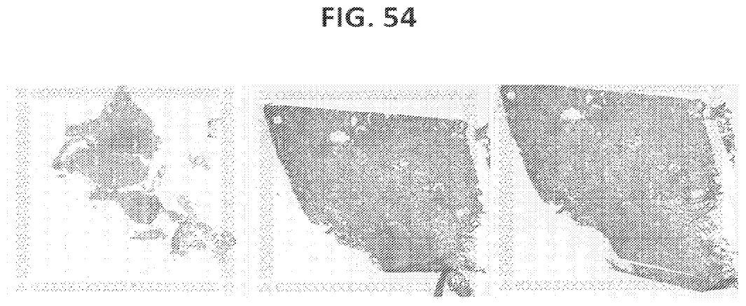

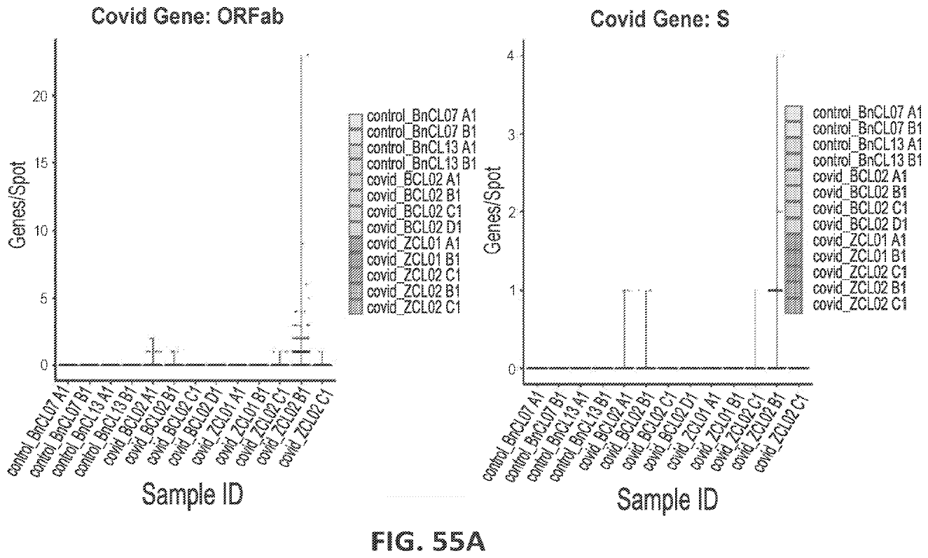

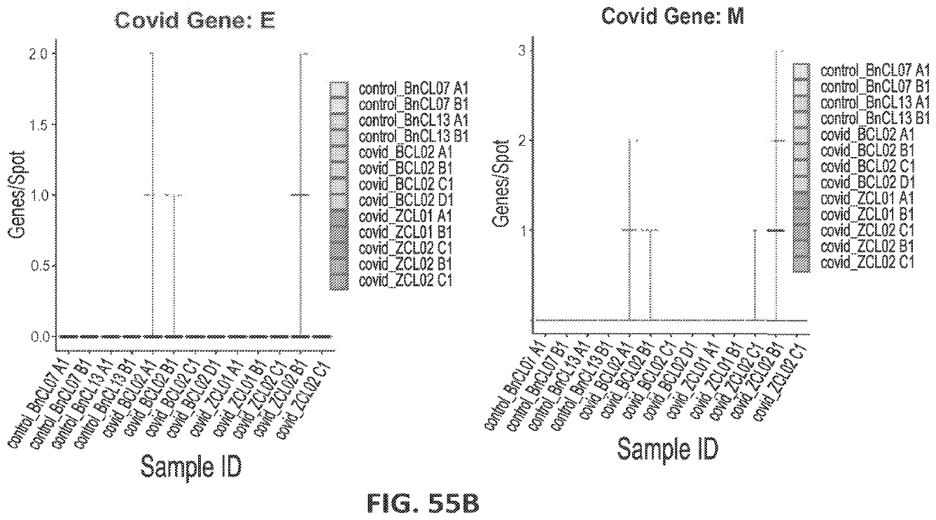

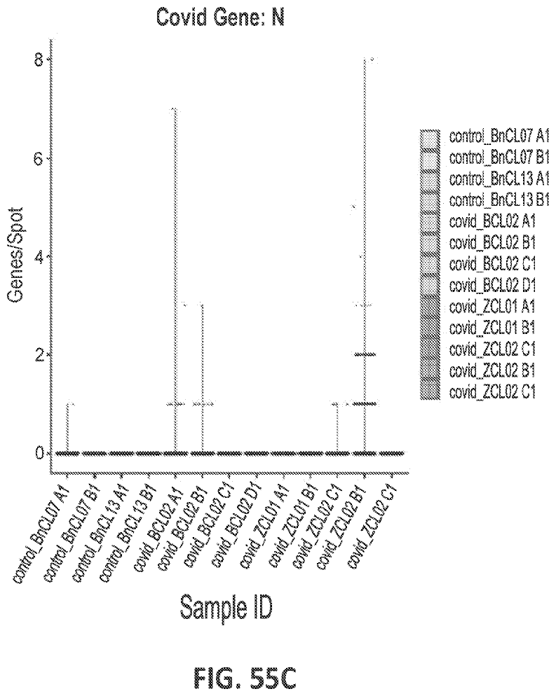

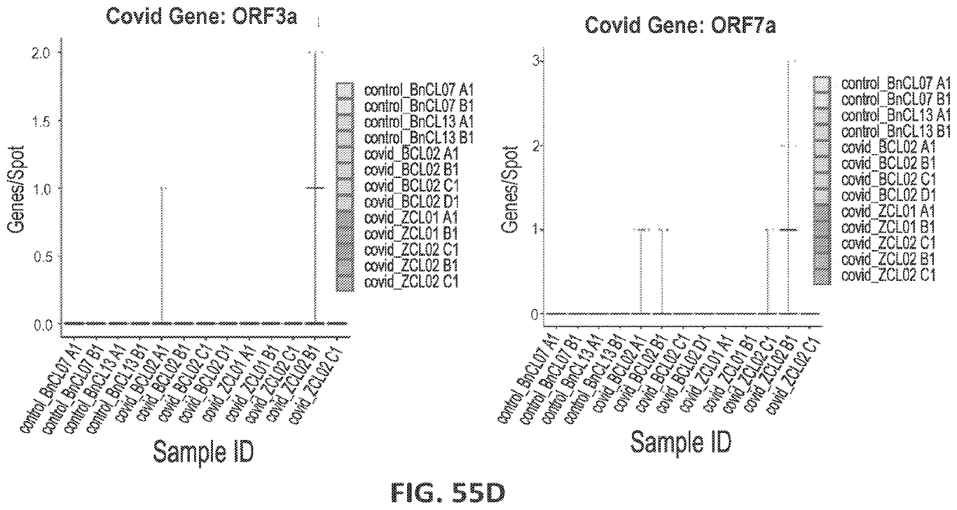

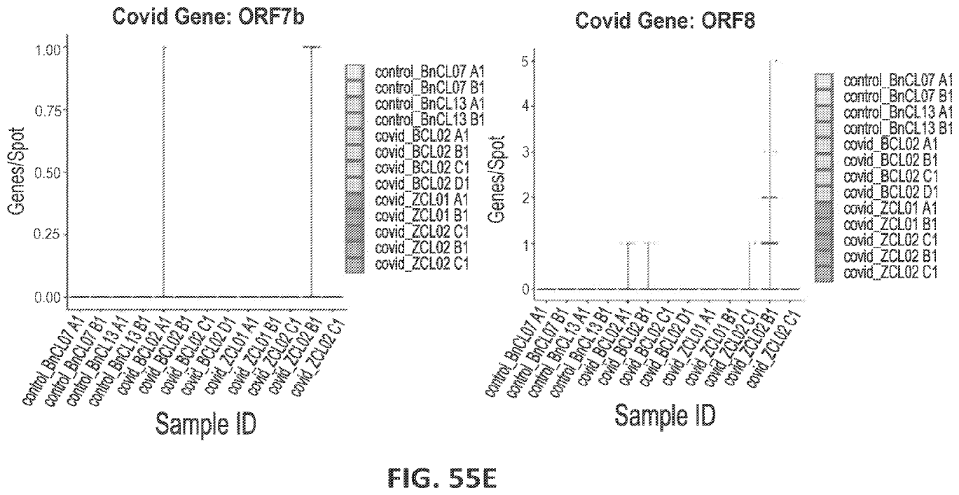

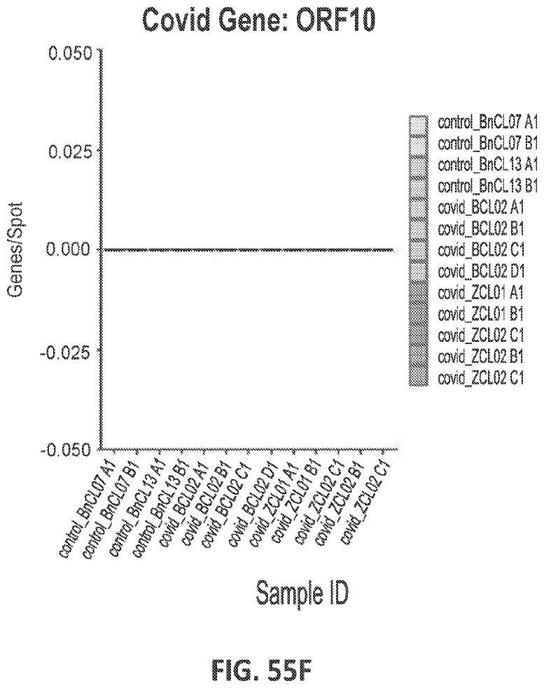

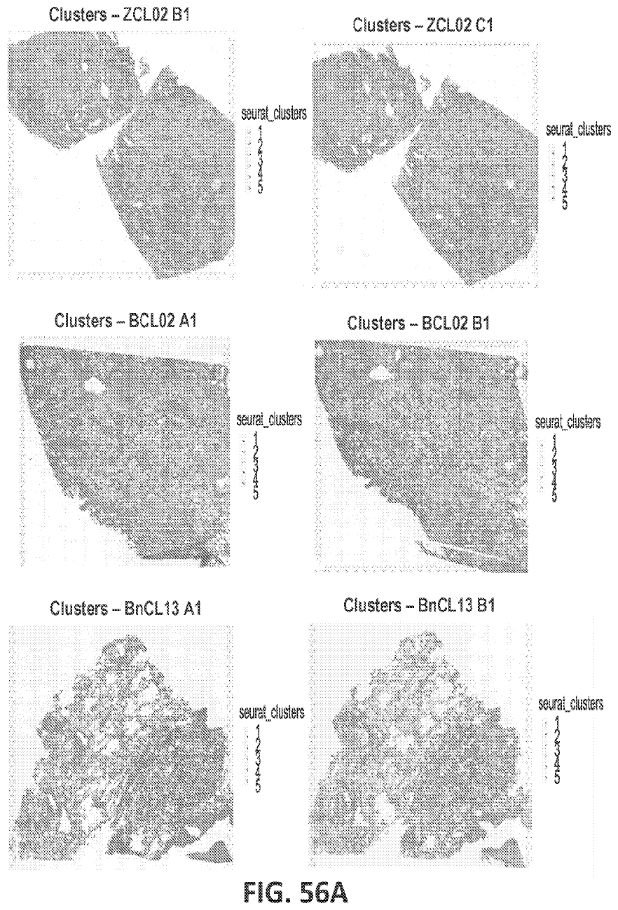

In addition, the number of total SARS-CoV-2-positive spots varied across COVID-positive samples, ranging from 14-1020 spots per section (FIG. 42, FIG. 54). The highest number of SARS-CoV-2-positive spots came from patient P1CL, with SARS-CoV-2-positive spots distributed throughout the section and most densely present in the center of the bottom half of the section (FIG. 42). Such inter-sample viral load heterogeneity has been observed by others and even heterogenous distribution within the same tissue sample. Varied abundances of the different SARS-CoV-2 gene transcripts was observed, with ORF1ab having the highest abundance (see FIG. 44, FIG. 55A-55F). In addition to detection of ORF1ab, S, ORF3a, E, M, ORF7a, ORF7b, ORF8 and N were detected in SARS-CoV-2-positive samples.

In no sample was ORF10 captured (see FIG. 55A-55F), in line with previous observations that ORF10 is consistently the lowest or absent sgRNA detected. Detection of ORF10 could be influenced by decay of the 3′end of SARS-CoV-2 gRNA and/or sgRNA, where ORF10 is the furthest ORF on the 3′ end, as nonsense-mediated decay at the 3′UTR in other single-stranded viruses has been observed. Perhaps this can occur more/differentially during the disease course and affect the abundance of ORF10. Nonetheless, the second highest expressed SARS-CoV-2 gene is N, in line with previous reports of N as the highest expressed sgRNA.

This method facilitates the co-capture of SARS-CoV-2 and human transcriptomes, where the relationship and interactions between the gene expression profiles can be explored. These data demonstrate that the viral RTL probes from Table 2-in the presence of host RTL probes—ligate and have the capacity to be spatially analyzed.

### Example 2. Validation by RNAScope and In Situ Sequencing

To validate the SARS-CoV-2 spatial transcriptomics (ST) probes, the number of positive ST spots for the S gene transcript were calculated and confirmed by RNAScope.

Optimal RNA integrity and assay conditions were assessed using Malat1 and Rplp0 housekeeping genes using the HS Library Preparation kit for CARTANA technology and following manufacturer's instructions on 5 μm tissue sections from representative sample P1CL. Since the control probes showed positive results, in situ sequencing was performed on two 5 μm consecutive sections from sample P1CL and one consecutive section from each negative control sample (P4CL and P5CL). Microscope slides (VWR) containing 5 μm tissues sections were stored at 4° C. until processing. FFPE sections were baked for 1 hour at 60° C. to partially melt paraffin and increase tissue adherence. Sections were deparaffinized using xylene for 2×7 minutes followed by an ethanol gradient to remove xylene and rehydrate the sections. Sections were permeabilized using citrate buffer pH 6.0 (Sigma Aldrich) for 45 minutes at 95° C. For library preparation, chimeric padlock probes (directly targeting RNA and containing an anchor sequence as well as a gene-specific barcode) for a custom panel of SARS-CoV-2 S and E genes were hybridized overnight at 37° C., then ligated followed by rolling circle amplification overnight at 30° C. using the HS Library Preparation kit from CARTANA technology and following manufacturer's instructions. Incubations were performed in SecureSeal™ chambers (Grace Biolabs). For tissue section mounting, Slow Fade Antifade Mountant (Thermo Fisher) was used for optimal handling and imaging. Quality control of the library preparation was performed by applying anchor probes to simultaneously detect all rolling circle amplification products from all genes in all panels. Anchor probes were labeled with Cy5 fluorophore (excitation at 650 nm and emission at 670 nm). All samples passed quality control and were sent for single cycle in situ barcode sequencing, imaging, and data processing. Briefly, adapter probes and a sequencing pool (containing 4 different fluorescent labels: Alexa Fluor® 488, Cy3, Cy5 and Alexa Fluor® 750) were hybridized to the in situ libraries to detect Covid gene-specific barcodes. This was followed by multicolor epifluorescence microscopy, scanning the whole area and thickness of the tissues. Raw data consisting of 20× magnification images from 5 fluorescent channels (DAPI, Alexa Fluor® 488, Cy3, Cy5 and Alexa Fluor® 750) and individual z-stacks, were flattened to 2D using maximum intensity projection with a Nikon Ti2 Nikon Ti2 (software NIS elements) utilizing Zyla 4.2 camera. After image processing, which includes image stitching, background filtering and a sub-pixel object registration algorithm, true signals were scored based on signal intensities from individual multicolor images. The results were summarized in a csv file and gene plots were generated using MATLAB.

RNAscope assay was performed on lung 5 μm FFPE sections on microscope slides (VWR) cut from depths consecutive to the sections mounted on Visium slides. The slides were baked in a dry oven for 1 h at 60° C. and then deparaffinized in xylene (2×5 min) and absolute ethanol (2×1 min) at room temperature. After drying, the sections were incubated in RNAscope Hydrogen Peroxide for 10 minutes at room temperature, followed by washing steps (2×) in distilled water. Target retrieval was performed using a 1× RNAscope Target Retrieval Reagent for 15 minutes, at a temperature constantly kept above 99° C. in a hot steamer. The slides were rinsed in distilled water, incubated in absolute ethanol for 3 minutes and dried at 60° C. After creating a hydrophobic barrier, the slides were left to dry overnight. The second day, the sections were incubated in RNAscope Protease Plus solution for 30 min at 40° C., followed by washing in distilled water. RNAscope V-nCov2019-S probe, RNAscope Positive Control probe (Hs—PPIB) and RNAscope Negative Control Probe (DapB) were hybridized to separate sections for 2 h at 40° C., then the slides were washed twice for 2 minutes in 1× Wash Buffer. The probe-specific signal was developed with an RNAscope 2.5 HD Detection Reagent—RED kit. Sequential hybridization of amplification reagents AMP1-4 proceeded at 40° C. for 30-15-30-15 minutes, while AMPS and AMP6 were applied at room temperature for 1 hour and 15 minutes, respectively, with two washing steps in 1× Wash Buffer after each incubation period. For signal detection, each section was incubated for 10 min at room temperature in 120 μl RED Working Solution, consisting of Fast RED-B and Fast RED-A reagents in a 1:60 ratio. All the protease digestion, probe hybridization, signal amplification and signal detection steps were performed in a HybEZ Humidity Control Tray, which was either placed into a HybEZ Oven for the 40° C. incubation steps or kept at room temperature. Following two washing steps in tap water, the slides were counterstained with 50% Gill's Hematoxylin staining solution for 2 min at room temperature, thoroughly rinsed with tap water, then soaked in 0.02% Ammonia water bluing solution and finally washed again in tap water. The slides were then dried completely at 60° C. and then quickly dipped into xylene before mounting them with VectaMount Permanent Mounting Medium. The RNAscope signal was imaged and evaluated with a Leica DM5500B microscope with a 20× and 40× magnification, using a Leica Application Suite X (LAS X) software platform.

RNAScope and spatial transcriptomics (ST) images were manually aligned with Adobe Photoshop 2022. All dots of chromogenic red signal as positive SARS-CoV-2 S gene signal were considered, since the majority of signal was above 1 dot per 10 nuclei area, in line with how others assessed RNAScope signal in SARS-CoV-2 viral low samples. The number of ST spots that were detected as the SARS-CoV-2 S gene and the RNAScope S gene signal were calculated. To adjust for the use of consecutive sections for ST and RNAScope experiments, rather than performing ST and RNAScope on the same section, the agreement of ST and RNAScope in 207×207 μm² block areas were evaluated. Using a computational pipeline, 53% of SARS-CoV-2 ST were confirmed by RNAScope, in rough agreement with a manual annotation of 60%. Since the manual and computation approaches were close in agreement, the computation approach was used to calculate the sensitivity of the SARS-CoV-2 S gene detection by ST. The computational validation was performed as follows: the RNAScope signal was detected with an ad hoc algorithm, which will be specified later; then both the binary ST and RNAScope signal images were aligned and cropped into 207×207 μm² blocks. The blocks containing RNAScope signals are defined as condition positive, within which those blocks where both ST and RNAScope signals are detected are recognized as true positives and the rest condition positive false negatives. With this definition, the sensitivity of ST verified by RNAScope was about 17.80%. RNAScope signals are detected with a chromatical analytic method. The image is first converted into Hue-Saturation-Value model, then the pixels whose hue is over 0.85, saturation over 0.25, and value over 0.40 are recognized as signal candidates. After performing a morphological opening operation, the collection of signal candidates are output as final RNAScope signals. To account for false positive signals in the COVID-negative samples, which can be due to the presence of RNAScope FastRed deposits and/or a false result from the computational method, the computational pipeline also was run on the negative control sections.

ISS consecutive section images and ST images were manually aligned with Adobe Photoshop 2022. Due to the use of non-consecutive sections, with ˜300 μm in between the ST and ISS sections, making an alignment of the tissue section challenging, the agreement between E and S gene signals for ST and ISS in block areas of 650 μm was evaluated. Within each block area, the presence of each gene from ST and ISS was manually annotated.

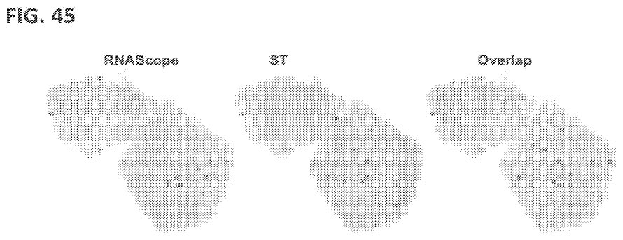

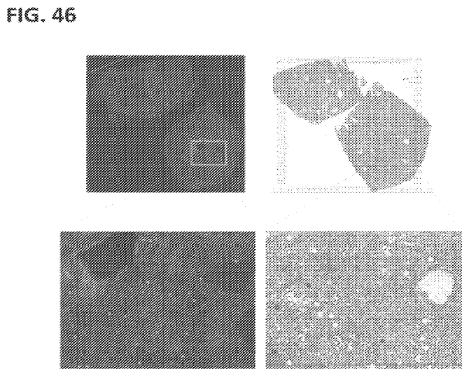

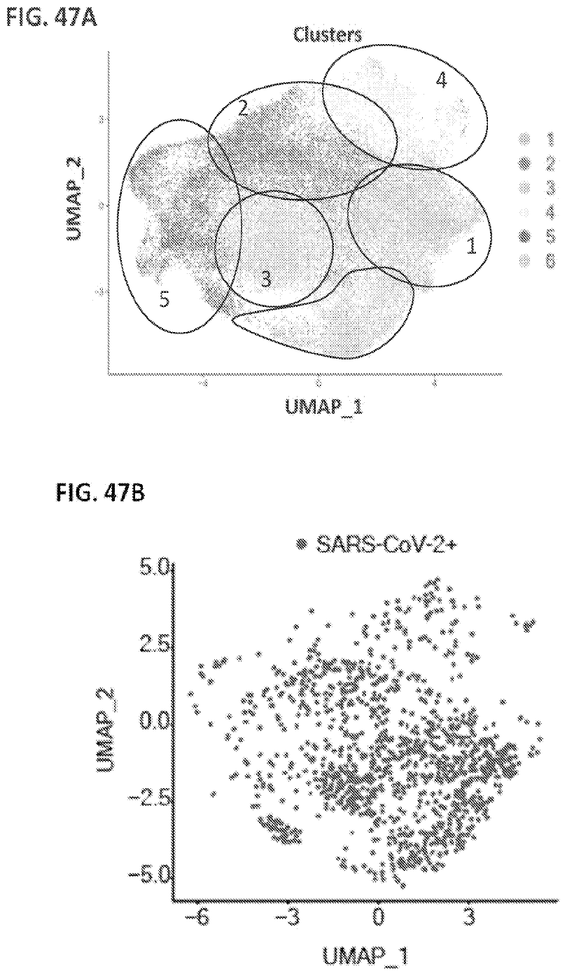

Comparing the two methods, 53.7% of the ST spot SARS-CoV-2 S gene signal was confirmed by RNAScope. Differences in detection of SARS-CoV-2 by ST compared to RNAScope may be due to the use of consecutive sections rather than both methods performed on the same section and influenced by the presence of RNAScope FastRed deposits. In addition, the sensitivity of ST detection of SARS-CoV-2 in comparison to RNAScope was determined to be 17.8%±%. RNAScope has high sensitivity, possibly at the level of single molecule detection, which could account for the lower sensitivity of ST as compared to RNAScope. When comparing the RNAScope and ST spatial signal, the signal intensity lines up in that the highest signal density in the lower half and middle area of the tissue section is observed (FIG. 45). With ST, the spatial distribution of 10 different SARS-CoV-2 genes was visualized throughout the tissue. Further, two of these genes with in situ sequencing (ISS) targeting the S and E gene transcripts were validated. The ISS analysis was not as robust as the RNAScope validation, due to performing ISS on only one COVID-positive sample and the two COVID-negative samples. Despite several sections (˜300 μm) in between the ST and ISS sections for sample P1CL, a similar expression trend between ST and ISS was observed (FIGS. 45 and 46) (E gene: sensitivity: 66.7%, specificity of 62.5%, 85%, FDR: 15%, Accuracy 65.7%).

### Example 3. Determination of Biomarkers that are Upregulated or Downregulated in SARS-CoV-2 Positive FFPE-Fixed Lung Sample Using Templated Ligation

Using the methods of templated ligation disclosed in Example 1, biomarkers that were dysregulated were identified using RTL probe pairs that detected increased and decreased expression of certain biomarkers in a SARS-CoV-2 positive FFPE-fixed lung sample. The same SARS-CoV-2-infected sample and negative control sample from Example 1 were evaluated. Compared to a normal lung tissue sample (i.e., a subject confirmed to be without a SARS-CoV-2 infection) the biomarkers listed in Table 9 were upregulated at least two fold in a sample infected with SARS-CoV-2, the biomarkers listed in Table 10 were downregulated at least two fold in a sample infected with SARS-CoV-2, and the biomarkers listed in Table 11 did not significantly change expression in a sample infected with SARS-CoV-2 compared to an uninfected lung tissue sample.

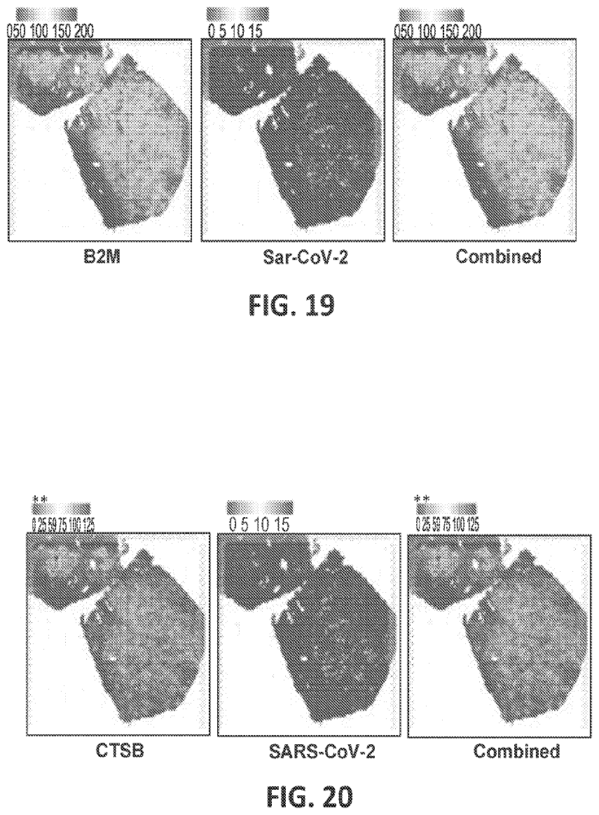

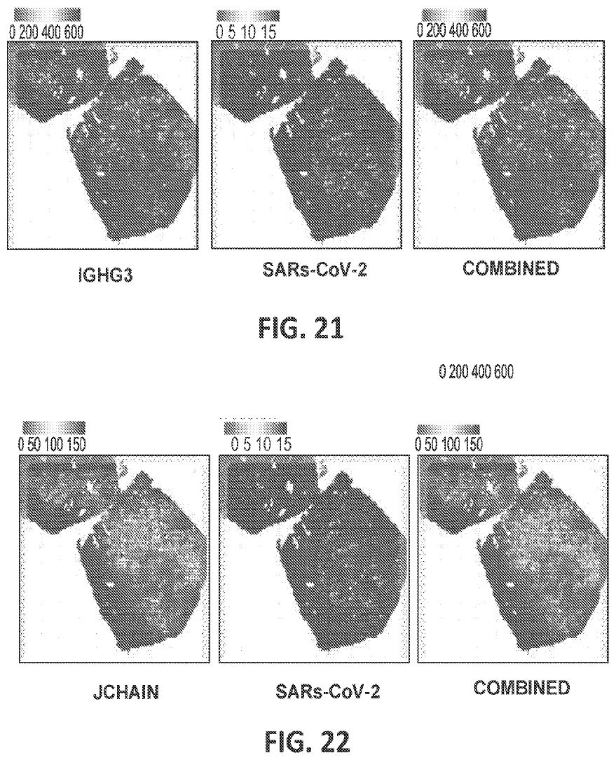

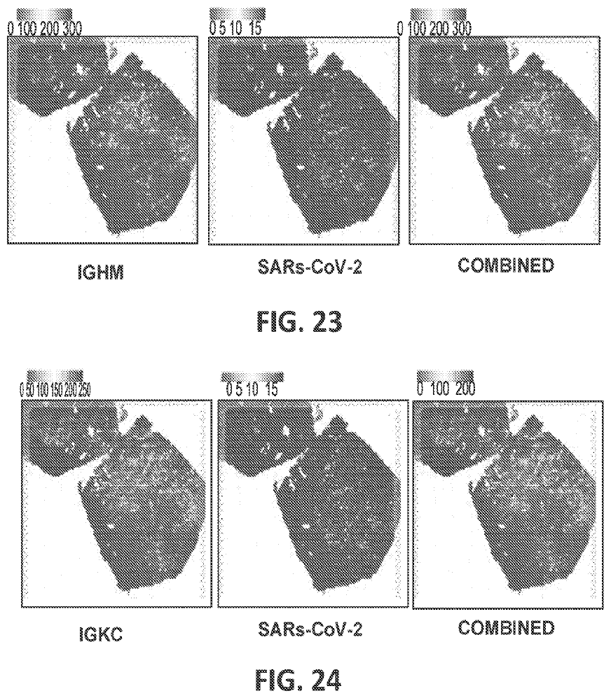

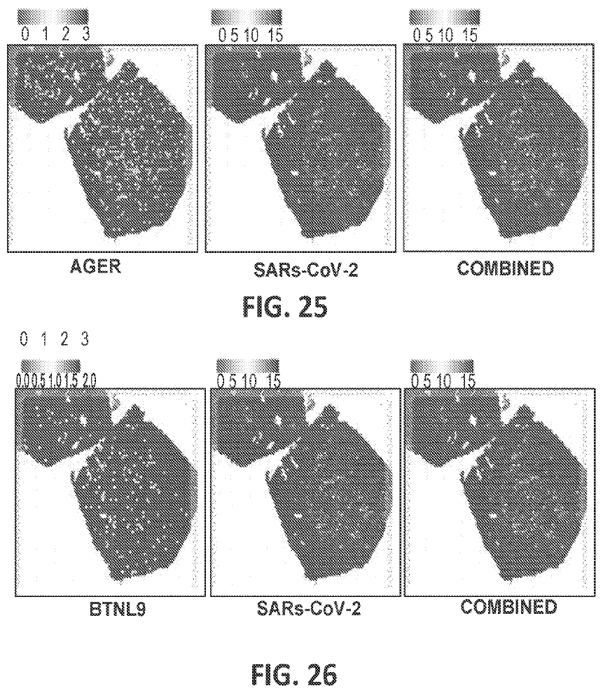

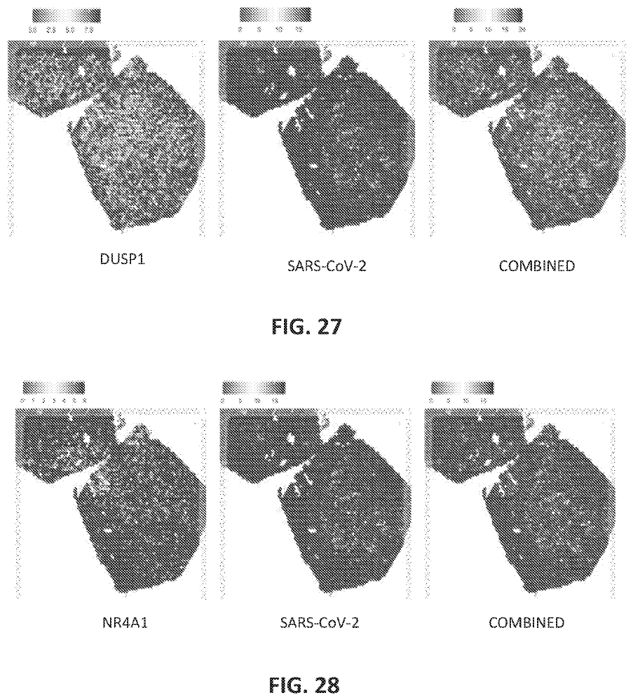

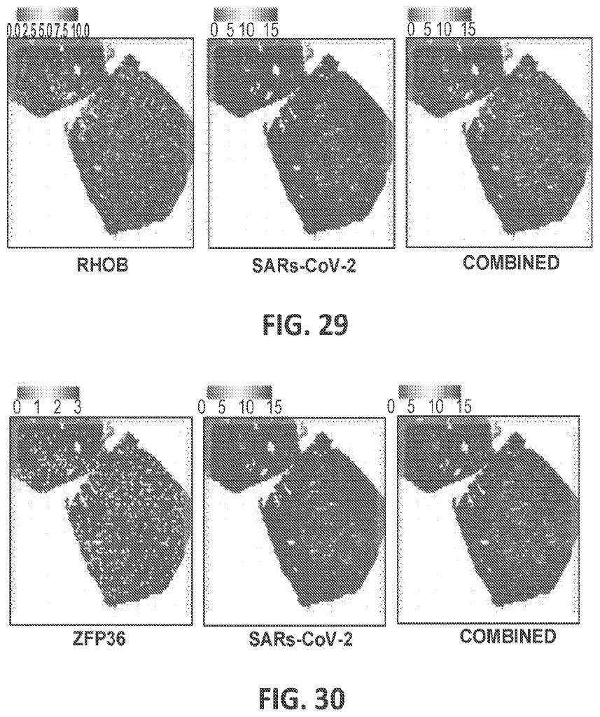

Additionally, images were generated to show overlap of certain biomarkers with SARS-CoV-2 (using a plurality of the 26 SARS-CoV-2 RTL probe pairs as shown in Table 2) expression in lung samples (FIGS. 19-30). Lung samples were analyzed for expression and co-localization of biomarkers using the methods of Example 1. As shown in FIGS. 19-30, biomarkers that were dysregulated (i.e., both upregulated and downregulated) were affected by the presence of SARS-CoV-2 infection. Specifically, each of B2M (FIG. 19), CTSB (FIG. 20), IGHG3 (FIG. 21), JCHAIN (FIG. 22), IGHM (FIG. 23), and IGKC (FIG. 24) was upregulated in the lung tissue sample and all six biomarkers were affected by the presence of SARS-CoV-2 infection. On the other hand, each of AGER (FIG. 25), BTNL9 (FIG. 26), DUSP1 (FIG. 27), NR4A1 (FIG. 28), RHOB (FIG. 29), and ZFP36 (FIG. 30)) was downregulated in the lung tissue sample, with each of these six biomarkers showing co-localized expression with SARS-CoV-2. These data demonstrate a proof of concept that the spatial location and abundance of one or more biomarkers could be determined to co-localize (or not) with SARS-CoV-2 in human lung tissue samples.

TABLE 9
SARS-CoV-2 biomarkers that were upregulated by more
than two-fold in infected human lung tissue.

| Analyte | Adjusted p-val | avg_log2FC | pct. 1 | pct. 2 |
|---|---|---|---|---|
| IGHG1 | 0 | 6.61137083248553 | 1 | 0.029 |
| IGLC2 | 0 | 5.95720432339386 | 1 | 0.029 |
| IGLV3-19 | 0 | 5.79200919523599 | 1 | 0 |
| IGLC3 | 0 | 5.74239926049774 | 1 | 0.055 |
| HBA2 | 0 | 5.55908176979226 | 1 | 0.391 |
| IGHG3 | 0 | 5.47100511247292 | 1 | 0.004 |
| IGHV3-30 | 0 | 5.37767392419177 | 1 | 0.023 |
| B2M | 0 | 5.34427256659984 | 1 | 0.588 |
| HLA-DRB1 | 0 | 4.84041974466282 | 1 | 0 |
| IGKC | 0 | 4.79527252508403 | 1 | 0.118 |
| IGHM | 0 | 4.77881066237413 | 1 | 0.027 |
| JCHAIN | 0 | 4.73883625727439 | 0.999 | 0.039 |
| FTH1 | 0 | 4.72591322430608 | 1 | 0.553 |
| FTL | 0 | 4.60248484380087 | 1 | 0.618 |
| IGHV4-28 | 0 | 4.58459493454973 | 1 | 0.003 |
| CTSB | 0 | 4.35881191768931 | 1 | 0.226 |
| IGHV1-69-2 | 0 | 4.27487051504403 | 0.998 | 0 |
| LYZ | 0 | 4.27144229289537 | 1 | 0.19 |
| S100A9 | 0 | 4.24825911450526 | 1 | 0.151 |
| CXCL9 | 0 | 4.21647490700732 | 0.999 | 0.014 |
| HLA-A | 0 | 4.15259114747133 | 1 | 0.676 |
| HLA-B | 0 | 4.12670297625816 | 1 | 0.477 |
| TIMP1 | 0 | 4.06506231340556 | 1 | 0.249 |
| IGKV1-6 | 0 | 4.03251514451959 | 0.997 | 0 |
| CTSD | 0 | 4.00515888081153 | 1 | 0.196 |
| HBB | 0 | 3.99870762680455 | 1 | 0.515 |
| TMSB10 | 0 | 3.90587371821456 | 1 | 0.68 |
| CD163 | 0 | 3.82174091171143 | 0.999 | 0.069 |
| MTRNR2L12 | 0 | 3.77944107821747 | 0.985 | 0.022 |
| NPIPB4 | 0 | 3.75960894784838 | 1 | 0.395 |
| IGHV3-33 | 0 | 3.74588276282881 | 0.992 | 0 |
| MT2A | 0 | 3.7289478781249 | 1 | 0.347 |
| COL3A1 | 0 | 3.60308663455463 | 0.999 | 0.347 |
| GAPDH | 0 | 3.60286194313011 | 1 | 0.303 |
| CD74 | 0 | 3.56907471194863 | 1 | 0.723 |
| IGHV1-24 | 0 | 3.54756853980406 | 0.986 | 0 |
| IGLV3-25 | 0 | 3.54017025101785 | 0.98 | 0 |
| SOD2 | 0 | 3.53381610562324 | 1 | 0.35 |
| IGLC7 | 0 | 3.4779427642762 | 0.988 | 0.017 |
| IGLV1-44 | 0 | 3.47333371958129 | 0.992 | 0.041 |
| IFITM3 | 0 | 3.43212414787272 | 1 | 0.286 |
| FN1 | 0 | 3.37594540541114 | 1 | 0.691 |
| SPARC | 0 | 3.36132002076333 | 1 | 0.542 |
| HIST1H2BC | 0 | 3.33075662740268 | 1 | 0.232 |
| SERF1A | 0 | 3.32928707201651 | 1 | 0.997 |
| HLA-DRA | 0 | 3.31434987480236 | 0.999 | 0.154 |
| EEF1A1 | 0 | 3.23223762734486 | 1 | 0.617 |
| ACTB | 0 | 3.23108572637969 | 1 | 0.917 |
| IGHV3-21 | 0 | 3.22964407192959 | 0.983 | 0.008 |
| XBP1 | 0 | 3.22073987191643 | 0.998 | 0.129 |
| S100A8 | 0 | 3.20917338242069 | 0.998 | 0.118 |
| IGLV2-8 | 0 | 3.1908650136105 | 0.977 | 0.002 |
| VIM | 0 | 3.17987017654638 | 1 | 0.452 |
| LDHA | 0 | 3.14147687094581 | 1 | 0.244 |
| IGLV3-1 | 0 | 3.14040837052446 | 0.978 | 0 |
| IFI30 | 0 | 3.13445915326003 | 0.998 | 0.069 |
| UBB | 0 | 3.12133380331495 | 1 | 0.214 |
| TMSB4X | 0 | 3.11953256279226 | 1 | 0.45 |
| IGLC1 | 0 | 3.11387693754514 | 0.975 | 0.007 |
| HMGN2 | 0 | 3.11351675646153 | 1 | 0.217 |
| HIST2H2BF | 0 | 3.10966830754806 | 0.999 | 0.197 |
| SERF2 | 0 | 3.08648219965903 | 1 | 0.262 |
| TPT1 | 0 | 3.07841075264788 | 0.999 | 0.262 |
| IGKV1-17 | 0 | 3.06901905070982 | 0.969 | 0 |
| PSAP | 0 | 3.04810552700504 | 1 | 0.352 |
| PLTP | 0 | 3.02715833274571 | 0.995 | 0.078 |
| HNRNPA2B1 | 0 | 3.01719005356705 | 0.998 | 0.165 |
| TMBIM6 | 0 | 2.99453501783457 | 0.998 | 0.134 |
| IGKV3OR2-268 | 0 | 2.98257978484682 | 0.976 | 0.019 |
| C1QC | 0 | 2.968062983073 | 0.993 | 0.13 |
| IGKVID-12 | 0 | 2.96647934087284 | 0.954 | 0.017 |
| CFL1 | 0 | 2.95786153963268 | 1 | 0.395 |
| CD63 | 0 | 2.95587309346814 | 0.999 | 0.179 |
| HIST2H2AC | 0 | 2.95300043913673 | 0.999 | 0.293 |
| HLA-DRB5 | 0 | 2.94437695673289 | 0.983 | 0 |
| ACTG1 | 0 | 2.92557020795001 | 1 | 0.832 |
| UBA52 | 0 | 2.92407409760325 | 0.999 | 0.135 |
| S100A6 | 0 | 2.91662050423785 | 1 | 0.693 |
| LUM | 0 | 2.9144571172295 | 0.996 | 0.22 |
| SRRM2 | 0 | 2.9060734567661 | 1 | 0.396 |
| EEF2 | 0 | 2.88777242915471 | 1 | 0.337 |
| COL1A1 | 0 | 2.86990858393683 | 0.986 | 0.111 |
| PPIB | 0 | 2.85208923918834 | 0.998 | 0.099 |
| IGKV1-5 | 0 | 2.82972627721957 | 0.927 | 0 |
| IGLV3-10 | 0 | 2.82808615690211 | 0.915 | 0 |
| NFKBIA | 0 | 2.828077752292 | 0.996 | 0.136 |
| COL1A2 | 0 | 2.82533072231303 | 0.998 | 0.424 |
| IGFBP7 | 0 | 2.79682844486919 | 0.993 | 0.385 |
| MT1G | 0 | 2.78967935875759 | 0.98 | 0.005 |
| CD44 | 0 | 2.78628963429987 | 0.993 | 0.117 |
| HLA-C | 0 | 2.78221212677781 | 0.99 | 0.003 |
| PSME2 | 0 | 2.75888661539762 | 0.997 | 0.138 |
| IFITM1 | 0 | 2.75487717628035 | 0.993 | 0.099 |
| HBA1 | 0 | 2.74571586228461 | 0.916 | 0.032 |
| IFITM2 | 0 | 2.73649388530258 | 0.998 | 0.174 |
| ARPC1B | 0 | 2.69823166481616 | 0.997 | 0.157 |
| OAZ1 | 0 | 2.67087739719609 | 0.997 | 0.161 |
| IGLV2-23 | 0 | 2.66735640766705 | 0.93 | 0.002 |
| AL136454.1 | 0 | 2.66555261121958 | 0.995 | 0.175 |
| ENO1 | 0 | 2.66103571631403 | 0.996 | 0.147 |
| TPI1 | 0 | 2.64944791245911 | 0.999 | 0.346 |
| HIST1H2BM | 0 | 2.6325102345446 | 0.985 | 0.035 |
| GNAS | 0 | 2.61397485888485 | 1 | 0.443 |
| HIST1H4B | 0 | 2.60756380361957 | 0.988 | 0.044 |
| TPM3 | 0 | 2.59611914575749 | 0.992 | 0.125 |
| CTSZ | 0 | 2.58541480067017 | 0.988 | 0.1 |
| ICAM1 | 0 | 2.57382556759815 | 0.995 | 0.214 |
| IGHV3-35 | 0 | 2.55908850596248 | 0.942 | 0.024 |
| IGKV3-20 | 0 | 2.554846434979 | 0.931 | 0.012 |
| HIST1H2AH | 0 | 2.55256348456674 | 0.986 | 0.041 |
| FAM153B | 0 | 2.53685184769412 | 0.999 | 0.355 |
| NPIPA1 | 0 | 2.53330219275565 | 0.985 | 0.053 |
| SERPINE1 | 0 | 2.52617321535965 | 0.983 | 0.177 |
| LGALS1 | 0 | 2.52379546850192 | 0.985 | 0.062 |
| FCER1G | 0 | 2.52203582978369 | 0.978 | 0.014 |
| FKBP1C | 0 | 2.51788834953962 | 0.99 | 0.209 |
| ATP5F1E | 0 | 2.51626171535724 | 0.993 | 0.104 |
| IGLV1-47 | 0 | 2.51537258892797 | 0.905 | 0 |
| MT1H | 0 | 2.50536350379433 | 0.964 | 0 |
| HIST1H2AJ | 0 | 2.49980155694694 | 0.981 | 0.023 |
| H3F3B | 0 | 2.49185091089089 | 1 | 0.647 |
| CD81 | 0 | 2.48930362103315 | 0.997 | 0.312 |
| EIF4A1 | 0 | 2.48658260072316 | 0.993 | 0.152 |
| HIST1H2BD | 0 | 2.48273074651884 | 0.984 | 0.098 |
| CD68 | 0 | 2.47108619802654 | 0.969 | 0.039 |
| DDX5 | 0 | 2.46295154000556 | 0.997 | 0.299 |
| SH3BGRL3 | 0 | 2.45847307866235 | 0.991 | 0.171 |
| IGHV1-2 | 0 | 2.45510857007033 | 0.892 | 0 |
| HIST2H3D | 0 | 2.4548126727611 | 0.98 | 0.039 |
| MSN | 0 | 2.45268278171346 | 0.995 | 0.235 |
| HIST1H2AC | 0 | 2.45077659816057 | 0.999 | 0.44 |
| HLA-DPA1 | 0 | 2.44315673935185 | 0.985 | 0.077 |
| SSR3 | 0 | 2.42816350870785 | 0.973 | 0.035 |
| IGHA1 | 0 | 2.41584602918285 | 0.934 | 0.102 |
| HIST1H1C | 0 | 2.39104708026269 | 0.992 | 0.204 |
| CYBB | 0 | 2.38587221478409 | 0.971 | 0.031 |
| WARS | 0 | 2.38386559302644 | 0.984 | 0.151 |
| CYBA | 0 | 2.38339146186116 | 0.981 | 0.07 |
| IGHV1-18 | 0 | 2.37257725274009 | 0.858 | 0 |
| VSIG4 | 0 | 2.37165950152718 | 0.962 | 0.03 |
| PCBP1 | 0 | 2.36674149583775 | 0.993 | 0.226 |
| NPC2 | 0 | 2.36490812540371 | 0.968 | 0.038 |
| HIST1H2BN | 0 | 2.35939183804766 | 0.979 | 0.045 |
| PKM | 0 | 2.35896533362916 | 0.99 | 0.155 |
| HMGB2 | 0 | 2.35168597214325 | 0.99 | 0.139 |
| SRSF5 | 0 | 2.35002793114606 | 0.99 | 0.214 |
| HLA-E | 0 | 2.3439117392159 | 1 | 0.588 |
| CTSH | 0 | 2.32839706914089 | 0.975 | 0.029 |
| HIST1H2AM | 0 | 2.29854159401539 | 0.97 | 0.031 |
| IGHV3-64D | 0 | 2.298531311373 | 0.894 | 0 |
| HIST1H2BJ | 0 | 2.29677363671892 | 0.969 | 0.042 |
| CALR | 0 | 2.29653997545858 | 0.987 | 0.149 |
| HSPA8 | 0 | 2.29421354516991 | 0.971 | 0.083 |
| CEBPB | 0 | 2.29130519415092 | 0.994 | 0.366 |
| ADAMTS2 | 0 | 2.2800512425164 | 0.959 | 0.01 |
| IGHV3-15 | 0 | 2.27556841103239 | 0.861 | 0 |
| CTSS | 0 | 2.27522940987465 | 0.965 | 0.018 |
| EEF1G | 0 | 2.27478682047393 | 0.981 | 0.121 |
| BTF3 | 0 | 2.26232702800531 | 0.971 | 0.054 |
| IGHV3-66 | 0 | 2.26020086652554 | 0.871 | 0 |
| TAP1 | 0 | 2.23420302687121 | 0.977 | 0.094 |
| APLP2 | 0 | 2.22586692178421 | 0.982 | 0.191 |
| TAGLN2 | 0 | 2.20998210616667 | 0.997 | 0.456 |
| IFI27 | 0 | 2.19543789095245 | 0.994 | 0.41 |
| CAPZB | 0 | 2.18515974263696 | 0.977 | 0.12 |
| FKBP1A | 0 | 2.17875920169291 | 0.968 | 0.155 |
| STAT1 | 0 | 2.17619338604533 | 0.958 | 0.026 |
| FKBP5 | 0 | 2.16783217791831 | 0.971 | 0.114 |
| HIST1H4D | 0 | 2.15909337853882 | 0.952 | 0.018 |
| TAPBP | 0 | 2.15569900382232 | 0.98 | 0.201 |
| GLUL | 0 | 2.15241078770902 | 0.978 | 0.231 |
| CLTA | 0 | 2.14735438888008 | 0.979 | 0.163 |
| TCIRG1 | 0 | 2.14613183064505 | 0.971 | 0.131 |
| CALM2 | 0 | 2.13562654368472 | 0.976 | 0.13 |
| HIST1H4A | 0 | 2.1326734273076 | 0.953 | 0.042 |
| PGAM1 | 0 | 2.12606977373964 | 0.972 | 0.096 |
| TMA7 | 0 | 2.12567517433578 | 0.977 | 0.132 |
| GABARAP | 0 | 2.11781080175423 | 0.975 | 0.133 |
| SQSTM1 | 0 | 2.09658346701865 | 0.998 | 0.52 |
| MIF | 0 | 2.09394933918196 | 0.954 | 0.042 |
| IGLV1-40 | 0 | 2.08803884455427 | 0.805 | 0 |
| EEF1B2 | 0 | 2.08690189558871 | 0.939 | 0.035 |
| H1FX | 0 | 2.08169273176611 | 0.976 | 0.185 |
| IGHV4-4 | 0 | 2.07310289942739 | 0.841 | 0.002 |
| NNMT | 0 | 2.07223852428544 | 0.957 | 0.07 |
| ARF1 | 0 | 2.06785646650036 | 0.982 | 0.248 |
| HIST1H2AG | 0 | 2.06439696925208 | 0.923 | 0.007 |
| CD99 | 0 | 2.04841337719474 | 0.97 | 0.161 |
| CCL21 | 0 | 2.04039323837796 | 0.828 | 0.089 |
| HIST1H4E | 0 | 2.03458218507706 | 0.959 | 0.11 |
| DCN | 0 | 2.03427852064846 | 0.998 | 0.625 |
| IGKV3-11 | 0 | 2.03262438654673 | 0.853 | 0.008 |
| LGALS3BP | 0 | 2.03074625631675 | 0.977 | 0.23 |
| CAPG | 0 | 2.02927147841233 | 0.936 | 0.045 |
| S100A10 | 0 | 2.02843166448938 | 0.982 | 0.28 |
| SRGN | 0 | 2.02561949799629 | 0.949 | 0.098 |
| SLC2A3 | 0 | 2.02483380481008 | 0.94 | 0.14 |
| COX7C | 0 | 2.02435159754094 | 0.968 | 0.132 |
| C1S | 0 | 2.0184447458559 | 0.967 | 0.196 |
| ALDOA | 0 | 2.01706857694053 | 0.973 | 0.208 |
| HSP90AB1 | 0 | 2.01638049167293 | 0.986 | 0.321 |
| ACADVL | 0 | 2.0155970092744 | 0.968 | 0.14 |
| NBPF15 | 0 | 2.01494930787898 | 0.977 | 0.246 |
| UBE2D3 | 0 | 2.0113226747513 | 0.971 | 0.171 |
| WASHC1 | 0 | 2.00839189733612 | 0.948 | 0.079 |
| BSG | 0 | 2.00500134009566 | 0.97 | 0.186 |
| ITM2B | 0 | 2.00251432451246 | 1 | 0.775 |

TABLE 10
SARS-CoV-2 biomarkers that were downregulated by
more than two-fold in infected human lung tissue.

| Gene | Adjusted p-val | Average log2FC |
|---|---|---|
| NR4A1 | 0 | −0.951599596284403 |
| AGER | 0 | −0.878655378491796 |
| BTNL9 | 0 | −0.669969499726119 |
| FOS | 1.18671393078663e−288 | −0.637591144809267 |
| RHOB | 2.48035897730576e−141 | −0.438893782374735 |
| DUSP1 | 3.68771717937347e−155 | −0.437425189443028 |
| ZFP36 | 9.28054630092445e−248 | −0.427114157373533 |
| TNXB | 1.42736020640144e−191 | −0.369685330181478 |
| HES1 | 2.88341520141929e−159 | −0.35112107428326 |
| HIF3A | 8.08395589496317e−88 | −0.277248815216033 |
| JUN | 2.92176588318484e−62 | −0.270482602442253 |
| CSAD | 1.0717346620216e−102 | 0.250756680003328 |
| MAF | 1.33599263113083e−96 | 0.253934131859321 |
| SHANK3 | 6.40109840389815e−52 | 0.254463802417495 |
| C4B | 3.75490136952504e−70 | 0.257979679212304 |
| CYTH2 | 1.26462452553306e−91 | 0.258699867568872 |
| AMOTL1 | 1.12614727603334e−92 | 0.259461665172639 |
| KLF2 | 1.56393773533071e−52 | 0.262687638772516 |
| ADARB1 | 4.46372986463634e−114 | 0.263039077144517 |
| CAPN7 | 1.49603785443674e−127 | 0.263427666035182 |
| ROBO4 | 1.73669656758052e−104 | 0.263725766631094 |
| PON2 | 1.14720874218681e−115 | 0.267498260151933 |
| APOL3 | 4.93864358138669e−137 | 0.268474362591298 |
| DMWD | 1.21759863945122e−128 | 0.269818235220045 |
| SLC12A4 | 1.24091318778857e−108 | 0.269849442819201 |
| BICD2 | 9.72317981378143e−119 | 0.277757301623246 |
| ACTN4 | 8.98669552428411e−95 | 0.281435025999081 |
| LMCD1 | 7.90877831799365e−138 | 0.282054688060135 |
| BAG1 | 5.43294757162237e−111 | 0.282596547977296 |
| C11orf96 | 3.73199520769859e−70 | 0.284038254918765 |
| PPFIBP1 | 1.50832938110668e−82 | 0.284295927710803 |
| BCAS2 | 1.44104551441462e−140 | 0.284942134835942 |
| PLVAP | 4.34586702443404e−124 | 0.289762451382912 |
| SPON2 | 7.6315168723514e−103 | 0.292338546228908 |
| SLC11A1 | 2.70144486548159e−95 | 0.293968292464669 |
| CHSY1 | 8.28582216593583e−146 | 0.295052017207644 |
| INAFM1 | 3.34342068616797e−172 | 0.295485956911199 |
| SF3A2 | 1.15956996423068e−125 | 0.296529566136421 |
| TNS2 | 3.11830422340529e−145 | 0.298371154097085 |
| MED13L | 3.51562598229805e−102 | 0.299506391694277 |
| KDM5B | 9.15932366300399e−132 | 0.299650492862121 |
| KDM6B | 1.79802663141594e−136 | 0.300254639214324 |
| SAMD4B | 1.12514185412581e−140 | 0.300299367438213 |
| COL4A2 | 8.76350155051399e−161 | 0.300892552511512 |
| SKI | 3.36176833543502e−167 | 0.301688920370417 |
| QRICH1 | 1.01002358722358e−208 | 0.303009243716012 |
| TNS1 | 1.86288982540494e−106 | 0.304028409592176 |
| CTIF | 1.80957270229452e−151 | 0.304124231056882 |
| SORBS3 | 3.84963717154887e−135 | 0.304518158944618 |
| EEF2K | 1.64065658340844e−189 | 0.305074632906124 |
| IL16 | 4.1226550877001e−148 | 0.30602955330731 |
| GBF1 | 8.98833294725477e−146 | 0.306505735173548 |
| PHACTR2 | 9.50274427432316e−186 | 0.306821369442855 |
| TRAK1 | 0 | 0.306932718244456 |
| MAML1 | 1.54008523312903e−140 | 0.309125634817859 |
| DVL3 | 1.22557827566634e−158 | 0.309813538924693 |
| KRT18 | 1.0475096007057e−99 | 0.313216059673308 |
| LIMD1 | 1.90174085647175e−163 | 0.31391459493227 |
| PBX2 | 4.49640077539004e−164 | 0.314865812186085 |
| TXNDC15 | 1.36368977537036e−183 | 0.315632070009118 |
| ACSL3 | 0 | 0.316979910855386 |
| SPAG9 | 1.73751668930614e−167 | 0.318709584187112 |
| MRNIP | 1.18187533617899e−197 | 0.319062126420049 |
| RERE | 4.79913346950546e−129 | 0.319073869016953 |
| BTG2 | 3.00264941414653e−114 | 0.320057221112307 |
| CD34 | 4.4653304408066e−156 | 0.320107454669165 |
| PTMA | 1.12377174860268e−146 | 0.320352718540304 |
| PLCB3 | 3.0405332145945e−176 | 0.320530584923907 |
| KLF3 | 3.84194336000703e−170 | 0.320602720099615 |
| STAT5B | 3.85151117328237e−176 | 0.320987443483877 |
| KDSR | 5.03872553845978e−180 | 0.321389151366226 |
| D2HGDH | 2.62303424044129e−151 | 0.32231457347709 |
| DCAF15 | 7.06726583968224e−202 | 0.322347259046499 |
| ABTB1 | 1.98293924159713e−160 | 0.32262860057883 |
| ACOT2 | 2.11982820081881e−201 | 0.323201233576876 |
| PLK2 | 2.99404234864468e−171 | 0.323369046701724 |
| INTS1 | 1.53330327198989e−185 | 0.325285460920755 |
| GBA2 | 2.67760588771838e−154 | 0.325310637871921 |
| F2R | 1.43611852381186e−159 | 0.325572005947978 |
| LAS1L | 1.83287696553375e−179 | 0.325819457487067 |
| ITGA3 | 5.03593252502092e−179 | 0.325858319315742 |
| MRC2 | 8.11171556005626e−185 | 0.326205192625312 |
| CDIPT | 1.50727643222347e−205 | 0.327042166236877 |
| SUPT6H | 1.72291845303505e−154 | 0.32705071770645 |
| RBMS2 | 1.08011511947025e−152 | 0.32734279639048 |
| KDM4B | 1.28884187760462e−197 | 0.327941766351838 |
| LMO2 | 2.28019420769651e−176 | 0.328125400177249 |
| EBPL | 2.34346046876297e−195 | 0.32820716627864 |
| BIN3 | 4.58602526160762e−164 | 0.328297788692693 |
| NDRG2 | 6.88401064309756e−134 | 0.328450109299609 |
| CTC1 | 1.23796303737966e−194 | 0.328711713478546 |
| ARHGAP12 | 5.81888505021512e−191 | 0.328885867369086 |
| MEF2A | 2.1155032931339e−147 | 0.329250582810696 |
| RNPEP | 2.93097257873988e−172 | 0.329735431449436 |
| CDC42BPB | 5.98393920409052e−149 | 0.329915830920303 |
| ZBTB17 | 2.09478165636721e−208 | 0.330094884777377 |
| GAS6 | 1.33061699941044e−173 | 0.330207416528773 |
| TIE1 | 1.46505855956562e−159 | 0.330491882468832 |
| TMA16 | 0 | 0.330738056578007 |
| ZC3H12A | 5.28474151606152e−175 | 0.331117707859936 |
| FXR1 | 0 | 0.331193149539605 |
| CDK10 | 1.32932264946722e−176 | 0.331418772734934 |
| FBRSL1 | 2.32091435702094e−151 | 0.331476045365771 |
| TMEM185B | 1.17679603033713e−188 | 0.331484182938668 |
| NOM1 | 1.35686209559121e−201 | 0.33157550271647 |
| ARHGEF10 | 5.24116363067378e−151 | 0.331611707118397 |
| STOM | 2.50757201781857e−153 | 0.331661819594659 |
| UXS1 | 5.47444613253315e−189 | 0.331737356220258 |
| STX11 | 1.47362439053006e−209 | 0.331876908922944 |
| SNX9 | 9.34809524177763e−172 | 0.331996163557697 |
| ZFAND2B | 3.00294474647764e−197 | 0.333643414469285 |
| PKIG | 2.08542455851914e−149 | 0.333854447119772 |
| SCAND1 | 0 | 0.33450215661109 |
| FILIP1L | 4.813818750118e−180 | 0.334613931601372 |
| UBALD2 | 2.022558432272e−171 | 0.334868760224975 |
| PFKM | 0 | 0.335260554967686 |
| PRKAA1 | 0 | 0.335425422647754 |
| USP36 | 5.57576462706763e−164 | 0.33544926057106 |
| RFTN1 | 1.2851023165397e−186 | 0.335549670439439 |
| NCKIPSD | 1.64514011669352e−196 | 0.335748719399944 |
| PRKD2 | 8.28605055417976e−177 | 0.335750455299262 |
| CAV2 | 1.37708177149846e−143 | 0.335834517076041 |
| STX6 | 8.09725158754362e−204 | 0.335889458830216 |
| SPEN | 1.09939784452263e−184 | 0.335899198330561 |
| NADSYN1 | 1.34818740093226e−172 | 0.33594452458826 |
| ADAM9 | 1.15256219879406e−145 | 0.336269339091808 |
| ANGEL1 | 0 | 0.336364045183023 |
| IFRD1 | 2.85558378933933e−187 | 0.336743821768028 |
| NDST1 | 3.03623424247299e−181 | 0.337240732517716 |
| RAP2B | 0 | 0.33726109172657 |
| RNF166 | 4.50875710305921e−159 | 0.337716204289033 |
| FBXO42 | 6.74892003915549e−205 | 0.337845599685461 |
| R3HCC1L | 8.850448191388e−206 | 0.338075072229773 |
| MON1B | 9.48638548536234e−196 | 0.338234962516765 |
| ALKBH5 | 3.23454684468205e−194 | 0.339554479307757 |
| DMAC2 | 1.83238017549321e−203 | 0.339973545046447 |
| EIF4ENIF1 | 2.9913164926438e−208 | 0.340661829887046 |
| SLC39A7 | 7.90716798427396e−206 | 0.341101341883583 |
| SAP30BP | 1.82133748450353e−173 | 0.341456819515882 |
| MARVELD1 | 3.41084780443486e−214 | 0.341603958187343 |
| CCDC61 | 1.59399174030374e−184 | 0.341751774084874 |
| IBTK | 2.84069156093541e−207 | 0.341862851425203 |
| EXOC3 | 1.2115901327604e−235 | 0.341866348734617 |
| PBXIP1 | 2.70951548793394e−154 | 0.342246360373365 |
| GSTM4 | 2.1398611518969e−195 | 0.342447554843048 |
| SERP1 | 1.64390001962365e−189 | 0.342661349928439 |
| TMEM255B | 6.95913405080346e−181 | 0.342715476582772 |
| PNPT1 | 2.68972328256176e−207 | 0.342830691156537 |
| PIP5K1C | 1.19819369216071e−203 | 0.343116045969159 |
| AKT1 | 6.05307857405598e−198 | 0.343232586476061 |
| PPP1R13B | 1.95649421964083e−196 | 0.343326006998096 |
| OAZ2 | 9.79336678666123e−163 | 0.343709181936329 |
| MYO1C | 1.06391776752462e−169 | 0.34402153753711 |
| CHP1 | 8.53556756278251e−167 | 0.34415163959963 |
| NPIPB6 | 2.99243934887648e−205 | 0.344296162607278 |
| FAM193A | 9.34471733758321e−179 | 0.344450494067148 |
| MFNG | 7.34057084764193e−171 | 0.344522652006884 |
| AKR7A2 | 1.01724757980587e−213 | 0.344638003657778 |
| CLSTN3 | 1.53399004950249e−198 | 0.345761650835713 |
| DMPK | 1.65010895115617e−204 | 0.345834295299062 |
| PEX19 | 6.32172780699694e−200 | 0.345992647501492 |
| NPDC1 | 5.49368030627979e−160 | 0.346001339065792 |
| ARID5A | 7.93889637885638e−196 | 0.346641929993659 |
| TRAPPC6B | 5.68017106326485e−203 | 0.34702404112755 |
| SECISBP2 | 3.5522464236175e−203 | 0.347124021009093 |
| PPM1D | 4.17032726404473e−202 | 0.347293788423263 |
| VASN | 0 | 0.34739454617323 |
| MARK2 | 1.37957178839349e−210 | 0.348149554883481 |
| ARHGAP17 | 1.64311093311531e−178 | 0.348533558530584 |
| DNAJB2 | 3.31121991886228e−185 | 0.348696208307006 |
| IL6R | 5.68006724759494e−201 | 0.348983494665141 |
| RSRC2 | 0 | 0.349160395705346 |
| RAPGEF6 | 2.30333092110148e−215 | 0.349375378186355 |
| CYBRD1 | 4.91982401406936e−192 | 0.349392835417083 |
| SLC35C2 | 0 | 0.349869568023197 |
| PPP6R3 | 7.0982176595992e−213 | 0.349871551511315 |
| MICAL2 | 4.3506945363263e−195 | 0.350170074317243 |
| FLOT1 | 3.72404494846224e−144 | 0.350849166011865 |
| ARRB1 | 5.1254020158745e−158 | 0.351523278105786 |
| GTF2F1 | 3.88771478115157e−182 | 0.351952359947161 |
| TIMM10B | 1.31224278419473e−188 | 0.351953555189605 |
| SNX12 | 0 | 0.35213706493129 |
| SRP14 | 4.48618648590419e−174 | 0.352178282880374 |
| BTG3 | 6.52250510913458e−215 | 0.352205162365703 |
| PSPC1 | 1.51573372265304e−215 | 0.352263853909427 |
| PPP2R5A | 0 | 0.352477919980312 |
| CASP8AP2 | 0 | 0.352498495927661 |
| AP2A1 | 3.5596680935396e−215 | 0.35258817457689 |
| ANAPC2 | 4.01409449437196e−202 | 0.352832982204365 |
| ACTR1B | 1.34341857295337e−252 | 0.35321684267751 |
| SLC7A6 | 0 | 0.353313405388409 |
| SVIL | 1.08187020742868e−197 | 0.353379671662019 |
| MBNL2 | 4.47568543279341e−222 | 0.353414704773024 |
| MTCH2 | 0 | 0.353716445425211 |
| PARVG | 3.15074471623435e−202 | 0.353760103572198 |
| PTAR1 | 1.88003521778356e−218 | 0.353780685689049 |
| TMEM134 | 2.9558445252112e−218 | 0.353789481628796 |
| MTERF4 | 4.30066935676103e−208 | 0.354136414494843 |
| RABGGTA | 6.21572009584891e−193 | 0.354311011067717 |
| GTF2H4 | 3.06628370882825e−218 | 0.354474561596565 |

TABLE 11
SARS-CoV-2 biomarkers that were not dysregulated by
more than 0.1-fold in infected human lung tissue.

| | | | Expression in presence |
|---|---|---|---|
| | Adjusted | Average | of SARS-CoV-2 |
| Gene | p-val | log2FC | infection |
| ZBTB16 | 1.000000e+00 | 0.02623837 | 2% down regulation |
| SCARB1 | 1.000000e+00 | 0.03582289 | 3% down regulation |
| AHNAK | 1.000000e+00 | 0.04630844 | 4% down regulation |
| ADAMTS1 | 3.389032e−16 | 0.06160682 | 6% down regulation |
| AFDN | 1.310373e−09 | 0.07819874 | 7% down regulation |
| LRRC32 | 2.254537e−15 | 0.08190498 | 8% down regulation |

As shown in Tables 9-11 and shown spatially in FIGS. 19-30, at least 400 biomarkers were upregulated or downregulated in a human lung tissue sample infected with SARS-CoV-2. These data demonstrate a proof of concept that biomarkers associated with SARS-CoV-2 can be identified using the RTL methods from Example 1 at the same time as detecting SARS-CoV-2 virus, in general, and specifically in lung tissue.

### Example 4. Identification of Spatial Clusters in FFPE-Fixed Lung Tissue Samples Using Templated Ligation

The Seurat SCTransform function was applied to normalize the individual filtered count matrices, and integrated in Seurat using SelectIntegrationFeatures, and IntegrateData. Principal Component Analysis (PCA), and UMAP was applied using 50 principal components, and 35 were further used in downstream analysis, and clustering. Batch effects were addressed, and removed using RunHarmony (group.by.vars as slide ID, and 150 iterations) applied on the PCA-computed matrix. Clustering was applied at a resolution of 0.4.

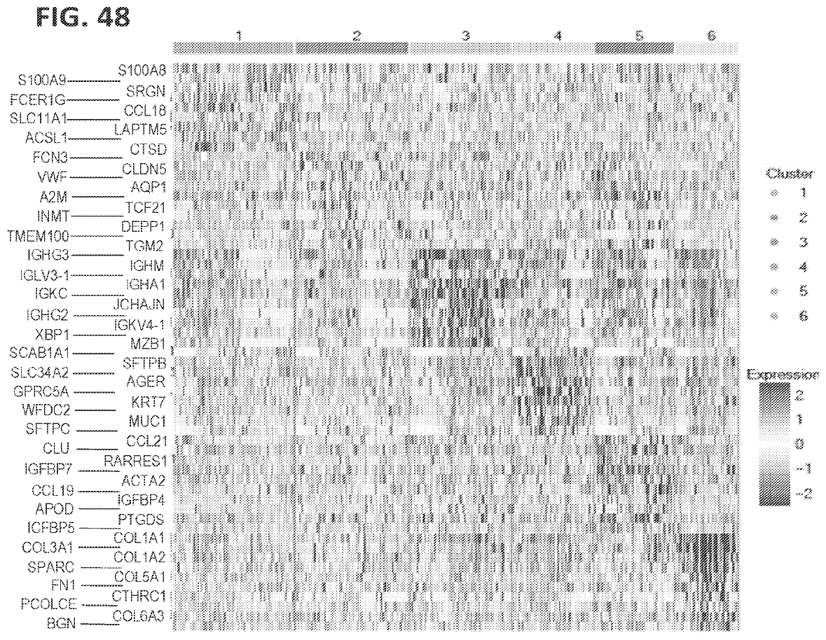

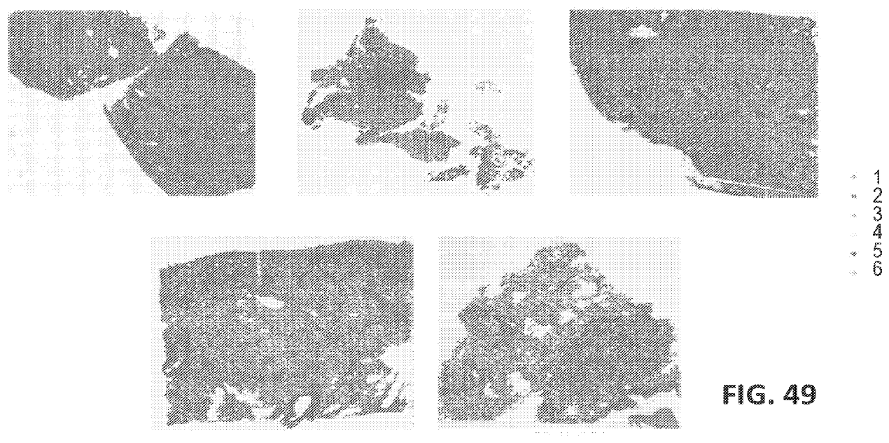

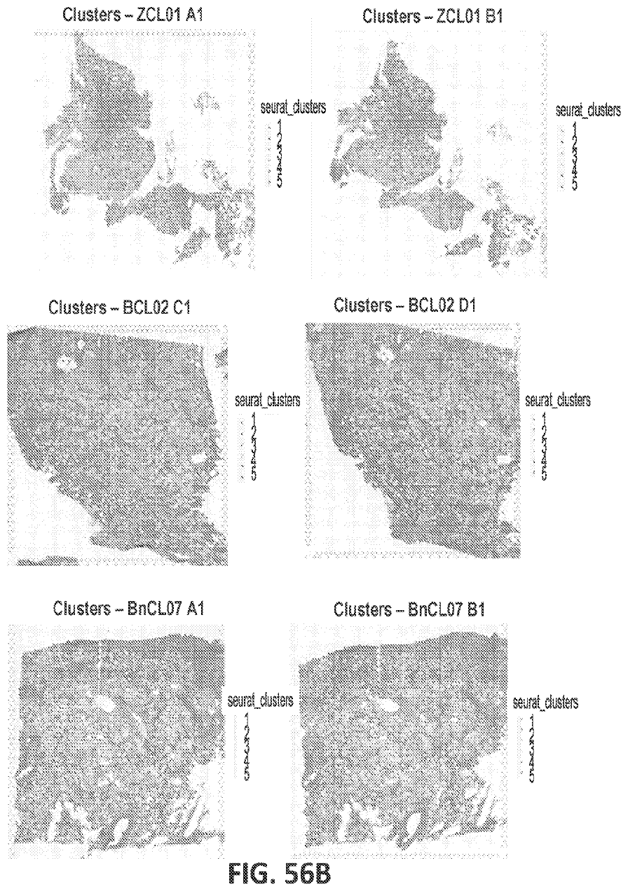

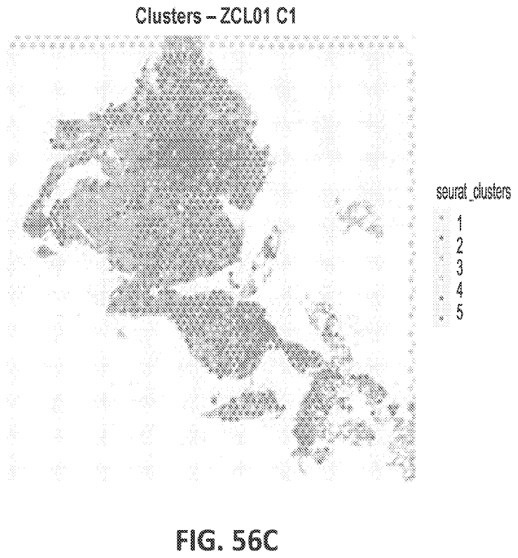

To explore the human lung cellular landscape in response to SARS-CoV-2 infection, unsupervised, joint graph-based clustering of spatial transcriptome data collected from both COVID-positive and COVID-negative sections was performed, and six distinct clusters were identified (FIG. 47A-47B). When overlaid on the H&E stained tissue sections, the distribution of the spatial clusters clearly fit with the observed tissue structural components (FIG. 48, FIG. 56A-56C). Investigation into the differentially expressed genes of each cluster revealed their cellular compositions (FIG. 49). As expected when working with spatial transcriptomics data, all six clusters comprised a mixture of different cell types. However, in five clusters, dominant differential expression (DE) gene signatures specific for myeloid cells (cluster 1 and 5), endothelial cells (cluster 2), B-cell/plasma cells (cluster 3), epithelial cells (cluster 4), and fibroblasts (cluster 6) were identified (FIGS. 48-49, 56A-56C). Cluster 5 was characterized by DE genes specific for endothelial, fibroblast and smooth muscle cells without a clear dominance of any of those cell types. Further sub clustering of this group resulted in three subclusters (subcluster 1: fibroblast-dominated, subcluster 2: smooth muscle cell-dominated, subcluster 3: mixture of endothelial and immune cells), in line with previous observations. Representative dysregulated genes identified in each cluster is provided below in Table 12, and representative dysregulated genes identified in each subcluster of Cluster 5 is provided below in Table 13.

TABLE 12
Representative Dysregulated Genes in Clusters 1-6 from both COVID-positive and COVID-negative sections.

| | | | | | | |
|---|---|---|---|---|---|---|
| gene | p_val | avg_log2FC | pct. 1 | pct. 2 | p_val_adj | description/cell type |

CLUSTER 1

| | | | | | | |
|---|---|---|---|---|---|---|
| S100A8 | 0 | 0.995289312 | 0.987 | 0.943 | 0 | Blood immune, predominantly |
| | | | | | | macrophages |
| S100A9 | 0 | 0.918333921 | 0.999 | 0.989 | 0 | Blood immune, predominantly |
| | | | | | | macrophages |
| SPP1 | 7.1E−128 | 0.859443185 | 0.72 | 0.592 | 1.2E−123 | Blood immune, predominantly |
| | | | | | | macrophages |
| SRGN | 0 | 0.790989829 | 0.95 | 0.837 | 0 | Blood immune, predominantly |
| | | | | | | macrophages and granulocytes |
| FCER1G | 0 | 0.762238591 | 0.933 | 0.799 | 0 | Blood immune, predominantly |
| | | | | | | macrophages and granulocytes |
| CCL18 | 2E−98 | 0.744414387 | 0.689 | 0.658 | 3.49E−94 | Blood immune, predominantly |
| | | | | | | macrophages |
| JCHAIN | 5.46E−64 | −0.584242866 | 0.619 | 0.674 | 9.53E−60 | |
| COL1A1 | 3.51E−77 | −0.586464921 | 0.701 | 0.811 | 6.14E−73 | Fibroblast marker |
| LUM | 1.1E−243 | −0.587399348 | 0.728 | 0.841 | 1.9E−239 | |
| IGHG2 | 1.58E−63 | −0.588577587 | 0.448 | 0.561 | 2.75E−59 | |
| ACTA2 | 1.1E−249 | −0.608748118 | 0.571 | 0.713 | 2E−245 | Myofibroblasts marker (canonical) |
| IGHM | 2.43E−46 | −0.616384482 | 0.61 | 0.699 | 4.24E−42 | |
| C7 | 0 | −0.636635415 | 0.6 | 0.74 | 0 | complement system markers |
| | | | | | | (fibroblasts and endothelial) |
| COL3A1 | 5.8E−148 | −0.655303301 | 0.775 | 0.896 | 1E−143 | |
| C1R | 0 | −0.65723444 | 0.892 | 0.968 | 0 | Fibroblasts |
| IGFBP4 | 1.2E−238 | −0.660461912 | 0.36 | 0.537 | 2.1E−234 | |
| IGFBP7 | 0 | −0.673644799 | 0.894 | 0.952 | 0 | |
| RARRES1 | 2.4E−186 | −0.675647688 | 0.282 | 0.442 | 4.2E−182 | |
| IGKC | 5.2E−101 | −0.679947857 | 0.771 | 0.857 | 9.09E−97 | |
| COL1A2 | 3.1E−206 | −0.699121907 | 0.803 | 0.917 | 5.4E−202 | Fibroblast marker |
| PTGDS | 0 | −0.700125319 | 0.728 | 0.847 | 0 | |
| FBLN1 | 0 | −0.706984572 | 0.639 | 0.8 | 0 | |
| A2M | 0 | −0.7823483 | 0.946 | 0.949 | 0 | |
| IGHG3 | 8.05E−91 | −0.82948686 | 0.601 | 0.723 | 1.41E−86 | |
| CLU | 0 | −0.843717445 | 0.822 | 0.913 | 0 | endothelial; epithelial, granulocytes |
| IGHA1 | 7.8E−180 | −0.934905345 | 0.694 | 0.807 | 1.4E−175 | |

CLUSTER 2

| | | | | | | |
|---|---|---|---|---|---|---|
| FCN3 | 0 | 0.587074847 | 0.7 | 0.509 | 0 | endothelial; some alveolar type 2 |
| CLDN5 | 0 | 0.575501489 | 0.745 | 0.607 | 0 | endothelial |
| VWF | 0 | 0.561851962 | 0.884 | 0.735 | 0 | endothelial; some alveolar type 2 |
| AQP1 | 0 | 0.54133989 | 0.788 | 0.636 | 0 | endothelial; alveolar type 1 and 2 |
| A2M | 4.78E−86 | 0.533654918 | 0.94 | 0.951 | 8.34E−82 | endothelial; alveolar type 2, |
| | | | | | | fibroblasts in some case |
| TCF21 | 0 | 0.518785836 | 0.822 | 0.643 | 0 | fibroblasts; also endothelial and |
| | | | | | | alveolar type 1 |
| HLA-DRA | 3E−224 | −0.563910926 | 0.704 | 0.813 | 5.3E−220 | |
| CCL21 | 2.61E−81 | −0.586761515 | 0.254 | 0.356 | 4.55E−77 | endothelial |
| CD163 | 4.9E−299 | −0.594258078 | 0.868 | 0.926 | 8.6E−295 | CD16 macrophages |
| HLA- | 1.2E−189 | −0.594280312 | 0.395 | 0.561 | 2.1E−185 | Fibroblast |
| DRB5 | | | | | | |
| CCL18 | 2.6E−135 | −0.60435764 | 0.572 | 0.689 | 4.5E−131 | |
| IFI30 | 7.3E−301 | −0.605400234 | 0.754 | 0.86 | 1.3E−296 | |
| CTSD | 3E−298 | −0.626041994 | 0.932 | 0.968 | 5.2E−294 | |
| CTSB | 5.1E−307 | −0.629088278 | 0.959 | 0.98 | 8.9E−303 | |
| LYZ | 4.1E−243 | −0.656995636 | 0.859 | 0.918 | 7.2E−239 | |
| COL1A1 | 1.7E−107 | −0.658010053 | 0.706 | 0.808 | 2.9E−103 | fibroblast |
| XBP1 | 1.1E−235 | −0.690911776 | 0.744 | 0.83 | 1.9E−231 | |
| IGHG2 | 3.88E−43 | −0.719665607 | 0.486 | 0.549 | 6.77E−39 | |
| IGKV4-1 | 1.3E−103 | −0.727606194 | 0.484 | 0.619 | 2.3E−99 | |
| IGHA1 | 5.87E−92 | −0.823451941 | 0.723 | 0.797 | 1.03E−87 | |
| CXCL9 | 7.5E−200 | −0.890006929 | 0.314 | 0.49 | 1.3E−195 | |
| IGKC | 2.7E−180 | −0.895160824 | 0.781 | 0.853 | 4.6E−176 | |
| JCHAIN | 4.8E−257 | −0.976128565 | 0.541 | 0.693 | 8.4E−253 | |
| IGLV3-1 | 4.4E−195 | −1.053768549 | 0.396 | 0.562 | 7.8E−191 | |
| IGHM | 7.5E−225 | −1.15713407 | 0.573 | 0.707 | 1.3E−220 | |
| IGHG3 | 5.6E−210 | −1.323163134 | 0.612 | 0.718 | 9.7E−206 | |

CLUSTER 3

| | | | | | | |
|---|---|---|---|---|---|---|
| IGHG3 | 0 | 1.951580069 | 0.864 | 0.659 | 0 | |
| IGHM | 0 | 1.700414355 | 0.841 | 0.644 | 0 | mix, low cell type specificity |
| IGLV3-1 | 0 | 1.621594271 | 0.716 | 0.487 | 0 | |
| IGHA1 | 0 | 1.620414716 | 0.917 | 0.752 | 0 | |
| IGKC | 0 | 1.59379671 | 0.961 | 0.811 | 0 | |
| JCHAIN | 0 | 1.490980991 | 0.811 | 0.629 | 0 | T-cells; low exp for alveolar type 2 |
| IGHG2 | 7.1E−254 | 1.375577348 | 0.673 | 0.506 | 1.2E−249 | |
| IGKV4-1 | 2.1E−303 | 1.363272409 | 0.75 | 0.556 | 3.6E−299 | |
| COL1A2 | 2.2E−12 | −0.303455005 | 0.952 | 0.879 | 3.84E−08 | Fibroblast |
| AQP9 | 1.36E−87 | −0.305870142 | 0.336 | 0.455 | 2.38E−83 | |
| CSF3R | 5.23E−72 | −0.313670996 | 0.35 | 0.454 | 9.14E−68 | |
| VCAN | 5.5E−109 | −0.313764128 | 0.554 | 0.663 | 9.7E−105 | |
| S100A9 | 1.07E−14 | −0.314772428 | 0.992 | 0.991 | 1.86E−10 | Blood immune, activated |
| | | | | | | macrophages |
| CCN1 | 2.04E−81 | −0.317613637 | 0.624 | 0.703 | 3.56E−77 | |
| SFTPB | 4.23E−39 | −0.32040958 | 0.81 | 0.858 | 7.39E−35 | |
| GPRC5A | 2.76E−74 | −0.330675906 | 0.516 | 0.618 | 4.81E−70 | |
| COL3A1 | 4.79E−17 | −0.337293082 | 0.934 | 0.855 | 8.37E−13 | |
| G0S2 | 4.38E−55 | −0.345008186 | 0.727 | 0.764 | 7.65E−51 | |
| ACSL1 | 6.3E−127 | −0.374552995 | 0.576 | 0.687 | 1.1E−122 | |
| AGER | 1.52E−67 | −0.388973067 | 0.686 | 0.76 | 2.66E−63 | |
| S100A8 | 1.23E−11 | −0.416603306 | 0.954 | 0.952 | 2.16E−07 | Blood immune, predominantly |
| | | | | | | macrophages |
| SPP1 | 3.24E−16 | −0.418534692 | 0.576 | 0.629 | 5.65E−12 | |
| KRT7 | 1.6E−126 | −0.420271744 | 0.636 | 0.741 | 2.7E−122 | |
| THBS1 | 4.04E−91 | −0.427015317 | 0.79 | 0.836 | 7.06E−87 | macrophages, fibroblasts |
| PTX3 | 1.42E−90 | −0.447207489 | 0.368 | 0.492 | 2.48E−86 | |
| SLC11A1 | 2.7E−175 | −0.454141087 | 0.546 | 0.689 | 4.7E−171 | |
| COL1A1 | 6.82E−09 | −0.468416717 | 0.873 | 0.768 | 0.000119 | fibroblast |
| SCGB1A1 | 1.42E−46 | −0.547178474 | 0.156 | 0.232 | 2.49E−42 | |

CLUSTER 4

| | | | | | | |
|---|---|---|---|---|---|---|
| SCGB1A1 | 2.4E−127 | 1.525296798 | 0.33 | 0.199 | 4.2E−123 | epithelial (club main, then ciliated) |
| SFTPB | 0 | 1.465732603 | 0.986 | 0.826 | 0 | epithelial (alveolar type 1, 2; club) |
| SLC34A2 | 0 | 1.266203174 | 0.893 | 0.573 | 0 | epithelial (Alveolar type 1, type 2) |
| AGER | 0 | 1.2374699 | 0.916 | 0.718 | 0 | epithelial (alveolar type 1) |
| GPRC5A | 0 | 1.173828571 | 0.865 | 0.554 | 0 | epithelial (alveolar type 1; some |
| | | | | | | alveolar type 2 and club cells) |
| KRT7 | 0 | 1.142426222 | 0.951 | 0.683 | 0 | epithelial (alveolar type 1; some |
| | | | | | | alveolar type 2, club, ciliated) |
| WFDC2 | 0 | 1.101802204 | 0.863 | 0.532 | 0 | epithelial (club, ciliated) |
| MUC1 | 0 | 1.055234864 | 0.765 | 0.422 | 0 | epithelial (alveolar type 2; type 1) |
| HILPDA | 1.63E−77 | −0.35664759 | 0.486 | 0.581 | 2.86E−73 | |
| RARRES1 | 1.34E−25 | −0.368615802 | 0.354 | 0.416 | 2.34E−21 | |
| XBP1 | 1.94E−22 | −0.373162561 | 0.823 | 0.81 | 3.38E−18 | |
| CD163 | 1.7E−59 | −0.376819034 | 0.902 | 0.916 | 2.96E−55 | |
| ADAMTS2 | 9.47E−55 | −0.377019789 | 0.465 | 0.538 | 1.65E−50 | |
| NFKBIA | 2.7E−34 | −0.382687655 | 0.702 | 0.72 | 4.71E−30 | |
| TIMP1 | 2.2E−133 | −0.397458041 | 0.999 | 1 | 3.9E−129 | |
| MT1G | 2.09E−41 | −0.397918674 | 0.568 | 0.627 | 3.66E−37 | |
| LYZ | 6.73E−38 | −0.422024288 | 0.912 | 0.905 | 1.18E−33 | |
| CCL19 | 2.9E−81 | −0.446739322 | 0.357 | 0.47 | 5.06E−77 | |
| IGFBP7 | 9.2E−111 | −0.450100292 | 0.924 | 0.942 | 1.6E−106 | |
| JCHAIN | 8.54E−25 | −0.505994824 | 0.648 | 0.665 | 1.49E−20 | |
| IGHM | 4.28E−14 | −0.561499135 | 0.66 | 0.683 | 7.48E−10 | |
| CCL21 | 1.43E−58 | −0.582523685 | 0.256 | 0.349 | 2.5E−54 | |
| IGLV3-1 | 1.97E−16 | −0.584902359 | 0.496 | 0.534 | 3.45E−12 | |
| IGHG3 | 3.64E−31 | −0.809521599 | 0.666 | 0.702 | 6.37E−27 | |
| CXCL9 | 8.03E−49 | −0.910796104 | 0.4 | 0.464 | 1.4E−44 | |
| CLU | 4.22E−07 | −0.330519909 | 0.909 | 0.891 | 0.007365 | |
| IGFBP4 | 1.72E−05 | −0.269286822 | 0.494 | 0.5 | 0.300278 | |
| COL3A1 | 0.006846 | −0.251542281 | 0.838 | 0.875 | 1 | |

CLUSTER 5

| | | | | | | |
|---|---|---|---|---|---|---|
| CCL21 | 0 | 1.445492887 | 0.54 | 0.302 | 0 | endothelial |
| CLU | 0 | 1.440413177 | 0.979 | 0.88 | 0 | Mix of immune, epithelial and |
| | | | | | | fibroblasts |
| RARRES1 | 1E−282 | 1.113374325 | 0.588 | 0.377 | 1.8E−278 | Fibroblasts |
| IGFBP7 | 0 | 1.098118825 | 0.988 | 0.931 | 0 | Endothelial |
| TYROBP | 1.3E−293 | −0.556590808 | 0.721 | 0.854 | 2.2E−289 | |
| C1QC | 4.6E−124 | −0.556857782 | 0.807 | 0.874 | 8E−120 | |
| WFDC2 | 1.7E−217 | −0.571111835 | 0.404 | 0.609 | 2.9E−213 | |
| SLC11A1 | 1.1E−207 | −0.574505191 | 0.507 | 0.689 | 2E−203 | |
| COL1A2 | 1.76E−29 | −0.642163291 | 0.935 | 0.885 | 3.07E−25 | Fibroblast marker |
| RNASE1 | 8.1E−262 | −0.675084228 | 0.825 | 0.906 | 1.4E−257 | |
| IFI6 | 2.8E−141 | −0.675163631 | 0.646 | 0.751 | 4.9E−137 | |
| LAPTM5 | 0 | −0.682800016 | 0.873 | 0.943 | 0 | |
| SLC34A2 | 1.1E−296 | −0.703762888 | 0.412 | 0.654 | 1.9E−292 | |
| GPRC5A | 5.2E−299 | −0.715292671 | 0.401 | 0.632 | 9.1E−295 | |
| KRT7 | 0 | −0.824266775 | 0.533 | 0.753 | 0 | |
| C1QB | 1.9E−225 | −0.829335274 | 0.783 | 0.865 | 3.3E−221 | |
| COL3A1 | 2.29E−77 | −0.935428165 | 0.918 | 0.862 | 4E−73 | |
| CCL18 | 3.3E−226 | −0.945512472 | 0.536 | 0.686 | 5.7E−222 | |
| APOE | 7E−118 | −0.960832679 | 0.658 | 0.713 | 1.2E−113 | |
| SFTPB | 0 | −0.97265939 | 0.717 | 0.871 | 0 | epithelial (alveolar type 2; type 1) |
| COL1A1 | 8.29E−66 | −0.997850237 | 0.83 | 0.781 | 1.45E−61 | Fibroblast marker |
| AGER | 0 | −1.051404726 | 0.549 | 0.779 | 0 | |
| SPP1 | 1.4E−291 | −1.204733092 | 0.42 | 0.652 | 2.5E−287 | |

CLUSTER 6

| | | | | | | |
|---|---|---|---|---|---|---|
| COL1A1 | 0 | 2.154042298 | 0.959 | 0.766 | 0 | Fibroblasts |
| COL3A1 | 0 | 2.050975117 | 0.98 | 0.855 | 0 | Fibroblasts |
| COL1A2 | 0 | 1.880281944 | 0.982 | 0.881 | 0 | Fibroblasts |
| SPARC | 0 | 1.500423814 | 0.998 | 0.981 | 0 | Vascular (fibroblasts, endothelial |
| | | | | | | smooth muscles) |
| COL5A1 | 0 | 1.420806074 | 0.859 | 0.552 | 0 | Fibroblasts |
| FN1 | 0 | 1.135764332 | 0.999 | 0.993 | 0 | Macrophages; maybe some |
| | | | | | | fibroblasts and endothelial as well |
| HSPA1B | 6.01E−66 | −0.437952417 | 0.831 | 0.831 | 1.05E−61 | |
| ID1 | 1.9E−117 | −0.438539841 | 0.501 | 0.66 | 3.3E−113 | lung injury repair key regulator |
| GPRC5A | 2.14E−82 | −0.439621059 | 0.493 | 0.613 | 3.73E−78 | |
| THBS1 | 1.1E−19 | −0.464566232 | 0.844 | 0.826 | 1.93E−15 | macrophages, fibroblasts |
| CLDN5 | 4.2E−143 | −0.466545711 | 0.497 | 0.653 | 7.3E−139 | endothelial |
| TM4SF1 | 9.1E−149 | −0.476899397 | 0.434 | 0.608 | 1.6E−144 | |
| VWF | 1.5E−174 | −0.478233626 | 0.623 | 0.783 | 2.5E−170 | |
| FCN3 | 4.4E−138 | −0.502486059 | 0.393 | 0.567 | 7.7E−134 | |
| TCF21 | 7.1E−179 | −0.505946632 | 0.526 | 0.699 | 1.2E−174 | |
| KRT7 | 7.1E−148 | −0.539985199 | 0.599 | 0.738 | 1.2E−143 | |
| SCGB1A1 | 4.66E−26 | −0.552402868 | 0.16 | 0.226 | 8.13E−22 | |
| AQP1 | 1.2E−161 | −0.58072802 | 0.539 | 0.683 | 2E−157 | endothelial, alveolar type1, type2 |
| TGM2 | 2.5E−272 | −0.605053586 | 0.826 | 0.924 | 4.3E−268 | |
| S100A9 | 1.4E−109 | −0.617790881 | 0.993 | 0.991 | 2.5E−105 | Blood immune, activated |
| | | | | | | macrophages |
| SFTPB | 7.4E−135 | −0.62030658 | 0.778 | 0.858 | 1.3E−130 | |
| PTX3 | 3.8E−101 | −0.635839311 | 0.333 | 0.487 | 6.63E−97 | |
| CLU | 1.8E−167 | −0.637513218 | 0.812 | 0.904 | 3.1E−163 | mix of immune, epithelial and |
| | | | | | | fibroblasts |
| AGER | 7.9E−167 | −0.729564137 | 0.64 | 0.76 | 1.4E−162 | |
| G0S2 | 3.4E−211 | −0.809960861 | 0.625 | 0.774 | 5.9E−207 | |
| S100A8 | 5.2E−215 | −0.944259427 | 0.901 | 0.959 | 9.1E−211 | Blood immune |

TABLE 13
Representative Dysregulated Genes in Subclusters 1-3 of Cluster 5.

| gene | p_val | avg_log2FC | pct. 1 | pct. 2 | p_val_adj |
|---|---|---|---|---|---|
| Cluster 5, sub-cluster-1 | | | | | |
| RARRES1 | 1.32E−73 | 1.212019 | 0.655 | 0.436 | 2.15E−69 |
| G0S2 | 1.02E−59 | 1.030531 | 0.838 | 0.759 | 1.65E−55 |
| PTX3 | 4.58E−32 | 0.985065 | 0.589 | 0.494 | 7.45E−28 |
| SAA2 | 3.81E−34 | 0.810404 | 0.274 | 0.133 | 6.2E−30 |
| C3 | 1.56E−58 | 0.684932 | 0.815 | 0.698 | 2.54E−54 |
| PTGDS | 2.89E−52 | 0.682451 | 0.918 | 0.812 | 4.7E−48 |
| FBLN1 | 8.37E−70 | 0.667616 | 0.864 | 0.752 | 1.36E−65 |
| APOD | 1.9E−23 | 0.584577 | 0.536 | 0.416 | 3.1E−19 |
| IGFBP4 | 1.19E−17 | 0.58008 | 0.676 | 0.602 | 1.94E−13 |
| PLA2G2A | 3.01E−53 | 0.576398 | 0.407 | 0.204 | 4.9E−49 |
| DCN | 4.57E−92 | 0.570039 | 0.996 | 0.985 | 7.44E−88 |
| MMP2 | 2.13E−70 | 0.567257 | 0.894 | 0.771 | 3.47E−66 |
| ACKR1 | 4.21E−21 | 0.553977 | 0.548 | 0.458 | 6.85E−17 |
| TIMP1 | 1.05E−43 | 0.542487 | 1 | 1 | 1.7E−39 |
| CFD | 2.28E−47 | 0.532237 | 0.773 | 0.643 | 3.71E−43 |
| THBS1 | 7.53E−19 | 0.526449 | 0.874 | 0.858 | 1.23E−14 |
| CCL14 | 3.14E−16 | 0.513183 | 0.409 | 0.323 | 5.11E−12 |
| IGF1 | 5.55E−40 | 0.500874 | 0.53 | 0.373 | 9.04E−36 |
| SELE | 4.16E−10 | 0.484803 | 0.246 | 0.182 | 6.77E−06 |
| C7 | 9.1E−37 | 0.471612 | 0.82 | 0.727 | 1.48E−32 |
| RHOB | 3.75E−23 | −0.39951 | 0.684 | 0.743 | 6.09E−19 |
| MFGE8 | 3.37E−38 | −0.42028 | 0.773 | 0.824 | 5.48E−34 |
| SORBS1 | 1.1E−63 | −0.42616 | 0.141 | 0.331 | 1.8E−59 |
| NR4A1 | 8.96E−21 | −0.45227 | 0.324 | 0.431 | 1.46E−16 |
| C11orf96 | 1.53E−32 | −0.48424 | 0.547 | 0.658 | 2.49E−28 |
| TPM1 | 3.31E−41 | −0.48497 | 0.597 | 0.726 | 5.39E−37 |
| CRYAB | 8.02E−28 | −0.53452 | 0.431 | 0.538 | 1.3E−23 |
| FLNA | 5.95E−47 | −0.54509 | 0.948 | 0.96 | 9.67E−43 |
| ADIRF | 4.51E−52 | −0.54509 | 0.336 | 0.518 | 7.34E−48 |
| MYLK | 3.38E−33 | −0.55553 | 0.448 | 0.586 | 5.5E−29 |
| CNN1 | 2.91E−85 | −0.57596 | 0.11 | 0.319 | 4.73E−81 |
| ACTG2 | 1.5E−62 | −0.72265 | 0.107 | 0.281 | 2.44E−58 |
| IGFBP5 | 3.29E−77 | −0.72642 | 0.609 | 0.772 | 5.36E−73 |
| DES | 5.8E−118 | −0.79112 | 0.154 | 0.427 | 9.4E−114 |
| MUSTN1 | 9.79E−85 | −0.80074 | 0.15 | 0.372 | 1.59E−80 |
| TPM2 | 3.6E−105 | −0.892 | 0.926 | 0.957 | 5.9E−101 |
| MYL9 | 2.2E−68 | −0.91316 | 0.72 | 0.816 | 3.58E−64 |
| TAGLN | 1.28E−74 | −1.0685 | 0.651 | 0.769 | 2.08E−70 |
| ACTA2 | 7.4E−103 | −1.28309 | 0.789 | 0.864 | 1.21E−98 |
| CCL21 | 1.2E−100 | −1.88773 | 0.488 | 0.659 | 1.9E−96 |
| Cluster 5, sub-cluster-2 | | | | | |
| ACTA2 | 1.4E−304 | 1.906385 | 0.983 | 0.775 | 2.3E−300 |
| TAGLN | 1.5E−251 | 1.680161 | 0.936 | 0.634 | 2.5E−247 |
| MYL9 | 7.5E−218 | 1.448269 | 0.942 | 0.708 | 1.2E−213 |
| TPM2 | 3.6E−237 | 1.34458 | 0.99 | 0.924 | 5.8E−233 |
| DES | 9.2E−238 | 1.176521 | 0.626 | 0.155 | 1.5E−233 |
| MUSTN1 | 1.3E−131 | 1.145805 | 0.489 | 0.159 | 2.1E−127 |
| ACTG2 | 3.1E−134 | 1.119978 | 0.416 | 0.106 | 5E−130 |
| CRYAB | 2.1E−107 | 0.961367 | 0.71 | 0.411 | 3.4E−103 |
| FLNA | 2.3E−149 | 0.94142 | 0.988 | 0.944 | 3.8E−145 |
| MYLK | 2.42E−81 | 0.927632 | 0.695 | 0.447 | 3.93E−77 |
| CNN1 | 6.4E−193 | 0.917501 | 0.49 | 0.107 | 1E−188 |
| TPM1 | 9.6E−111 | 0.838745 | 0.834 | 0.594 | 1.6E−106 |
| C11orf96 | 2.78E−96 | 0.829099 | 0.793 | 0.536 | 4.52E−92 |
| ADIRF | 1.66E−91 | 0.810055 | 0.634 | 0.34 | 2.7E−87 |
| RHOB | 4.15E−87 | 0.764122 | 0.846 | 0.671 | 6.75E−83 |
| NR4A1 | 2.7E−45 | 0.727707 | 0.518 | 0.322 | 4.39E−41 |
| SOD3 | 1.24E−84 | 0.720701 | 0.937 | 0.801 | 2.02E−80 |
| SORBS1 | 3.9E−156 | 0.720303 | 0.5 | 0.135 | 6.4E−152 |
| BGN | 6.59E−51 | 0.646117 | 0.811 | 0.646 | 1.07E−46 |
| TGM2 | 1.21E−78 | 0.644919 | 0.989 | 0.974 | 1.96E−74 |
| PDPN | 3.25E−44 | −0.50462 | 0.302 | 0.546 | 5.29E−40 |
| SLC4A1 | 1.95E−16 | −0.51262 | 0.096 | 0.211 | 3.17E−12 |
| SELE | 7.65E−19 | −0.51802 | 0.12 | 0.249 | 1.25E−14 |
| XBP1 | 5.03E−18 | −0.52025 | 0.734 | 0.808 | 8.18E−14 |
| ACKR1 | 1.14E−18 | −0.53306 | 0.414 | 0.543 | 1.85E−14 |
| LUM | 1.48E−36 | −0.53337 | 0.697 | 0.877 | 2.4E−32 |
| LYVE1 | 6.78E−29 | −0.53435 | 0.29 | 0.476 | 1.1E−24 |
| CTSB | 2.04E−33 | −0.53676 | 0.964 | 0.967 | 3.32E−29 |
| DCN | 3.48E−67 | −0.56892 | 0.978 | 0.996 | 5.66E−63 |
| JCHAIN | 1.25E−15 | −0.58457 | 0.529 | 0.636 | 2.04E−11 |
| C3 | 4.48E−32 | −0.58657 | 0.668 | 0.803 | 7.29E−28 |
| CCL14 | 3.42E−29 | −0.59306 | 0.231 | 0.415 | 5.57E−25 |
| MMP2 | 3.96E−61 | −0.60305 | 0.688 | 0.892 | 6.44E−57 |
| SAA2 | 1.72E−12 | −0.66263 | 0.152 | 0.248 | 2.8E−08 |
| IGHG3 | 1.93E−12 | −0.66707 | 0.622 | 0.701 | 3.14E−08 |
| PTGDS | 6.7E−57 | −0.75392 | 0.721 | 0.921 | 1.09E−52 |
| G0S2 | 6.65E−19 | −0.82788 | 0.784 | 0.821 | 1.08E−14 |
| CXCL9 | 7.58E−18 | −0.84552 | 0.347 | 0.475 | 1.23E−13 |
| RARRES1 | 1.35E−39 | −1.00095 | 0.406 | 0.627 | 2.2E−35 |
| CCL21 | 1.04E−27 | −1.1799 | 0.407 | 0.568 | 1.69E−23 |
| Cluster 5, sub-cluster-3 | | | | | |
| CCL21 | 0 | 2.937339 | 0.999 | 0.472 | 0 |
| CXCL9 | 2.42E−37 | 0.979592 | 0.621 | 0.427 | 3.94E−33 |
| LYZ | 1.47E−41 | 0.827321 | 0.904 | 0.826 | 2.4E−37 |
| MMRN1 | 6.38E−88 | 0.814399 | 0.532 | 0.194 | 1.04E−83 |
| HLA-DRB5 | 6.06E−46 | 0.798135 | 0.663 | 0.447 | 9.86E−42 |
| IGHD | 1.52E−21 | 0.773716 | 0.224 | 0.1 | 2.48E−17 |
| PI15 | 7.51E−32 | 0.73733 | 0.464 | 0.271 | 1.22E−27 |
| CD163 | 1.57E−34 | 0.721885 | 0.899 | 0.86 | 2.55E−30 |
| FLT4 | 4.5E−151 | 0.700439 | 0.475 | 0.103 | 7.3E−147 |
| PDPN | 1.82E−59 | 0.691318 | 0.716 | 0.471 | 2.96E−55 |
| TBX1 | 7.4E−165 | 0.652191 | 0.402 | 0.063 | 1.2E−160 |
| IGHM | 9.95E−32 | 0.646655 | 0.793 | 0.623 | 1.62E−27 |
| B2M | 4.1E−38 | 0.644171 | 0.98 | 0.942 | 6.67E−34 |
| HIST1H2BN | 1.46E−35 | 0.62793 | 0.695 | 0.501 | 2.37E−31 |
| IGFBP5 | 4.82E−33 | 0.581753 | 0.813 | 0.636 | 7.85E−29 |
| HLA-DRA | 1.37E−31 | 0.563063 | 0.821 | 0.689 | 2.22E−27 |
| CTSS | 2.11E−35 | 0.562357 | 0.706 | 0.522 | 3.43E−31 |
| ADAMTS2 | 1.93E−30 | 0.560252 | 0.655 | 0.49 | 3.14E−26 |
| CYBB | 5.12E−32 | 0.554348 | 0.7 | 0.539 | 8.33E−28 |
| HNRNPA2B1 | 1.44E−31 | 0.548971 | 0.876 | 0.798 | 2.34E−27 |
| AEBP1 | 6.52E−36 | −0.45998 | 0.799 | 0.901 | 1.06E−31 |
| CFD | 2.29E−21 | −0.46442 | 0.605 | 0.752 | 3.72E−17 |
| CRYAB | 3.37E−23 | −0.48933 | 0.306 | 0.487 | 5.47E−19 |
| PLA2G2A | 3.26E−18 | −0.51545 | 0.216 | 0.364 | 5.31E−14 |
| SOD3 | 1.65E−29 | −0.52832 | 0.742 | 0.838 | 2.68E−25 |
| PTGIS | 2.07E−30 | −0.53668 | 0.283 | 0.491 | 3.37E−26 |
| C1R | 1.65E−44 | −0.54702 | 0.99 | 0.992 | 2.68E−40 |
| C3 | 1.8E−18 | −0.58428 | 0.738 | 0.786 | 2.93E−14 |
| APOD | 4.24E−19 | −0.62331 | 0.363 | 0.519 | 6.89E−15 |
| MYL9 | 1.43E−31 | −0.67275 | 0.646 | 0.765 | 2.33E−27 |
| THBS1 | 1.14E−18 | −0.67603 | 0.854 | 0.872 | 1.86E−14 |
| SAA2 | 2.92E−18 | −0.74345 | 0.108 | 0.25 | 4.75E−14 |
| IGFBP4 | 8.3E−20 | −0.79966 | 0.544 | 0.67 | 1.35E−15 |
| FBLN1 | 1.57E−48 | −0.85184 | 0.749 | 0.842 | 2.55E−44 |
| TAGLN | 3.24E−40 | −0.89459 | 0.544 | 0.708 | 5.27E−36 |
| ACTA2 | 6.91E−37 | −0.91785 | 0.703 | 0.828 | 1.12E−32 |
| G0S2 | 1.04E−34 | −0.98842 | 0.724 | 0.828 | 1.7E−30 |
| PTX3 | 6.89E−26 | −1.04314 | 0.423 | 0.581 | 1.12E−21 |
| RARRES1 | 1.97E−23 | −1.14294 | 0.477 | 0.605 | 3.2E−19 |
| CLU | 2.7E−103 | −1.22926 | 0.929 | 0.987 | 4.4E−99 |

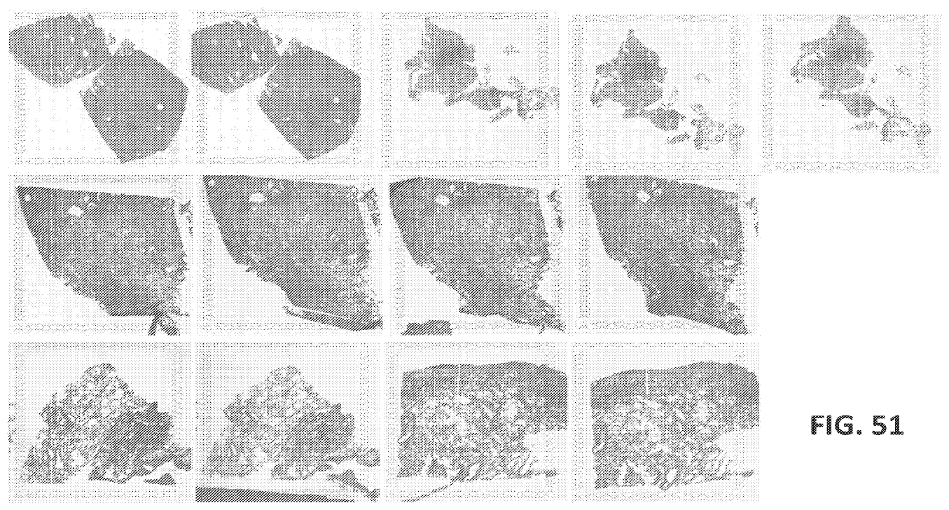

From looking into the structural components of the tissue sections, all three COVID-positive lung samples included in this study represented the late-phase pneumonia stage (between 13-17 days post-infection) and showed consistent histopathological features with organizing diffuse alveolar damage, extensive fibrosis, leukocyte infiltration accompanied by low viral load, relative to earlier phases of the disease progression (FIG. 51).

The substantial structural differences between the COVID-positive and COVID-negative lung sections were also indicated in the transcriptome data. DE genes in the COVID-positive lung sections were dominated by signatures of plasma cells (IGHG3, IGKC, IGHM, JCHAIN, IGHG2, IGKV4-1, IGLV3-1, IGHA1) and activated fibroblasts (COL1A1, COL1A2, COL3A1), inflammatory cytokines (CXCL9, CCL18) and complement factors (C1QB, C1QC), reflecting the overall tissue response to a prolonged SARS-CoV-2 infection.

In the myeloid-cell rich cluster 1 of the COVID-positive tissue samples, selective up-(CD163, F13A1, CD14, LYZ, APOE, C1QA, B2M) or down-regulation (PPARG, VCAN, FCN1, YAP1, FCGR3A) of certain marker genes was observed, previously used to annotate monocyte-macrophage lineage subsets in single cell transcriptomic datasets of COVID-positive lung tissue, implicating a shift in myeloid cell subtypes during disease progression. A representative list of genes that were differentially expressed in each cluster is provided below in Table 14.

TABLE 14
Differences between the COVID-positive and COVID-
negative lung sections in each Cluster

| gene | p_val | avg_log2FC | pct. 1 | pct. 2 | p_val_adj |
|---|---|---|---|---|---|
| CLUSTER 1 | | | | | |
| IGKC | 0 | 5.010481514 | 0.99 | 0.483 | 0 |
| COL3A1 | 0 | 4.454426978 | 0.999 | 0.479 | 0 |
| IGHG3 | 0 | 4.08741285 | 0.987 | 0.092 | 0 |
| IGHM | 0 | 3.835979926 | 0.962 | 0.146 | 0 |
| COL1A1 | 0 | 3.740716588 | 0.984 | 0.328 | 0 |
| JCHAIN | 0 | 3.703499863 | 0.945 | 0.188 | 0 |
| COL1A2 | 0 | 3.554696969 | 0.999 | 0.545 | 0 |
| IFI6 | 0 | 3.29564751 | 0.951 | 0.395 | 0 |
| APOE | 0 | 3.20652216 | 0.844 | 0.479 | 0 |
| IGHA1 | 0 | 3.104601263 | 0.964 | 0.336 | 0 |
| IGKV4-1 | 0 | 2.862125625 | 0.858 | 0.09 | 0 |
| CXCL9 | 0 | 2.760762892 | 0.757 | 0.098 | 0 |
| IGHG2 | 0 | 2.742301843 | 0.757 | 0.041 | 0 |
| IFI27 | 0 | 2.695044298 | 0.995 | 0.618 | 0 |
| HLA-DRB5 | 0 | 2.646603943 | 0.906 | 0 | 0 |
| C1QC | 0 | 2.629008667 | 0.998 | 0.712 | 0 |
| SPP1 | 6.2036E−268 | 2.541871766 | 0.725 | 0.713 | 1.0092E−263 |
| PLTP | 0 | 2.530921018 | 0.957 | 0.355 | 0 |
| CCL18 | 0 | 2.350762193 | 0.925 | 0.379 | 0 |
| LUM | 0 | 2.330333572 | 0.977 | 0.4 | 0 |
| ACSL1 | 0 | −1.032193331 | 0.724 | 0.926 | 0 |
| AKAP12 | 0 | −1.052241286 | 0.092 | 0.573 | 0 |
| VCAN | 0 | −1.118220753 | 0.567 | 0.894 | 0 |
| BCL6 | 0 | −1.144933781 | 0.464 | 0.874 | 0 |
| SLC11A1 | 0 | −1.200205259 | 0.698 | 0.951 | 0 |
| TIMP1 | 0 | −1.213678066 | 1 | 1 | 0 |
| LMCD1 | 0 | −1.220515453 | 0.311 | 0.807 | 0 |
| TNC | 0 | −1.234244023 | 0.623 | 0.944 | 0 |
| IFITM2 | 0 | −1.242405076 | 0.976 | 1 | 0 |
| RIPOR2 | 0 | −1.266237273 | 0.234 | 0.779 | 0 |
| SLC25A37 | 0 | −1.343800611 | 0.472 | 0.878 | 0 |
| CCN1 | 0 | −1.471514766 | 0.587 | 0.898 | 0 |
| CSF3R | 0 | −1.473688267 | 0.373 | 0.789 | 0 |
| S100A9 | 0 | −1.631606023 | 0.998 | 1 | 0 |
| SERPINE1 | 0 | −1.668466951 | 0.729 | 0.967 | 0 |
| HSPA1B | 0 | −1.683333453 | 0.782 | 0.998 | 0 |
| G0S2 | 0 | −2.186735525 | 0.571 | 0.991 | 0 |
| S100A8 | 0 | −2.204821723 | 0.98 | 0.997 | 0 |
| THBS1 | 0 | −2.585315433 | 0.678 | 0.998 | 0 |
| PTX3 | 0 | −2.664077326 | 0.173 | 0.96 | 0 |
| CLUSTER 2 | | | | | |
| IGKC | 0 | 4.706764568 | 0.963 | 0.523 | 0 |
| COL3A1 | 0 | 4.219696882 | 1 | 0.613 | 0 |
| IGHG3 | 0 | 3.582585604 | 0.977 | 0.097 | 0 |
| COL1A1 | 0 | 3.541502144 | 0.942 | 0.373 | 0 |
| COL1A2 | 0 | 3.322486434 | 1 | 0.689 | 0 |
| IGHM | 0 | 3.264523984 | 0.863 | 0.162 | 0 |
| JCHAIN | 0 | 3.243791091 | 0.776 | 0.207 | 0 |
| IGHA1 | 0 | 3.14918231 | 0.985 | 0.351 | 0 |
| IFI6 | 0 | 2.912564362 | 0.89 | 0.382 | 0 |
| APOE | 0 | 2.739869207 | 0.857 | 0.347 | 0 |
| IGKV4-1 | 0 | 2.67338782 | 0.769 | 0.08 | 0 |
| IGHG2 | 0 | 2.603560058 | 0.813 | 0.025 | 0 |
| LUM | 0 | 2.347439889 | 0.955 | 0.51 | 0 |
| IFI27 | 0 | 2.216009184 | 0.973 | 0.664 | 0 |
| C1QC | 0 | 2.200955995 | 0.965 | 0.612 | 0 |
| FN1 | 0 | 2.039245063 | 1 | 0.981 | 0 |
| CXCL9 | 0 | 2.003231886 | 0.495 | 0.057 | 0 |
| SPP1 | 1.6332E−224 | 1.917916591 | 0.637 | 0.497 | 2.6569E−220 |
| PTGDS | 0 | 1.910457754 | 0.971 | 0.581 | 0 |
| CD74 | 0 | 1.826781073 | 0.995 | 0.97 | 0 |
| ADAMTS9 | 0 | −0.832176012 | 0.162 | 0.59 | 0 |
| ANGPTL4 | 0 | −0.8719618 | 0.377 | 0.771 | 0 |
| ACSL1 | 3.1247E−248 | −0.879909407 | 0.538 | 0.793 | 5.0833E−244 |
| RIPOR2 | 0 | −0.913716295 | 0.157 | 0.619 | 0 |
| SLC11A1 | 0 | −0.976127411 | 0.574 | 0.85 | 0 |
| CSF3R | 7.5141E−279 | −0.984552036 | 0.288 | 0.625 | 1.2224E−274 |
| TIMP1 | 0 | −1.003030891 | 0.999 | 1 | 0 |
| IFITM2 | 0 | −1.010566325 | 0.89 | 1 | 0 |
| SLC25A37 | 0 | −1.040115435 | 0.378 | 0.786 | 0 |
| MT2A | 3.0095E−70 | −1.055978654 | 0.998 | 0.998 | 4.89585E−66 |
| TNC | 0 | −1.144134032 | 0.548 | 0.897 | 0 |
| HSP90AA1 | 0 | −1.181832985 | 0.893 | 0.999 | 0 |
| G0S2 | 0 | −1.321776284 | 0.735 | 0.973 | 0 |
| CCN1 | 0 | −1.375794335 | 0.529 | 0.789 | 0 |
| S100A9 | 3.1395E−142 | −1.450788397 | 0.979 | 0.998 | 5.1074E−138 |
| SERPINE1 | 0 | −1.480069375 | 0.646 | 0.93 | 0 |
| HSPA1B | 0 | −1.625722971 | 0.68 | 0.985 | 0 |
| S100A8 | 5.4483E−200 | −1.746881432 | 0.93 | 0.972 | 8.8633E−196 |
| THBS1 | 0 | −2.138142179 | 0.748 | 0.995 | 0 |
| PTX3 | 0 | −2.236182146 | 0.289 | 0.903 | 0 |
| CLUSTER 3 | | | | | |
| IGHG3 | 0 | 4.483097181 | 0.998 | 0.295 | 0 |
| IGKC | 0 | 4.174537983 | 0.997 | 0.807 | 0 |
| IGHM | 0 | 3.951102022 | 0.949 | 0.381 | 0 |
| COL3A1 | 0 | 3.772073633 | 1 | 0.655 | 0 |
| JCHAIN | 0 | 3.685205745 | 0.878 | 0.527 | 0 |
| IGHG2 | 0 | 3.48763352 | 0.797 | 0.143 | 0 |
| IGKV4-1 | 0 | 3.267233086 | 0.864 | 0.267 | 0 |
| COL1A1 | 0 | 3.110461466 | 0.974 | 0.442 | 0 |
| IGLV3-1 | 0 | 3.015383334 | 0.836 | 0.202 | 0 |
| CXCL9 | 3.8287E−269 | 3.005781785 | 0.643 | 0.087 | 6.2285E−265 |
| IGHA1 | 0 | 2.867478562 | 0.988 | 0.616 | 0 |
| COL1A2 | 0 | 2.839303261 | 0.999 | 0.75 | 0 |
| IFI6 | 0 | 2.805426443 | 0.903 | 0.384 | 0 |
| XBP1 | 0 | 2.266365162 | 0.979 | 0.644 | 0 |
| LUM | 0 | 2.225462048 | 0.981 | 0.559 | 0 |
| APOE | 1.5094E−239 | 2.224779912 | 0.79 | 0.477 | 2.4554E−235 |
| IFI27 | 0 | 2.206057957 | 0.986 | 0.633 | 0 |
| HLA-DRB5 | 0 | 2.194557609 | 0.781 | 0 | 0 |
| C1QC | 0 | 2.191053288 | 0.977 | 0.629 | 0 |
| MZB1 | 0 | 2.112443193 | 0.913 | 0.152 | 0 |
| AGER | 1.7454E−160 | −0.958517274 | 0.633 | 0.913 | 2.8394E−156 |
| VCAN | 5.0414E−203 | −0.974116056 | 0.494 | 0.81 | 8.2014E−199 |
| KRT7 | 3.3175E−252 | −1.000864241 | 0.569 | 0.922 | 5.3969E−248 |
| ACSL1 | 2.2779E−194 | −1.020070211 | 0.52 | 0.813 | 3.7057E−190 |
| SLC25A37 | 9.9069E−245 | −1.030700495 | 0.423 | 0.807 | 1.6117E−240 |
| TNC | 2.0467E−251 | −1.051140586 | 0.586 | 0.901 | 3.3295E−247 |
| ANGPTL4 | 8.1004E−298 | −1.085505695 | 0.348 | 0.793 | 1.3178E−293 |
| CSF3R | 1.5098E−210 | −1.08830181 | 0.281 | 0.646 | 2.4561E−206 |
| IFITM2 | 0 | −1.101284205 | 0.948 | 1 | 0 |
| MT2A | 1.03075E−75 | −1.143775014 | 1 | 0.999 | 1.67682E−71 |
| TIMP1 | 0 | −1.217901808 | 0.999 | 1 | 0 |
| SLC11A1 | 0 | −1.295303469 | 0.469 | 0.874 | 0 |
| S100A9 | 1.2198E−118 | −1.391063482 | 0.991 | 0.998 | 1.9843E−114 |
| SERPINE1 | 2.5482E−214 | −1.415249717 | 0.679 | 0.934 | 4.1454E−210 |
| CCN1 | 1.0668E−223 | −1.472783924 | 0.577 | 0.827 | 1.7354E−219 |
| HSPA1B | 0 | −1.487503806 | 0.775 | 0.991 | 0 |
| S100A8 | 1.4466E−153 | −1.755650282 | 0.946 | 0.985 | 2.3534E−149 |
| G0S2 | 0 | −1.759799164 | 0.668 | 0.98 | 0 |
| THBS1 | 0 | −2.309370793 | 0.742 | 0.996 | 0 |
| PTX3 | 0 | −2.522272614 | 0.236 | 0.93 | 0 |
| CLUSTER 4 | | | | | |
| IGKC | 0 | 4.981647337 | 0.985 | 0.546 | 0 |
| COL3A1 | 0 | 4.492824037 | 1 | 0.554 | 0 |
| COL1A1 | 0 | 3.897060391 | 0.979 | 0.341 | 0 |
| IGHG3 | 0 | 3.862477387 | 0.982 | 0.113 | 0 |
| IGHM | 0 | 3.644209161 | 0.944 | 0.164 | 0 |
| COL1A2 | 0 | 3.521411221 | 1 | 0.642 | 0 |
| IGHA1 | 0 | 3.485760358 | 0.991 | 0.381 | 0 |
| JCHAIN | 0 | 3.466510873 | 0.884 | 0.234 | 0 |
| IFI6 | 0 | 3.334891074 | 0.95 | 0.417 | 0 |
| APOE | 0 | 3.082877648 | 0.937 | 0.395 | 0 |
| IGHG2 | 0 | 3.040478877 | 0.886 | 0.031 | 0 |
| IGKV4-1 | 0 | 3.039566157 | 0.891 | 0.1 | 0 |
| IFI27 | 0 | 2.642104352 | 0.992 | 0.628 | 0 |
| C1QC | 0 | 2.52323011 | 0.983 | 0.594 | 0 |
| SPP1 | 0 | 2.421716237 | 0.791 | 0.506 | 0 |
| CCL18 | 0 | 2.254637626 | 0.884 | 0.329 | 0 |
| LUM | 0 | 2.235048277 | 0.979 | 0.46 | 0 |
| C1QB | 0 | 2.165048344 | 0.932 | 0.694 | 0 |
| ISG15 | 0 | 2.053696726 | 0.852 | 0.125 | 0 |
| CD74 | 0 | 2.048369647 | 0.999 | 0.963 | 0 |
| LMCD1 | 9.64E−216 | −0.928338482 | 0.369 | 0.715 | 1.5682E−211 |
| RIPOR2 | 7.0358E−288 | −0.939358368 | 0.185 | 0.631 | 1.1446E−283 |
| IFITM2 | 4.7886E−295 | −0.941796066 | 0.953 | 0.999 | 7.79E−291 |
| ANGPTL4 | 3.6145E−262 | −0.948644148 | 0.366 | 0.76 | 5.88E−258 |
| CSF3R | 1.8696E−170 | −0.950972255 | 0.393 | 0.683 | 3.0414E−166 |
| SLC11A1 | 1.4075E−202 | −0.98395105 | 0.614 | 0.85 | 2.2898E−198 |
| SLC25A37 | 9.9287E−253 | −1.008768675 | 0.448 | 0.792 | 1.6152E−248 |
| HSP90AA1 | 0 | −1.029007181 | 0.935 | 0.998 | 0 |
| TNC | 1.3943E−265 | −1.075307005 | 0.581 | 0.869 | 2.2683E−261 |
| TIMP1 | 0 | −1.124028971 | 0.998 | 0.999 | 0 |
| CCN1 | 5.6045E−145 | −1.138914115 | 0.654 | 0.789 | 9.1174E−141 |
| MT2A | 8.91135E−60 | −1.144188341 | 0.999 | 0.995 | 1.4497E−55 |
| SCGB1A1 | 2.8107E−103 | −1.445171739 | 0.231 | 0.503 | 4.5725E−99 |
| S100A9 | 7.1905E−121 | −1.568795203 | 0.991 | 0.996 | 1.1698E−116 |
| HSPA1B | 0 | −1.573318402 | 0.755 | 0.981 | 0 |
| G0S2 | 0 | −1.671264557 | 0.618 | 0.955 | 0 |
| SERPINE1 | 0 | −1.76757579 | 0.591 | 0.894 | 0 |
| S100A8 | 3.9786E−208 | −2.012125598 | 0.937 | 0.979 | 6.4724E−204 |
| THBS1 | 0 | −2.255522525 | 0.71 | 0.985 | 0 |
| PTX3 | 0 | −2.273609074 | 0.207 | 0.872 | 0 |
| CLUSTER 5 | | | | | |
| IGHG3 | 0 | 4.335974196 | 0.968 | 0.127 | 0 |
| IGKC | 0 | 4.059159616 | 0.955 | 0.566 | 0 |
| IGHM | 0 | 3.704777742 | 0.874 | 0.189 | 0 |
| JCHAIN | 0 | 3.580148303 | 0.79 | 0.275 | 0 |
| IGHA1 | 0 | 2.812773386 | 0.971 | 0.405 | 0 |
| COL3A1 | 0 | 2.75636641 | 1 | 0.755 | 0 |
| CXCL9 | 2.3922E−294 | 2.671801709 | 0.626 | 0.106 | 3.8917E−290 |
| IGHG2 | 0 | 2.62579929 | 0.62 | 0.059 | 0 |
| IFI6 | 0 | 2.616502841 | 0.836 | 0.266 | 0 |
| IGLV3-1 | 0 | 2.467545607 | 0.752 | 0.062 | 0 |
| IGKV4-1 | 0 | 2.344723833 | 0.704 | 0.15 | 0 |
| IFI27 | 0 | 2.224277351 | 0.955 | 0.492 | 0 |
| C1QC | 0 | 2.182556359 | 0.955 | 0.513 | 0 |
| COL1A1 | 0 | 2.165839096 | 0.942 | 0.606 | 0 |
| CTSB | 0 | 2.159868629 | 0.995 | 0.91 | 0 |
| COL1A2 | 0 | 2.149025864 | 1 | 0.807 | 0 |
| HLA-DRB5 | 0 | 2.033141532 | 0.713 | 0.001 | 0 |
| CD74 | 0 | 2.003238091 | 0.996 | 0.963 | 0 |
| B2M | 4.8666E−230 | 1.989485535 | 0.921 | 0.999 | 7.917E−226 |
| XBP1 | 0 | 1.929531828 | 0.912 | 0.56 | 0 |
| CSF3R | 2.348E−165 | −0.811807383 | 0.228 | 0.561 | 3.8198E−161 |
| TNC | 2.0183E−145 | −0.833116562 | 0.553 | 0.814 | 3.2834E−141 |
| MT1A | 5.8855E−291 | −0.878233855 | 0.07 | 0.493 | 9.5746E−287 |
| LMCD1 | 8.4732E−176 | −0.900916564 | 0.351 | 0.687 | 1.3784E−171 |
| S100A9 | 2.91835E−84 | −0.91208115 | 0.977 | 0.994 | 4.74757E−80 |
| ADM | 2.6011E−247 | −0.96284599 | 0.548 | 0.888 | 4.2314E−243 |
| SLC11A1 | 1.3997E−239 | −0.96970953 | 0.371 | 0.777 | 2.277E−235 |
| APOD | 6.2975E−217 | −0.985508215 | 0.364 | 0.769 | 1.0245E−212 |
| HSP90AA1 | 0 | −1.000889997 | 0.936 | 0.999 | 0 |
| S100A8 | 3.74124E−91 | −1.012088972 | 0.934 | 0.971 | 6.08626E−87 |
| CCN1 | 2.5566E−180 | −1.048301327 | 0.615 | 0.851 | 4.1591E−176 |
| MT2A | 1.1615E−136 | −1.058365294 | 1 | 0.998 | 1.8895E−132 |
| SERPINE2 | 2.3961E−195 | −1.070059602 | 0.12 | 0.464 | 3.8979E−191 |
| MT1X | 9.6002E−247 | −1.15247581 | 0.772 | 0.973 | 1.5618E−242 |
| SERPINE1 | 7.2555E−192 | −1.168992884 | 0.652 | 0.928 | 1.1803E−187 |
| ANGPTL4 | 0 | −1.256006759 | 0.336 | 0.826 | 0 |
| G0S2 | 4.2809E−276 | −1.441292677 | 0.734 | 0.973 | 6.9642E−272 |
| HSPA1B | 0 | −1.44170791 | 0.72 | 0.992 | 0 |
| THBS1 | 0 | −1.833581718 | 0.809 | 0.99 | 0 |
| PTX3 | 0 | −1.921789361 | 0.372 | 0.936 | 0 |
| CLUSTER 6 | | | | | |
| IGKC | 0 | 4.715600723 | 0.995 | 0.475 | 0 |
| IGHG3 | 0 | 3.875517818 | 0.969 | 0.075 | 0 |
| COL1A1 | 0 | 3.560640869 | 0.997 | 0.782 | 0 |
| COL3A1 | 0 | 3.560106004 | 1 | 0.889 | 0 |
| IGHM | 0 | 3.549989595 | 0.951 | 0.181 | 0 |
| JCHAIN | 0 | 3.409784191 | 0.927 | 0.203 | 0 |
| COL1A2 | 0 | 3.061981726 | 1 | 0.898 | 0 |
| IGHA1 | 0 | 3.006613193 | 0.969 | 0.336 | 0 |
| APOE | 1.0191E−198 | 2.822286577 | 0.868 | 0.594 | 1.6579E−194 |
| IFI6 | 0 | 2.803412862 | 0.951 | 0.42 | 0 |
| IGKV4-1 | 6.305E−288 | 2.770836913 | 0.861 | 0.094 | 1.0257E−283 |
| CXCL9 | 7.6997E−177 | 2.590816035 | 0.714 | 0.137 | 1.2526E−172 |
| LUM | 0 | 2.583378092 | 0.995 | 0.621 | 0 |
| IGHG2 | 1.5529E−252 | 2.582187146 | 0.785 | 0.035 | 2.5262E−248 |
| HLA-DRB5 | 9.2081E−306 | 2.321398063 | 0.856 | 0.001 | 1.498E−301 |
| CCL18 | 8.5726E−270 | 2.32013568 | 0.916 | 0.345 | 1.3946E−265 |
| C1QC | 0 | 2.179129002 | 0.993 | 0.754 | 0 |
| HLA-DRA | 3.9185E−292 | 2.170290633 | 0.941 | 0.659 | 6.3746E−288 |
| C1QB | 1.4032E−235 | 2.140431582 | 0.96 | 0.784 | 2.2828E−231 |
| LYZ | 6.3779E−254 | 2.066736936 | 0.961 | 0.934 | 1.0375E−249 |
| HSPG2 | 2.02145E−27 | −0.802144934 | 0.725 | 0.746 | 3.28849E−23 |
| VWF | 5.32794E−88 | −0.808289715 | 0.577 | 0.841 | 8.6675E−84 |
| RHOB | 1.2509E−129 | −0.870598932 | 0.481 | 0.823 | 2.035E−125 |
| SLC25A37 | 1.1278E−105 | −0.893955878 | 0.495 | 0.78 | 1.8348E−101 |
| INMT | 3.00265E−35 | −0.895832803 | 0.26 | 0.418 | 4.88471E−31 |
| TIMP1 | 2.3513E−173 | −0.968217227 | 1 | 1 | 3.8251E−169 |
| MCAM | 1.0689E−136 | −0.972381641 | 0.228 | 0.632 | 1.739E−132 |
| GREM1 | 1.08658E−45 | −1.002061516 | 0.157 | 0.355 | 1.76765E−41 |
| HSP90AA1 | 1.9658E−204 | −1.08537822 | 0.895 | 0.995 | 3.1979E−200 |
| ADAMTS9 | 6.6915E−239 | −1.192138489 | 0.148 | 0.676 | 1.0886E−234 |
| SERPINE1 | 1.1215E−123 | −1.336766202 | 0.712 | 0.926 | 1.8244E−119 |
| HSPA1B | 6.1049E−208 | −1.337957631 | 0.8 | 0.98 | 9.9314E−204 |
| S100A9 | 3.26901E−95 | −1.348668841 | 0.992 | 1 | 5.31802E−91 |
| COL4A1 | 4.3096E−139 | −1.378383629 | 0.737 | 0.946 | 7.0108E−135 |
| ANGPTL4 | 2.0427E−224 | −1.50562651 | 0.352 | 0.838 | 3.3231E−220 |
| G0S2 | 3.2557E−180 | −1.644036435 | 0.569 | 0.889 | 5.2964E−176 |
| FABP4 | 1.5897E−271 | −1.779065203 | 0.064 | 0.546 | 2.5861E−267 |
| S100A8 | 5.798E−149 | −1.830334311 | 0.884 | 0.981 | 9.4321E−145 |
| THBS1 | 1.352E−277 | −1.854108782 | 0.811 | 0.997 | 2.1995E−273 |
| PTX3 | 0 | −2.586780907 | 0.206 | 0.928 | 0 |

Besides many consensus fibrosis markers (COL1A1, COL3A1, COL5A1, SPP1, FN1, POSTN), the CTHRC1 gene, recently described as a distinct marker of pathological, pulmonary fibrosis-associated fibroblasts, was determined to be highly expressed in the fibroblast-rich cluster 6 of COVID-positive lungs. At the same time, markers of the alveolar fibroblast, thought to be a cellular source of the CTHRC1-positive fibroblast subpopulation, were either mildly downregulated (TCF21, PDGFRA) or upregulated (NPNT1) in the same cluster.

In order to investigate the biological changes occurring in the epithelial cell compartments, further sub clustering of cluster 4 was performed, resulting in the alveolar epithelium-enriched subcluster 1 and airway epithelium-dominated subcluster 2.

In subcluster 1, COVID-positive lung tissue showed markedly (SFTPC, SLC34A2, MUC1, LYZ) or moderately (LAMP3, PGC, NAPSA, CEBPA, LPCAT1, SDC1, NKX2-1, PIGR, ABCA3, ALPL) elevated expression of type 2 alveolar epithelial (AT2) cell markers, while the type 1 alveolar epithelial (AT1) cell markers appeared to be either slightly down-(KRT7, AGER, FSTL3, SCNN1G) or upregulated (CYP4B1, ICAM1, AQP4, RTKN2, EMP2, GPRC5A, AQP3, CAV1). Table 14 shows representative dysregulated genes in subcluster-1 and subcluster-2 in Cluster 4.

TABLE 15
Representative Dysregulated Genes in subclusters-1 and -2 in Cluster 4.

| gene | p_val | avg_log2FC | pct.1 | pct.2 | p_val_adj |
|---|---|---|---|---|---|
| Cluster 4, sub-cluster-1 | | | | | |
| IGKC | 0 | 4.909729 | 0.984 | 0.564 | 0 |
| COL3A1 | 0 | 4.415231 | 1 | 0.589 | 0 |
| IGHG3 | 0 | 3.864097 | 0.981 | 0.105 | 0 |
| COL1A1 | 0 | 3.861137 | 0.979 | 0.356 | 0 |
| IGHM | 0 | 3.618463 | 0.941 | 0.167 | 0 |
| IGHA1 | 0 | 3.464974 | 0.991 | 0.393 | 0 |
| COL1A2 | 0 | 3.448387 | 1 | 0.668 | 0 |
| JCHAIN | 0 | 3.42514 | 0.88 | 0.247 | 0 |
| IFI6 | 0 | 3.268165 | 0.949 | 0.433 | 0 |
| APOE | 0 | 3.046922 | 0.937 | 0.415 | 0 |
| IGHG2 | 0 | 3.034266 | 0.887 | 0.033 | 0 |
| IGKV4-1 | 0 | 3.029127 | 0.889 | 0.106 | 0 |
| IFI27 | 0 | 2.587863 | 0.992 | 0.653 | 0 |
| C1QC | 0 | 2.47184 | 0.982 | 0.626 | 0 |
| SPP1 | 5.4E−298 | 2.43368 | 0.789 | 0.513 | 8.8E−294 |
| CCL18 | 0 | 2.237443 | 0.88 | 0.346 | 0 |
| LUM | 0 | 2.176405 | 0.978 | 0.487 | 0 |
| C1QB | 0 | 2.096072 | 0.931 | 0.722 | 0 |
| ISG15 | 0 | 2.024884 | 0.849 | 0.131 | 0 |
| CTSD | 0 | 2.010291 | 0.972 | 0.974 | 0 |
| ETS2 | 1.3E−214 | −0.88238 | 0.39 | 0.767 | 2.1E−210 |
| RIPOR2 | 1.4E−241 | −0.92201 | 0.189 | 0.627 | 2.2E−237 |
| IFITM2 | 5.9E−262 | −0.93387 | 0.951 | 1 | 9.6E−258 |
| CSF3R | 1.7E−141 | −0.94065 | 0.395 | 0.677 | 2.8E−137 |
| HSP90AA1 | 0 | −0.96552 | 0.933 | 0.997 | 0 |
| LMCD1 | 4.1E−202 | −0.97035 | 0.371 | 0.733 | 6.6E−198 |
| SLC25A37 | 5.9E−205 | −0.98657 | 0.448 | 0.782 | 9.6E−201 |
| SLC11A1 | 7.6E−179 | −0.98852 | 0.614 | 0.856 | 1.2E−174 |
| ANGPTL4 | 6.4E−242 | −0.99131 | 0.365 | 0.774 | 1E−237 |
| TNC | 1.9E−221 | −1.06168 | 0.578 | 0.87 | 3.1E−217 |
| TIMP1 | 5.3E−302 | −1.12894 | 0.998 | 1 | 8.7E−298 |
| CCN1 | 1E−114 | −1.13547 | 0.658 | 0.781 | 1.7E−110 |
| MT2A | 1.94E−45 | −1.16505 | 0.999 | 0.997 | 3.15E−41 |
| S100A9 | 7.38E−89 | −1.54111 | 0.991 | 0.996 | 1.2E−84 |
| HSPA1B | 0 | −1.56294 | 0.749 | 0.978 | 0 |
| G0S2 | 0 | −1.713 | 0.619 | 0.967 | 0 |
| SERPINE1 | 4E−279 | −1.81869 | 0.591 | 0.905 | 6.6E−275 |
| S100A8 | 9.3E−179 | −1.99853 | 0.935 | 0.983 | 1.5E−174 |
| THBS1 | 0 | −2.2745 | 0.713 | 0.992 | 0 |
| PTX3 | 0 | −2.33601 | 0.208 | 0.879 | 0 |
| Cluster 4, sub-cluster-2 | | | | | |
| IGKC | 1.3E−99 | 5.444736 | 0.996 | 0.471 | 2.04E−95 |
| COL3A1 | 2.1E−104 | 4.903067 | 1 | 0.407 | 3.5E−100 |
| COL1A1 | 1.6E−103 | 4.058134 | 0.988 | 0.278 | 2.6E−99 |
| IGHG3 | 3E−112 | 4.007092 | 0.992 | 0.148 | 4.9E−108 |
| COL1A2 | 7.4E−102 | 3.896713 | 1 | 0.532 | 1.2E−97 |
| IFI6 | 1.22E−89 | 3.873799 | 0.968 | 0.349 | 1.98E−85 |
| IGHM | 6.6E−110 | 3.865704 | 0.984 | 0.148 | 1.1E−105 |
| JCHAIN | 6.02E−98 | 3.787164 | 0.935 | 0.177 | 9.79E−94 |
| IGHA1 | 2.3E−100 | 3.651533 | 0.996 | 0.331 | 3.81E−96 |
| APOE | 6.58E−83 | 3.229997 | 0.927 | 0.315 | 1.07E−78 |
| IGKV4-1 | 1.7E−102 | 3.095617 | 0.919 | 0.074 | 2.77E−98 |
| IGHG2 | 4.2E−103 | 3.076683 | 0.883 | 0.024 | 6.9E−99 |
| IFI27 | 5.2E−99 | 2.907565 | 0.996 | 0.521 | 8.48E−95 |
| C1QC | 2.47E−95 | 2.748667 | 0.992 | 0.463 | 4.01E−91 |
| LUM | 1.67E−98 | 2.545162 | 0.988 | 0.347 | 2.71E−94 |
| C1QB | 1.71E−71 | 2.456812 | 0.943 | 0.577 | 2.78E−67 |
| SPP1 | 4.03E−51 | 2.333462 | 0.814 | 0.479 | 6.56E−47 |
| ISG15 | 7.69E−95 | 2.331568 | 0.891 | 0.098 | 1.25E−90 |
| CD74 | 1.89E−93 | 2.32906 | 0.996 | 0.968 | 3.08E−89 |
| SPARC | 1.07E−90 | 2.323533 | 1 | 0.823 | 1.74E−86 |
| SCGB3A2 | 1.67E−07 | −0.90974 | 0.668 | 0.831 | 0.002719 |
| IFITM2 | 1.6E−29 | −0.95534 | 0.972 | 0.997 | 2.6E−25 |
| BCL6 | 1.59E−37 | −0.96555 | 0.518 | 0.865 | 2.59E−33 |
| MT1X | 8.31E−30 | −0.9659 | 0.85 | 0.947 | 1.35E−25 |
| SLC11A1 | 6.02E−22 | −0.9729 | 0.619 | 0.823 | 9.79E−18 |
| MT2A | 4.6E−11 | −1.01018 | 1 | 0.987 | 7.48E−07 |
| CSF3R | 6.28E−25 | −1.03156 | 0.368 | 0.706 | 1.02E−20 |
| RIPOR2 | 3.59E−38 | −1.04542 | 0.134 | 0.651 | 5.83E−34 |
| HSP90AA1 | 1.35E−54 | −1.05799 | 0.964 | 1 | 2.2E−50 |
| SLC25A37 | 3.48E−37 | −1.09837 | 0.457 | 0.833 | 5.67E−33 |
| TNC | 1.56E−35 | −1.13229 | 0.615 | 0.868 | 2.53E−31 |
| TIMP1 | 1.57E−48 | −1.13788 | 0.996 | 0.997 | 2.56E−44 |
| CCN1 | 1.03E−26 | −1.21588 | 0.603 | 0.823 | 1.67E−22 |
| SERPINE1 | 1.22E−28 | −1.48069 | 0.591 | 0.847 | 1.99E−24 |
| G0S2 | 1.82E−47 | −1.5187 | 0.611 | 0.907 | 2.96E−43 |
| HSPA1B | 9.13E−73 | −1.52953 | 0.834 | 0.992 | 1.48E−68 |
| S100A9 | 3.52E−27 | −1.67488 | 0.988 | 0.997 | 5.72E−23 |
| S100A8 | 1.52E−22 | −1.97534 | 0.955 | 0.96 | 2.48E−18 |
| PTX3 | 2.88E−61 | −1.98851 | 0.186 | 0.844 | 4.68E−57 |
| THBS1 | 2.85E−68 | −2.2364 | 0.668 | 0.952 | 4.63E−64 |

These changes point at a hyperplastic expansion of type 2 alveolar epithelial (AT2) cells in the diseased tissue, consistent with microscopic observations made in lungs with longer disease duration and characterized by low viral load.

### Example 5. Co-Localization of SARS-CoV-2 and Human Gene Expression

Differentially expressed (DE) genes were identified using ‘FindMarkers’ in Seurat, with default settings on the SCT normalized matrix, except min.cells.group set to 2 to include at least 2 spots from each group. Both upregulated, and downregulated DE genes were identified, with an adjusted p-value of 0.005. Cell-type specific annotation of the DE genes was performed manually, by using the Human Single Cell Atlas, PanglaoDB, and recently published single cell transcriptomic data of the human lung as main resources. For the colocalization analysis, comparison between the SARS-CoV-2 positive spots, and the SARS-CoV-2 negative spots from the COVID-positive sections was done. The DE genes for this analysis were obtained in a similar approach as above, with an additional filter of average log FC+/−1.0.

In accordance with the available literature, a relatively low viral load in the COVID-positive sections with all used experimental techniques (ST, RNAscope, ISS) was detected, possibly explained by the late disease progression phase in the infected samples (FIGS. 41A, 41B and 46). SARS-CoV-2 positive spots appeared throughout different morphological areas, without obvious enrichment in any of the spatial clusters (FIG. 49). While type 2 alveolar epithelial cells and macrophages have been described as the main cellular targets of the new coronavirus, viral infection of many other lung cell types have also been discussed in recent studies. Since the resolution of spatial transcriptomics does not yet reach the single cell level, data collected on the spot level most often represent the transcriptome of a mixture of different cell types, which might explain the lack of any preferential cluster for the SARS-CoV-2-positive spots.

Further, viral entry factors ACE2, TMPRSS2, PCK5, or PCSK7 were differentially expressed between COVID-positive and COVID-negative lung sections. However, CTSB, CTSL, and NRP1 were upregulated in COVID-19 positive lung sections compared to COVID-negative lung sections, with all three reported as SARS-CoV-2 entry factors. CTSL and CTSB observed to have increased expression in COVID19 lungs compared to control lungs, with SARS-CoV-2 infection demonstrated to promote CTSL expression that then in turn enhances viral infection. Differences in the expression of the viral entry factors could be related to the progression of the disease since ACE2 was upregulated in high viral load lungs (associated with earlier states of disease progression) compared to low viral loads in lung, such that SARS-CoV-2 induced downregulation of ACE2 that promotes lung injury and induced increasing expression of CTSL occurs later in the disease progression.

In order to identify gene expression changes caused by the active presence of the virus mRNA in lung cells, human gene expression patterns in SARS-CoV-2 positive and negative spots in COVID-positive sections were compared in this study (Table 16).

TABLE 16
Identification of dysregulated genes in similar pathways

| | | avg | | | | | |
|---|---|---|---|---|---|---|---|
| Gene | p_val | log2FC | pct. 1 | pct. 2 | p_val_adj | Gene name | Related pathway |
| RNASE1 | 1.3E−270 | −1.457 | 0.76 | 0.947 | 2E−266 | Ribonuclease A | RNA degradation |
| | | | | | | Family Member 1 | |
| SRRM2 | 0 | 1.0625 | 0.997 | 0.939 | 0 | Serine/Arginine | RNA processing; |
| | | | | | | Repetitive Matrix 2 | nuclear, necessary for |
| | | | | | | | mRNA splicing |
| HNRNPA2B1 | 0 | 1.0488 | 0.985 | 0.836 | 0 | Heterogeneous | RNA processing; |
| | | | | | | Nuclear | packaging of nascent |
| | | | | | | Ribonucleoprotein | pre-RNA |
| | | | | | | A2/B1 | |
| SMAP2 | 1.6E−293 | 1.0165 | 0.925 | 0.533 | 3E−289 | Small | potential regulator of |
| | | | | | | ArfGAP2/stromal | autophagy (antiviral |
| | | | | | | membrane- | process) |
| | | | | | | associated protein 2 | |
| NFKBIA | 0 | 1.3547 | 0.978 | 0.696 | 0 | NFKB Inhibitor Alpha | NFKB pathway |
| NFKB2 | 2.6E−285 | 1.002 | 0.919 | 0.546 | 4E−281 | Nuclear Factor | NFKB pathway |
| | | | | | | Kappa B Subunit 2 | |
| CXCL9 | 0 | 1.8697 | 0.968 | 0.65 | 0 | C-X-C Motif | chemokine |
| | | | | | | Chemokine Ligand 9 | |
| CCL17 | 0 | 1.2985 | 0.696 | 0.135 | 0 | C-C Motif | chemokine |
| | | | | | | Chemokine Ligand | |
| | | | | | | 17 | |
| CCL21 | 3.5E−271 | 1.1547 | 0.827 | 0.344 | 6E−267 | C-C Motif | chemokine |
| | | | | | | Chemokine Ligand | |
| | | | | | | 21 | |
| CFD | 3.7E−193 | −1.057 | 0.482 | 0.808 | 6E−189 | Complement Factor | complement factor |
| | | | | | | D | |
| C7 | 1.6E−168 | −1.099 | 0.413 | 0.761 | 3E−164 | Complement C7 | complement factor |
| C1QB | 5E−170 | −1.463 | 0.915 | 0.918 | 8E−166 | Complement C1q | complement factor |
| | | | | | | B Chain | |
| ADAMTS2 | 0 | 1.2716 | 0.95 | 0.597 | 0 | ADAM | extracellular matrix |
| | | | | | | Metallopeptidase | component - |
| | | | | | | With | procollagen processing |
| | | | | | | Thrombospondin | |
| | | | | | | Type 1 Motif 2 | |
| FBLN1 | 1.5E−177 | −1.093 | 0.557 | 0.831 | 2E−173 | Fibulin 1 | extracellular matrix |
| | | | | | | | component |
| COL1A2 | 3E−161 | −1.193 | 1 | 0.999 | 5E−157 | Collagen Type I | extracellular matrix |
| | | | | | | Alpha 2 Chain | component |
| COL3A1 | 6.1E−138 | −1.224 | 1 | 1 | 1E−133 | Collagen Type III | extracellular matrix |
| | | | | | | Alpha 1 Chain | component |
| BGN | 1.1E−195 | −1.248 | 0.425 | 0.785 | 2E−191 | Biglycan | extracellular matrix |
| | | | | | | | component |
| COL1A1 | 2E−130 | −1.38 | 0.981 | 0.962 | 3E−126 | Collagen Type I | extracellular matrix |
| | | | | | | Alpha 1 Chain | component |
| SPP1 | 1.9E−234 | −2.022 | 0.201 | 0.712 | 3E−230 | Secreted | extracellular matrix |
| | | | | | | Phosphoprotein | component |
| | | | | | | 1/Osteopontin | |
| AQP1 | 1.1E−151 | −1.006 | 0.373 | 0.713 | 2E−147 | Aquaporin 1 | epithel/endothel- |
| | | | | | | | related; expressed in |
| | | | | | | | capillary endothel |
| | | | | | | | under alveolar |
| | | | | | | | basement membrane |
| MUC1 | 1.4E−160 | −1.077 | 0.14 | 0.573 | 2E−156 | Mucin 1 | epithel-related, AT2 |
| | | | | | | | marker |
| SLC34A2 | 6.1E−209 | −1.371 | 0.191 | 0.683 | 1E−204 | Solute Carrier Family | epithel-related, AT2 |
| | | | | | | 34 Member 2 | marker |
| SFTPC | 7E−151 | −1.167 | 0.064 | 0.495 | 1E−146 | Surfactant Protein C | epithel-related, AT2 |
| | | | | | | | marker |
| SFTPB | 4.3E−305 | −2.059 | 0.316 | 0.837 | 7E−301 | Surfactant Protein B | epithel-related, AT2 |
| | | | | | | | marker |
| AGER | 4.6E−206 | −1.539 | 0.261 | 0.712 | 7E−202 | Advanced | epithel-related, AT1 |
| | | | | | | Glycosylation End- | marker |
| | | | | | | Product Specific | |
| | | | | | | Receptor | |
| GPRC5A | 1.2E−168 | −1.218 | 0.164 | 0.603 | 2E−164 | G Protein-Coupled | epithel-related, AT1 |
| | | | | | | Receptor Class C | marker |
| | | | | | | Group 5 Member A; | |
| | | | | | | Retinoic acid- | |
| | | | | | | induced protein 3, | |
| | | | | | | AT1 marker | |
| IGHV5-10-1 | 0 | 1.1512 | 0.62 | 0.15 | 0 | Immunoglobulin | immunoglobulin |
| | | | | | | Heavy Variable | |
| | | | | | | 5-10-1 | |
| IGKC | 1.43E−78 | −1.219 | 1 | 0.98 | 2E−74 | Immunoglobulin | immunoglobulin |
| | | | | | | Kappa Constant | |
| IGKV4-1 | 6.6E−146 | −1.639 | 0.673 | 0.831 | 1E−141 | Immunoglobulin | immunoglobulin |
| | | | | | | Kappa Variable 4-1 | |
| IGHA1 | 4E−236 | −1.891 | 0.882 | 0.977 | 6E−232 | Immunoglobulin | immunoglobulin |
| | | | | | | Heavy Constant | |
| | | | | | | Alpha 1 | |
| IGHG2 | 1.3E−275 | −2.231 | 0.269 | 0.788 | 2E−271 | Immunoglobulin | immunoglobulin |
| | | | | | | Heavy Constant | |
| | | | | | | Gamma | |
| ISG15 | 4.6E−133 | −1.051 | 0.617 | 0.791 | 7E−129 | Interferon- | interferon-related |
| | | | | | | stimulated gene 15 | |
| IFI6 | 2E−206 | −1.843 | 0.835 | 0.909 | 3E−202 | Interferon Alpha | interferon-related |
| | | | | | | Inducible Protein 6 | |
| HLA-DRB5 | 0 | 1.1239 | 0.978 | 0.815 | 0 | Major | leukocyte-related |
| | | | | | | Histocompatibility | |
| | | | | | | Complex, Class II, DR | |
| | | | | | | Beta 5 | |
| RARRES2 | 6.5E−154 | −1.024 | 0.402 | 0.729 | 1E−149 | Retinoic Acid | myeloid cell-related; |
| | | | | | | Receptor Responder | expressed by |
| | | | | | | 2/chemerin | fibroblasts; |
| | | | | | | | chemoattractant, |
| | | | | | | | secreted and activated |
| | | | | | | | by serine proteases, |
| | | | | | | | stimulates chemotaxis |
| | | | | | | | of macrophages |
| CD163 | 0 | 1.0974 | 0.997 | 0.916 | 0 | Cluster of | myeloid cell-related; |
| | | | | | | Differentiation 163 | macrophage subtype |
| | | | | | | | marker (Delorey) |
| APOE | 1.3E−287 | −2.046 | 0.445 | 0.861 | 2E−283 | Apolipoprotein E | myeloid cell-related; |
| | | | | | | | inflammatory |
| | | | | | | | monocyte subtype |
| | | | | | | | marker (Delorey) |
| SERPINE1 | 2.1E−271 | 1.1267 | 0.96 | 0.672 | 3E−267 | Plasminogen | protease inhibitor |
| | | | | | | activator inhibitor-1 | |
| PI15 | 0 | 1.1702 | 0.81 | 0.26 | 0 | Peptidase Inhibitor | protease inhibitor |
| | | | | | | 15 | |
| WFDC2 | 3.2E−147 | −1.01 | 0.224 | 0.628 | 5E−143 | WAP Four-Disulfide | protease inhibitor |
| | | | | | | Core Domain 2 | |
| A2M | 1.6E−115 | −1.125 | 0.973 | 0.937 | 3E−111 | Alpha-2- | protease inhibitor |
| | | | | | | Macroglobulin | |
| ARHGEF1 | 3.7E−301 | 1.0836 | 0.936 | 0.565 | 6E−297 | Rho Guanine | T cell-related |
| | | | | | | Nucleotide Exchange | |
| | | | | | | Factor 1 | |
| TCIRG1 | 3.6E−301 | 1.0385 | 0.967 | 0.692 | 6E−297 | T Cell Immune | T-cell related |
| | | | | | | Regulator 1 | |
| MT1G | 5.8E−286 | 1.1782 | 0.954 | 0.692 | 9E−282 | Metallothionein 1G | redox protein |

Notably, these tissues represent a late phase of the disease progression with corresponding cellular and molecular changes, which might explain some of the differences between these results and previously published studies, comparing morphologically healthy lung areas with actively infected ones. By comparing viral active (SARS-CoV-2-positive spots) areas to viral inactive (SARS-CoV-2-negative spots) areas within 55 μm spatially resolved areas in COVID-positive tissue sections, one can visualize/analyze highly localized human cellular responses to SARS-CoV-2 and how they differ based on the presence or absence of SARS-CoV-2.

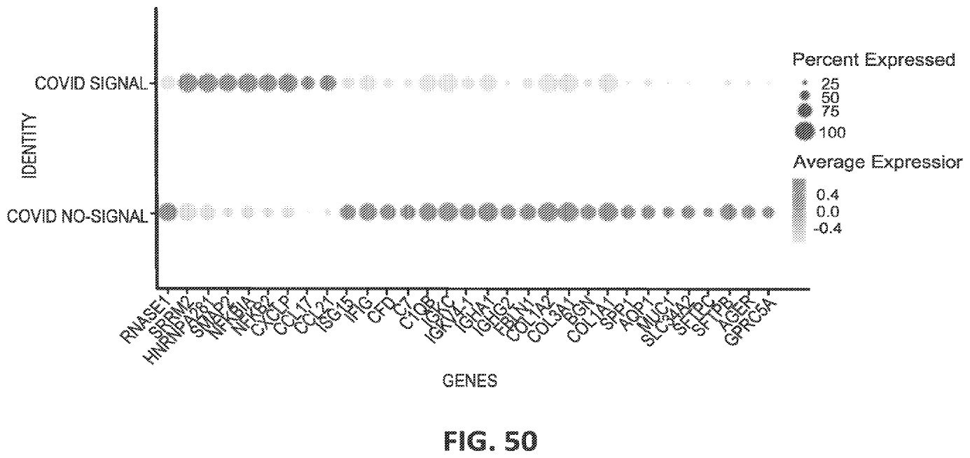

The analysis revealed several genes involved in RNA metabolism to be differentially expressed in ST spots expressing viral mRNA (FIG. 50, Table 16). Serine/Arginine Repetitive Matrix 2 (SRRM2), a component of the spliceosome, were identified as upregulated in SARS-CoV-2 spots (FIG. 50, Table 16). While this gene has not yet been directly connected to COVID-19 pathogenesis, it was previously described to have a central role in nuclear speckle formation. This bimolecular complex was shown to be essential for the replication, splicing and trafficking of Herpes Simplex Virus and Influenza A virus, which suggest a potentially similar role in processing the SARS-CoV-2 virus. On the other hand, Heterogeneous Nuclear Ribonucleoprotein A2/B1 (HNRNP A2/B1), another upregulated gene known to be involved in the packaging of nascent pre-mRNA, has been described in the formation of cytoplasmic stress granules responsible for the assembly of the nucleocapsid protein and genomic RNA of SARS-Cov-2 (Table 16). Another study showed the NSP1 (nonstructural protein 1) of SARS-Cov-2 to facilitate viral RNA processing and block effective IFNbeta expression through directly binding hnRNPA2/B1 and redistributing it between the nucleus and cytoplasm. Besides the mentioned protein-protein interactions, directly modulating the expression of SMMR2 and hnRNPA2/B1 in the host cells might be a potential alternative mechanism for the SARS-Cov-2 virus to ensure proper synthesis and assembly of the viral components and further spread of viral particles. Notably, the RNasel gene was downregulated in SARS-Cov-2 positive spots, potentially blocking degradation of viral RNA in the environment of actively infected cells (FIG. 50, Table 16).

Among different mechanisms of the host cell immune repertoire against viral infection, autophagy is a potent tool to destroy infected cells and limit the spread of the virus. Many viruses, including SARS-Cov-2, have developed strategies to antagonize the autophagy pathway and thus escape host cell immunity, Small GTPase proteins ARF1 and ARF6 play a role in the early steps of the autophagosome formation, and it has recently been postulated that ARF6 might be bound and inhibited by the SARS-Cov-2 protein NSP15 (Non-structural Protein 15). SMAP2, a GTPase activating protein regulating ARF1 and ARF6 proteins, was upregulated in SARS-Cov-2 positive spots (FIG. 50, Table 16). By catalyzing the GTP hydrolysis of ARF1 and ARF6 and thus rendering them in an inactive state, an increase in SMAP2 expression and activity would mean a plausible alternative mechanism for the SARS-Cov-2 virus to block the autophagy pathway and promote viral replication and dissemination.

Many recent publications highlighted the NFKB pathway being directly induced by components of the SARS-COV-2 virus, and as a central signaling pathway responsible for further progression of the COVID-19 disease. In line with these studies, Nuclear Factor Kappa B Subunit 2 (NFKB2) and NFKB Inhibitor Alpha (NFKBIA) were upregulated in virus-active spots (FIG. 50, Table 16). NFKB activation initiates an uncontrolled, self-reinforcing, cyclic release of pro-inflammatory cytokines, leading to a phenomenon commonly referred to as cytokine storm. In the samples, CXCL9, CCL17 and CCL21 were upregulated in SARS-CoV-2 positive ST spots, all of which have previously been shown to be induced in an NFKB-dependent manner, directly mediated by the ORF7 protein of the SARS-CoV-2 virus (FIG. 50, Table 16). Moreover, CCL17 has been proposed as a potential predictive biomarker to distinguish between mild/moderate and severe/critical disease, and CXCL9 to be part of a biomarker panel associated with mortality in patients with COVID-19. Notably, certain complement factors (C1QB, CFD, C7) and interferon response genes (1F16, ISG15) were upregulated in COVID-positive lungs compared to COVID-negative lungs, in line with previous studies; however, these genes were downregulated in the SARS-CoV-2 positive ST spots of the infected lungs (FIG. 50, Table 16), which points to spatially localized differences in host response to the virus as previously observed in terms of interferon response genes. Activation of the complement cascade by SARS-CoV-2 viral components via the lectin-mediated or alternative pathway (involving CFD) is an early step in COVID-19 pathogenesis, however, sustained activity of this molecular pathway plays a central role in the maintenance of the unbalanced inflammatory response throughout the entire course of the disease. It was proposed that SARS-CoV-2 can direct a reduction in IFN response in active viral SARS-Cov-2 areas, such as through Nsp3 cleavage of ISG15 resulting in an attenuated type I IFN response, while areas without SARS-CoV-2 infection are able to mount a type I IFN response due to lack of viral-induced IFN suppression. As complement genes are part of the IFN-stimulated gene response, they may be also influenced by IFN attenuated response, although the relationship between IFN and complement activation remains to be further investigated. Downregulation of C1QB, CFD and C7 and IFI6, ISG15 in the SARS-Cov-2 positive spots might represent an earlier cellular phase of disease progression where a viral evasion strategy delays complement and IFN activation, in comparison to other, already altered tissue areas where such responses have been activated. However, future studies are needed to further investigate the role of interferon response and complement gene expression during SARS-CoV-2 infection stages.

The analysis has highlighted some differentially expressed genes that are reflective of compositional and structural differences between the environment of cells actively expressing SARS-CoV-2 gene sets, compared to diseased tissue areas that represent the effects of earlier presence of the virus and consequently activated molecular processes, even in larger distances from the active viral spots (FIG. 50, Table 16). The downregulation of certain immunoglobulin genes (IGKC, IGKV4-1, IGHA1, IGHG2) and extracellular matrix components (FBLN, COL1A2, COL3A1, BGN, COL1A1, SPP1) in the SARS-CoV-2 positive ST spots can be explained by the viral infection preceding both the extensive plasma cell infiltration and fibroblast activation in time (FIG. 50, Table 16). It is also possible, however, that areas with a cellular and molecular environment closely resembling the healthy state of the lungs, are more permissive for the replication of viral components. In agreement with the available literature, several AT2 (SFTPB, SFTPC, MUC1, SLC34A2), AT1 (GPRC5A, AGER) and the alveolar endothelial cell marker AQP1 were downregulated in the SARS-CoV-2 positive ST spots (FIG. 50, Table 16). These differences can either represent functional impairment (decreased surfactant and mucin production) or increased apoptosis of alveolar epithelial cells, which are known to be the primary cellular targets of the SARS-CoV-2 in the lungs.

### Example 6. Determination of Biomarkers that are Upregulated or Downregulated in SARS-CoV-2 Positive FFPE-Fixed Heart Tissue Samples Using Templated Ligation

It has been suggested that a SARS-CoV-2 infection can significantly affect patients' cardiovascular systems. See e.g., Chang et al., Am J Med Sci. 2021 January; 361(1): 14-22, which is incorporated by reference in its entirety. Using the methods of templated ligation described in Example 1, cardiac dysregulated biomarkers were identified using RTL probe pairs in two consecutive FFPE-fixed heart tissue sections from the same sample from one SARS-CoV-2-positive subject post-mortem. Compared to a normal heart tissue sample (i.e., a subject confirmed to be without a SARS-CoV-2 infection), the biomarkers listed in Table 17 were upregulated in a sample infected with SARS-CoV-2, the biomarkers listed in Table 18 were downregulated in a sample infected with SARS-CoV-2, and the biomarkers listed in Table 19 did not significantly change expression in a sample infected with SARS-CoV-2 compared to an uninfected heart tissue sample.

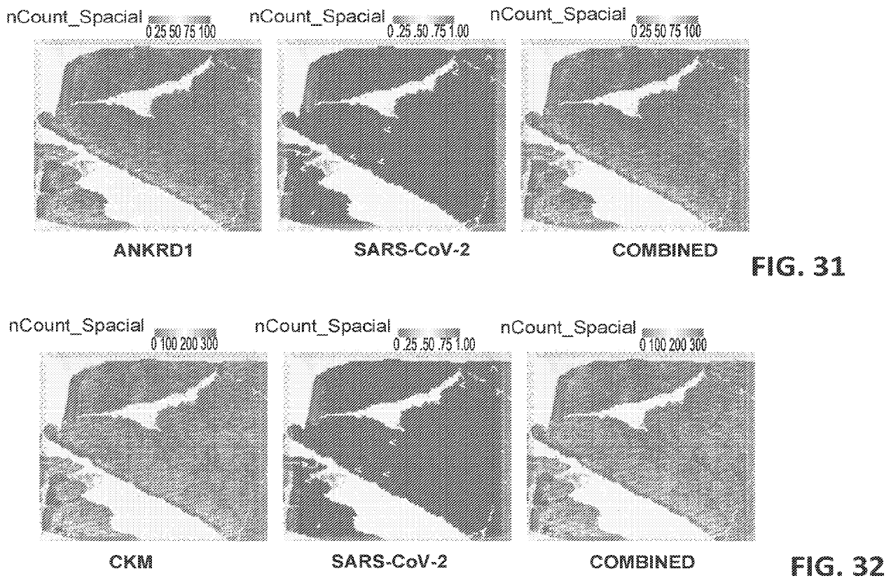

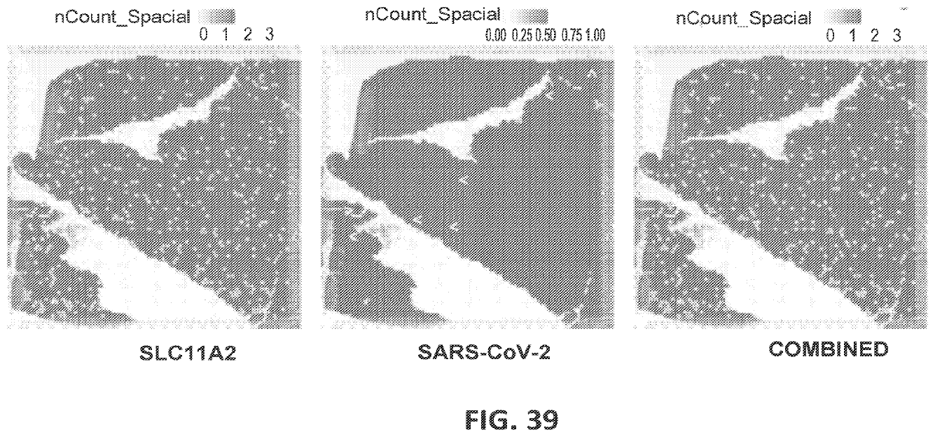

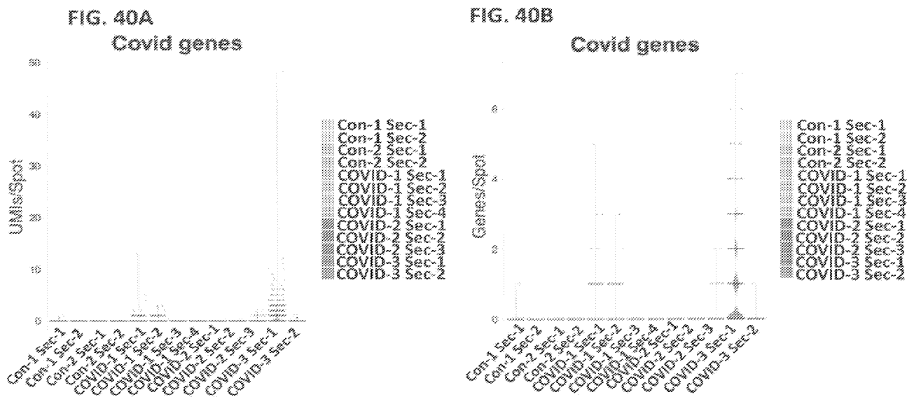

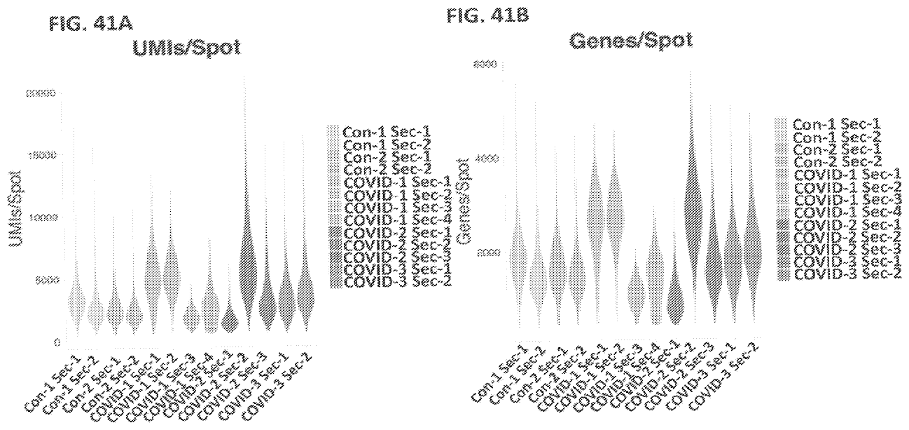

Representative images were generated to show increased or decreased expression of certain SARS-CoV-2 biomarkers. As shown in FIGS. 31-37, biomarkers that were dysregulated (i.e., either upregulated or downregulated—see white arrows) were dysregulated, and as shown in FIGS. 38-39, biomarkers that were not dysregulated (i.e. neither upregulated nor downregulated) did not manifest a change in expression in both heart samples. Further, FIGS. 31-39 show detection of SARS-CoV-2 in heart samples (see white arrows in each center panel of FIGS. 31-39).

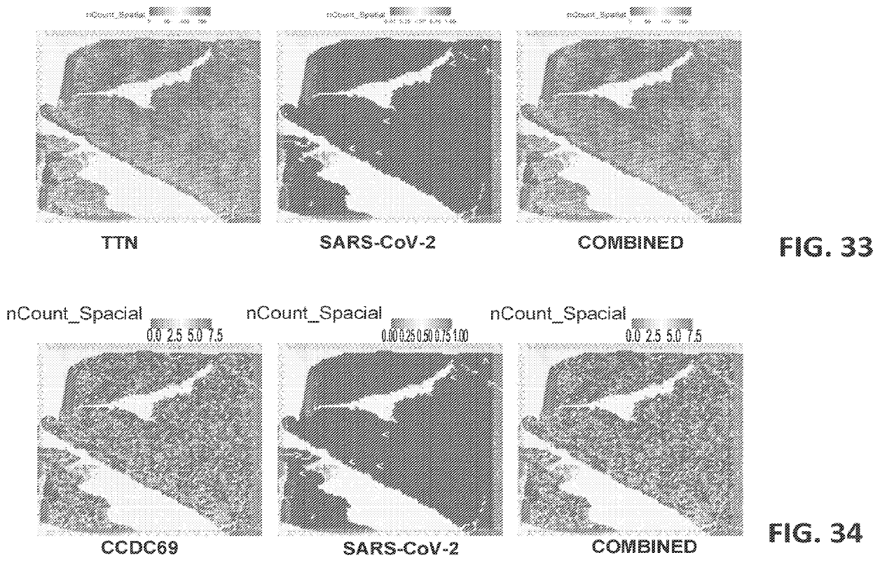

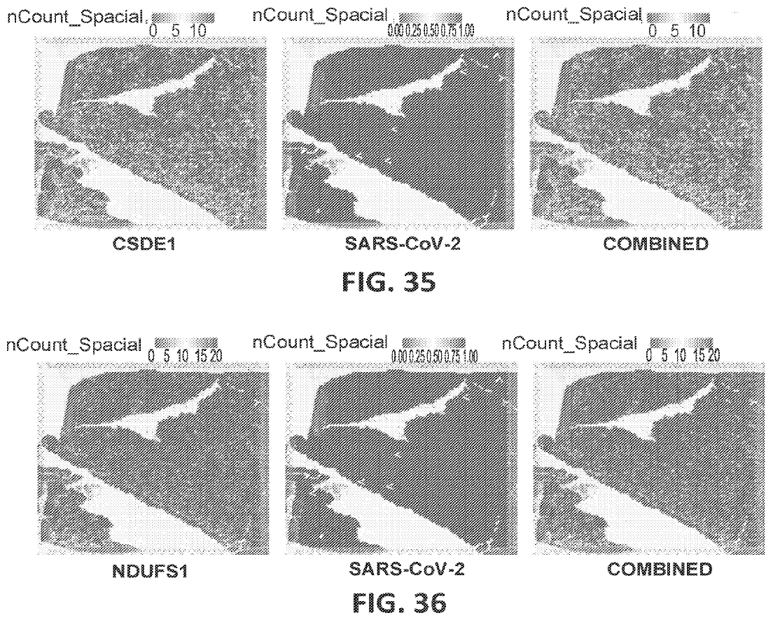

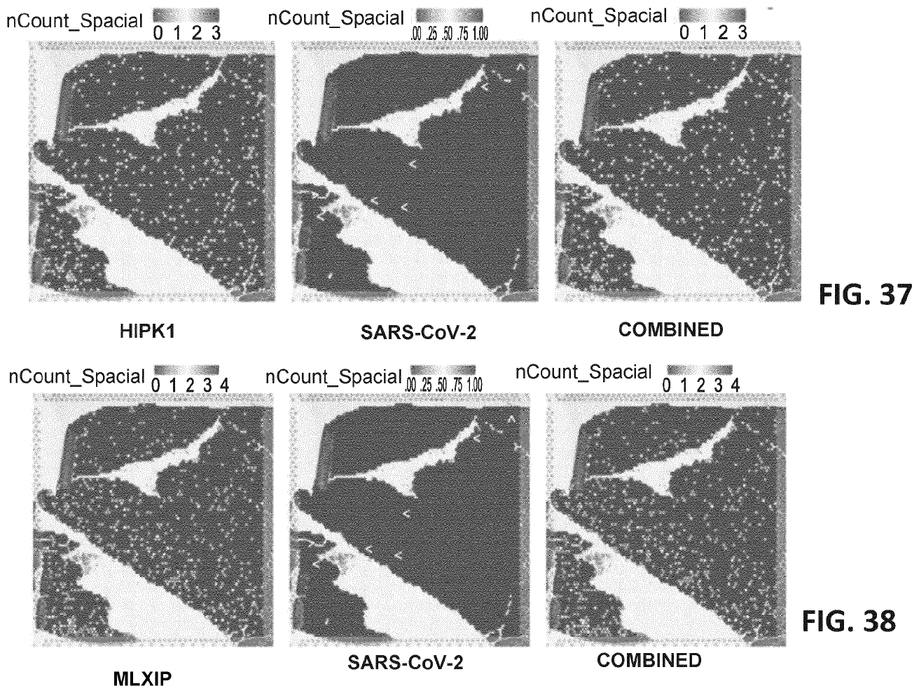

Specifically, ANKRD1 (FIG. 31), CKM (FIG. 32), and TTN (FIG. 33) was upregulated in the lung tissue sample. On the other hand, CCDC69 (FIG. 34), CSDE1 (FIG. 35), NDUFS1 (FIG. 36), was downregulated in the heart tissue sample. Lastly, HIPK1 (FIG. 37), MLXIP (FIG. 38), and SLC11A2 (FIG. 39) were dysregulated (i.e., neither upregulated nor downregulated) in the heart sample. These results were duplicated in a second, consecutive heart tissue section from the same heart sample from the subject. These data demonstrate a proof of concept that the spatial location and abundance of one or more biomarkers could be determined using post-mortem human heart tissue samples from a deceased subject positive for a SARS-CoV-2 infection.

TABLE 17
SARS-CoV-2 biomarkers that were upregulated
in infected human heart tissue.

| | | | Expression in heart |
|---|---|---|---|
| | | Adjusted | sample in presence of |
| Biomarker | Average log2FC | p-value | SARS-CoV-2 infection |
| CKM | 2.88227496975356 | 0 | Over-expressed |
| ANKRD1 | 2.54149658249469 | 0 | Over-expressed |
| TTN | 2.26027908036998 | 0 | Over-expressed |
| MYL3 | 2.16590910281584 | 0 | Over-expressed |
| HRC | 2.07491139182994 | 0 | Over-expressed |
| TNNT2 | 2.05498711115158 | 0 | Over-expressed |
| TNNC1 | 2.00628888329577 | 0 | Over-expressed |
| PTGDS | 1.99245870654789 | 0 | Over-expressed |
| TNNI3 | 1.98145317042249 | 0 | Over-expressed |
| MYH7 | 1.94552157231216 | 0 | Over-expressed |
| MYL2 | 1.94027570222944 | 0 | Over-expressed |
| DES | 1.9251587697057 | 0 | Over-expressed |
| S100A1 | 1.88992890193397 | 0 | Over-expressed |
| CKMT2 | 1.88416165234797 | 0 | Over-expressed |
| TPM1 | 1.8677222267199 | 0 | Over-expressed |
| ACTC1 | 1.85297448698737 | 0 | Over-expressed |
| HSPB7 | 1.72959689134539 | 0 | Over-expressed |
| TCAP | 1.71258574445591 | 0 | Over-expressed |
| SLC25A4 | 1.6835000127953 | 0 | Over-expressed |
| MB | 1.65822713189408 | 0 | Over-expressed |
| CRYAB | 1.62974681614178 | 0 | Over-expressed |
| COX5A | 1.61377991843883 | 0 | Over-expressed |
| ATP2A2 | 1.58053288534726 | 0 | Over-expressed |
| PPP1R12B | 1.57137600736896 | 0 | Over-expressed |
| ATP5IF1 | 1.56703416103922 | 0 | Over-expressed |
| KIF1C | 1.52386370243914 | 0 | Over-expressed |
| ACTN2 | 1.51498934748068 | 0 | Over-expressed |
| PLN | 1.49937266562295 | 0 | Over-expressed |
| NMRK2 | 1.4980608325694 | 0 | Over-expressed |
| PPP1R1A | 1.49245771261905 | 0 | Over-expressed |
| MYBPC3 | 1.45791960987819 | 0 | Over-expressed |
| BSG | 1.43715024400527 | 0 | Over-expressed |
| PKP2 | 1.43216090636157 | 0 | Over-expressed |
| COX6A2 | 1.43019068781104 | 0 | Over-expressed |
| MYL9 | 1.40247161593318 | 0 | Over-expressed |
| ZNF106 | 1.39990051406625 | 0 | Over-expressed |
| EEF1A2 | 1.39642779220132 | 0 | Over-expressed |
| CSRP3 | 1.38828687362106 | 0 | Over-expressed |
| LMOD2 | 1.37875865450768 | 0 | Over-expressed |
| MYL7 | 1.32560006152869 | 0 | Over-expressed |
| MYOZ2 | 1.31242588616362 | 0 | Over-expressed |
| PTP4A3 | 1.31000702499905 | 0 | Over-expressed |
| NRAP | 1.30804943474527 | 0 | Over-expressed |
| NEBL | 1.27287122107308 | 0 | Over-expressed |
| HHATL | 1.26889713258382 | 0 | Over-expressed |
| ATP5F1B | 1.26846520805058 | 0 | Over-expressed |
| SQSTM1 | 1.25928110005669 | 0 | Over-expressed |
| ADPRHL1 | 1.24549054063886 | 0 | Over-expressed |
| PDE4DIP | 1.24204942958993 | 0 | Over-expressed |
| KCNIP2 | 1.23761947998211 | 0 | Over-expressed |
| IDH2 | 1.23037712546255 | 0 | Over-expressed |
| SORBS1 | 1.22520651150723 | 0 | Over-expressed |
| SYNPO | 1.22063559467667 | 0 | Over-expressed |
| LDB3 | 1.20207174433015 | 0 | Over-expressed |
| UQCRC1 | 1.194503035799 | 0 | Over-expressed |
| AZGP1 | 1.19384532825162 | 0 | Over-expressed |
| CHCHD10 | 1.18549731684756 | 0 | Over-expressed |
| SMYD1 | 1.18070055191951 | 0 | Over-expressed |
| MYH7B | 1.16861095316455 | 0 | Over-expressed |
| SMPX | 1.1489684734054 | 0 | Over-expressed |
| ACADVL | 1.14865475566349 | 0 | Over-expressed |
| ATP5MC3 | 1.13628989898018 | 0 | Over-expressed |
| PFKFB2 | 1.13299895118079 | 0 | Over-expressed |
| HSPB6 | 1.11716469223531 | 0 | Over-expressed |
| TRDN | 1.11044543769637 | 0 | Over-expressed |
| MYOM1 | 1.10960358826915 | 0 | Over-expressed |
| ANK1 | 1.10917821230105 | 0 | Over-expressed |
| NEXN | 1.10290146537071 | 0 | Over-expressed |
| MDH1 | 1.10204645604763 | 0 | Over-expressed |
| CKB | 1.0962702863272 | 0 | Over-expressed |
| NDUFA13 | 1.09587912314164 | 0 | Over-expressed |
| HSPB8 | 1.0937306730977 | 0 | Over-expressed |
| ATP5PF | 1.09270611966407 | 0 | Over-expressed |
| ASAH1 | 1.08828082427066 | 0 | Over-expressed |
| ATP5F1D | 1.08384270865194 | 0 | Over-expressed |
| TRIM54 | 1.08174836861866 | 0 | Over-expressed |
| PALLD | 1.08161847387342 | 0 | Over-expressed |
| CTSD | 1.08098155737482 | 0 | Over-expressed |
| MYOM3 | 1.07741424738391 | 0 | Over-expressed |
| PFKM | 1.07568439970884 | 0 | Over-expressed |
| NFE2L1 | 1.07185226680172 | 0 | Over-expressed |
| FLNC | 1.07026081453049 | 0 | Over-expressed |
| MGST3 | 1.06741114851949 | 0 | Over-expressed |
| UQCR11 | 1.06497223656622 | 0 | Over-expressed |
| SYNM | 1.05966778645175 | 0 | Over-expressed |
| ECH1 | 1.05768668424609 | 0 | Over-expressed |
| ACO2 | 1.04992972656357 | 0 | Over-expressed |
| ACSL1 | 1.04961656166642 | 0 | Over-expressed |
| NDUFB10 | 1.04912180544762 | 0 | Over-expressed |
| NDUFS6 | 1.04584490197215 | 0 | Over-expressed |
| POPDC2 | 1.0447494617084 | 0 | Over-expressed |
| ABLIM1 | 1.04258058581308 | 0 | Over-expressed |
| COX5B | 1.03507090635749 | 0 | Over-expressed |
| SLC25A3 | 1.03038735643075 | 0 | Over-expressed |
| HSPB3 | 1.01097516275857 | 0 | Over-expressed |
| MLIP | 1.00445017045827 | 0 | Over-expressed |

TABLE 18
SARS-CoV-2 biomarkers that were downregulated in infected human heart tissue.

| | | | Expression in heart |
|---|---|---|---|
| | | | sample in presence of |
| Biomarker | Average log2FC | Adjusted p-value | SARS-CoV-2 infection |
| CCDC69 | 0.499768299867895 | 0 | Under-expressed |
| CSDE1 | 0.49739846005331 | 0 | Under-expressed |
| NDUFS1 | 0.497149330806946 | 0 | Under-expressed |
| DMTN | 0.496740015234827 | 0 | Under-expressed |
| PLIN5 | 0.496537559746383 | 0 | Under-expressed |
| KLHL21 | 0.496368468593684 | 0 | Under-expressed |
| UBXN6 | 0.496060206528791 | 0 | Under-expressed |
| BEX3 | 0.495580092780251 | 0 | Under-expressed |
| PDE1C | 0.495556456177007 | 0 | Under-expressed |
| NCKAP1 | 0.49293756070528 | 0 | Under-expressed |
| ECSIT | 0.49031833186772 | 0 | Under-expressed |
| NDUFS4 | 0.488387805284633 | 0 | Under-expressed |
| ACADM | 0.487723425316719 | 0 | Under-expressed |
| RBM24 | 0.487509900586008 | 0 | Under-expressed |
| TXN2 | 0.486279517050888 | 0 | Under-expressed |
| 1.00 MAF | 0.485429300144561 | 0 | Under-expressed |
| PARM1 | 0.484614953965752 | 0 | Under-expressed |
| SMYD2 | 0.482972891302775 | 0 | Under-expressed |
| PAM | 0.481520953883893 | 0 | Under-expressed |
| GADD45GIP1 | 0.480295094527887 | 0 | Under-expressed |
| PDHB | 0.476238459484273 | 0 | Under-expressed |
| MRAS | 0.473203574003944 | 0 | Under-expressed |
| SNX3 | 0.471783090566742 | 0 | Under-expressed |
| TBC1D4 | 0.470838011973909 | 0 | Under-expressed |
| PHLDB2 | 0.470084464369016 | 0 | Under-expressed |
| NKX2-5 | 0.470045242102793 | 0 | Under-expressed |
| CRLS1 | 0.469809488462613 | 0 | Under-expressed |
| PET100 | 0.469729857666363 | 0 | Under-expressed |
| GTF3A | 0.46942467442764 | 0 | Under-expressed |
| CCDC9B | 0.468285115280717 | 0 | Under-expressed |
| FLII | 0.467303482445616 | 0 | Under-expressed |
| CPTP | 0.46545150946177 | 0 | Under-expressed |
| ALDH2 | 0.462694245824134 | 0 | Under-expressed |
| EIF4G1 | 0.4625372263878 | 0 | Under-expressed |
| ETFDH | 0.460616117821288 | 0 | Under-expressed |
| CD81 | 0.460203933505524 | 0 | Under-expressed |
| PMP22 | 0.459308532239243 | 0 | Under-expressed |
| POMGNT1 | 0.45818413967512 | 0 | Under-expressed |
| RBM20 | 0.456978457928134 | 0 | Under-expressed |
| DVL1 | 0.45673265766743 | 0 | Under-expressed |
| RRAGD | 0.456229164351205 | 0 | Under-expressed |
| NCAM1 | 0.45487112298858 | 0 | Under-expressed |
| CTNNA3 | 0.453990936690823 | 0 | Under-expressed |
| HSPA9 | 0.453541087244094 | 0 | Under-expressed |
| SLC39A14 | 0.452478536224251 | 0 | Under-expressed |
| TCEA3 | 0.450954798197644 | 0 | Under-expressed |
| RNF207 | 0.450787717656615 | 0 | Under-expressed |
| FITM2 | 0.450367242082194 | 0 | Under-expressed |
| CHRM2 | 0.448381303667194 | 0 | Under-expressed |
| CYB5R3 | 0.448245055777138 | 0 | Under-expressed |
| PTDSS1 | 0.44556966938682 | 0 | Under-expressed |
| LAMB2 | 0.445096861484143 | 0 | Under-expressed |
| HIPK3 | 0.444038503243751 | 0 | Under-expressed |
| RHOD | 0.443734502083528 | 0 | Under-expressed |
| HDLBP | 0.443276408613703 | 0 | Under-expressed |
| KLHL24 | 0.442151032594716 | 0 | Under-expressed |
| EMC3 | 0.440647832139026 | 0 | Under-expressed |
| SEPTIN5 | 0.440294150727879 | 0 | Under-expressed |
| MASP1 | 0.439754361378425 | 0 | Under-expressed |
| NDUFS2 | 0.438218270311542 | 0 | Under-expressed |
| CCDC141 | 0.43816554910103 | 0 | Under-expressed |
| DNAJA4 | 0.437304030321797 | 0 | Under-expressed |
| C11orf21 | 0.436076527360092 | 0 | Under-expressed |
| LARGE1 | 0.433898660238736 | 0 | Under-expressed |
| PCBD1 | 0.433511190981426 | 0 | Under-expressed |
| CSDC2 | 0.432762434188547 | 0 | Under-expressed |
| ALKBH5 | 0.432458558530208 | 0 | Under-expressed |
| MYPN | 0.432112631942704 | 0 | Under-expressed |
| MTFR1L | 0.430188429215674 | 0 | Under-expressed |
| BAD | 0.428766221193802 | 0 | Under-expressed |
| RTRAF | 0.427993801582703 | 0 | Under-expressed |
| GSTM3 | 0.427436909129065 | 0 | Under-expressed |
| HOMER1 | 0.426138849379217 | 0 | Under-expressed |
| FKBP3 | 0.425325292434066 | 0 | Under-expressed |
| DCAF6 | 0.425314412587644 | 0 | Under-expressed |
| CYB5R1 | 0.424971106853058 | 0 | Under-expressed |
| ASB2 | 0.42487216322268 | 0 | Under-expressed |
| AFG3L2 | 0.424772116403007 | 0 | Under-expressed |
| TMEM134 | 0.424518266217449 | 0 | Under-expressed |
| MGAT4B | 0.424256690310209 | 0 | Under-expressed |
| WSB2 | 0.423390454084568 | 0 | Under-expressed |
| SLC29A1 | 0.422969354984263 | 0 | Under-expressed |
| TRAP1 | 0.422784135983897 | 0 | Under-expressed |
| SPEG | 0.421609314838095 | 0 | Under-expressed |
| GAMT | 0.421329207561897 | 0 | Under-expressed |
| CHPF | 0.421208152017896 | 0 | Under-expressed |
| GUCD1 | 0.420048982206932 | 0 | Under-expressed |
| ARIH2 | 0.419764566237942 | 0 | Under-expressed |
| WFS1 | 0.419505364966516 | 0 | Under-expressed |
| PPP1R12C | 0.419246113200945 | 0 | Under-expressed |
| NFIC | 0.41916292562013 | 0 | Under-expressed |
| NIBAN1 | 0.417802553788592 | 0 | Under-expressed |
| MAST4 | 0.416082775215313 | 0 | Under-expressed |
| PYGM | 0.41570458721948 | 0 | Under-expressed |
| CITED4 | 0.414629713854831 | 0 | Under-expressed |
| TRIM63 | 0.41402073903663 | 0 | Under-expressed |
| NDUFA6 | 0.412549896897131 | 0 | Under-expressed |
| LAPTM4B | 0.412530367786058 | 0 | Under-expressed |
| TAF10 | 0.412380837147967 | 0 | Under-expressed |
| TOM1L2 | 0.412095204181453 | 0 | Under-expressed |
| TMEM120A | 0.411937142054942 | 0 | Under-expressed |
| DST | 0.411596109201933 | 0 | Under-expressed |
| DRAP1 | 0.410414991516609 | 0 | Under-expressed |
| AKAP1 | 0.410134321925265 | 0 | Under-expressed |
| C10orf71 | 0.406266692416382 | 0 | Under-expressed |
| TIMM8B | 0.404137711759467 | 0 | Under-expressed |
| BAG1 | 0.402818264785107 | 0 | Under-expressed |
| GNG7 | 0.402152465047532 | 0 | Under-expressed |
| CNBP | 0.399967974978962 | 0 | Under-expressed |
| TUFM | 0.399684644821933 | 0 | Under-expressed |
| PTGES3L | 0.399650028635333 | 0 | Under-expressed |
| UBE2B | 0.39726749166139 | 0 | Under-expressed |
| PAIP2B | 0.395653253386674 | 0 | Under-expressed |
| ITGA7 | 0.395126728478028 | 0 | Under-expressed |
| KLHDC3 | 0.394405216805488 | 0 | Under-expressed |
| PATJ | 0.393452984526798 | 0 | Under-expressed |
| GATA6 | 0.393422363202024 | 0 | Under-expressed |
| FHOD3 | 0.393305156072137 | 0 | Under-expressed |
| SLC2A4RG | 0.393160711207896 | 0 | Under-expressed |
| PPP1R13B | 0.391346101698276 | 0 | Under-expressed |
| ISCU | 0.389994062441203 | 0 | Under-expressed |
| NDUFAF3 | 0.388772903515874 | 0 | Under-expressed |
| PDCD5 | 0.387655572182626 | 0 | Under-expressed |
| PRDX5 | 0.387605305912183 | 0 | Under-expressed |
| BCKDHA | 0.386735346478775 | 0 | Under-expressed |
| CORO6 | 0.385358480229394 | 0 | Under-expressed |
| ATP5PB | 0.384966221638449 | 0 | Under-expressed |
| PPIF | 0.384928589491583 | 0 | Under-expressed |
| NUCKS1 | 0.383942015623345 | 0 | Under-expressed |
| MAPT | 0.383822539869195 | 0 | Under-expressed |
| FMC1 | 0.383419102355255 | 1.58426507997188e−295 | Under-expressed |
| CUL4A | 0.381918062675314 | 0 | Under-expressed |
| RILP | 0.379344761639368 | 0 | Under-expressed |
| ATP1B1 | 0.3791534229869 | 8.29665366953652e−275 | Under-expressed |
| CDK16 | 0.378187783791425 | 0 | Under-expressed |
| SERPINB6 | 0.377309174748246 | 0 | Under-expressed |
| POLR2I | 0.377270594356809 | 0 | Under-expressed |
| NENF | 0.376452494854332 | 0 | Under-expressed |
| LDHD | 0.376445171225206 | 0 | Under-expressed |
| KTN1 | 0.372394050383806 | 0 | Under-expressed |
| SCN1B | 0.372069431015718 | 0 | Under-expressed |
| GPD1L | 0.371721041255548 | 1.66543480276256e−295 | Under-expressed |
| SPAG7 | 0.371262043903082 | 0 | Under-expressed |
| REX1BD | 0.37098741809554 | 0 | Under-expressed |
| MLYCD | 0.370152975179954 | 0 | Under-expressed |
| SLMAP | 0.368681582946904 | 0 | Under-expressed |
| INSYN1 | 0.366439926027358 | 0 | Under-expressed |
| HK1 | 0.365443882694908 | 0 | Under-expressed |
| PPDPF | 0.365344816252008 | 1.33033623856196e−285 | Under-expressed |
| GLRX5 | 0.364948877930317 | 0 | Under-expressed |
| KIAA0232 | 0.36461780515502 | 0 | Under-expressed |
| EDF1 | 0.364548976646078 | 4.37039064253458e−293 | Under-expressed |
| FBXO32 | 0.36453879418733 | 0 | Under-expressed |
| COA8 | 0.364526824859386 | 0 | Under-expressed |
| FXR2 | 0.361781866337786 | 0 | Under-expressed |
| DDT | 0.361668561830036 | 0 | Under-expressed |
| DLD | 0.361047115259077 | 0 | Under-expressed |
| MAN2A2 | 0.360966826362404 | 0 | Under-expressed |
| ADCY5 | 0.360893882634829 | 0 | Under-expressed |
| CAND2 | 0.360405081535826 | 0 | Under-expressed |
| CES2 | 0.360047959470036 | 0 | Under-expressed |
| ILVBL | 0.359452014375179 | 0 | Under-expressed |
| AVEN | 0.359444772367859 | 0 | Under-expressed |
| FIS1 | 0.35902867376291 | 0 | Under-expressed |
| IFI6 | 0.358978658174078 | 0 | Under-expressed |
| SPHKAP | 0.358880013173552 | 0 | Under-expressed |
| FASTK | 0.358693867401765 | 0 | Under-expressed |
| ADCY6 | 0.358208201437131 | 0 | Under-expressed |
| INSR | 0.357981252799352 | 0 | Under-expressed |
| ADHFE1 | 0.356523598994366 | 0 | Under-expressed |
| KANK1 | 0.356240874695723 | 0 | Under-expressed |
| PSMD8 | 0.355929024710766 | 0 | Under-expressed |
| PYGB | 0.355659771333728 | 0 | Under-expressed |
| RNF150 | 0.355507225339956 | 0 | Under-expressed |
| KIF21A | 0.355401586995948 | 0 | Under-expressed |
| OCIAD1 | 0.354910039770875 | 0 | Under-expressed |
| PSD3 | 0.352921658176173 | 0 | Under-expressed |
| PDHX | 0.351817276195081 | 0 | Under-expressed |
| UBE2QL1 | 0.351701423318032 | 0 | Under-expressed |
| EIF3A | 0.351412000125386 | 3.62194090244114e−318 | Under-expressed |
| GPSM1 | 0.35133169820471 | 0 | Under-expressed |
| CACNA2D3 | 0.350392205724202 | 0 | Under-expressed |
| CLUH | 0.349901137116161 | 0 | Under-expressed |
| PPP2CB | 0.349648673712831 | 2.65540252707167e−267 | Under-expressed |
| GMPR | 0.349573871000859 | 0 | Under-expressed |
| ADA2 | 0.349439505922554 | 0 | Under-expressed |
| PHACTR1 | 0.349410514778085 | 0 | Under-expressed |
| C1orf122 | 0.349204692590235 | 0 | Under-expressed |
| LBH | 0.349100790195254 | 0 | Under-expressed |
| DVL3 | 0.347598559378665 | 0 | Under-expressed |
| TSPO | 0.347527071527065 | 0 | Under-expressed |
| REEP5 | 0.347487342673393 | 4.69887698362718e−314 | Under-expressed |
| ETFA | 0.347227299871973 | 0 | Under-expressed |
| VEGFB | 0.346823369919245 | 0 | Under-expressed |
| CFLAR | 0.345090729733853 | 5.83112576442482e−289 | Under-expressed |
| CAV2 | 0.344942802380072 | 5.10040847184263e−295 | Under-expressed |
| ARL6IP5 | 0.344554097270546 | 1.162976937573e−285 | Under-expressed |
| NDUFS3 | 0.343964435965068 | 0 | Under-expressed |
| NDUFS7 | 0.343237151727435 | 0 | Under-expressed |
| SRF | 0.343020034688695 | 0 | Under-expressed |
| NDUFA7 | 0.342133120088093 | 0 | Under-expressed |
| LRIG1 | 0.341473217892926 | 0 | Under-expressed |
| TMEM65 | 0.341285595806596 | 0 | Under-expressed |
| SLC25A12 | 0.340688735207629 | 0 | Under-expressed |
| CIRBP | 0.340267764121694 | 1.33936858780741e−267 | Under-expressed |
| XIRP2 | 0.339981683082121 | 0 | Under-expressed |
| ALDOC | 0.339685144008008 | 1.67960610201549e−305 | Under-expressed |
| RWDD1 | 0.339299669670759 | 0 | Under-expressed |
| ROGDI | 0.339232773611562 | 0 | Under-expressed |
| IVD | 0.33917822654183 | 0 | Under-expressed |
| HDGF | 0.338334857712582 | 1.61388777231767e−307 | Under-expressed |
| TMEM159 | 0.337918452997439 | 2.89101227149489e−299 | Under-expressed |
| 1.00 MLF | 0.337164431871858 | 0 | Under-expressed |
| NDUFB5 | 0.33628550179894 | 1.31445907013373e−311 | Under-expressed |
| CCNI | 0.335064471323274 | 3.39898622559998e−231 | Under-expressed |
| RBM38 | 0.334226376028133 | 0 | Under-expressed |
| FAM162A | 0.333823901443869 | 0 | Under-expressed |
| JUP | 0.333432770787512 | 0 | Under-expressed |
| ASB15 | 0.33269897108612 | 0 | Under-expressed |
| DCXR | 0.332335639834166 | 0 | Under-expressed |
| NDUFA12 | 0.331933537016998 | 3.67903086240403e−24 | Under-expressed |
| CUEDC1 | 0.331705592627131 | 0 | Under-expressed |
| SLC27A1 | 0.331699139477468 | 0 | Under-expressed |
| PXMP2 | 0.331344088820414 | 0 | Under-expressed |
| HAGH | 0.330558095784112 | 0 | Under-expressed |
| PSMB6 | 0.330133567178819 | 0 | Under-expressed |
| PTPA | 0.329447380766853 | 0 | Under-expressed |
| PKD1 | 0.328465243236463 | 0 | Under-expressed |
| ACOT11 | 0.328150371886488 | 0 | Under-expressed |
| GTF3C5 | 0.327858593528713 | 0 | Under-expressed |
| WIPI1 | 0.326816618477828 | 0 | Under-expressed |
| TIMM21 | 0.326651917199313 | 0 | Under-expressed |
| MTSS2 | 0.326612286191716 | 0 | Under-expressed |
| CISD3 | 0.326445383533443 | 0 | Under-expressed |
| FAM3A | 0.325536949496938 | 0 | Under-expressed |
| HLA-DRB5 | 0.325428996807887 | 0 | Under-expressed |
| FITM1 | 0.324602137108623 | 1.34811722594038e−294 | Under-expressed |
| NES | 0.324084318481541 | 3.90621481890006e−300 | Under-expressed |
| CRAT | 0.324068809590612 | 0 | Under-expressed |
| DGLUCY | 0.323105407488237 | 0 | Under-expressed |
| HNRNPDL | 0.322913792547626 | 1.1006412269825e−203 | Under-expressed |
| RAMP1 | 0.32290814566697 | 4.14021138121212e−265 | Under-expressed |
| PCMT1 | 0.322721311400323 | 0 | Under-expressed |
| DAG1 | 0.32201243369291 | 0 | Under-expressed |
| FH | 0.319893661811924 | 0 | Under-expressed |
| UQCC1 | 0.31943745407322 | 0 | Under-expressed |
| B4GALNT3 | 0.319111138978069 | 0 | Under-expressed |
| CPNE4 | 0.319078499620844 | 0 | Under-expressed |
| PTGES2 | 0.319066983166235 | 0 | Under-expressed |
| DCUN1D2 | 0.318688576544183 | 0 | Under-expressed |
| MEF2A | 0.318255617590556 | 5.54378709557317e−318 | Under-expressed |
| ACSS1 | 0.317498438184566 | 0 | Under-expressed |
| CANX | 0.316902779354904 | 8.79566228682066e−250 | Under-expressed |
| ZNF330 | 0.315535960601197 | 0 | Under-expressed |
| KIF1B | 0.315369581391904 | 2.44473081376769e−288 | Under-expressed |
| AKAP13 | 0.314771634238169 | 0 | Under-expressed |
| TRIM8 | 0.31467580800712 | 2.22665543223633e−281 | Under-expressed |
| NEK7 | 0.312882220470231 | 6.64417218144061e−284 | Under-expressed |
| MPST | 0.312745689312796 | 0 | Under-expressed |
| TXN | 0.31251556321345 | 4.70490862398716e−314 | Under-expressed |
| VPS13D | 0.31089320293841 | 0 | Under-expressed |
| SDC4 | 0.309922320697645 | 0 | Under-expressed |
| EXOC6B | 0.309835215751282 | 7.10703736731804e−302 | Under-expressed |
| RAB5B | 0.308928733150153 | 2.11076131808253e−262 | Under-expressed |
| ALKBH7 | 0.30833480144725 | 0 | Under-expressed |
| ATP1A3 | 0.308233833607798 | 0 | Under-expressed |
| PSMB1 | 0.30783336924675 | 4.83552756784667e−266 | Under-expressed |
| GYS1 | 0.306923309401194 | 0 | Under-expressed |
| RABL6 | 0.305759623447717 | 0 | Under-expressed |
| HINT1 | 0.305684856724598 | 2.7876508421827e−248 | Under-expressed |
| PACRG | 0.305328598244701 | 0 | Under-expressed |
| NAPA | 0.304441626888693 | 3.81516892023366e−261 | Under-expressed |
| FAM234A | 0.303834839577928 | 1.09903364611586e−311 | Under-expressed |
| CLGN | 0.303818781086694 | 0 | Under-expressed |
| SGCG | 0.303102595383369 | 0 | Under-expressed |
| DSTN | 0.302466874801031 | 1.87576227317157e−275 | Under-expressed |
| HSDL2 | 0.301709632165782 | 0 | Under-expressed |
| AHSA1 | 0.301489019083355 | 2.30415524898758e−258 | Under-expressed |
| DMD | 0.300528125108828 | 0 | Under-expressed |
| WDTC1 | 0.300032210306643 | 5.63036884195285e−293 | Under-expressed |
| STUB1 | 0.299557327142089 | 2.26678745165466e−267 | Under-expressed |
| MYL4 | 0.299461009164636 | 0 | Under-expressed |
| ARL6IP4 | 0.299295567456637 | 7.41023586539858e−226 | Under-expressed |
| EIF4G2 | 0.299288833356433 | 2.76147706794871e−199 | Under-expressed |
| THSD4 | 0.299121729525624 | 0 | Under-expressed |
| APOBEC2 | 0.298667106745222 | 0 | Under-expressed |
| SLC25A23 | 0.298409333868205 | 0 | Under-expressed |
| NDUFA2 | 0.298307986471454 | 3.12376766362394e−253 | Under-expressed |
| LYNX1 | 0.298055451813547 | 0 | Under-expressed |
| DCAF8 | 0.297774652728241 | 3.0609198172651e−288 | Under-expressed |
| TSPYL1 | 0.29725168870335 | 1.43799923886266e−314 | Under-expressed |
| USP2 | 0.296992636349967 | 0 | Under-expressed |
| PCM1 | 0.296427811022891 | 1.3603213480014e−241 | Under-expressed |
| FLOT1 | 0.294981622112233 | 4.57961355420762e−238 | Under-expressed |
| PIM3 | 0.294565664935479 | 0 | Under-expressed |
| ATAD1 | 0.294334094529594 | 0 | Under-expressed |
| HIF3A | 0.294194692693352 | 3.17417481425912e−172 | Under-expressed |
| SCP2 | 0.293293538729558 | 1.14595127279148e−261 | Under-expressed |
| TNIP1 | 0.293136880215731 | 1.88274730911864e−254 | Under-expressed |
| RAB21 | 0.293086879036313 | 7.63007205917789e−259 | Under-expressed |
| UROS | 0.292534546006936 | 0 | Under-expressed |
| PPP1R3B | 0.292079494130423 | 0 | Under-expressed |
| ECI1 | 0.291435217972265 | 0 | Under-expressed |
| MLPH | 0.290683667705705 | 0 | Under-expressed |
| CCDC8 | 0.29051680706392 | 4.28125571084885e−311 | Under-expressed |
| SF3B5 | 0.289769331246489 | 1.59497866514018e−266 | Under-expressed |
| TRIM55 | 0.28962326815314 | 0 | Under-expressed |
| UBE2D2 | 0.289292117828469 | 5.56871244839871e−252 | Under-expressed |
| SMIM4 | 0.28924116495702 | 1.72793716152676e−269 | Under-expressed |
| TECRL | 0.288818121740565 | 0 | Under-expressed |
| C4orf3 | 0.287946450117463 | 2.31469998376446e−209 | Under-expressed |
| TENM2 | 0.287220611257563 | 0 | Under-expressed |
| PAM16 | 0.287168014083018 | 0 | Under-expressed |
| C1orf198 | 0.287008065618693 | 0 | Under-expressed |
| FAM78A | 0.286733516919229 | 0 | Under-expressed |
| HSF1 | 0.28636277417773 | 1.73467090717052e−304 | Under-expressed |
| LGALS1 | 0.284205197858385 | 2.27501010656131e−152 | Under-expressed |
| PABPC4 | 0.284182842589516 | 2.53037896862281e−272 | Under-expressed |
| GAS6 | 0.283623366296438 | 2.1610713463947e−206 | Under-expressed |
| POLDIP2 | 0.283060349686228 | 1.42067145508007e−268 | Under-expressed |
| SELENOH | 0.283047233495901 | 7.88718057209894e−267 | Under-expressed |
| USP22 | 0.282530002948904 | 1.11878892777791e−172 | Under-expressed |
| LRRC20 | 0.282460220182018 | 0 | Under-expressed |
| HOMER2 | 0.282272727435397 | 0 | Under-expressed |
| SLC16A1 | 0.282061608357536 | 0 | Under-expressed |
| PGP | 0.281895424025813 | 0 | Under-expressed |
| MXI1 | 0.281281392924588 | 1.79210692005638e−217 | Under-expressed |
| BCO2 | 0.280691246473582 | 1.45082854916075e−307 | Under-expressed |
| SLC5A1 | 0.279945186571692 | 0 | Under-expressed |
| CCDC85C | 0.279940386574378 | 0 | Under-expressed |
| PPP1R3A | 0.279824600083873 | 0 | Under-expressed |
| COPS5 | 0.279758557688949 | 1.98075210415047e−265 | Under-expressed |
| FBXL17 | 0.279423205548004 | 0 | Under-expressed |
| DPF3 | 0.279310186959989 | 0 | Under-expressed |
| C3orf18 | 0.279225684692421 | 0 | Under-expressed |
| METRNL | 0.278694023230956 | 6.15988663479453e−315 | Under-expressed |
| NUCB1 | 0.278650293030928 | 2.82255984913699e−161 | Under-expressed |
| CALU | 0.278073830680665 | 1.16385471009236e−166 | Under-expressed |
| RAF1 | 0.277540901110912 | 2.08905787711811e−271 | Under-expressed |
| KEAP1 | 0.277177565672873 | 0 | Under-expressed |
| STXBP6 | 0.277161238039407 | 1.64994637408113e−294 | Under-expressed |
| PSMB7 | 0.277104584523533 | 9.0416077445539e−241 | Under-expressed |
| RNF115 | 0.27703194814316 | 4.88691742155174e−268 | Under-expressed |
| HEBP2 | 0.276186517165889 | 8.46834801385698e−219 | Under-expressed |
| SCPEP1 | 0.275442206307843 | 1.12499527384249e−245 | Under-expressed |
| AP3D1 | 0.275343124906877 | 4.16177929143683e−242 | Under-expressed |
| UQCC2 | 0.274145169733347 | 0 | Under-expressed |
| MLEC | 0.273935090774291 | 3.1459787897719e−175 | Under-expressed |
| CLCN3 | 0.273506193140766 | 7.02540146710168e−301 | Under-expressed |
| BDH1 | 0.27340357427858 | 0 | Under-expressed |
| COA4 | 0.273148224034165 | 1.94692785849691e−288 | Under-expressed |
| PRKAG2 | 0.272808506589004 | 0 | Under-expressed |
| TRAK2 | 0.272768029011008 | 3.15271441582677e−226 | Under-expressed |
| AGPAT3 | 0.272646264988099 | 1.15534243807735e−287 | Under-expressed |
| GRPEL1 | 0.272535837223708 | 0 | Under-expressed |
| MYO18B | 0.271844233759446 | 0 | Under-expressed |
| GPT | 0.271696274495495 | 0 | Under-expressed |
| ITPK1 | 0.271397260179918 | 3.55416764248408e−266 | Under-expressed |
| BAG6 | 0.271082861612371 | 1.44465429457896e−222 | Under-expressed |
| AAK1 | 0.270586520445279 | 2.65546662706342e−314 | Under-expressed |
| CMBL | 0.270129501284974 | 7.57385213186757e−299 | Under-expressed |
| PPARGC1A | 0.269813842914016 | 0 | Under-expressed |
| CHMP3 | 0.269359042451315 | 6.16990833544009e−227 | Under-expressed |
| CAP2 | 0.269293273347698 | 2.37011176063749e−309 | Under-expressed |
| TNNT1 | 0.269078128196521 | 0 | Under-expressed |
| RERE | 0.267416693379024 | 3.76094021804115e−190 | Under-expressed |
| AKT2 | 0.267024936758625 | 8.60721936059225e−287 | Under-expressed |
| RALY | 0.266972244942442 | 5.79242754237606e−198 | Under-expressed |
| SNRNP25 | 0.266738932183209 | 5.54557015055346e−264 | Under-expressed |
| RBX1 | 0.266718434451191 | 1.15742031896335e−218 | Under-expressed |
| PRXL2A | 0.266047523651236 | 8.56931082106802e−298 | Under-expressed |
| PCYOX1 | 0.265636670905619 | 1.35103233334457e−240 | Under-expressed |
| TMX4 | 0.265062304487344 | 1.48033747339442e−221 | Under-expressed |
| MAVS | 0.265050212836759 | 8.65067098827656e−265 | Under-expressed |
| TXNL1 | 0.264759048191066 | 1.73348452166939e−275 | Under-expressed |
| TBRG4 | 0.264580586985169 | 0 | Under-expressed |
| PRUNE1 | 0.26387187261811 | 0 | Under-expressed |
| TOB1 | 0.263418070233078 | 3.14517521222184e−316 | Under-expressed |
| SLC1A3 | 0.263266497360799 | 9.2414698054697e−290 | Under-expressed |
| SSU72 | 0.26324447881628 | 2.47045639742173e−219 | Under-expressed |
| PCDHGB1 | 0.263098572708846 | 0 | Under-expressed |
| ATXN10 | 0.262186420056748 | 2.37592076176723e−226 | Under-expressed |
| DDB1 | 0.261623009696116 | 5.23062355264742e−227 | Under-expressed |
| SORT1 | 0.261335020971648 | 2.83144228409101e−208 | Under-expressed |
| GPAT3 | 0.260387716988977 | 0 | Under-expressed |
| CD36 | 0.260306899788392 | 1.79659371333268e−143 | Under-expressed |
| PCDH7 | 0.260246136833025 | 5.30630218870987e−259 | Under-expressed |
| UBR3 | 0.260197300747466 | 1.25925185846583e−265 | Under-expressed |
| RNH1 | 0.259780816232214 | 1.06487328845186e−228 | Under-expressed |
| SPATA20 | 0.259123713959323 | 2.79320637775261e−293 | Under-expressed |
| APLP2 | 0.259088802240933 | 7.41838724073883e−155 | Under-expressed |
| DMAC2 | 0.259046883244466 | 1.36424044675133e−263 | Under-expressed |
| WBP2 | 0.258998072503176 | 3.98163847166297e−213 | Under-expressed |
| PIN1 | 0.258952014015159 | 2.35618788853355e−225 | Under-expressed |
| HACD1 | 0.258897977347659 | 1.34720885934994e−312 | Under-expressed |
| PXDNL | 0.258521048339185 | 8.41168827958797e−292 | Under-expressed |
| COQ10A | 0.258262717004685 | 0 | Under-expressed |
| IDH3A | 0.257101934523137 | 3.06490904135078e−264 | Under-expressed |
| PPP2R3A | 0.256806059810536 | 0 | Under-expressed |
| SIRT5 | 0.256666843370354 | 8.31387048122584e−297 | Under-expressed |
| NPTN | 0.256286738767836 | 5.35632083651723e−170 | Under-expressed |
| VCP | 0.255967046639106 | 7.33040372479702e−206 | Under-expressed |
| TSC22D4 | 0.254557043461666 | 1.20620782955845e−286 | Under-expressed |
| REXO2 | 0.254100230064823 | 1.91585127947316e−149 | Under-expressed |
| SMCO1 | 0.253990134530533 | 0 | Under-expressed |
| ABCB6 | 0.25394016754794 | 0 | Under-expressed |
| FKBP8 | 0.253931643040083 | 7.19433281664503e−169 | Under-expressed |
| CTNNAL1 | 0.253843349782497 | 7.05611802818144e−251 | Under-expressed |
| SAT2 | 0.252530493762943 | 8.77091343620807e−262 | Under-expressed |
| THRA | 0.252409741579307 | 1.65086072582884e−215 | Under-expressed |
| SYNGR1 | 0.251924523899127 | 3.34534704670797e−246 | Under-expressed |
| KLF15 | 0.251777638366386 | 7.42778546884042e−268 | Under-expressed |
| PLEKHA5 | 0.251547849552459 | 2.6372323049908e−282 | Under-expressed |
| POLR2F | 0.251153071721936 | 4.74271572214454e−207 | Under-expressed |
| TOMM40L | 0.25110906789258 | 8.2175474516683e−308 | Under-expressed |
| OPA1 | 0.250699453576237 | 2.72113038075396e−255 | Under-expressed |
| MAPKAP1 | 0.250444633420945 | 1.78876725582501e−257 | Under-expressed |
| GABARAPL2 | 0.250242469899528 | 2.89340898247986e−158 | Under-expressed |

TABLE 19
SARS-CoV-2 biomarkers that were not dysregulated by
more than 0.1-fold in infected human heart tissue.

| Gene | Average log2FC | Adjusted p-value |
|---|---|---|
| SLC11A2 | 0.00792763477601914 | 9.25238957250028e−08 |
| MLXIP | 0.0089067261560325 | 8.84897625632249e−15 |
| HIPK1 | 0.00162826203205044 | 7.09980717497307e−06 |
| PLEKHM3 | 0.00759406844456435 | 7.09547659911147e−07 |
| GPN2 | 0.000551177792642493 | 7.0634363350592e−11 |
| UBE2K | 0.00415266946994852 | 6.41700961769647e−10 |
| YLPM1 | 0.0091860055249296 | 6.11855392764706e−16 |
| IKZF5 | 0.00353117211658115 | 6.07282470052178e−09 |
| HTRA2 | 0.0047690298586676 | 5.92344108603489e−05 |
| FAM174C | 0.00848122275365953 | 5.915604446841e−14 |
| TGFBRAP1 | 0.00535020866654495 | 5.8081728846652e−07 |
| RFC1 | 0.00199090977408439 | 4.94896084888657e−12 |
| ZNF318 | 0.00553254036295554 | 3.97929909689529e−13 |
| WDR43 | 0.00844011257923724 | 3.69005930305416e−05 |
| PDE6D | 0.00300454166036224 | 3.4681160719856e−11 |
| VPS11 | 0.0021167229679615 | 3.13878316890422e−09 |
| ZC3H14 | 0.00525022559142085 | 3.11586240184667e−08 |
| RTKN | 0.000798932556712678 | 3.07518776631607e−21 |
| NTN4 | 0.00779054085341452 | 2.92682012648582e−12 |
| RPA1 | 0.00116900821681151 | 2.64959485706794e−09 |
| AATF | 0.00199123997024202 | 2.6474173207212e−11 |
| NGDN | 0.00444790566482457 | 2.59236199841177e−06 |
| ABHD16A | 0.00651876977478624 | 2.54961689885472e−35 |
| SEC11C | 0.00832668763371694 | 2.53464475097182e−06 |
| PARP3 | 0.00011306969826326 | 2.49566077654796e−09 |
| PPP1R35 | 0.00268828476089278 | 2.46224718591177e−23 |
| ZNF785 | 0.00947199912713814 | 2.31982365843524e−11 |
| NEU1 | 0.00995937483197033 | 2.07345013917991e−06 |
| ZNF317 | 0.00110856578888209 | 1.9749042082208e−05 |
| EOGT | 0.00197052189739093 | 1.77197380192717e−11 |
| ASNSD1 | 0.00398254141069443 | 1.76467136683241e−05 |
| GNL3L | 0.00702043454639925 | 1.69009266478809e−15 |
| C16orf58 | 0.00871489540585582 | 1.66303331240731e−06 |
| NMT2 | 0.00993652951845975 | 1.62355831863014e−09 |
| SLC30A5 | 0.00882849596107597 | 1.50205444741741e−09 |
| RER1 | 0.00488160322239434 | 1.46246551955025e−07 |
| ARMC7 | 0.00723759710682984 | 1.43488523077054e−25 |
| DCTN2 | 0.00950732082217343 | 1.40092561293831e−10 |
| GOLIM4 | 0.00959612561204377 | 1.38856340156272e−11 |
| SHTN1 | 0.00163895820742994 | 1.37226839519451e−06 |
| PHF6 | 0.00175498307129093 | 1.2441106015078e−29 |
| MZF1 | 0.00904808119025773 | 1.24214884034193e−20 |
| COQ10B | 0.00626874777119774 | 1.23453095626494e−13 |
| GPC4 | 0.00748843713981563 | 1.18918805415077e−08 |
| GADD45G | 0.00201061854451089 | 1.14552320392552e−06 |
| UBN2 | 0.00644245487403648 | 1.13795380492755e−05 |
| QSOX2 | 0.00729625388674615 | 1.04040625741777e−15 |
| GALNS | 0.00988013403273758 | 1.01502444810068e−05 |
| MET | 0.00131512290305245 | 1.00419521000653e−08 |
| ERVK3-1 | 0.00647246925963561 | 0.044412750439087 |
| VPS16 | 0.00342000246206207 | 0.0344729808782798 |
| ATF7IP | 0.00867359615975788 | 0.0310928897940967 |
| ZNF587B | 0.00671812888233743 | 0.0197172108183389 |
| ABCC9 | 0.0068396832845781 | 0.0192106859036152 |
| SNAP29 | 0.00193497414192847 | 0.0174965872347277 |
| VPS41 | 0.0020804283375634 | 0.0173736652708153 |
| ATRX | 0.00654520727184976 | 0.0157367219470507 |
| DAXX | 0.00241112637096635 | 0.0147018362209209 |
| TASOR2 | 0.00628572928601023 | 0.0124264889193053 |
| RRP36 | 0.00604128021521252 | 0.00915578369295199 |
| LIN7A | 0.00596730975595371 | 0.00802612904691442 |
| AP3M1 | 0.00520438593679978 | 0.00709956675607419 |
| UTP23 | 0.00206962188620829 | 0.0059767016238832 |
| MIGA1 | 0.00199392473484569 | 0.00373170837489408 |
| TBC1D16 | 0.000430517345945958 | 0.00352925409163199 |
| UBQLN1 | 0.00364449576535553 | 0.00350135555194025 |
| PHACTR4 | 0.00665050088216809 | 0.00193736974313111 |
| BRD3 | 0.00737613126690259 | 0.00162537252658569 |
| DEAF1 | 0.00103946427137858 | 0.00117587464011847 |
| NCAPD3 | 0.00684630998509808 | 0.001139454881284 |
| ARMCX1 | 0.00637535235350198 | 0.00106181526289399 |
| BRF1 | 0.00691056983915081 | 0.000947787959564358 |
| CD2BP2 | 0.00804402898750964 | 0.000878945750718095 |
| ZNF235 | 0.00937143913834899 | 0.000728629100920445 |
| D2HGDH | 0.00730072598781126 | 0.000625789169342439 |
| RYK | 0.001446409021127 | 0.000398481579315092 |
| CUL9 | 0.00568182057535548 | 0.000217574483746901 |
| COPS7B | 0.000907470408452071 | 0.000135756481529473 |

As shown in Tables 17-19 and shown spatially in FIGS. 31-42, at least 500 biomarkers were upregulated or downregulated in a human heart tissue sample in a subject infected with SARS-CoV-2. These data demonstrate a proof of concept that biomarkers associated with SARS-CoV-2 can be identified using the RTL methods from Example 1 at the same time as detecting SARS-CoV-2 virus, in a subject in general, and specifically in heart tissue of an infected subject. Additionally, SARS-CoV-2 virus can be detected in tissues, specifically heart tissues, of an infected subject (see white arrows in center panels of FIGS. 31-39).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-46. (canceled)

47. A method for determining location of a coronavirus nucleic acid in a biological sample, the method comprising:
(a) contacting the biological sample with a substrate comprising a plurality of capture probes, wherein each capture probe of the plurality comprises a spatial barcode and a capture domain;
(b) contacting the biological sample with a plurality of first coronavirus probes and second coronavirus probes, wherein a first coronavirus probe and a second coronavirus probe each comprise a sequence that is substantially complementary to sequences of the coronavirus nucleic acid, and wherein the second probe comprises a coronavirus capture probe capture domain that is complementary to all or a portion of the capture domain of the capture probe; (c)
(c) hybridizing the first coronavirus probe and the second coronavirus probe to the coronavirus nucleic acid;
(d) ligating the first coronavirus probe to the second coronavirus probe, thereby generating a ligation product;
(e) releasing the ligation product from the coronavirus nucleic acid; and
(f) determining (i) all or part of the sequence of the ligation product, or a complement thereof, and (ii) the spatial barcode, or a complement thereof; and using the determined sequences of (i) and (ii) to determine the location of the coronavirus nucleic acid in the biological sample.

48. The method of claim 47, wherein the plurality of first coronavirus probes and second coronavirus probes comprises two or more coronavirus probe pairs.

49. The method of claim 47, wherein the plurality of first coronavirus probes and second coronavirus probes comprises two or more of SEQ ID NOs: 29-82.

50. The method of claim 47, wherein the first coronavirus probe and the second coronavirus probe are substantially complementary to adjacent sequences of the coronavirus nucleic acid.

51. The method of claim 47, where the first coronavirus probe and the second coronavirus probe hybridize to sequences that are not adjacent to each other on the coronavirus nucleic acid.

52. The method of claim 51, wherein the first coronavirus probe is extended with a DNA polymerase, thereby filling a gap between the first coronavirus probe and the second coronavirus probe.

53. The method of claim 47, wherein the ligating the first coronavirus probe and the second coronavirus probe utilizes enzymatic ligation or chemical ligation, wherein the enzymatic ligation utilizes a ligase, wherein the ligase is one or more of a T4 RNA ligase, a PBCV-1 DNA ligase, a Chlorella virus DNA ligase, a single stranded DNA ligase, or a T4 DNA ligase.

54. The method of claim 47, wherein the first coronavirus probe comprises a primer sequence.

55. The method of claim 47, wherein the first coronavirus probe and/or the second coronavirus probe is a DNA probe.

56. The method of claim 47, wherein releasing the ligation product from the coronavirus nucleic acid comprises contacting the biological sample with an RNase H enzyme.

57. The method of claim 47, wherein the determining step (f) comprises amplifying all or part of the ligation product.

58. The method of claim 57, wherein the ligation product comprises (i) all or part of the sequence of the ligation product, or a complement thereof, and (ii) the spatial barcode, or a complement thereof.

59. The method of claim 57, wherein the determining step (f) comprises sequencing (i) all or a part of the sequence of the ligation product, or a complement thereof, and (ii) the spatial barcode, or a complement thereof.

60. The method of claim 47, wherein the coronavirus nucleic acid is from SARS-CoV-2.

61. The method of claim 47, further comprising determining the location of a host nucleic acid in the biological sample, the method comprising:
(a) contacting a first host probe and a second host probe with the biological sample, wherein the first host probe and the second host probe each comprise one or more sequences that are substantially complementary to sequences of the host nucleic acid, and wherein the second host probe comprises a host capture probe capture domain that is complementary to all or a portion of the capture domain of the capture probe;
(b) hybridizing the first host probe and the second host probe to the host nucleic acid;
(c) ligating the first host probe and the second host probe, thereby generating a host ligation product;
(d) releasing the host ligation product from the host nucleic acid;
(e) hybridizing the host ligation product to the host capture probe capture domain; and
(f) determining (i) all or part of the sequence of the host ligation product, or a complement thereof, and (ii) the spatial barcode, or a complement thereof; and using the determined sequences of (i) and (ii) to identify determine the location of the host nucleic acid in the biological sample.

62. The method of claim 61, wherein:
the steps of contacting the biological sample with the plurality of first coronavirus probes and second coronavirus probes and contacting the first host probe and the second host probe with the biological sample are performed substantially concurrently,
the steps of hybridizing the first coronavirus probe and the second coronavirus probe to the coronavirus nucleic acid and hybridizing the first host probe and the second host probe to the host nucleic acid are performed substantially concurrently,
the steps of ligating the first coronavirus probe and the second coronavirus probe and ligating the first host probe and the second host probe are performed substantially concurrently,
the steps of releasing the ligation product from the coronavirus nucleic acid and releasing the host ligation product from the host nucleic acid are performed substantially concurrently, and/or
the steps of hybridizing the ligation product to the coronavirus capture probe capture domain and hybridizing the host ligation product to the host capture probe capture domain are performed substantially concurrently.

63. The method of claim 61, wherein the biological sample is contacted with 5000 or more probe pairs.

64. The method of claim 61, wherein the host nucleic acid is one or more of IGHG1, IGLC2, IGLV3-19, IGLC3, HBA2, IGHG3, IGHV3-30, B2M, HLA-DRB1, IGKC, IGHM, JCHAIN, FTH1, FTL, IGHV4-28, CTSB, IGHV1-69-2, LYZ, S100A9, CXCL9, HLA-A, HLA-B, TIMP1, IGKV1-6, CTSD, HBB, TMSB10, CD163, MTRNR2L12, NPIPB4, IGHV3-33, MT2A, COL3A1, GAPDH, CD74, IGHV1-24, IGLV3-25, SOD2, IGLC7, IGLV1-44, IFITM3, FN1, SPARC, HIST1H2BC, SERF1A, HLA-DRA, EEF1A1, ACTB, IGHV3-21, XBP1, S100A8, IGLV2-8, VIM, LDHA, IGLV3-1, IFI30, UBB, TMSB4X, IGLC1, HMGN2, HIST2H2BF, SERF2, TPT1, IGKV1-17, PSAP, PLTP, HNRNPA2B1, TMBIM6, IGKV3OR2-268, C1QC, IGKV1D-12, CFL1, CD63, HIST2H2AC, HLA-DRB5, ACTG1, UBA52, S100A6, LUM, SRRM2, EEF2, COL1A1, PPIB, IGKV1-5, IGLV3-10, NFKBIA, COL1A2, IGFBP7, MT1G, CD44, HLA-C, PSME2, IFITM1, HBA1, IFITM2, ARPC1B, OAZ1, IGLV2-23, AL136454.1, ENO1, TPI1, HIST1H2BM, GNAS, HIST1H4B, TPM3, CTSZ, ICAM1, IGHV3-35, IGKV3-20, HIST1H2AH, FAM153B, NPIPA1, SERPINE1, LGALS1, FCER1G, FKBP1C, ATP5F1E, IGLV1-47, MT1H, HIST1H2AJ, H3F3B, CD81, EIF4A1, HIST1H2BD, CD68, DDX5, SH3BGRL3, IGHV1-2, HIST2H3D, MSN, HIST1H2AC, HLA-DPA1, SSR3, IGHA1, HIST1H1C, CYBB, WARS, CYBA, IGHV1-18, VSIG4, PCBP1, NPC2, HIST1H2BN, PKM, HMGB2, SRSF5, HLA-E, CTSH, HIST1H2AM, IGHV3-64D, HIST1H2BJ, CALR, HSPA8, CEBPB, ADAMTS2, IGHV3-15, CTSS, EEF1G, BTF3, IGHV3-66, TAP1, APLP2, TAGLN2, IFI27, CAPZB, FKBP1A, STAT1, FKBP5, HIST1H4D, TAPBP, GLUL, CLTA, TCIRG1, CALM2, HIST1H4A, PGAM1, TMA7, GABARAP, SQSTM1, MIF, IGLV1-40, EEF1B2, H1FX, IGHV4-4, NNMT, ARF1, HIST1H2AG, CD99, CCL21, HIST1H4E, DCN, IGKV3-11, LGALS3BP, CAPG, S100A10, SRGN, SLC2A3, COX7C, CIS, ALDOA, HSP90AB1, ACADVL, NBPF15, UBE2D3, WASHC1, BSG, ITM2B, NR4A1, AGER, BTNL9, FOS, RHOB, DUSP1, ZFP36, TNXB, HES1, HIF3A, JUN, CSAD, MAF, SHANK3, C4B, CYTH2, AMOTL1, KLF2, ADARB1, CAPN7, ROBO4, PON2, APOL3, DMWD, SLC12A4, BICD2, ACTN4, LMCD1, BAG1, C11orf96, PPFIBP1, BCAS2, PLVAP, SPON2, SLC11A1, CHSY1, INAFM1, SF3A2, TNS2, MED13L, KDM5B, KDM6B, SAMD4B, COL4A2, SKI, QRICH1, TNS1, CTIF, SORBS3, EEF2K, IL16, GBF1, PHACTR2, TRAK1, MAML1, DVL3, KRT18, LIMD1, PBX2, TXNDC15, ACSL3, SPAG9, MRNIP, RERE, BTG2, CD34, PTMA, PLCB3, KLF3, STAT5B, KDSR, D2HGDH, DCAF15, ABTB1, ACOT2, PLK2, INTS1, GBA2, F2R, LASIL, ITGA3, MRC2, CDIPT, SUPT6H, RBMS2, KDM4B, LMO2, EBPL, BIN3, NDRG2, CTC1, ARHGAP12, MEF2A, RNPEP, CDC42BPB, ZBTB17, GAS6, TIE1, TMA16, ZC3H12A, FXR1, CDK10, FBRSL1, TMEM185B, NOM1, ARHGEF10, STOM, UXS1, STX11, SNX9, ZFAND2B, PKIG, SCAND1, FILIP1L, UBALD2, PFKM, PRKAA1, USP36, RFTN1, NCKIPSD, PRKD2, CAV2, STX6, SPEN, NADSYN1, ADAM9, ANGEL1, IFRD1, NDST1, RAP2B, RNF166, FBXO42, R3HCC1L, MON1B, ALKBH5, DMAC2, EIF4ENIF1, SLC39A7, SAP30BP, MARVELD1, CCDC61, IBTK, EXOC3, PBXIP1, GSTM4, SERP1, TMEM255B, PNPT1, PIP5K1C, AKT1, PPP1R13B, OAZ2, MYO1C, CHP1, NPIPB6, FAM193A, MFNG, AKR7A2, CLSTN3, DMPK, PEX19, NPDC1, ARID5A, TRAPPC6B, SECISBP2, PPM1D, VASN, MARK2, ARHGAP17, DNAJB2, IL6R, RSRC2, RAPGEF6, CYBRD1, SLC35C2, PPP6R3, MICAL2, FLOT1, ARRB1, GTF2F1, TIMM10B, SNX12, SRP14, BTG3, PSPC1, PPP2R5A, CASP8AP2, AP2A1, ANAPC2, ACTR1B, SLC7A6, SVIL, MBNL2, MTCH2, PARVG, PTAR1, TMEM134, MTERF4, RABGGTA, GTF2H4, or a byproduct, a degradation product, or a precursor thereof, and any combination thereof.

65. A kit comprising:
(a) a substrate comprising a plurality of capture probes, wherein a capture probe of the plurality of capture probes comprises a spatial barcode and a capture domain;
(b) a plurality of first coronavirus probes and second coronavirus probes, wherein a first coronavirus probe and a second coronavirus probe each comprise a sequence that is substantially complementary to sequences of a coronavirus nucleic acid; and wherein one of the first coronavirus probe or the second coronavirus probe comprises a coronavirus capture probe capture domain that is complementary to the capture domain of the capture probe on the substrate; and
(c) instructions for performing the method of claim 47.

66. A composition comprising:
(a) a spatial array comprising a plurality of capture probes, wherein a capture probe of the plurality of capture probes comprises a spatial barcode and a capture domain;
(b) a plurality of first host probes and second host probes, wherein a first host probe and a host second probe each comprise a sequence that is substantially complementary to sequences of a host nucleic acid; and wherein one of the first host probe or the second host probe comprises a host capture probe capture domain that is complementary to the capture domain on the capture probe; and
(c) a plurality of first coronavirus probes and second coronavirus probes, wherein a first coronavirus probe and a second coronavirus probe each comprise a sequence that is substantially complementary to sequences of a coronavirus nucleic acid; and wherein one of the first coronavirus probe or the second coronavirus probe comprises a coronavirus capture probe capture domain that is complementary to the capture domain of the capture probe.

67. The composition of claim 66, further comprising a biological sample on the spatial array wherein the biological sample comprises the host nucleic acid.
